SEQUENCE LISTING (2 sequences)

SEQ ID NO: 1 - protein, 3125 aa, Megathura crenulata
MTPEELKTYLDERSSRARAFASFRLKGFGGSANVFVYVCIPDDNDRNDDHCEKAGDFFVLGGPSEMKWQFYRPYLFDLSDTVHKMGMKLDGHYTVKAELFSVNGTALPDDLLPHPVVVHHPEKGFTDPPVKHHQSANLLVRKNINDLTREEVLNLREAFHKFQEDRSVDGYQATAEYHGLPARCPRPDAKDRYACCVHGMPIFPHWHRLFVTQVEDALVGRGATIGIPYWGLPYWDWTEPMTHIPGLAGNKTYVDSHGASHTNPFHSSVIAFEENAPHTKRQIDQRLFKPATFGHHTDLFNQILYAFEQEDYCDFEVQFEITHNTIHAWTGGSEHFSMSSLHYTAFDPLFYFHHSNVDRLWAVWQALQMRRHKPYRAHCAISLEHMHLKPFAFSSPLNNNEKTHANAMPNKIYDYENVLHYTYEDLTFGGISLENIEKMIHENQQEDRIYAGFLLAGIRTSANVDIFIKTTDSVQHKAGTFAVLGGSKEMKWGFDRVFKFDITHVLKDLDLTADGDFEVTVDITEVDGTKLASSLIPHASVIREHARGKLNRVKFDKVPRSRLIRKNVDRLSPEEMNELRKALALLKEDKSAGGFQQLGAFHGEPKWCPSPEASKKFACCVHGMSVFPHWHRLLTVQSENALRRHGYDGALPYWDWTSPLNHLPELADHEKYVDPEDGVEKHNPWFDGHIDTVDKTTTRSVQNKLFEQPEFGHYTSIAKQVLLALEQDNFCDFEIQYEIAHNYIHALVGGAQPYGMASLRYTAFDPLFYLHHSNTDRIWAIWQALQKYRGKPYNVANCAVTSMREPLQPFGLSANINTDHVTKEHSVPFNVFDYKTNFNYEYDTLEFNGLSISQLNKKLEAIKSQDRFFAGFLLSGFKKSSLVKFNICTDSSNCHPAGEFYLLGDENEMPWAYDRVFKYDITEKLHDLKLHAEDHFYIDYEVFDLKPASLGKDLFKQPSVIHEPRIGHHEGEVYQAEVTSANRIRKNIENLSLGELESLRAAFLEIENDGTYESIAKFHGSPGLCQLNGNPISCCVHGMPTFPHWHRLYVVVVENALLKKGSSVAVPYWDWTKRIEHLPHLISDATYYNSRQHHYETNPFHHGKITHENEITTRDPKDSLFHSDYFYEQVLYALEQDNFCDFEIQLEILHNALHSLLGGKGKYSMSNLDYAAFDPVFFLHHATTDRIWAIWQDLQRFRKRPYREANCAIQLMHTPLQPFDKSDNNDEATKTHATPHDGFEYQNSFGYAYDNLELNHYSIPQLDHMLQERKRHDRVFAGFLLHNIGTSADGHVFVCLPTGEHTKDCSHEAGMFSILGGQTEMSFVFDRLYKLDITKALKKNGVHLQGDFDLEIEITAVNGSHLDSHVIHSPTILFEAGTDSAHTDDGHTEPVMIRKDITQLDKRQQLSLVKALESMKADHSSDGFQAIASFHALPPLCPSPAASKRFACCVHGMATFPQWHRLYTVQFQDSLRKHGAVVGLPYWDWTLPRSELPELLTVSTIHDPETGRDIPNPFIGSKIEFEGENVHTKRDINRDRLFQGSTKTHHNWFIEQALLALEQTNYCDFEVQFEIMHNGVHTWVGGKEPYGIGHLHYASYDPLFYIHHSQTDRIWAIWQSLQRFRGLSGSEANCAVNLMKTPLKPFSFGAPYNLNDHTHDFSKPEDTFDYQKFGYIYDTLEFAGWSIRGIDHIVRNRQEHSRVFAGFLLEGFGTSATVDFQVCRTAGDCEDAGYFTVLGGEKEMPWAFDRLYKYDITETLDKMNLRHDEIFQIEVTITSYDGTVLDSGLIPTPSIIYDPAHHDISSHHLSLNKVRHDLSTLSERDIGSLKYALSSLQADTSADGFAAIASFHGLPAKCNDSHNNEVACCIHGMPTFPHWHRLYTLQFEQALRRHGSSVAVPYWDWTKPIHNIPHLFTDKEYYDVWRNKVMPNPFARGYVPSHDTYTVRDVQEGLFHLTSTGEHSALLNQALLALEQHDYCDFAVQFEVMHNTIHYLVGGPQVYSLSSLHYASYDPIFFIHHSFVDKVWAVWQALQEKRGLPSDRADCAVSLMTQNMRPFHYEINHNQFTKKHAVPNDVFKYELLGYRYDNLEIGGMNLHEIEKEIKDKQHHVRVFAGFLLHGIRTSADVQFQICKTSEDCHHGGQIFVLGGTKEMAWAYNRLFKYDITHALHDAHITPEDVFHPSEPFFIKVSVTAVNGTVLPASILHAPTIIYEPGLGDHHEDHHSSSMAGHGVRKEINTLTTAEVDNLKDAMRAVMADHGPNGYQAIAAFHGNPPMCPMPDGKNYSCCTHGMATFPHWHRLYTKQMEDALTAHGARVGLPYWDGTTAFTALPTFVTDEEDNPFHHGHIDYLGVDTTRSPRDKLFNDPERGSESFFYRQVLLALEQTDFCQFEVQFEITHNAIHSWTGGLTPYGMSTLEYTTYDPLFWLHHANTDRIWAIWQALQEYRGLPYDHANCEIQAMKRPLRPFSDPINHNAFTHSNAKPTDVFEYSRFNFQYDNLRFHGMTIKKLEHELEKQKEEDRTFAAFLLHGIKKSADVSFDVCNHDGECHFAGTFAILGGEHEMPWSFDRLFRYDITQVLKQMHLEYDSDFTFHMRIIDTSGKQLPSDLIKMPTVEHSPGGKHHEKHHEDHHEDILVRKNIHSLSHHEAEELRDALYKLQNDESHGGYEHIAGFHGYPNLCPEKGDEKYPCCVHGMSIFPHWHRLHTIQFERALKKHGSHLGIPYWDWTQTISSLPTFFADSGNNNPFFKYHIRSINQDTVRDVNEAIFQQTKFGEFSSIFYLALQALEEDNYCDFEVQYEILHNEVHALIGGAEKYSMSTLEYSAFDPYFMIHHASLDKIWIIWQELQKRRVKPAHAGSCAGDIMHVPLHPFNYESVNNDDFTRENSLPNAVVDSHRFNYKYDNLNLHGHNIEELEEVLRSLRLKSRVFAGFVLSGIRTTAVVKVYIKSGTDSDDEYAGSFVILGGAKEMPWAYERLYRFDITETVHNLNLTDDHVKFRFDLKKYDHTELDASVLPAPIIVRRPNNAVFDIIEIPIGKDVNLPPKVVVKRGTKIMFMSVDEAVTTPMLNLGSYTAMFKCKVPPFSFHAFELGKMYSVESGDYFMTASTTELCNDNNLRIHVHVDDE

SEQ ID NO: 2 - protein, 3421 aa, Megathura crenulata
MWTILALLTATLLFEGAFSVDTVVRKNVDSLSSDEVLALEKALDDLQQDDSNQGYQAIAGYHGVPTMCVDKHEKNVACCLHGMPSFPLWHRLYVVQLERALIRKKATISIPYWDWTSELTHLPELVSHPLFVGTEGGKAHDNSWYRADITFLNKKTSRAVDDRLFEKVQPGHHTRLMEGILDALEQDEFCKFEIQFELAHNAIHYLVGGRHTYSMSHLEYTSYDPLFFLHHSNTDRIFAIWQRLQQLRGKDPNSADCAHNLIHTPMEPFDRDTNPLDLTREHAKPADSFDYGRLGYQYDDLSLNGMSPEELNVYLGERAAKERTFASFILSGFGGSANVVVYVCRPAHDEISDDQCIKAGDFFLLGGPTEMKWGFYRAYHFDVTDSVASIDDDGHGHYYVKSELFSVNGSALSNDILRQPTLVHRPAKGHFDKPPVPVAQANLAVRKNINDLTAEETYSLRKAMERFQNDKSVDGYQATVEFHALPARCPRPDAKDRFACCVHGMATFPHWHRLFVTQVEDALLRRGSTIGLPNWDWTMPMDHLPELATSETYLDPVTGETKNNPFHHAQVAFENGVTSRNPDAKLFMKPTYGDHTYLFDSMIYAFEQEDFCDFEVQYELTHNAIHAWVGGSEKYSMSSLHYTAFDPIFYLHHSNVDRLWAIWQALQIRRGKSYKAHCASSQEREPLKPFAFSSPLNNNEKTYHNSVPTNVYDYVGVLHYRYDDLQFGGMTMSELEEYIHKQTQHDRTFAGFFLSYIGTSASVDIFINREGHDKYKVGSFVVLGGSKEMKWGFDRMYKYEITEALKTLNVAVDDGFSITVEITDVDGSPPSADLIPPPAIIFERADAKDFGHSRKIRKAVDSLTVEEQTSLRRAMADLQDDKTSGGFQQIAAFHGEPKWCPSPEAEKKFACCVHGMAVFPHWHRLLTVQGENALRKHGFTGGLPYWDWTRSMSALPHFVADPTYNDAISSQEEDNPWHHGHIDSVGHDTTRDVRDDLYQSPGFGHYTDIAKQVLLAFEQDDFCDFEVQFEIAHNFIHALVGGNEPYSMSSLRYTTYDPIFFLHRSNTDRLWAIWQALQKYRGKPYNTANCAIASMRKPLQPFGLDSVINPDDETREHSVPFRVFDYKNNFDYEYESLAFNGLSIAQLDRELQRRKSHDRVFAGFLLHEIGQSALVKFYVCKHNVSDCDHYAGEFYILGDEAEMPWRYDRVYKYEITQQLHDLDLHVGDNFFLKYEAFDLNGGSLGGSIFSQPSVIFEPAAGSHQADEYREAVTSASHIRKNIRDLSEGEIESIRSAFLQIQKEGIYENIAKFHGKPGLCEHDGHPVACCVHGMPTFPHWHRLYVLQVENALLERGSAVAVPYWDWTEKADSLPSLINDATYFNSRSQTFDPNPFFRGHIAFENAVTSRDPQPELWDNKDFYENVMLALEQDNFCDFEIQLELIHNALHSRLGGRAKYSLSSLDYTAFDPVFFLHHANVDRIWAIWQDLQRYRKKPYNEADCAVNEMRKPLQPFNNPELNSDSMTLKHNLPQDSFDYQNRFRYQYDNLQFNHFSIQKLDQTIQARKQHDRVFAGFILHNIGTSAVVDIYICVEQGGEQNCKTKAGSFTILGGETEMPFHFDRLYKFDITSALHKLGVPLDGHGFDIKVDVRAVNGSHLDQHILNEPSLLFVPGERKNIYYDGLSQHNLVRKEVSSLTTLEKHFLRKALKNMQADDSPDGYQAIASFHALPPLCPSPSAAHRHACCLHGMATFPQWHRLYTVQFEDSLKRHGSIVGLPYWDWLKPQSALPDLVTQETYEHLFSHKTFPNPFLKANIEFEGEGVTTERDVDAEHLFAKGNLVYNNWFCNQALYALEQENYCDFEIQFEILHNGIHSWVGGSKTHSIGHLHYASYDPLFYIHHSQTDRIWAIWQALQEHRGLSGKEAHCALEQMKDPLKPFSFGSPYNLNKRTQEFSKPEDTFDYHRFGYEYDSLEFVGMSVSSLHNYIKQQQEADRVFAGFLLKGFGQSASVSFDICRPDQSCQEAGYFSVLGGSSEMPWQFDRLYKYDITKTLKDMKLRYDDTFTIKVHIKDIAGAELDSDLIPTPSVLLEEGKHGINVRHVGRNRIRMELSELTERDLASLKSAMRSLQADDGVNGYQAIASFHGLPASCHDDEGHEIACCIHGMPVFPHWHRLYTLQMDMALLSHGSAVAIPYWDWTKPISKLPDLFTSPEYYDPWRDAVVNNPFAKGYIKSEDAYTVRDPQDILYHLQDETGTSVLLDQTLLALEQTDFCDFEVQFEVVHNAIHYLVGGRQVYALSSQHYASYDPAFFIHHSFVDKIWAVWQALQKKRKRPYHKADCALNMMTKPMRPFAHDFNHNGFTKMHAVPNTLFDFQDLFYTYDNLEIAGMNVNQLEAEINRRKSQTRVFAGFLLHGIGRSADVRFWICKTADDCHASGMIFILGGSKEMHWAYDRNFKYDITQALKAQSIHPEDVFDTDAPFFIKVEVHGVNKTALPSSAIPAPTIIYSAGEGHTDDHGSDHIAGSGVRKDVTSLTASEIENLRHALQSVMDDDGPNGFQAIAAYHGSPPMCHMHDGRDVACCTHGMASFPHWHRLFVKQMEDALAAHGAHIGIPYWDWTSAFSHLPALVTDHEHNPFHHGHIAHRNVDTSRSPRDMLFNDPEHGSESFFYRQVLLALEQTDFCQFEVQFEITHNAIHSWTGGHTPYGMSSLEYTAYDPLFYLHHSNTDRIWAIWQALQKYRGFQYNAAHCDIQVLKQPLKPFSESRNPNPVTRANSRAVDSFDYERLNYQYDTLTFHGHSISELDAMLQERKKEERTFAAFLLHGFGASADVSFDVCTPDGHCAFAGTFAVLGGELEMPWSFERLFRYDITKVLKQMNLHYDSEFHFELKIVGTDGTELPSDRIKSPTIEHHGGDHHGGDTSGHDHSERHDGFFRKEVGSLSLDEANDLKNALYKLQNDQGPNGYESIAGYHGYPFLCPEHGEDQYACCVHGMPVFPHWHRLHTIQFERALKEHGSHLGIPYWDWTKSMIALPAFFADSSNSNPFYKYHIMKAGHDTARSPSDLLFNQPQLHGYDYLYYLALSTLEEDNYCDFEVHYEILHNAVHLWLGGTETYSMSSLAFSAYDPVFMILHSGLDRLWIIWQELQKLRKKPYNAAKCAGHMMDEPLHPFNYESANHDSFTRANAKPSTVFDSHKFNYHYDNPDVRGNSIQEISAIIHDLRNQPRVFAGFVLSGIYTSANVKIYLVREGHDDENVGSFVVLGGPKEMPWAYERIFKYDITEVANRLNMHHDDTFNFRLEVQSYTGEMVTHHLPEPLIIYRPAKQEYDVLVIPLGSGHKLPPKVIVKRGTRIMFHPVDDTVNRPVVDLGSHTALYNCVVPPFTYNGYELDHAYSLRDGHYYIAGPTKDLCTSGNVRIHIHIEDE

(19) United States
(12) Patent Application Publication
(10) Pub. No.: US 2016/0339089 A1
(43) Pub. Date: Nov. 24, 2016

(54) IMMUNOGENIC/THERAPEUTIC GLYCOCONJUGATE COMPOSITIONS AND USES THEREOF

(71) Applicant: OBI PHARMA, INC., TAIPEI CITY (TW)

(72) Inventors: Cheng-Der Tony YU, Taipei City (TW); Yih Huang HSIEH, Taipei City (TW); I-Ju CHEN, Taipei City (TW); Wei-Han LEE, Taipei City (TW); Nan-Hsuan WANG, Taipei City (TW)

(21) Appl. No.: 14/855,260

(22) Filed: Sep. 15, 2015

Related U.S. Application Data
(60) Provisional application No. 62/050,567, filed on Sep. 15, 2014.

(51) Int. Cl. A61K 39/00; C07K 16/18; A61K 39/385

(52) U.S. Cl. CPC A61K 39/0011; A61K 39/385; C07K 16/18; A61K 2039/6081; A61K 2039/627; C07K 2317/10; C07K 2317/21; A61K 2039/575; A61K 2039/645; A61K 2039/55577

(57) ABSTRACT

The present disclosure encompasses immunogenic/therapeutic compositions including Globo H-KLH glycoconjugates (OBI-822) and/or therapeutic adjuvants (OBI-821/OBI-834) as well as methods of making and using the same to treat proliferative diseases such as cancer. The therapeutic conjugates include an antigen linked to a carrier. In particular the therapeutic conjugates include a Globo H moiety and a KLH moiety and/or a derivatized KLH moiety subunit optionally linked via a linker. The therapeutic compositions are in part envisaged to act as cancer vaccines for boosting the body's natural ability to protect itself, through the immune system from dangers posed by damaged or abnormal cells such as cancer cells. Exemplary immune response can be characterized by reduction of the severity of disease, including but not limited to, prevention of disease, delay in onset of disease, decreased severity of symptoms, decreased morbidity and delayed mortality.

### RELATED APPLICATIONS

This application claims the benefit of U.S. Provisional Application No. 62/050,567, filed on Sep. 15, 2014, the entire disclosure of which is incorporated herein by reference.

### FIELD OF THE INVENTION

The present disclosure is directed to compositions and methods for cancer immunotherapy and immunogenic/therapeutic glycoconjugates able to elicit anti-cancer immune responses in particular.

### BACKGROUND

The carbohydrate antigen Globo H (Fuc α 1→2 Gal β 1→3 GalNAc β 1→3 Gal α 1→4 Gal β 1→4 Glc) was first isolated as a ceramide-linked Glycolipid and identified in 1984 by Hakomori et al. from breast cancer MCF-7 cells. (Bremer E G, et al. (1984) J Biol Chem 259:14773-14777). Further studies with anti-Globo H monoclonal antibodies showed that Globo H was present on many other cancers, including prostate, gastric, pancreatic, lung, ovarian and colon cancers and only minimal expression on luminal surface of normal secretory tissue which is not readily accessible to immune system. (Ragupathi G, et al. (1997) Angew Chem Int Ed 36:125-128). In addition, it has been established that the serum of breast cancer patient contains high level of anti-Globo H antibody. (Gilewski T et al. (2001) Proc Natl Acad Sci USA 98:3270-3275; Huang C-Y, et al. (2006) Proc Natl Acad Sci USA 103:15-20; Wang C-C, et al. (2008) Proc Natl Acad Sci USA 105(33):11661-11666). Patients with Globo H-positive tumors showed a shorter survival in comparison to patients with Globo H-negative tumors. (Chang, Y-J, et al. (2007) Proc Natl Acad Sci USA 104(25):10299-10304). These findings render Globo H, a hexasaccharide epitope, an attractive tumor marker and a feasible target for cancer vaccine development.

### SUMMARY OF THE INVENTION

While vaccines have been developed to elicit antibody responses against Globo H, their anti-cancer efficacies are unsatisfactory due to low antigenicity of Globo H. There is a need for a new vaccine capable of eliciting high levels of immune responses targeting Globo H.

KLH contains glycosylated polypeptide subunits that can assemble to form decameric (10-mer), didecameric (20-mer), and larger particles. These multimeric structures have been characterized by ultracentrifugation techniques that yield sedimentation coefficients of 11-19S for the dissociated subunits and 92-107S for the didecameric multimers. A variety of factors may affect the size distribution of molluscam hemocyanins, including KLH. These factors include ionic strength, pH, temperature, pO₂, and the availability of certain divalent cations, notably calcium and magnesium.

The current inventors have developed a composition with surprising increased efficacy that is primarily comprised of dimers, trimers as well as other multimers of KLH linked to a plurality of Globo H moieties.

Accordingly, the present disclosure generally encompasses therapeutic and/or prophylactic compositions including Globo H, as well as, immunotherapeutics, vaccines, dosage forms, kits, and methods of manufacture, and treatment thereof.

In one embodiment, the invention encompasses an isolated therapeutic conjugate comprising a Globo H moiety linked to a keyhole limpet hemocyanin (KLH) moiety subunit. In certain embodiments, the linkage is a covalent bond.

In another embodiment, the invention encompasses an isolated therapeutic conjugate comprising a Globo H moiety covalently linked to a keyhole limpet hemocyanin (KLH) moiety subunit, wherein the KLH is a derivatized KLH. As used herein the term “covalently linked” when referring to Globo-H and KLH means: Globo-His directly covalently linked to KLH, or Globo-H is covalently linked to derivatized KLH (as set forth herein), or Globo-H is covalently linked to KLH through a linker group (as set forth herein), or Globo-H is covalently linked to KLH through both a linker group and a derivatized KLH.

In certain illustrative embodiments, the derivatized KLH of the invention has the following structure:

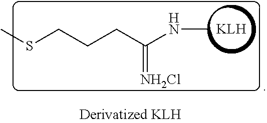

In one embodiment, the invention encompasses an isolated therapeutic conjugate comprising a Globo H moiety covalently linked to a keyhole limpet hemocyanin (KLH) moiety subunit through a linker molecule.

In one embodiment, the Globo H moieties are bound to a lysine residue of a KLH moiety subunit.

In one embodiment, there are total of exactly or about 145, 146, 147, 148, 149, 150, 151, 152, 153, 154, 155, 156, 157, 158, 159, 160 total lysine residues per KLH moiety subunit which are available for or actually directly or indirectly bind a Globo H moiety.

In another embodiment, the invention encompasses an isolated therapeutic conjugate comprising a Globo H moiety covalently linked to a keyhole limpet hemocyanin (KLH) moiety subunit via a 4-(4-N-maleimidomethyl)cyclohexane-1-carboxyl hydrazide (MMCCH) linker group. The MMCCH linker of the invention has the following structure:

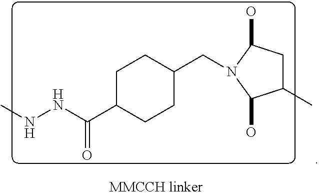

In another illustrative embodiment, the invention encompasses an isolated therapeutic conjugate having the following general structure:

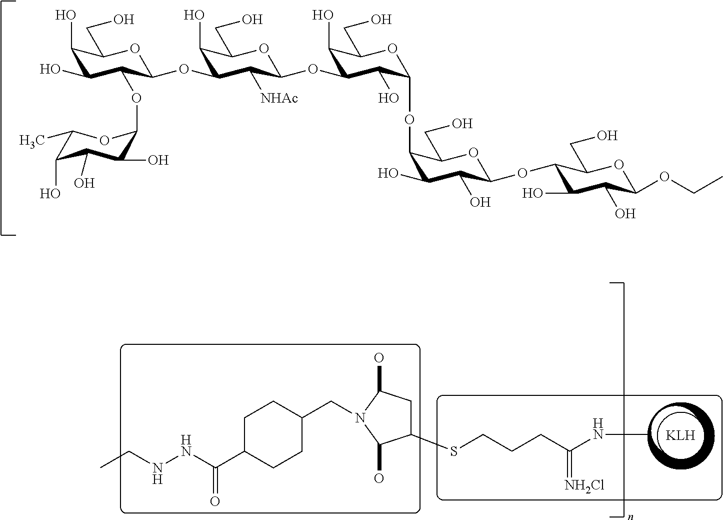

wherein n is an integer from about 1 to about 160. In certain embodiments, a monomeric KLH moiety can include from about 1 to about 160 Globo H moieties. One of ordinary skill in the art will recognize that the structures are illustrated as the iminium hydrochloride salts but can also exist or co-exist as the imine form. Accordingly, the invention encompasses both the imine as well as salts thereof including, for example, the iminium hydrochloride salt. In certain embodiments, a monomeric KLH moiety can include from about 1 to about 125 Globo H moieties. In certain embodiments, a monomeric KLH moiety can include about 1 to about 100 Globo H moieties. In certain embodiments, a monomeric KLH moiety can include from 1 to about 75 Globo H moieties. In certain embodiments, a monomeric KLH moiety can include from about 1 to about 50 Globo H moieties. In certain embodiments, a monomeric KLH moiety can include from about 1 to about 25 Globo H moieties. In certain embodiments, a monomeric KLH moiety can include from about 1 to about 10 Globo H moieties.

In certain embodiments, the Globo H moieties are conjugated to the KLH moieties covalently to certain amino acid residues. In certain embodiments, the amino acid residues can include or exclude arginine, lysine, histidine, asparagine, proline, glutamine or a combination thereof.

In another embodiment the Globo H moieties are bound to lysine conjugation sites on a monomeric KLH moiety subunit.

In another embodiment, there are exactly or about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109 or 110 lysine conjugation sites on each monomeric KLH moiety subunit available for binding to or actually bound to a Globo H moiety. In another embodiment, there are 62, 66, 67, 68, 70, 72, 76, 86, 87, 88, 90, 92, 93, 100 such lysine conjugation sites on each KLH moiety subunit.

In certain therapeutic composition embodiments containing a mixture of moiety subunits (e.g., KLH1 and KLH2 or variants thereof), total available lysine (for both subunits) as are counted together across the different subunit types the and may be or are exactly about 290, 291, 292, 293, 294, 295, 296, 297, 298, 299, 300, 301, 302, 303, 304, 305, 306, 307, 308, 309 or 310 in number. In such embodiments, there are exactly or about 130, 131, 132, 133, 134, 135, 136, 137, 138, 139, 140, 141, 142, 143, 144, 145, 146, 147, 148, 149, 150, 151, 152, 153, 154, 155, 156, 157, 158, 159 or 160 lysine conjugation sites together across the different subunits (e.g., KLH1 and KLH2 or variants thereof). In other such embodiments, there are 136, 137, 141, 140, 143, 147 or 155 lysine conjugation sites.

In another illustrative embodiment, the invention encompasses an isolated immunogenic/therapeutic conjugate having the following general structure:

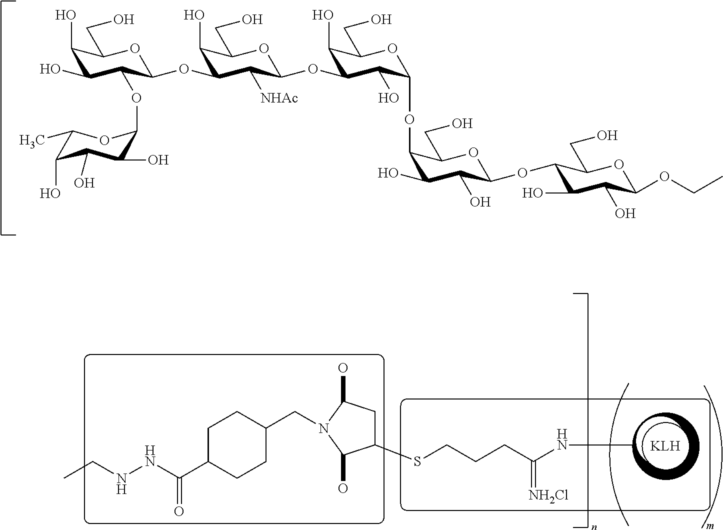

wherein n is independently an integer from about 1 to about 3000 and m is independently an integer from about 1 to about 20. In certain embodiments, when m is greater than 1, KLH moieties can aggregate to form multimeric structures. In certain embodiments, the aggregation is a covalent bond. In certain other embodiments, the aggregation is not a covalent bond (e.g., the aggregation is formed by H-bonding or hydrophobic interactions). In certain embodiments, a monomeric KLH moiety (i.e., where m=1) can include from about 1 to about 160 Globo H moieties. In certain embodiments, a dimeric KLH moiety (i.e., where m=2) can include from about 1 to about 300 Globo H moieties. In certain embodiments, a trimeric KLH moiety (i.e., where m=3) can include from about 1 to about 450 Globo H moieties. In certain embodiments, a tetrameric KLH moiety (i.e., where m=4) can include from about 1 to about 600 Globo H moieties. In certain embodiments, a pentameric KLH moiety (i.e., where m=5) can include from about 1 to about 750 Globo H moieties. In certain embodiments, a hexameric KLH moiety (i.e., where m=6) can include from about 1 to about 900 Globo H moieties. In certain embodiments, a didecameric KLH moiety (i.e., where m=20) can include from about 1 to about 3000 Globo H moieties.

In another illustrative embodiment, the invention encompasses an isolated immunogenic/therapeutic conjugate having the following general structure:

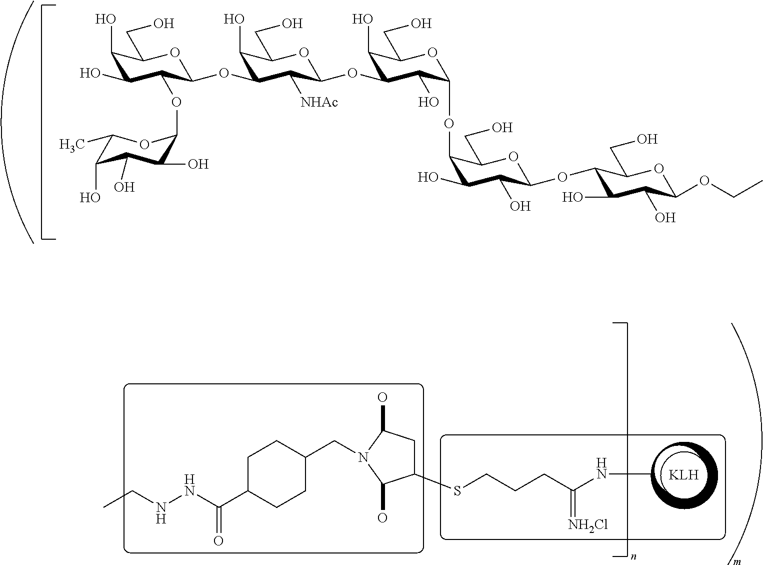

wherein n is independently an integer from about 1 to about 150 and m is independently an integer from about 1 to about 20.

In another embodiments, the invention encompasses an isolated therapeutic conjugate having the following general structure:

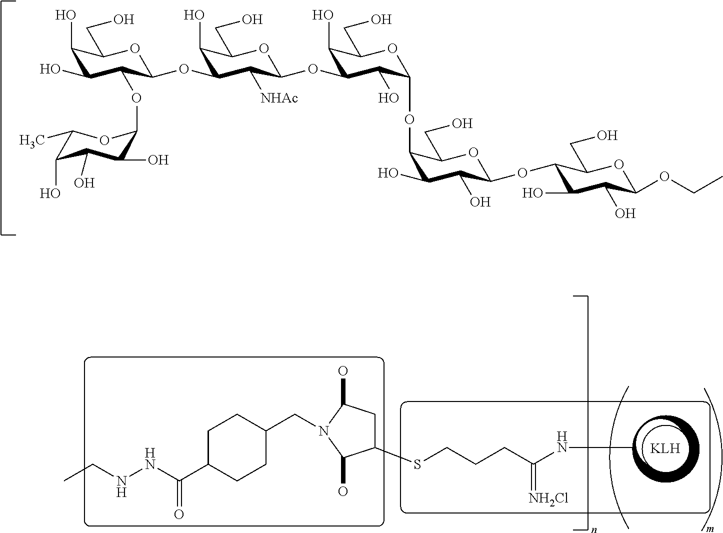

wherein n independently is an integer from about 1 to about 160, and wherein m is independently an integer from about 1 to about 20. In certain embodiments, m is an integer from about 1 to about 5. In certain embodiments, m is an integer from about 1 to about 3. In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, m is 11. In certain embodiments, m is 12. In certain embodiments, m is 13. In certain embodiments, m is 14. In certain embodiments, m is 15. In certain embodiments, m is 16. In certain embodiments, m is 17. In certain embodiments, m is 18. In certain embodiments, m is 19. In certain embodiments, m is 20. In certain embodiments, for any of the above embodiments, when m is 1 to 20, each n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, or 160, respectively.

In certain embodiments, there is more than one Globo H moiety attached to each monomeric KLH moiety. In certain illustrative embodiments, the more than one Globo H moiety attached to each KLH moiety is attached via a linker. In other illustrative embodiments, the more than one Globo H moieties attached to each KLH moiety are attached via a linker and attached to a derivatized KLH moiety.

In another embodiment, the ratio of Globo H moieties to KLH moiety subunits is at least 1. In another embodiment, the ratio of Globo H moieties to KLH moiety subunits is at least 10. In another embodiment, the ratio of Globo H moieties to KLH moiety is at least 25. In another embodiment, the ratio of Globo H moieties to KLH moiety subunits is at least 50. In a further embodiment, the ratio of Globo H moieties to KLH moiety subunits is at least 100. In a further embodiment, the ratio of Globo H moieties to KLH moiety subunits is at least 150. In yet another embodiment, the ratio of Globo H moieties to KLH moiety subunits is at least 500. In yet a further embodiment, the ratio of Globo H moieties to KLH moiety subunits is at least 750. In still another embodiment, the ratio of Globo H moieties to KLH moiety subunits is at least 1000. In still another embodiment, the ratio of Globo H moieties to KLH moiety subunits is at least 1500. In still another embodiment, the ratio of Globo H moieties to KLH moiety subunits is at least 2000.

In various embodiments, the invention encompasses a single monomer of KLH to multiple KLH subunits (e.g., 2, 3, 4, 5, 6, 7, 8, 9, 10, 11, 12, 13, 14, 15, 16, 17, 18, 19, 20) each having attached multiple Globo H moieties. In certain embodiments, the ratio of Globo H moieties to KLH moiety is the same. In other embodiments, the ratio of Globo H moieties to KLH moiety is different.

Another embodiment of the invention encompasses a composition comprising at least two KLH moieties. For example, a derivatized KLH moiety in the form of a dimer. In another embodiment, the at least two KLH moieties are the same. In another embodiment, the at least two KLH moieties are different. In a further embodiment, the at least two KLH moieties have the same Globo H moiety to KLH moiety subunit ratio. In still a further embodiment, the at least two KLH moieties have a different Globo H moiety to KLH moiety subunit ratio.

Another embodiment of the invention encompasses a therapeutic composition comprising at least three KLH moieties, for example, a derivatized KLH moiety in the form of a trimer. In certain embodiments, the at least three KLH moieties are the same. In another embodiment, the at least three KLH moieties are not the same. In a further embodiment, the at least three KLH moieties have the same Globo H moiety to KLH moiety subunit ratio. In still a further embodiment, the at least three KLH moieties have a different Globo H moiety to KLH moiety subunit ratio.

Another embodiment of the invention encompasses a therapeutic composition comprising at least four KLH moieties, for example, a derivatized KLH moiety in the form of a tetramer. In certain embodiments, the at least four KLH moieties are the same. In another embodiment, the at least four KLH moieties are not the same. In a further embodiment, the at least four KLH moieties have the same Globo H moiety to KLH moiety subunit ratio. In still a further embodiment, the at least four KLH moieties have a different Globo H moiety to KLH moiety subunit ratio.

Another embodiment of the invention encompasses a therapeutic composition comprising at least five KLH moieties, for example, a derivatized KLH moiety in the form of a pentamer. In certain embodiments, the at least five KLH moieties are the same. In another embodiment, the at least five KLH moieties are not the same. In a further embodiment, the at least five KLH moieties have the same Globo H moiety to KLH moiety subunit ratio. In still a further embodiment, the at least five KLH moieties have a different Globo H moiety to KLH moiety subunit ratio.

Another embodiment of the invention encompasses a therapeutic composition comprising at least six KLH moieties, for example, a derivatized KLH moiety in the form of a hexamer. In certain embodiments, the at least six KLH moieties are the same. In another embodiment, the at least six KLH moieties are not the same. In a further embodiment, the at least six KLH moieties have the same Globo H moiety to KLH moiety subunit ratio. In still a further embodiment, the at least six KLH moieties have a different Globo H moiety to KLH moiety subunit ratio.

Another embodiment of the invention encompasses a therapeutic composition comprising at least twenty KLH moieties, for example, a derivatized KLH moiety in the form of a didecamer. In certain embodiments, the at least twenty KLH moieties are the same. In another embodiment, the at least twenty KLH moieties are not the same. In a further embodiment, the at least twenty KLH moieties have the same Globo H moiety to KLH moiety subunit ratio. In still a further embodiment, the at least twenty KLH moieties have a different Globo H moiety to KLH moiety subunit ratio.

In one embodiment, the Globo H moiety comprises (Fuc α 1→2 Gal β 1→3 GalNAc β1→3 Gal α 1→4 Gal β 1→4 Glc). In a further embodiment, the KLH moiety subunit is a KLH-1 or KLH-2 moiety or a combination thereof. As used herein, the term “KLH” refers to KLH-1, KLH-2, and/or combinations thereof.

In another embodiment, the KLH moiety subunit is at least 99% identical to a corresponding naturally occurring KLH moiety subunit.

In another embodiment, the KLH moiety subunit is at least 95% identical to a corresponding naturally occurring KLH moiety subunit.

In another embodiment, the KLH moiety subunit is at least 90% identical to a corresponding naturally occurring KLH moiety subunit.

In another embodiment, the KLH moiety subunit is at least 80% identical to a corresponding naturally occurring KLH moiety subunit.

In another embodiment, the KLH moiety subunit is at least 70% identical to a corresponding naturally occurring KLH moiety subunit.

In another embodiment, the KLH moiety subunit is at least 60% identical to a corresponding naturally occurring KLH moiety subunit.

In another embodiment, the Globo H moiety is covalently linked to a keyhole limpet hemocyanin (KLH) moiety subunit via a linker. In yet a further embodiment, the Globo H moiety is covalently linked to a keyhole limpet hemocyanin (KLH) moiety subunit by a 4-(4-N-maleimidomethyl) cyclohexane-1-carboxyl hydrazide (MMCCH) linkage. In another further embodiment, the Globo H moiety is covalently linked to a derivatized keyhole limpet hemocyanin (KLH) moiety subunit and is linked by a 4-(4-N-maleimidomethyl) cyclohexane-1-carboxyl hydrazide (MMCCH) linkage.

In another embodiment, the isolated therapeutic conjugate has an epitope ratio based on a KLH monomer having molecular weight of about 350 KDa to about 400 KDa of at least or about 150. In another embodiment, the isolated therapeutic conjugate has an epitope ratio of at least or about 100. In a further embodiment, the isolated therapeutic conjugate has an epitope ratio of at least or about 75. In still a further embodiment, the isolated therapeutic conjugate has an epitope ratio of at least or about 50. In yet a further embodiment, the isolated therapeutic conjugate has an epitope ratio of at least or about 25. In still another embodiment, the isolated therapeutic conjugate has an epitope ratio of at least or about 15. In still another embodiment, the isolated therapeutic conjugate has an epitope ratio of at least or about 5. In still another embodiment, the isolated therapeutic conjugate has an epitope ratio of at least or about 1.

Another embodiment of the invention encompasses a pharmaceutical composition comprising KLH moiety subunits, wherein each KLH moiety subunit comprises one or more Globo H moieties covalently linked to a keyhole limpet hemocyanin (KLH) moiety subunit. In certain embodiments, the pharmaceutical composition comprises dimers of at least two KLH moiety subunits, wherein each KLH moiety subunits comprises one or more Globo H moieties covalently linked to a KLH moiety subunit. In certain embodiments, the pharmaceutical composition comprises trimers of at least three KLH moiety subunits, wherein each KLH moiety subunits comprises one or more Globo H moieties covalently linked to a KLH moiety subunit. In certain embodiments, the pharmaceutical composition comprises at least four KLH moiety subunits, wherein each KLH moiety subunit comprises one or more Globo H moieties covalently linked to a KLH moiety subunit. In certain embodiments, the pharmaceutical composition comprises a mixture of KLH moiety subunits (e.g., monomers, dimers, trimers, tetramers, pentamers, hexamers etc.), wherein each KLH moiety subunits comprises multiple Globo H moieties covalently linked to a KLH moiety subunit.

Another aspect of the invention relates to a pharmaceutical composition comprising monomers, dimers, trimers, tetramers, pentamers, hexamers or combinations thereof of KLH moieties, wherein each KLH comprises one or more Globo H moiety covalently linked to a keyhole limpet hemocyanin (KLH) moiety subunit.

In one embodiment the invention, the epitope ratios of the therapeutic conjugates in the composition ranges from about 1 to 3000. In a further embodiment, the epitope ratios of the therapeutic conjugates in the composition range from about 75 to 2000. In still another embodiment, the epitope ratios of the therapeutic conjugates in the composition range from about 100 to 1000. In yet a further embodiment the average epitope ratio of the therapeutic conjugates in the composition ranges from about 150 to 500.

In another embodiment, about 1% to 99% of the therapeutic conjugates in the composition are KLH monomers. In a further embodiment, about 0% to 99% of the therapeutic conjugates in the composition are KLH dimers. In still another embodiment, about 0% to 99% of the therapeutic conjugates in the composition are KLH trimers. In yet another embodiment, about 0% to 99% of the therapeutic conjugates in the composition are KLH tetramers. In a further embodiment, about 1% to 99% of the therapeutic conjugates in the composition are KLH pentamers. In yet another embodiment, about 0% to 99% of the therapeutic conjugates in the composition include 6 KLH subunits. In yet another embodiment, about 0% to 99% of the therapeutic conjugates in the composition include 7 KLH subunits. In yet another embodiment, about 0% to 99% of the therapeutic conjugates in the composition include 8 KLH subunits. In yet another embodiment, about 0% to 99% of the therapeutic conjugates in the composition include 9 KLH subunits. In yet another embodiment, about 0% to 99% of the therapeutic conjugates in the composition include 10 KLH subunits. In yet another embodiment, about 0% to 99% of the therapeutic conjugates in the composition include 11 KLH subunits. In yet another embodiment, about 0% to 99% of the therapeutic conjugates in the composition include 12 KLH subunits. In yet another embodiment, about 0% to 99% of the therapeutic conjugates in the composition include 13 KLH subunits. In yet another embodiment, about 0% to 99% of the therapeutic conjugates in the composition include 14 KLH subunits. In yet another embodiment, about 0% to 99% of the therapeutic conjugates in the composition include 15 KLH subunits. In yet another embodiment, about 0% to 99% of the therapeutic conjugates in the composition include 16 KLH subunits. In yet another embodiment, about 0% to 99% of the therapeutic conjugates in the composition include 17 KLH subunits. In yet another embodiment, about 0% to 99% of the therapeutic conjugates in the composition include 18 KLH subunits. In yet another embodiment, about 0% to 99% of the therapeutic conjugates in the composition include 19 KLH subunits. In yet another embodiment, about 0% to 99% of the therapeutic conjugates in the composition include 20 KLH subunits. In still another embodiment, about 1% to 99% of the therapeutic conjugates in the composition are monomers, dimers, trimers, tetramers or combinations thereof. In still another embodiment, about 99% of the therapeutic conjugates in the composition are monomers, dimers, trimers, tetramers or combinations thereof.

In certain embodiments, certain exemplary composition embodiments and methods of use thereof can include or exclude (e.g. proviso out) any one or more of the other representative compound and/or composition embodiments described herein.

In another embodiment, the pharmaceutical composition comprises an adjuvant. As used herein, the terms “immunologic adjuvant” refers to a substance used in conjunction with an immunogen which enhances or modifies the immune response to the immunogen. Specifically, the terms “adjuvant” and “immunoadjuvant” are used interchangeably in the present invention and refer to a compound or mixture that may be non-immunogenic when administered to a host alone, but that augments the host's immune response to another antigen when administered conjointly with that antigen. Adjuvant-mediated enhancement and/or extension of the duration of the immune response can be assessed by any method known in the art including without limitation one or more of the following: (i) an increase in the number of antibodies produced in response to immunization with the adjuvant/antigen combination versus those produced in response to immunization with the antigen alone; (ii) an increase in the number of T cells recognizing the antigen or the adjuvant; and (iii) an increase in the level of one or more Type I cytokines.

The adjuvant of can be administered as part of a pharmaceutical or vaccine composition comprising an antigen or as a separate formulation, which is administered conjointly with a second composition containing an antigen. In any of these compositions glycosphingolipids (GSLs) can be combined with other adjuvants and/or excipients/carriers. These other adjuvants include, but are not limited to, oil-emulsion and emulsifier-based adjuvants such as complete Freund's adjuvant, incomplete Freund's adjuvant, MF59, or SAF; mineral gels such as aluminum hydroxide (alum), aluminum phosphate or calcium phosphate; microbially-derived adjuvants such as cholera toxin (CT), pertussis toxin, Escherichia coli heat-labile toxin (LT), mutant toxins (e.g., LTK63 or LTR72), Bacille Calmette-Guerin (BCG), Corynebacterium parvum, DNA CpG motifs, muramyl dipeptide, or monophosphoryl lipid A; particulate adjuvants such as immunostimulatory complexes (ISCOMs), liposomes, biodegradable microspheres, or saponins (e.g., QS-21); cytokines such as IFN-γ, IL-2, IL-12 or GM-CSF; synthetic adjuvants such as nonionic block copolymers, muramyl peptide analogues (e.g., N-acetyl-muramyl-L-threonyl-D-isoglutamine [thr-MDP], N-acetyl-nor-muramyl-L-alanyl-D-isoglutamine, N-acetylmuramyl-L-alanyl-D-isoglutaminyl-L-alanine-2-[1′-2′-dipalmitoyl-sn-glycero-3-hydroxyphosphoryloxy]-ethylamine), polyphosphazenes, or synthetic polynucleotides, and surface active substances such as lysolecithin, pluronic polyols, polyanions, peptides, hydrocarbon emulsions, or keyhole limpet hemocyanins (KLH), Toll-Like Receptor molecules, LPS, lipoproteins, lipopeptides, flagellin, double-stranded RNA, viral DNA, unmethylated CpG islands, levamisole, bacillus Calmette-Guerin, Isoprinosine, Zadaxin, PD-1 antagonists, PD-1 antibodies, CTLA antagonists, CTLA antibodies, interleukin, cytokines, GM-CSF, glycolipid, aluminum salt based, aluminum phosphate, alum, aluminum hydroxide, liposomes, TLR2 agonists, lipopeptide, nanoparticles, monophosphoryl lipid A, OBI-821 saponin, saponin, OBI-834 adjuvant, C34 adjuvant, oil in water nano-emulsions, and bacteria-like particle. Preferably, these additional adjuvants are also pharmaceutically acceptable for use in humans.

In another embodiment, the pharmaceutical composition comprises a cytokine selected from the group consisting of IL-2, IL-12, IL-18, IL-2, IFN-γ, TNF, IL-4, IL-10, IL-13, IL-21, GM-CSF and TGF-β. In a further embodiment, the pharmaceutical composition comprises a chemokine.

In a further embodiment, the immunogenic/therapeutic agent is administered as a pharmaceutical composition.

In still another embodiment, the pharmaceutical composition comprises monoclonal antibodies, chemotherapeutics, hormonal therapeutic agents, retinoid receptor modulators, cytotoxic/cytostatic agents, antineoplastic agents, antiproliferative agents, anti-mTOR agents, anti-Her2 agents, anti-EGFR agents, prenyl-protein transferase inhibitors, HMG-CoA reductase inhibitors, nitrogen mustards, nitroso ureas, angiogenesis inhibitors, bevacizumab, inhibitors of cell proliferation and survival signaling pathway, apoptosis inducing agents, agents that interfere with cell cycle checkpoints, agents that interfere with receptor tyrosine kinases (RTKs), integrin blockers, NSAIDs, PPAR agonists, inhibitors of inherent multidrug resistance (MDR), anti-emetic agents, agents useful in the treatment of anemia, agents useful in the treatment of neutropenia, immunologic-enhancing drugs, biphosphonates, aromatase inhibitors, agents inducing terminal differentiation of neoplastic cells, γ-secretase inhibitors, cancer vaccines (e.g., active immunotherapy), monoclonal antibody therapeutics (e.g., passive immunotherapy), and any combination thereof.

In another embodiment, the therapeutic compositions of the invention can further include PD-1/PD-L1 inhibitors (cytotoxic T cell lymphocyte (CTLs) immunotherapy), CTLA-4 immunotherapy, CDK4/6 inhibitors (target therapy), PI3K inhibitors (target therapy), mTOR inhibitors (target therapy), AKT inhibitors (target therapy), Pan-Her inhibitors (target therapy). These inhibitors can be modified to generate the respective monoclonal antibody as well. Such antibodies can be included in therapeutic compositions of the invention.

In another embodiment, the pharmaceutical composition comprises a pharmaceutically acceptable carrier. In a further embodiment, the pharmaceutical composition is a cancer vaccine. In still another embodiment, the pharmaceutical composition is formulated for subcutaneous administration. In still another embodiment, the pharmaceutical composition is formulated for intramuscular administration. In still another embodiment, the pharmaceutical composition is formulated for intra-arterial administration. In still another embodiment, the pharmaceutical composition is formulated for intravenous administration.

Another embodiment of the invention encompasses a method of treating a patient in need thereof comprising administering to the patient a therapeutically effective amount of the therapeutic composition comprising Globo H and KLH. In one embodiment, the patient has been diagnosed with or is suspected of having cancer. In another embodiment, the cancer is an epithelial cancer. In a further embodiment, the cancer is breast cancer. In still another embodiment, the therapeutically effective amount of a Globo-H moiety in the pharmaceutical/therapeutic composition may range from about 0.001 μg/kg to about 250 mg/kg. In yet a further embodiment, the therapeutically effective amount Globo-H moiety in the pharmaceutical/therapeutic composition comprises about 10 μg/kg to about 50 μg/kg of one therapeutic conjugate per dose. In yet a further embodiment, the therapeutically effective amount Globo-H moiety in the pharmaceutical/therapeutic composition comprises about 0.10 μg/kg to about 0.75 μg/kg of one therapeutic conjugate per dose.

In still another embodiment, the therapeutically effective amount of the Globo-H-KLH complex in the therapeutic composition may range from about 0.001 μg/kg to about 250 mg/kg. In yet a further embodiment, the therapeutically effective amount of the Globo-H-KLH complex in the therapeutic composition comprises about 10 μg/kg to about 50 μg/kg of one therapeutic conjugate per dose. In yet a further embodiment, the therapeutically effective amount of the Globo-H-KLH complex in the therapeutic composition comprises about 0.60 μg/kg to about 4.50 μg/kg of one therapeutic conjugate per dose.

In still another embodiment, the method is capable of extending progression free survival over a control placebo by about or at least 1 week. In still another embodiment, the method is capable of extending progression free survival over a control placebo by about or at least 2 weeks. In still another embodiment, the method is capable of extending progression free survival over a control placebo by about or at least 1 month. In still another embodiment, the method is capable of extending progression free survival over a control placebo by about or at least 3 months. In still another embodiment, the method is capable of extending progression free survival over a control placebo by about or at least 6 months. In yet another embodiment, the method is capable of extending or overall survival over a control placebo by about or at least 12 months.

### BRIEF DESCRIPTION OF THE FIGURES

A more complete understanding of the invention may be obtained by reference to the accompanying drawings, when considered in conjunction with the subsequent detailed description. The embodiments illustrated in the drawings are intended only to exemplify the invention and should not be construed as limiting the invention to the illustrated embodiments.

FIG. 1A and FIG. 1B show the chemical structure Globo H and as well as several exemplary Globo H analogs. Glc stands for glucose, Gal stands for galactose, GalNAc stands for N-acetylgalactosamine, and Fuc stands for fucose. FIG. 1C shows an exemplary Globo H-KLH subunit conjugated by way of an MMCCH linker.

FIG. 2A shows an exemplary Globo H-KLH subunit conjugation synthesis pathway. FIG. 2B shows Globo H-KLH dimers and trimers of the invention compared to Globo H conjugates disclosed in Slovin et al (1999), Proc Natl Acad Sci USA 96:5710-5 and Gilewski et al (2001), Proc Natl Acad Sci USA 98: 3270-5.

FIG. 3 shows the result of multi-angle laser scattering spectrometry (MALS) of native KLH (8.3 MDa).

FIG. 4A shows the result of size exclusion chromatography of KLH using multi-angle laser scattering spectrometry (MALS) as detector. FIG. 4B shows the mass distribution analysis of KLH. FIG. 4C shows the result of size exclusion chromatography of Globo H-KLH glycoconjugate (OBI-822 Lot no. 14001) using multi-angle laser scattering spectrometry (MALS) as detector. FIG. 4D shows the mass distribution analysis of Globo H-KLH glycoconjugate.

FIG. 5A shows the chronological expansion of B cell populations in Lewis rats immunized with an exemplary Globo H-KLH glycoconjugate (7.5 μg and 25 μg). FIG. 5B shows the chronological expansion of CD3 T cell populations in Lewis rats immunized with an exemplary Globo H-KLH glycoconjugate (7.5 μg and 25 μg). FIG. 5C shows the chronological expansion of CD4 T cell populations in Lewis rats immunized with an exemplary Globo H-KLH glycoconjugate (7.5 μg and 25 μg). FIG. 5D shows the chronological expansion of CD8 T cell populations in Lewis rats immunized with an exemplary Globo H-KLH glycoconjugate (7.5 μg and 25 μg). Data were presented as percentage of cell numbers in indicated group normalized to the percentage of cell numbers of PBS group. Multiple comparisons were analyzed using two-way ANOVA, followed by Bonferroni's post hoc tests. *, p<0.05, **, p<0.01, and ***, p<0.001 compared with PBS.

FIG. 6A shows the chronological changes in reciprocal titers of IgM antibodies in the blood from Lewis rats immunized with an exemplary Globo H-KLH glycoconjugate (7.5 μg and 25 μg). FIG. 6B shows the chronological changes in reciprocal titers of IgG antibodies in the blood from Lewis rats immunized with an exemplary Globo H-KLH glycoconjugate (7.5 μg and 25 μg).

FIG. 7 shows the IgM antibody titers in mice in response to the conjugation ratio between Globo H and KLH (0.17:1 and 0.07:1).

FIGS. 8A and 8B illustrate the immunogenicity of C57BL/6 mice that were immunized with PBS, Globo H-KLH glycoconjugate+saponin adjuvant (OBI-822+OBI-821) and Globo H-KLH glycoconjugate+C34 adjuvant (OBI-822+OBI-834). The sera were collected on day 42 for ELISA analysis to determine the anti-Globo H IgM and IgG antibodies production. FIG. 8A shows the IgM production. FIG. 8B shows the IgG production. The subcutaneous (SC) administration of Globo H-KLH glycoconjugate was 2 μg and adjuvant was 20 μg.

FIGS. 9A and 9B illustrate six groups of immunocompetent (6-8 weeks old), pathogen-free (SPF) C57BL/6 female mice were injected with 5.0×10⁶ in 0.1 mL Lewis Lung carcinoma (LL/2, ATCC CRL-1642) cells, syngeneic for C57BL/6 mice, were injected subcutaneously into the abdominal region toward the lateral side of experimental mice on day 16. Three testing conditions (PBS only, OBI-822+OBI-821 adjuvant and OBI-822+OBI-834 adjuvant) were administered subcutaneously in 0.2 mL/mouse (at both left and right abdominal sties, 0.1 mL/site) or intraperitoneally in the dosing volume of 10 mL/kg on days 0, 5, 11, 19, 29, 34 and 39. The pictures including tumor with whole body were taken after sacrifice on day 42

FIG. 10 illustrates LL/2 (Lewis lung carcinoma cell line) tumor growth on Globo H-KLH glycoconjugate immunized C57BL/6 mice that were subcutaneously vaccinated with PBS, OBI-822+OBI-821 adjuvant and OBI-822+OBI-834 adjuvant on day 0, 5, 11, 19, 29, 34 and 39. LL/2 cells (5.0×10⁶ in 0.1 mL) were subcutaneously injected into each mouse on day 16. Tumor sizes were monitored on day 16, 19, 23, 26, 30, 34, 37, 40 and 42.

FIG. 11 illustrates the chemical structure of Globo H derivative. FIG. 11 illustrates the Globo H derivative [Chemical formula: C(56) H(91) N(5) O(33) S(1), Monoisotopic MW addition: 1393.5317 Da]. FIG. 11B illustrates the neutral loss forms of Globo H derivative. Chemical formula 1: C(18) H(28) N(4) O(4) S(1), Monoisotopic MW addition: 396.1831 Da; Chemical formula 2: C(24) H(38) N(4) O(9) S(1), Monoisotopic MW addition: 558.2360 Da; Chemical formula 3: C(30) H(48) N(4) O(14) S(1), Monoisotopic MW addition: 720.2888 Da; Chemical formula 4: C(36) H(58) N(4) O(19) S(1), Monoisotopic MW addition: 882.3416 Da; Chemical formula 5: C(44) H(71) N(5) O(24) S(1), Monoisotopic MW addition: 1085.4210 Da.

FIGS. 12A and 12B illustrate the chemical structure of MMCCH derivative. FIG. 12A illustrates the chemical structure of MMCCH derivative [Chemical formula: C(16) H(24) N(4) O(3) S(1), Monoisotopic MW addition: 352.1569 Da]. FIG. 12B illustrates the deamidated MMCCH derivative [Chemical formula: C(16) H(22) N(2) O(4) S(1), Monoisotopic MW addition: 338.1300 Da].

FIG. 13A shows a summary of Globo H and MMCCH derivative peptide identification on lysine residue. FIG. 13B shows MMCCH derivative analysis.

FIGS. 14A and 14B illustrate the identification details of Globo-H conjugated peptides on lysine residue for sample 1 (1^{st} LC-MS/MS) for KLH1 (FIG. 14A) and KLH2 (FIG. 14B).

FIGS. 15A and 15B illustrate the identification details of Globo-H conjugated peptides on lysine residue for sample 2 (1^{st} LC-MS/MS) for KLH1 (FIG. 15A) and KLH2 (FIG. 15B).

FIGS. 16A and 16B illustrate the identification details of Globo-H conjugated peptides on lysine residue for sample 3 (1^{st} LC-MS/MS) for KLH1 (FIG. 16A) and KLH2 (FIG. 16B).

FIGS. 17A and 17B illustrate the identification details of Globo-H conjugated peptides on lysine residue for sample 4 (1^{st} LC-MS/MS) for KLH1 (FIG. 17A) and KLH2 (FIG. 17B).

FIGS. 18A and 18B illustrate the identification details of Globo-H conjugated peptides on lysine residue for sample 1 (2^{nd} LC-MS/MS) for KLH1 (FIG. 18A) and KLH2 (FIG. 18B).

FIGS. 19A and 19B illustrate the identification details of Globo-H conjugated peptides on lysine residue for sample 2 (2^{nd} LC-MS/MS) for KLH1 (FIG. 19A) and KLH2 (FIG. 19B).

FIGS. 20A and 20B illustrate the identification details of Globo-H conjugated peptides on lysine residue for sample 3 (2^{nd} LC-MS/MS) for KLH1 (FIG. 20A) and KLH2 (FIG. 20B).

FIGS. 21A and 21B illustrate the identification details of Globo-H conjugated peptides on lysine residue for sample 4 (2^{nd} LC-MS/MS) for KLH1 (FIG. 21A) and KLH2 (FIG. 21B).

FIGS. 22A, 22B, 22C, 22D, 22E, 23A, 23B, 23C, and 23D illustrate the identification details of MMCCH-conjugated peptides on lysine residue for sample 1 (1^{st} LC-MS/MS) for KLH1 (22A-22E) and KLH2 (23A-23D).

FIGS. 24A, 24B, 24C, 24D, 24E, 25A, 25B, 25C, and 25D illustrate the identification details of MMCCH-conjugated peptides on lysine residue for sample 2 (1^{st} LC-MS/MS) for KLH1 (FIGS. 24A-24E) and KLH2 (FIG. 25A-25D).

FIGS. 26A, 26B, 26C, 26D, 26E, 27A, 27B, 27C, and 27D illustrate the identification details of MMCCH-conjugated peptides on lysine residue for sample 3 (1^{st} LC-MS/MS) for KLH1 (FIG. 26A-E) and KLH2 (FIG. 27A-D).

FIGS. 28A, 28B, 28C, 28D, 28E, 29A, 29B, 29C, and 29D illustrate the identification details of MMCCH-conjugated peptides on lysine residue for sample 4 (1^{st} LC-MS/MS) for KLH1 (FIG. 28A-E) and KLH2 (FIG. 29A-D).

FIGS. 30A, 30B, 30C, 30D, 30E, 31A, 31B, 31C, and 31D illustrate the identification details of MMCCH-conjugated peptides on lysine residue for sample 1 (2^{nd} LC-MS/MS) for KLH1 (FIG. 30A-E) and KLH2 (FIG. 31A-D).

FIGS. 32A, 32B, 32C, 32D, 32E, 33A, 33B, 33C, and 33D illustrate the identification details of MMCCH-conjugated peptides on lysine residue for sample 2 (2^{nd} LC-MS/MS) for KLH1 (FIG. 32A-E) and KLH2 (FIG. 33A-D).

FIGS. 34A, 34B, 34C, 34D, 34E, 35A, 35B, 35C, and 35D illustrate the identification details of MMCCH-conjugated peptides on lysine residue for sample 3 (2^{nd} LC-MS/MS) for KLH1 (FIG. 34A-E) and KLH2 (FIG. 35A-D).

FIGS. 36A, 36B, 36C, 36D, 37A, 37B, 37C, and 37D illustrate the identification details of MMCCH-conjugated peptides on lysine residue for sample 4 (2^{nd} LC-MS/MS) for KLH1 (FIG. 36A-D) and KLH2 (FIG. 37A-D). FIGS. 38A-D illustrate the summary of Globo-H conjugated lysine sites identification for (1^{st} LC-MS/MS) for KLH1 (FIG. 38A) and KLH2 (FIG. 38B); (2^{nd} LC-MS/MS) for KLH1 (FIG. 38C) and KLH2 (FIG. 38D).

FIGS. 39A-D illustrate the summary of MMCCH-conjugated lysine sites identification for (1^{st} LC-MS/MS) for KLH1 (FIG. 39A) and KLH2 (FIG. 39B) and (2^{nd} LC-MS/MS) for KLH1 (FIG. 39C) and KLH2 (FIG. 39D).

FIGS. 40A and 40B illustrate a summary of the Globo-H conjugation analysis in the first (FIG. 40A) and second (FIG. 40B) LC-MS/MS runs.

FIG. 41 shows a summary of an exemplary Globo H and MMCCH derivative peptide identification on Histidine (H), Asparagine (N), Proline (P), Glutamine (Q) and Arginine (R) residues.

FIGS. 42A and 42B illustrate the identification details of Globo H and MMCCH-conjugated peptides on HNPQR residues for sample 1 (LC-MS/MS) for KLH1 (FIG. 42A) and KLH2 (FIG. 42B).

FIGS. 43A and 43B illustrate the identification details of Globo H and MMCCH-conjugated peptides on HNPQR residues for sample 2 (LC-MS/MS) for KLH1 (FIG. 43A) and KLH2 (FIG. 43B).

FIGS. 44A and 44B illustrate the identification details of Globo H and MMCCH-conjugated peptides on HNPQR residues for sample 3 (LC-MS/MS) for KLH1 (FIG. 44A) and KLH2 (FIG. 44B).

FIGS. 45A and 45B illustrate the identification details of Globo H and MMCCH-conjugated peptides on HNPQR residues for sample 4 (LC-MS/MS) for KLH1 (FIG. 45A) and KLH2 (FIG. 45B).

FIG. 46 illustrates a summary of exemplary Globo H and MMCCH derivative analysis on Histidine (H), Asparagine (N), Proline (P), Glutamine (Q) and Arginine (R) residues

FIG. 47 illustrates the general steps of the chemical synthesis of Globo H.

### DETAILED DESCRIPTION OF THE INVENTION

The practice of the present invention will employ, unless otherwise indicated, conventional techniques of molecular biology, microbiology, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press, 1989); DNA Cloning, Volumes I and II (D. N. Glover ed., 1985); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Antibodies: A Laboratory Manual, by Harlow and Lanes (Cold Spring Harbor Laboratory Press, 1988); and Handbook Of Experimental Immunology, Volumes I-IV (D. M. Weir and C. C. Blackwell, eds., 1986).

The use of synthetic carbohydrate conjugates to elicit antibodies was first demonstrated by Goebel and Avery in 1929. (Goebel, W. F., and Avery, O. T., J. Exp. Med., 1929, 50, 521; Avery, O. T., and Goebel, W. F., J. Exp. Med., 1929, 50, 533.) Carbohydrates were linked to carrier proteins via the benzenediazonium glycosides. Immunization of rabbits with the synthetic antigens generated polyclonal antibodies. Other workers (Allen, P. Z., and Goldstein, I. J., Biochemistry, 1967, 6, 3029; Rude, E., and Delius, M. M., Carbohydr. Res., 1968, 8, 219; Himmelspach, K., et al., Eur. J. Immunol., 1971, 1, 106; Fielder, R. J., et al., J. Immunol., 1970, 105, 265) developed similar techniques for conjugation of carbohydrates to protein carriers.

Glycoconjugates may be used in active immunotherapy generated from vaccinations to specifically target known target agents on tumor cells. The response to carbohydrate antigens normally does not enlist the use of T-cells, which would aid in the body's rejection of the tumor. While the probability of complete tumor rejection as a result of vaccination with a conjugate is thought to be unlikely, such treatments will boost immune surveillance and recurrence of new tumor colonies can be reduced. (Dennis, J., Oxford Glycosystems Glyconews Second, 1992; Lloyd, K. O., in Specific Immunotherapy of Cancer with Vaccines, 1993, New York Academy of Sciences, 50-58). Toyokuni and Singhal have described a synthetic glycoconjugate (Toyokuni, T., et al., J. Am. Chem. Soc., 1994, 116, 395) that stimulated a measurable IgG titer, a result which is significant since an IgG response is generally associated with enlistment of helper T cells.

A synthetic Globo H vaccine in combination with an immunological adjuvant was shown to induce mainly IgM and to a lesser extent IgG antibodies in both prostate and metastatic breast cancer patients. In a phase I clinical trial, the vaccine also showed minimal toxicity with transient local skin reactions at the vaccination site. (Gilewski T et al. (2001) Proc Natl Acad Sci USA 98:3270-3275; Ragupathi G, et al. (1997) Angew Chem Int Ed 36:125-128; Slovin S F et al (1997) Proc Natl Acad Sci USA 96:5710-5715). Mild flu-like symptoms which have been observed in some of the patients were probably associated with the side effect of QS-21. A pentavalent vaccine containing five prostate and breast cancer associated carbohydrate antigens-Globo-H, GM2, STn, TF and Tn-conjugated to maleimide-modified carrier protein KLH has been reported to produce anti-Globo H sera with higher titers of IgG than IgM in ELISA assays. (Zhu J. et al. (2009) J. Am. Chem. Soc. 131(26):9298-9303).

Accordingly, the present disclosure is directed to immunogenic/therapeutic compounds, compositions, and/or pharmaceutical formulation compositions targeted to/mediated by Globo H, as well as, immunotherapeutics, vaccines, dosage forms, kits, and methods of manufacture, and treatment thereof.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes for example, the inherent variation of error for a measuring device, the method being employed to determine the value, or the variation that exists among the study subjects. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

As used herein, the term “alkyl” refers to a straight or branched monovalent hydrocarbon containing, unless otherwise stated, 1→20 carbon atoms, e.g., C₁-C₈ or C₁-C₄, which can be substituted or unsubstituted. Examples of alkyl include, but are not limited to, methyl, ethyl, n-propyl, i-propyl, n-butyl, i-butyl, and t-butyl.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrequired elements or method steps. It is contemplated that any embodiment discussed in this specification can be implemented with respect to any method or composition of the invention, and vice versa. Furthermore, compositions of the invention can be used to achieve methods of the invention.

“Treating” or “treating” is referred to herein as administration of a therapeutic composition to a subject with the purpose to cure, alleviate, relieve, remedy, prevent, or ameliorate a disorder, symptoms of the disorder, a disease state secondary to the disorder, or predisposition toward the disorder.

An “effective amount” is an amount of a therapeutic composition that is capable of producing a medically desirable result as delineated herein in a treated subject. The medically desirable result may be objective (i.e., measurable by some test or marker) or subjective (i.e., subject gives an indication of or feels an effect).

“Disease amenable to treatment with a therapeutic composition” as referred to herein means any procedures, conditions, disorders, ailments and/or illnesses which can be treated by the administration of the therapeutic compositions disclosed herein.

A “proliferative disorder” is one in which too many of some type of cell are produced resulting in deterioration of health. A proliferative disorder can be benign or malignant. Proliferative disorders can include for example, cancer.

As used herein, “cancer” that can be treated by the therapeutic compositions disclosed herein, includes cells with an abnormal growth state. Cancer cells can be characterized by loss of normal control mechanisms and thus are able to expand continuously, invade adjacent tissues, migrate to distant parts of the body, and promote the growth of new blood vessels from which the cells derive nutrients. As used herein, a cancer can be malignant or benign. Cancer can develop from any tissue within the body. As cells grow and multiply, they form a mass of tissue, called a tumor. The term tumor can include an abnormal growth or mass. Tumors can be cancerous (malignant) or noncancerous (benign). Cancerous tumors can invade neighboring tissues and spread throughout the body (metastasize). Benign tumors, however, generally do not invade neighboring tissues and do not spread throughout the body. Cancer can be divided into those of the blood and blood-forming tissues (leukemia and lymphoma) and “solid” tumors. “Solid” tumors can include carcinomas or sarcomas.

Cancers that may be treated by the therapeutic compositions of the invention include those classified by site include cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, ovary; vagina; vulva; and other female genital); cancers of the male genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); lymphomas (Hodgkin's disease and non-Hodgkin's lymphoma), multiple myeloma, and leukemia (lymphocytic leukemia; myeloid leukemia; monocytic leukemia; and other leukemia).

Other cancers, classified by histological type, that may be suitable targets for the therapeutic compositions according to the present invention include, but are not limited to, neoplasm, malignant; Carcinoma, NOS; Carcinoma, undifferentiated, NOS; Giant and spindle cell carcinoma; Small cell carcinoma, NOS; Papillary carcinoma, NOS; Squamous cell carcinoma, NOS; Lymphoepithelial carcinoma; Basal cell carcinoma, NOS; Pilomatrix carcinoma; Transitional cell carcinoma, NOS; Papillary transitional cell carcinoma; Adenocarcinoma, NOS; Gastrinoma, malignant; Cholangiocarcinoma; Hepatocellular carcinoma, NOS; Combined hepatocellular carcinoma and cholangiocarcinoma; Trabecular adenocarcinoma; Adenoid cystic carcinoma; Adenocarcinoma in adenomatous polyp; Adenocarcinoma, familial polyposis coli; Solid carcinoma, NOS; Carcinoid tumor, malignant; Bronchioloalveolar adenocarcinoma; Papillary adenocarcinoma, NOS; Chromophobe carcinoma; Acidophil carcinoma; Oxyphilic adenocarcinoma; Basophil carcinoma; Clear cell adenocarcinoma, NOS; Granular cell carcinoma; Follicular adenocarcinoma, NOS; Papillary and follicular adenocarcinoma; Nonencapsulating sclerosing carcinoma; Adrenal cortical carcinoma; Endometroid carcinoma; Skin appendage carcinoma; Apocrine adenocarcinoma; Sebaceous adenocarcinoma; Ceruminous adenocarcinoma; Mucoepidermoid carcinoma; Cystadenocarcinoma, NOS; Papillary cystadenocarcinoma, NOS; Papillary serous cystadenocarcinoma; Mucinous cystadenocarcinoma, NOS; Mucinous adenocarcinoma; Signet ring cell carcinoma; Infiltrating duct carcinoma; Medullary carcinoma, NOS; Lobular carcinoma; Inflammatory carcinoma; Paget's disease, mammary; Acinar cell carcinoma; Adenosquamous carcinoma; Adenocarcinoma w/ squamous metaplasia; Thymoma, malignant; Ovarian stromal tumor, malignant; Thecoma, malignant; Granulosa cell tumor, malignant; Androblastoma, malignant; Sertoli cell carcinoma; Leydig cell tumor, malignant; Lipid cell tumor, malignant; Paraganglioma, malignant; Extra-mammary paraganglioma, malignant; Pheochromocytoma; Glomangiosarcoma; Malignant melanoma, NOS; Amelanotic melanoma; Superficial spreading melanoma; Malig melanoma in giant pigmented nevus; Epithelioid cell melanoma; Blue nevus, malignant; Sarcoma, NOS; Fibrosarcoma, NOS; Fibrous histiocytoma, malignant; Myxosarcoma; Liposarcoma, NOS; Leiomyosarcoma, NOS; Rhabdomyosarcoma, NOS; Embryonal rhabdomyosarcoma; Alveolar rhabdomyosarcoma; Stromal sarcoma, NOS; Mixed tumor, malignant, NOS; Mullerian mixed tumor; Nephroblastoma; Hepatoblastoma; Carcinosarcoma, NOS; Mesenchymoma, malignant; Brenner tumor, malignant; Phyllodes tumor, malignant; Synovial sarcoma, NOS; Mesothelioma, malignant; Dysgerminoma; Embryonal carcinoma, NOS; Teratoma, malignant, NOS; Struma ovarii, malignant; Choriocarcinoma; Mesonephroma, malignant; Hemangiosarcoma; Hemangioendothelioma, malignant; Kaposi's sarcoma; Hemangiopericytoma, malignant; Lymphangiosarcoma; Osteosarcoma, NOS; Juxtacortical osteosarcoma; Chondrosarcoma, NOS; Chondroblastoma, malignant; Mesenchymal chondrosarcoma; Giant cell tumor of bone; Ewing's sarcoma; Odontogenic tumor, malignant; Ameloblastic odontosarcoma; Ameloblastoma, malignant; Ameloblastic fibrosarcoma; Pinealoma, malignant; Chordoma; Glioma, malignant; Ependymoma, NOS; Astrocytoma, NOS; Protoplasmic astrocytoma; Fibrillary astrocytoma; Astroblastoma; Glioblastoma, NOS; Oligodendroglioma, NOS; Oligodendroblastoma; Primitive neuroectodermal; Cerebellar sarcoma, NOS; Ganglioneuroblastoma; Neuroblastoma, NOS; Retinoblastoma, NOS; Olfactory neurogenic tumor; Meningioma, malignant; Neurofibrosarcoma; Neurilemmoma, malignant; Granular cell tumor, malignant; Malignant lymphoma, NOS; Hodgkin's disease, NOS; Hodgkin's; paragranuloma, NOS; Malignant lymphoma, small lymphocytic; Malignant lymphoma, large cell, diffuse; Malignant lymphoma, follicular, NOS; Mycosis fungoides; Other specified non-Hodgkin's lymphomas; Malignant histiocytosis; Multiple myeloma; Mast cell sarcoma; Immunoproliferative small intestinal disease; Leukemia, NOS; Lymphoid leukemia, NOS; Plasma cell leukemia; Erythroleukemia; Lymphosarcoma cell leukemia; Myeloid leukemia, NOS; Basophilic leukemia; Eosinophilic leukemia; Monocytic leukemia, NOS; Mast cell leukemia; Megakaryoblastic leukemia; Myeloid sarcoma; and Hairy cell leukemia.

“Epithelial cancers” as defined herein refers to cancer(s) that develops from epithelium or related tissues in the skin, hollow viscera, and other organs. Epithelial cancers include but are not limited to breast cancer, lung cancer, liver cancer, buccal cancer, stomach cancer, colon cancer, nasopharyngeal cancer, dermal cancer, renal cancer, brain tumor, prostate cancer, ovarian cancer, cervical cancer, endometrial cancer, intestinal cancer, pancreatic cancer, and bladder cancer.

“Patient” or “Subject” as used herein refers to a mammalian subject diagnosed with or suspected of having or developing a proliferative disease such as cancer. Exemplary patients may be humans, apes, dogs, pigs, cattle, cats, horses, goats, sheep, rodents and other mammalians that can benefit develop proliferative diseases such as cancer.

As used herein, “substantially purified” or “substantially isolated” refers to a molecule (e.g. a compound) in a state that it is separated from substantially all other molecules normally associated with it in its native state. Preferably, a substantially purified molecule is the predominant species present in a preparation. Particularly, a substantially purified molecule may be greater than 60% free, preferably 75% free, more preferably 90% free, and most preferably 95% free from the other molecules (exclusive of solvent) present in the natural mixture. The term “substantially purified” or “substantially isolated” is not intended to include molecules or substances present in their native state. In certain embodiments, the term “substantially purified” or “substantially isolated” includes purifying one KLH moiety from another KLH moiety (e.g., substantially purifying or substantially isolating a KLH dimer moiety from a KLH trimer moiety). In another embodiment, the term “substantially purified” or “substantially isolated” does not include purifying one KLH moiety from another KLH moiety (e.g. KLH dimers and KLH trimmers are included in a substantially purified or substantially isolated composition) but impurities are substantially removed.

“Administering” is referred to herein as providing a therapeutic composition of the invention to a patient. By way of example and not limitation, composition administration, e.g., injection, may be performed by intravenous (i.v.) injection, sub-cutaneous (s.c.) injection, intradermal (i.d.) injection, intraperitoneal (i.p.) injection, or intramuscular (i.m.) injection. One or more such routes may be employed. Parenteral administration can be, for example, by bolus injection or by gradual perfusion over time. Alternatively, or concurrently, administration may be by the oral route. Additionally, administration may also be by surgical deposition of a bolus or positioning of a medical device.

“A patient in need thereof” is referred to herein as a patient diagnosed with or suspected of having a proliferative disorder. In one embodiment, the patient has or is likely to develop cancer.

As used herein, the term “antigen” is defined as any substance capable of eliciting an immune response, with or without the help of a protein carrier and/or an adjuvant. Preferably the antigen of the inventive compositions includes a carbohydrate and more preferably glycan-antigen and most preferably a Globo H moiety.

As used herein, the term “immunogenicity” refers to the ability of an immunogen, antigen, or vaccine to stimulate an immune response.

As used herein, the term “immunotherapy” refers to an array of treatment strategies based upon the concept of modulating the immune system to achieve a prophylactic and/or therapeutic goal.

As used herein, the term “epitope” is defined as the parts of an antigen molecule which contact the antigen binding site of an antibody or a T cell receptor.

The “therapeutic compositions” of the invention include “immunogenic conjugates and/or therapeutic conjugates and/or “therapeutic antibodies.” The therapeutic conjugates include at least one antigen linked to a carrier. Preferably, the linkage of the therapeutic conjugate is covalent. In one embodiment of the therapeutic conjugate, the antigen is a glycan such as Globo H moiety, and the carrier is a KLH moiety and/or a KLH moiety subunit. As such, the term therapeutic conjugate encompasses one or more KLH moiety subunits linked to one or more Globo H moieties. In one embodiment, the term therapeutic conjugate encompasses a one or more KLH moieties linked to about or at least 1, 10, 10² or 10³ Globo H moieties. In another embodiment, the term therapeutic conjugate encompasses one or more KLH moieties linked to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, or more Globo H moieties. Another embodiment encompasses isolated dimers, trimers, tetramers, pentamers or hexamers of such Globo H linked KLH moiety subunits, or combinations thereof.

In one embodiment, the therapeutic conjugate is: Fucα(1→2) Galβ(1→3) GalNAcβ(1→3) Galα(1→4) Galβ(1→4) Gluβ(1-O-ethylhydrazyl-1-carbonyl-cyclohexyl-4-(methyl-N-maleimido)-3-(thiobutyl-imidyl)-Keyhole Limpet Hemocyanin (KLH) also referred to as OBI-822.

“Therapeutic antibodies” are defined to be as antibodies (as further defined below) that specifically bind the inventive therapeutic conjugates and preferably the Globo H moiety portion of the therapeutic conjugates.

As used herein, the term “vaccine” refers to a therapeutic composition that contains a therapeutic conjugate that is used to confer immunity against a disease associated with the antigen. Cancer vaccines are designed to boost the body's natural ability to protect itself, through the immune system, from dangers posed by damaged or abnormal cells such as cancer cells. A protective immune response is one that reduces the severity of disease, including but not limited to, prevention of disease, delay in onset of disease, decreased severity of symptoms, decreased morbidity, and delayed mortality. Preferably, a vaccine is capable of activating both humoral immune response (e.g. stimulation of the production of antibodies by B lymphocytes) and cellular immune response (e.g. an immune response that is mediated by T-lymphocytes and/or other cells, such as NK cells and macrophages). Standard assays have been developed to determine the immune response such as enzyme-linked immunosorbent assay (ELISA), flow cytometry, cell proliferation assay, CTL assays, and ADCC/CDC assays.

As used herein, the term “glycan” refers to a polysaccharide, or oligosaccharide. Glycan is also used herein to refer to the carbohydrate portion of a glycoconjugate, such as a glycoprotein, glycolipid, glycopeptide, glycoproteome, peptidoglycan, lipopolysaccharide or a proteoglycan. Glycans usually consist solely of O-glycosidic linkages between monosaccharides. For example, cellulose is a glycan (or more specifically a glucan) composed of β-1,4-linked D-glucose, and chitin is a glycan composed of β-1,4-linked N-acetyl-D-glucosamine. Glycans can be homo or heteropolymers of monosaccharide residues, and can be linear or branched. Glycans can be found attached to proteins as in glycoproteins and proteoglycans. They are generally found on the exterior surface of cells. O- and N-linked glycans are very common in eukaryotes but may also be found, although less commonly, in prokaryotes. N-Linked glycans are found attached to the R-group nitrogen (N) of asparagine in the sequon. The sequon is an Asn-X-Ser or Asn-X-Thr sequence, where X is any amino acid except praline. The preferred glycan is a Globo H moiety.

Globo H is a hexasaccharide, which is a member of a family of antigenic carbohydrates that are highly expressed on a various types of cancers, especially cancers of breast, prostate, pancreas, stomach, ovary, colon, and lung. In illustrative embodiments, certain patients exhibited no anti-Globo H antibody levels at time zero, and after immunization with the therapeutic composition of the invention high titers were detected. In other illustrative embodiments, certain patients exhibited anti-Globo H antibody levels at time zero, and after immunization with the therapeutic composition of the invention high titers were detected. In certain embodiments, the anti-Globo H antibody is expressed on the cancer cell surface as a glycolipid and possibly as a glycoprotein. In other embodiments, the serum of breast cancer patients contained high levels of antibodies against the Globo H epitope. In certain embodiments, this epitope is also targeted by the monoclonal antibodies Mbr1, VK9 and anti-SSEA-3 in immunohistochemistry studies. Although certain normal tissues also react with Mbr1, including normal breast, pancreas, small bowel, and prostate tissue, the antigen in these tissues is predominantly localized at the secretary borders where access to the immune system is restricted.

“Globo H moiety” is defined herein to be a glycan (i.e., a molecule containing a sugar moiety) that is Globo H or a fragment or analog thereof. Globo H is a glycan containing the hexasaccharide epitope (Fuc α 1→2 Gal β 1→3 GalNAc β 1→3 Gal α 1→4 Gal β 1→4 Glc), and optionally, a non-sugar moiety. Its fragment is a glycan containing a fragment of the hexasaccharide epitope and, if applicable, the non-sugar moiety. These oligosaccharides can be prepared by routine methods. (See Huang et al., Proc. Natl. Acad. Sci. USA 103:15-20 (2006)). If desired, they can be linked to a non-sugar moiety. U.S. patent application Ser. No. 12/485,546 relates to a method of producing antibody specific to Globo H or its fragment by administering to a non-human mammal (e.g., mouse, rabbit, goat, sheep, or horse) the immune composition described above and isolating from the mammalian antibody that binds to Globo H or its fragment.

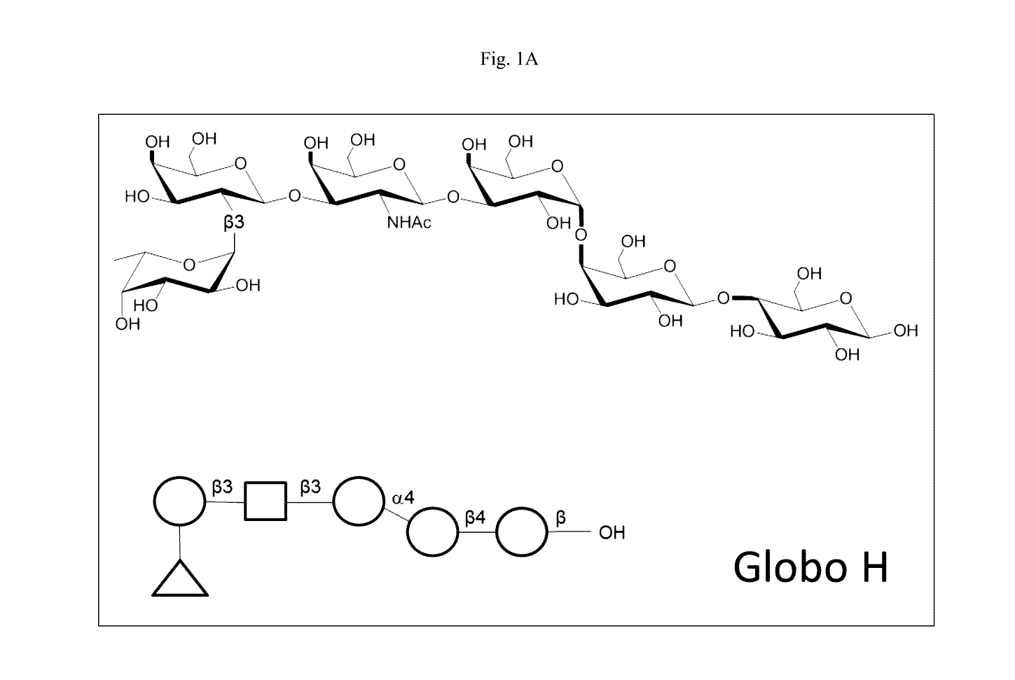

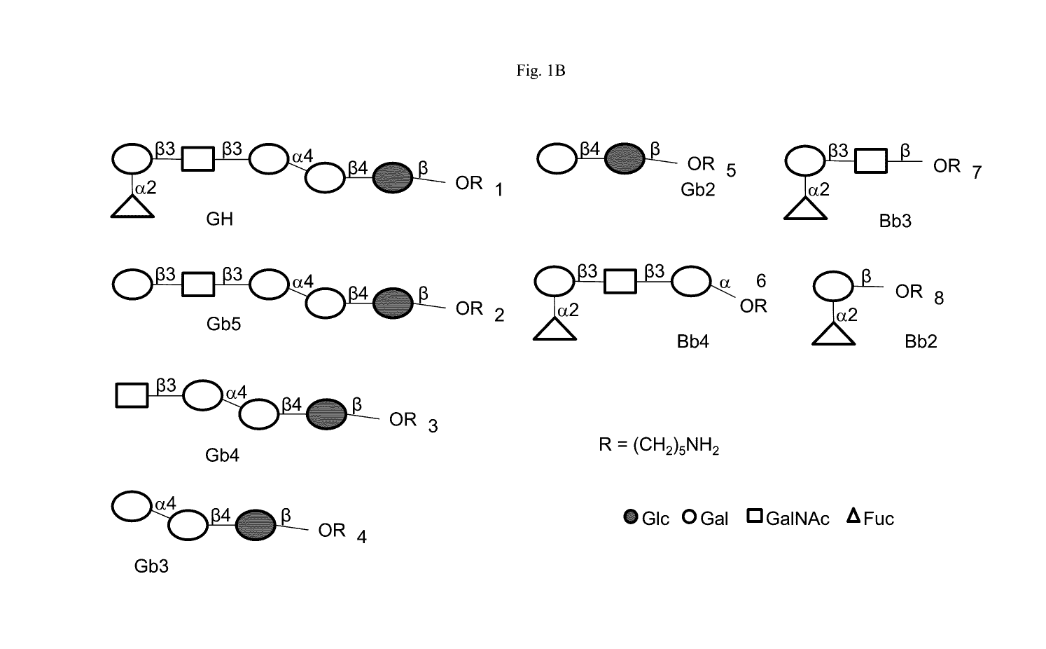

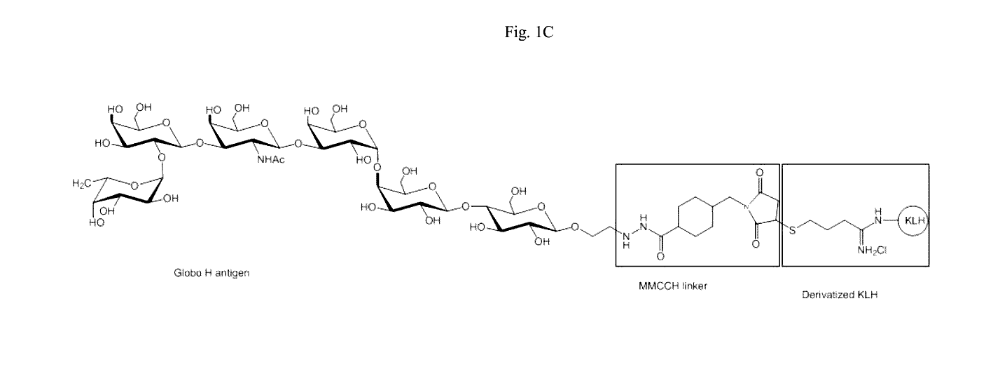

Analogs of Globo H can be generated using glycan microarray and include those disclosed in Wang et al., Proc Natl Acad Sci USA. 2008 Aug. 19; 105(33): 11661-11666 and shown in FIG. 1.

Globo H analogs preferably bind antibodies VK-9, Mbr1, and anti-SSEA-3. Preferably, the Globo H Analogs bind with a particular dissociation constant (K_{D,surf}). The Langmuir isotherm can be used for analyzing the binding curves to generate the dissociation constants on surface (K_{D,surf}). At the equilibrium conditions during incubation, the mean fluorescence of the replicate spots (F_{obs}) can be described by:

F_{obs}=Fₘₐₓ[P]/(K_{D,surf}+[P])

where Fₘₐₓ is the maximum fluorescence intensity, a measure of the amount of active carbohydrate on the surface, [P] is the total antibody concentration, and K_{D,surf} is the equilibrium dissociation constant for surface carbohydrate and the antibody. As described in Wang et al. In some embodiments the preferred (K_{D,surf}) of Globo H analogs is at least, about or exactly 0.4, 0.5., 0.6, 0.7, 0.8, 0.9, 1.0, 1.1, 1.2, 1.3, 1.4, 1.5 or 1.6 nM with respect to the VK-9, Mbr1, and anti-SSEA-3 antibodies described in Wang et al.

“Keyhole Limpet Hemocyanin” (KLH) is a large, multisubunit, oxygen-carrying, metalloprotein found in the hemolymph of the giant keyhole limpet, Megathura crenulata. KLH is heterogeneous glycosylated protein consisting of subunits with a molecular weight of about 350,000 to about 390,000 in aggregates with molecular weights of about 400 kDa (e.g., a KLH monomer) to about 8000 kDa (e.g., a KLH didecamer). Each domain of a KLH subunit contains two copper atoms that together bind a single oxygen molecule. When oxygen is bound to hemocyanin, the molecule takes on a distinctive transparent, opalescent blue color. In certain embodiments, the KLH protein is potently immunogenic yet safe in humans. In certain embodiments, KLH may be purified from the hemolymph of Megathura crenulata by a series of steps that typically includes ammonium sulfate precipitation and dialysis, and may involve chromatographic purification to obtain the highest purity. In certain embodiments, KLH purification may also include endotoxin removal, but this step may be unnecessary because the endotoxin can serve as an adjuvant when injected for antibody production. Preferably, a high quality KLH preparation with the clear opalescent blue color is the best indicator of KLH solubility. In certain embodiments, the KLH monomeric units assemble into a large multimer (decamer or didecamer) with a total molecular weight of about 4,000 kDa to 8,000 kDa. “Keyhole Limpet Hemocyanin moiety” or “KLH moiety” is defined herein to be a KLH1 (SEQ ID NO. 1) or KLH2 (SEQ ID NO. 2) protein or a protein substantially identical thereto or a mixture thereof. Substantially identical in this context means each KLH moiety has an amino sequence at least, about or exactly: 100, 99, 98, 97, 96, 95, 94, 93, 92, 91, 90, 89, 88, 87, 86, 85, 84, 83, 82, 81, 80, 79, 78, 77, 76 or 75 percent identical to that of native wild type KLH. In certain embodiments, the KLH of the invention has enhanced immunogenic activity, particularly enhanced anti-tumor activity. In certain embodiments, the KLH in the composition of the present invention comprises an intact, non-degraded subunit of approximately 400,000 in molecular weight. In other embodiments, the KLH of the invention comprises higher KLH multimers.

In certain embodiments, the higher KLH multimers have molecular weights of approximately 8-10 million with sedimentation coefficients of about 92-107S. The amount of higher KLH multimers present is based on sedimentation-equilibrium and/or sedimentation-velocity ultracentrifugation analyses. In other embodiments, the KLH of the invention demonstrates an enhanced immunogenic activity, particularly enhanced anti-tumor activity. The enhanced immunogenic activity is seen for example, but not limited, (a) with injection of KLH (without adjuvant), (b) with KLH used as an adjuvant, (c) with KLH used as a carrier immunogen for haptens or weakly immunogenic antigens, and (d) with KLH used as an anti-tumor agent. The KLH composition of the invention exhibits enhanced anti-tumor activity for many tumors, including, but not limited to, bladder, breast, ovarian tumors, etc. In certain embodiments, two KLH moieties can form a dimer via a covalent linkage between KLH monomers. Without being limited by theory, it is believed that the covalent linkage between KLH moieties is through a disulfide bond. In certain embodiments, two or more KLH moieties can form a dimer, trimer, tetramer, pentamer, hexamer, etc. via a covalent linkage between KLH monomers, dimers, trimers, etc. Without being limited by theory, it is believed that the covalent linkage between KLH moieties is through a disulfide bond.

There are a variety of methods for linking of a KLH moiety to an antigen, including direct conjugation and conjugation with a bifunctional linker group such as 4-(4-N-maleimidomethyl) cyclohexane-1-carboxyl hydrazide (MMCCH). Such linkage techniques are disclosed in U.S. Pat. No. 6,544,952. In some embodiments, to prepare the therapeutic conjugates of the invention, for example, the Globo H allyl glycoside is converted to an aldehyde by ozonolysis and the aldehyde group is attached to the NH groups on the crosslinker MMCCH, giving Globo H-MMCCH; the carrier protein, KLH, is subjected to thiolation to produce KLH-SH; and the sulfhydryl groups on thiolated KLH are then attached to the maleimide group on the MMCCH, producing Globo H-KLH conjugates.

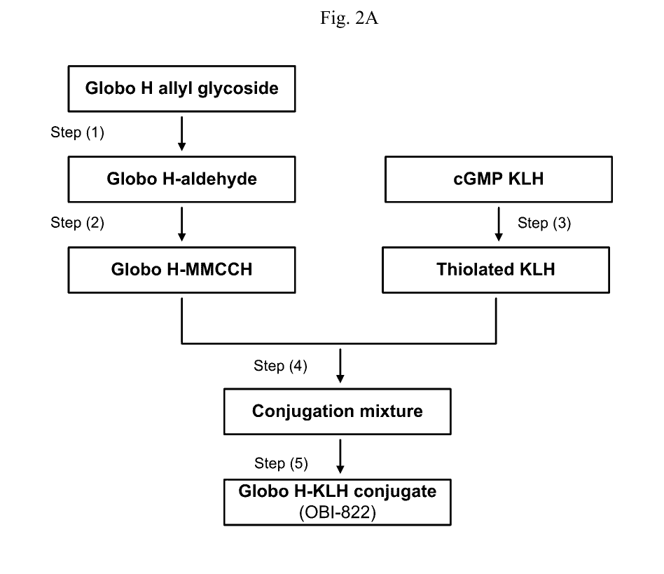

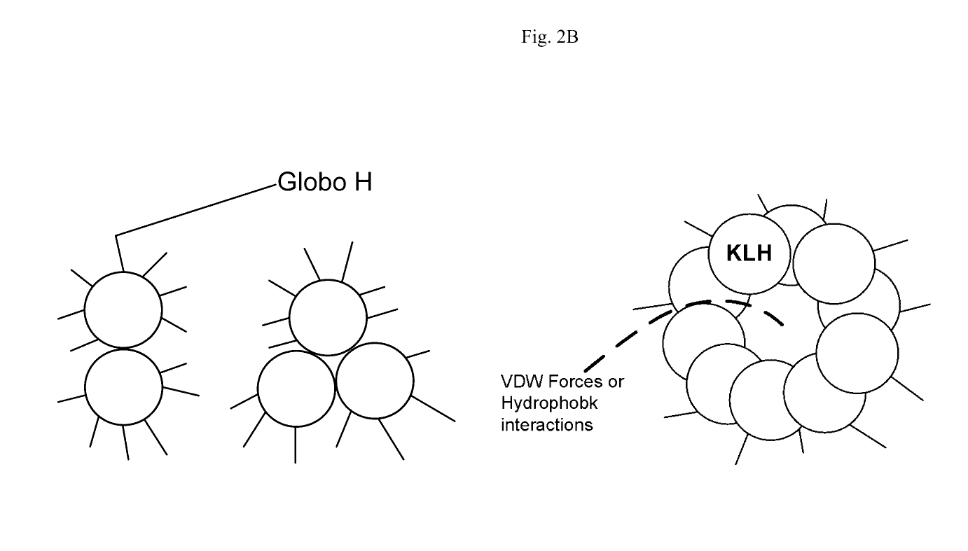

In one embodiment, Globo H allyl glycoside is prepared via chemical synthesis. A thiolating reagent, 2-iminothiolane and cGMP-grade KLH and 4-(4-N-maleimidomethyl)-cyclohexane-1-carboxyl hydrazide (MMCCH) linker are also used. In some embodiments the following steps are carried out: (1) Conversion of Globo H allyl glycoside to the Globo H-aldehyde; (2) Coupling of Globo H-aldehyde with MMCCH to Globo H-MMCCH, separately; (3) Chemical thiolation of KLH; (4) Conjugation of Globo H-MMCCH to the thiolated KLH; and (5) Purification of the Globo H-KLH glycoconjugate (OBI-822). The Globo H-KLH subunit conjugation pathway showed in FIG. 2A.

In certain embodiments, during conjugation of a Globo H moiety protein to a KLH moiety, a KLH moiety protein in certain embodiments shows a reduction in molecular weight compared to the intact molecule preferably due to Globo H moiety subunit dissociation. In other embodiments, the conjugation methods disclosed herein result in a KLH subunit dissociation not previously reported. While not wishing to be bound to any particular theory, it is envisaged that the high glycosylation level of the inventive Globo H moiety-KLH moiety subunit conjugates results in the formation hydrogen bonding between the Globo H moieties. As such, in certain embodiments, the Van Der Waals forces and hydrophobic interactions between the KLH moiety subunits are displaced by Globo H hydrogen bonding and this leads to KLH moiety subunit separation. Following conjugation, the KLH moiety subunits of a Globo H moiety-KLH moiety conjugate preferably aggregate to form novel monomers, dimers, trimers, tetramers, pentamers, hexamers or any combination thereof. The resulting exemplary therapeutic Globo H moiety-KLH moiety conjugates, with an unexpectedly large epitope ratio, have surprising and unexpected superior immunogenic attributes. In certain embodiments, the Globo H moieties are conjugated to lysines on KLH1 and KLH2. In other embodiments, the Globo H moieties are not conjugated to lysines on KLH1 and KLH2. In certain embodiments, the Globo H-conjugated lysine sites are found conserved in the peptide mapping analysis suggesting the Globo H-KLH glycoconjugate composition is unique in its structure.

In one embodiment, therapeutic compositions of the invention include one or more KLH moiety subunits wherein at least one such subunit is conjugated to at least, about or exactly 1, 10, 100 or 1000 times: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159 or 160 or more Globo H moieties.

The inventors found using mass spectrometric analysis that the Globo H moieties are conjugated to lysine residues of KLH. In certain embodiments, it is therefore preferred that the Globo H moieties are conjugated to lysine residues.

In one embodiment, there are total of exactly or about 145, 146, 147, 148, 149, 150, 151, 152, 153, 154, 155, 156, 157, 158, 159, 160 total lysine residues per KLH moiety subunit. In another embodiment there are exactly or about 150 or 156 lysine residues per KLH moiety subunit. In another embodiment, there are exactly or about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109 or 110 lysine conjugation sites on each KLH moiety subunit available for binding to or actually bound to a Globo H moiety. In another embodiment, there are 62, 66, 67, 68, 70, 72, 76, 86, 87, 88, 90, 92, 93, 100 such lysine conjugation sites on each KLH moiety subunit. Lysine conjugation sites are those lysine residues in the KLH moiety which are available for binding or actually bind to a Globo H moiety and/or a linker to a Globo H moiety such as for example an MMCCH linker.

In certain therapeutic embodiments containing a mixture of moiety subunits (e.g., KLH1 and KLH2 or variants thereof), total available lysine (for both subunits) as are counted together across the different subunit types the and may be or are exactly about 290, 291, 292, 293, 294, 295, 296, 297, 298, 299, 300, 301, 302, 303, 304, 305, 306, 307, 308, 309 or 310 in number. In such embodiments, there are or may be exactly or about 130, 131, 132, 133, 134, 135, 136, 137, 138, 139, 140, 141, 142, 143, 144, 145, 146, 147, 148, 149, 150, 151, 152, 153, 154, 155, 156, 157, 158, 159 or 160 lysine conjugation sites together across the different subunits (e.g., KLH1 and KLH2 or variants thereof). In other such embodiments, there are 136, 137, 141, 140, 143, 147 or 155 lysine conjugation sites.

In a most preferred embodiment there are 136, 137, 140, 141, 143, 147 or 155 and lysine conjugation sites among the total 306 lysine residues in KLH1/KLH2.

In certain embodiments, the therapeutic compositions of the invention contain a mixture of KLH moiety subunit-Globo H moiety conjugates wherein such conjugates remain monomers or form dimers, trimers, tetramers, pentamers, hexamers or any combination thereof. In another embodiment, the therapeutic compositions of the invention include isolated KLH moiety subunit-Globo H moiety conjugate monomers, dimers, trimers or tetramers or combinations of thereof. In a further embodiment, the therapeutic compositions of the invention include only KLH moiety subunit-Globo H moiety conjugate dimers and trimers.

In another embodiment, the therapeutic compositions contain at least two KLH moiety subunits wherein each of the two KLH-moiety subunits is linked to different glycans. Other tumor-associated glycan antigens linkable to KLH moiety subunits can include but are not limited to GM2, GD2, GD3, fucosyl, GM1, sTn, sialyl-Lewis^{x}, Lewis^{x}, sialyl Lewis^{a}, Lewis^{a}, sTn, TF, polysialic acid, Lewis^{y}, mucins, T antigen, and the like. In some embodiments only, at least or about 10, 20, 30, 40, 50, 60, 70, 80, 90 or 100 percent of the KLH moiety subunits in a therapeutic composition are linked to a Globo H moiety whereas the remaining KLH moiety subunits in the therapeutic composition are linked to other tumor-associated glycan antigens.

As used herein, “epitope ratio” relating to the therapeutic conjugates disclosed herein refers to for example, the relationship of antigen epitopes to carrier molecules in a therapeutic conjugate. Preferably, it refers to the relationship of Globo H moieties to KLH moieties. Most preferably the epitope ratio of a therapeutic conjugate is calculated using the following formula=(actual Globo H moiety weight/Globo H moiety molecular weight)/(actual KLH moiety weight/KLH moiety molecular weight) combination. Epitope ratios are readily determinable by those of skill in the art. Preferably, the weights of Globo H are determined for example by high performance anion exchange chromatography with pulsed amperometric detection (HPAEC-PAD).

Preferably, the epitope ratios of the therapeutic conjugates of the invention are about, at least or exactly: 1, 10, 25, 50, 75, 100, 125, 150, 175, 200, 225, 250, 275, 300, 325, 350, 375, 400, 425, 450, 475, 500, 525, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975 or 3000.

In one embodiment, the therapeutic compositions of the invention include a mixture of therapeutic conjugates having a range of epitope ratios. In one embodiment, the range, the mean or the median epitope ratios of the therapeutic conjugates in the therapeutic composition is about 10 to about 3200, about 800 to about 2500, about 1000 to about 2000, about 1250 to about 1750 or about 1400 to about 1600. In another embodiment, the range, the mean or the median epitope ratios of the therapeutic conjugates in the therapeutic composition is about 10 to about 150, about 40 to about 125, about 50 to about 100, about 62 to about 87 or about 70 to about 80. In another embodiment, the range, the mean or the median epitope ratios of the therapeutic conjugates in the therapeutic composition is about 20 to about 300, about 80 to about 250, about 100 to about 200, about 125 to about 175 or about 140 to about 160. In another embodiment, the range, the mean or the median epitope ratios of the therapeutic conjugates in the therapeutic composition is about 30 to about 450, about 120 to about 375, about 150 to about 300, about 185 to about 260 or about 210 to about 240. In some the pharmaceutical compositions at least or about 30, 40, 50, 60, 70, 80, 90, 95, 98, 99, or 100% of the therapeutic conjugates exist as monomers, or as dimers, trimers, tetramers, pentamers, hexamers or combinations thereof.

### Antibodies to Therapeutic Conjugates

In certain illustrative embodiments, the invention also encompasses isolated therapeutic antibodies, which specifically bind the therapeutic conjugates disclosed herein with affinity, as well as their use in the treatment and/or diagnosis of proliferative disease.

As used herein, the terms “antibody” and “antibodies” (immunoglobulins) encompass monoclonal antibodies (including full-length monoclonal antibodies), polyclonal antibodies, multispecific antibodies (e.g., bispecific antibodies) formed from at least two intact antibodies, human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity, disulfide-linked Fvs (sdFv), and anti-idiotypic (anti-Id) antibodies (including, e.g., anti-Id antibodies to antibodies of the invention), intrabodies, and epitope-binding fragments of any of the above. In particular, antibodies include immunoglobulin molecules and immunologically active fragments of immunoglobulin molecules, i.e., molecules that contain an antigen-binding site Immunoglobulin molecules can be of any type (e.g., IgG, IgE, IgM, IgD, IgA and IgY), class (e.g., IgG1, IgG2, IgG3, IgG4, IgA1 and IgA2) or subclass.

“Affinity” of an antibody for an epitope, e.g., the Globo H moiety of a therapeutic conjugate, to be used in the treatment(s) described herein is a term well understood in the art and means the extent, or strength, of binding of antibody to epitope. Affinity may be measured and/or expressed in a number of ways known in the art, including, but not limited to, equilibrium dissociation constant (KD or Kd), apparent equilibrium dissociation constant (KD′ or Kd′), and IC50 (amount needed to effect 50% inhibition in a competition assay). It is understood that, for purposes of this invention, an affinity is an average affinity for a given population of antibodies which bind to an epitope. Values of KD′ reported herein in terms of mg IgG per mL or mg/mL indicates mg 1 g per mL of serum, although plasma can be used. When antibody affinity is used as a basis for administration of the treatment methods described herein, or selection for the treatment methods described herein, antibody affinity can be measured before and/or during treatment, and the values obtained can be used by a clinician in assessing whether a human patient is an appropriate candidate for treatment.

As used herein, the term “specifically binding,” refers to the interaction between binding pairs (e.g., an antibody and an antigen). In various instances, specifically binding can be embodied by an affinity constant of at least or about 10-6 moles/liter, about 10-7 moles/liter, or about 10-8 moles/liter, or less.

Exemplary antibodies against the Globo H may be prepared by collecting body fluid from the immunized subj ect examined for the increase of desired antibodies such as the serum, and by separating serum from the blood by any conventional method.

Antibodies are generally raised by multiple injections of the relevant antigen and an adjuvant. It may be useful to conjugate the relevant antigen to a protein that is immunogenic in the species to be immunized, e.g., keyhole limpet hemocyanin.

Methods for immunizing animals with antigens are known in the art. Intraperitoneal injection or subcutaneous injection of antigens is a standard method for immunization of mammals. More specifically, antigens may be diluted and suspended in an appropriate amount of phosphate buffered saline (PBS), physiological saline, etc. If desired, the antigen suspension may be mixed with an appropriate amount of an adjuvant, and then administered to the subject.

In certain embodiments, subjects can be boosted until the titer plateaus by several administrations of antigen mixed with an appropriately amount of adjuvant. An appropriate carrier may also be used for immunization. After immunization as above, serum is examined by a method for an increase in the amount of desired antibodies.

### Biological Assays

In one embodiment, when administered to a patient, the therapeutic compositions containing therapeutic conjugates of the invention are able to induce anti-Globo H antibody titers at least or about 1, 2, 3, 4, 5, 6, 7, 8, 9, 10, 11, 12, 13, 14, 15, 16, 17, 18, 19, 20, 50, 100, 250, 500, 1000, 1500, 2000, 2500, 3000, 4000, or 5000 fold greater that the same anti-Globo H antibody titer prior to the administration (i.e., a pre-treatment baseline titer) in the same experiment. In certain embodiments the anti-Globo H antibodies are IgM antibodies. In another embodiment, the anti-Globo H antibodies are IgG antibodies.

The therapeutic compositions of the invention are capable of inducing both humoral and cellular responses in a subject. In certain embodiments, the vaccine composition of the invention induces production of Globo H moiety-specific IgG and IgM antibodies and expansion of B cells and T cells (e.g. CD3⁺T cells, CD4⁺T cells and/or CD8⁺T cells). Typically, these immune responses occur chronologically after administration. In a particular example, after administration, the B cell production appears at about day 3, 4, 5, 6, 7, 8, 9, 10, 20, 30, or 60 days, followed by production of IgG and IgM antibodies at about day 10, 20, 30, 60, or 90 and subsequent T cell production at about day 24, 30, 40, 50, 60, 90, 120, 150, or 180. The vaccine composition of the invention potentially provides a long term immunological protective effect which could prevent the growth of small quantities of cancer cells, thereby being ideal for minimal residual disease so as to achieve disease stabilization and survival improvement.

“Antibody-dependent cell-mediated cytotoxicity” and “ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell. In one embodiment, such cells are human cells. While not wishing to be limited to any particular mechanism of action, these cytotoxic cells that mediate ADCC generally express Fc receptors (FcRs). The primary cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. No. 5,500,362 or U.S. Pat. No. 5,821,337 may be performed. Useful effector cells for such assays include peripheral blood mononuclear cells (PBMC) and Natural Killer (NK) cells. Alternatively, or additionally, ADCC activity of the therapeutic conjugates of the invention may be assessed in vivo, e.g., in an animal model such as that disclosed in Clynes et al., Proc. Natl. Acad. Sci. (USA), 95:652-656 (1998).

In certain embodiments, the administration of the pharmaceutical composition can generate an immune response, including generation of antibodies that specifically bind to Globo H. In certain embodiments, the antibodies are developed to target one or more of GloboH expressed on the surface of cancer cells and trigger CDC and/or ADCC to kill these cells. In certain embodiments, the antibodies predominantly include IgG antibodies. In certain embodiments, the immunogenic compositions provided herein mainly induce IgG1, IgG2b, IgG2c and IgG3.

“Complement dependent cytotoxicity” or “CDC” refers to the ability of a therapeutic conjugate to initiate complement activation and lyse a target in the presence of complement. The complement activation pathway is initiated by the binding of the first component of the complement system (Clq) to a molecule (e.g., an antibody) complexed with a cognate antigen. To assess complement activation, a CDC assay, e.g., as described in Gazzano-Santaro et al., J. Immunol. Methods, 202:163 (1996), may be performed.

In another embodiment, when administered to a patient the therapeutic compositions containing therapeutic conjugates of the invention are able to induce the production in a patient/subject of anti-Globo H immune sera, which specifically binds to Globo H positive cancer cell lines, for example, MCF-7 cells.

### Combinations

Therapeutic compositions can include other anti-cancer/anti-proliferative drugs as well as adjuvants and other immunomodulatory molecules such as cytokines or chemokines. In certain embodiments, the combination can be a co-administration of separate agent/compositions or co-formulation. These agents can be delivered in a kit together in separate containers or a single container. The agents may be combined at the time of administration or at least or about 1, 2, 3, 4, 5, 6, 7, 8, 9, 10, 11, 12, 13, 14, 15, 16, 17, 18, 19, 20, 21, 22, 23 or 24 minutes, hours or days prior to administration.

Adjuvants are pharmacological or immunological agents that modify the effects of other agents. They can be an inorganic or organic chemical, macromolecule or whole cancer cells or portions thereof which enhance the immune response to given antigen. Adjuvants include complete and incomplete Freund's adjuvant, Toll-Like Receptor molecules and mimetics thereof, LPS, lipoproteins, lipopeptides, flagellin, double-stranded RNA, unmethylated CpG islands, levamisole, bacillus Calmette-Guerin, octreotide, isoprinosine and Zadaxin, various forms of DNA and RNA classically released by bacteria and viruses, PD-1 antagonists and CTLA antagonists. In one embodiment, the adjuvant is a saponin adjuvant.

In certain embodiment, the saponin adjuvant is OBI-821 saponin, which is substantially pure. In other embodiments, the OBI-821 saponin is a biologically active fragments thereof. The adjuvant may also encompass impure forms of OBI-821 saponins. The purified OBI-821 saponins exhibit enhanced adjuvant effect when administered with a vaccine described herein or admixed with other substantially pure saponin or non-saponin adjuvants.

OBI-821 saponins are naturally occurring glycosides, extracted in high purify from the bark of the Quillaja saponaria Molina tree, by high pressure liquid chromatography (HPLC), low pressure liquid silica chromatography, and hydrophilic interactive chromatography (HILIC) as described in, for example, U.S. Pat. No. 5,057,540 and U.S. Pat. No. 6,524,584, the content of which is incorporate by reference in its entirety. High-pressure liquid chromatography analysis shows that OBI-821 are a mixture of structurally related isomeric compounds. Different purified isomeric compounds of OBI-821 saponins have been identified and disclosed herein.

In certain embodiments, OBI-821 saponin comprise at least one isolated compound of formula I as follows:

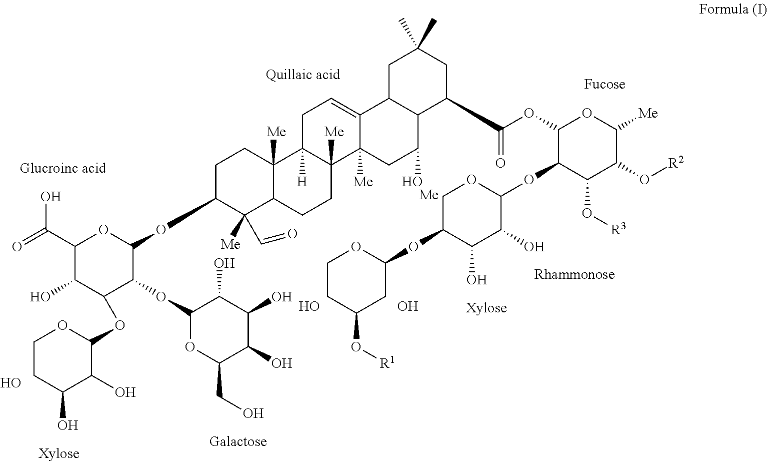

wherein

R1 is β-D-Apiose or β-D-Xylose; and

R2 and R3 are independently H, alkyl,

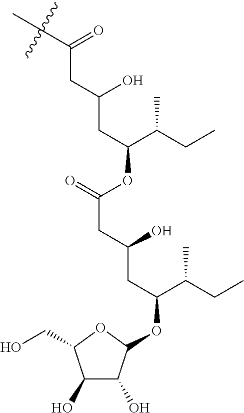

(fatty acyl moiety for Compound 1989), or

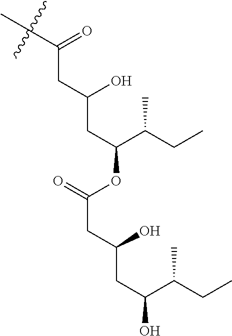 [SMILES: *C(=O)CC(O)C[C@H](OC(=O)C[C@@H](O)C[C@H](O)[C@H](C)CC)[C@H](C)CC]

(fatty acyl moiety for Compound 1857).

OBI-821 saponin can also comprise an isolated compound of formula I, wherein
(i) R¹ is β-D-Apiose, R² is the fatty acyl moiety for the 1989 compound depicted above, and R³ is H (1989 compound V1A);
(ii) R¹ is β-D-Apiose, R² is H, and R³ is the fatty acyl moiety fatty acyl moiety for the 1989 compound depicted above (1989 compound V1B);
(iii) R¹ is β-D-Xylose, R² is the fatty acyl moiety fatty acyl moiety for the 1989 compound depicted above, and R³ is H (1989 compound V2A); or
(iv) R¹ is β-D-Xylose, R² is H, and R³ is the fatty acyl moiety fatty acyl moiety for the 1989 compound depicted above (1989 compound V2B). Collectively, 1989 compound V1A, 1989 compound V1B, 1989 compound V2A and 1989 compound V2B are called “1989 compounds mixture.”

Table 1 summarizes the functional groups of 1989 compounds and the mole % of each 1857 compound in the 1857 compounds mixture.

TABLE 1

| Mole % | R¹ | R² | R³ |
|---|---|---|---|
| 1989 Compound V1A 64.5% | 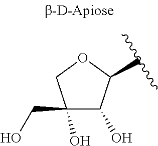 [SMILES: *[C@@H]1OC[C@](O)(CO)[C@H]1O] | 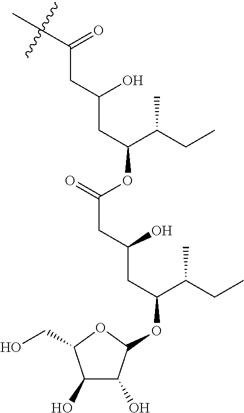 | H |
| 1989 Compound V1B 1.5% | 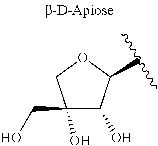 [SMILES: *[C@@H]1OC[C@](O)(CO)[C@H]1O] | H | 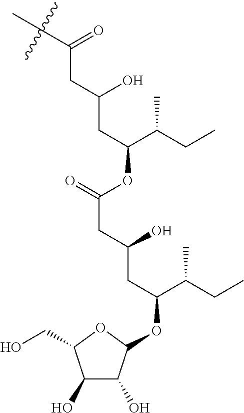 |
| 1989 Compound V2A 33.3% | 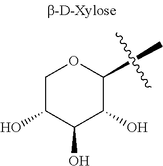 | 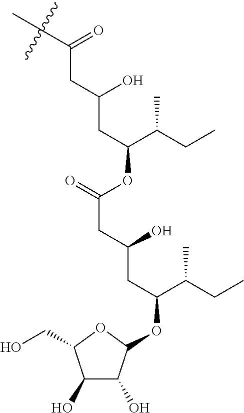 | H |
| 1989 Compound V2B 0.7% | 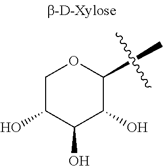 | H | 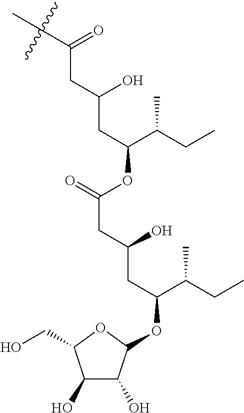 |

OBI-821 saponin can comprise an isolated compound of formula I where:
(i) R¹ is β-D-Apiose, R² is the fatty acyl moiety for the 1857 compound depicted above, and R³ is H (1857 compound V1A);
(ii) R¹ is β-D-Apiose, R² is H, and R³ is the fatty acyl moiety for the 1857 compound depicted above (1857 compound V1B);
(iii) R¹ is β-D-Xylose, R² is the fatty acyl moiety for the 1857 compound depicted above, and R³ is H (1857 compound V2A); or
(iv) R¹ is β-D-Xylose, R² is H, and R³ is the fatty acyl moiety for the 1857 compound depicted above (1857 compound V2B). Collectively, 1857 compound V1A, 1857 compound V1B, 1857 compound V2A and 1857 compound V2B are called “1857 compounds mixture.”

Table 2 summarizes the functional groups of 1857 compounds and the mole % of each 1857 compound in the 1857 compounds mixture. HPLC.

TABLE 2

| Mole % | R¹ | R² | R³ |
|---|---|---|---|
| 1857 Compound V1A 64.7% | 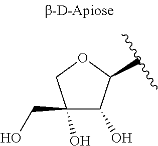 [SMILES: *[C@@H]1OC[C@](O)(CO)[C@H]1O] | 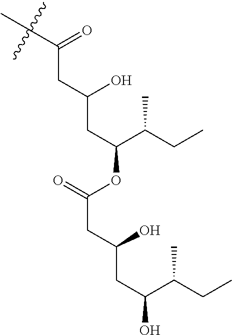 [SMILES: *C(=O)CC(O)C[C@H](OC(=O)C[C@@H](O)C[C@H](O)[C@H](C)CC)[C@H](C)CC] | H |
| 1857 Compound V1B 1.3% | 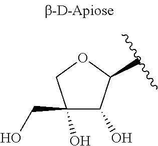 [SMILES: *[C@@H]1OC[C@](O)(CO)[C@H]1O] | H | 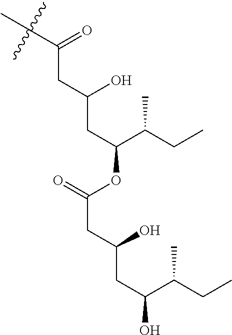 [SMILES: *C(=O)CC(O)C[C@H](OC(=O)C[C@@H](O)C[C@H](O)[C@H](C)CC)[C@H](C)CC] |
| 1857 Compound V2A 33.4% | 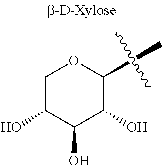 | 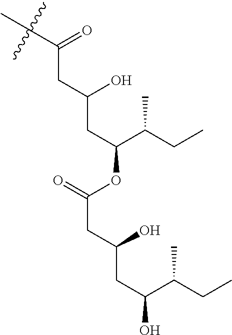 [SMILES: *C(=O)CC(O)C[C@H](OC(=O)C[C@@H](O)C[C@H](O)[C@H](C)CC)[C@H](C)CC] | H |
| 1857 Compound V2B 0.6% | 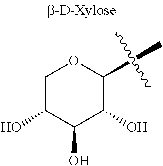 | H | 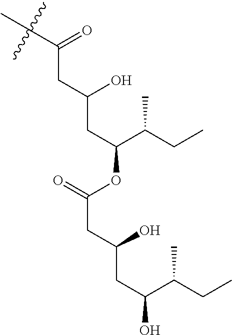 [SMILES: *C(=O)CC(O)C[C@H](OC(=O)C[C@@H](O)C[C@H](O)[C@H](C)CC)[C@H](C)CC] |

OBI-821 saponin comprises one or more of the following compounds:
(i) 1857 compound V1A;
(ii) 1857 compound V1B;
(iii) 1857 compound V2A;
(iv) 1857 compound V2B;
(v) 1989 compound V1A;
(vi) 1989 compound V1B;
(vii) 1989 compound V2A; or
(viii) 1989 compound V2B.

The percentages of the 1857 compounds mixture and the 1989 compound mixture in OBI-821 saponin can range as follows:
(i) about 1 mole % to about 15 mole % of OBI-821 comprising an 1857 compounds mixture; and
(ii) about 85 mole % to about 99 mole % of OBI-821 comprising an 1989 compounds mixture.

All of the mole % can be varied by 0.1% increment (e.g. about 87% to about 90%, about 90.5% to about 97%, about 3.5% to about 11%, about 10% to about 14%).

The 1989 compounds mixture may comprise about 60-70 mole % of 1989 compound V1A; about 1→5 mole % of 1989 compound V1B; about 30-40 mole % of 1989 compound V2A; and about 0.1-3 mole % of 1989 compound V2B. All of the mole % can be varied by 0.1 increment (e.g. 65%, 2.5%, 35.6%).

The 1857 compounds mixture may comprise about 60-70 mole % of 1857 compound V1A; about 1→5 mole % of 1857 compound V1B; about 30-40 mole % of 1857 compound V2A; and, about 0.1-3 mole % of 1857 compound V2B. All of the mole % can be varied by 0.1 increment (e.g., 65%, 2.5%, 35.6%).

In another embodiment, the substantially pure OBI-821 is purified from a crude Quillaja saponaria extract, wherein said OBI-821 is characterized by a single predominant peak which comprises 90% or more of the total area of all peaks of a chromatogram, excluding the solvent peak, when analyzed on reverse phase-HPLC on a Symmetry C18 column having 5 um particle size, 100 Å pore, 4.6 mm ID×25 cm L with a elution program comprising mobile phase of A:B 95%:5% to 75%:25% in 11 minutes, which mobile phase A is distilled water with 0.1% trifluoroacetic acid, and mobile phase B is acetonitrile with 0.1% trifluoroacetic acid at a flow rate of 1 ml/min.

In one embodiment, the pharmaceutical composition comprises the compound of formula (I)

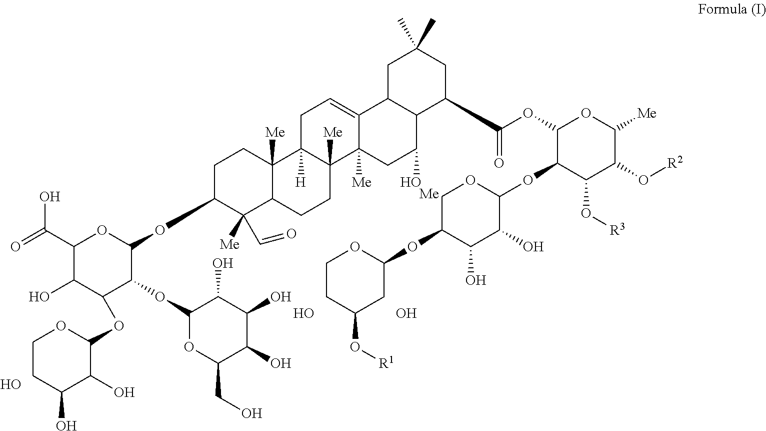

wherein,
R¹ is β-D-Apiose or β-D-Xylose; and
R² and R³ are independently H, alkyl, or

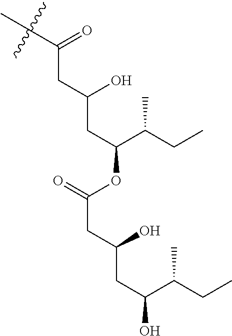 [SMILES: *C(=O)CC(O)C[C@H](OC(=O)C[C@@H](O)C[C@H](O)[C@H](C)CC)[C@H](C)CC]

(Fatty acyl moiety for the 1857 Compound), and a pharmaceutically acceptable carrier.

The vaccine can comprise a carbohydrate antigen or its immunogenic fragment and an OBI-821 saponin. In yet another embodiment, the vaccine comprises a carbohydrate antigen or its immunogenic fragment; a carrier protein and an OBI-821 saponin. In another embodiment, the vaccine comprises a carbohydrate antigen selected from Globo H, KLH, and an OBI-821 saponin. Non limiting examples of carrier protein include KLH.

The terms “a-galactosyl-ceramide” and “a-GalCer” refer to a glycolipid that stimulates natural killer T cells to produce both T helper 1 (TH1) and TH2 cytokine, as described in U.S. Pat. No. 8,268,969, the content of which is incorporate by reference in its entirety. In certain embodiment, OBI-834 (known as C34) adjuvant is characterized by the following exemplary structure:

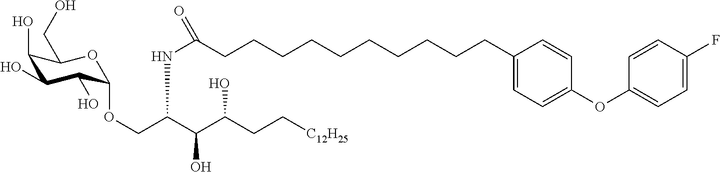

As used herein, the term “cytokine” refers to any of numerous small, secreted proteins that regulate the intensity and duration of the immune response by affecting immune cells differentiation process usually involving changes in gene expression by which a precursor cell becomes a distinct specialized cell type. Cytokines have been variously named as lymphokines, interleukins, and chemokines, based on their presumed function, cell of secretion, or target of action. For example, some common interleukins include, but are not limited to, IL-2, IL-12, IL-18, IL-2, IFN-γ, TNF, IL-4, IL-10, IL-13, IL-21, GM-CSF, and TGF-β.

As used herein, the term “chemokine” refers to any of various small chemotactic cytokines released at the site of infection that provide a means for mobilization and activation of lymphocytes. Chemokines attract leukocytes to infection sites. Chemokines have conserved cysteine residues that allow them to be assigned to four groups. The groups, with representative chemokines, are C-C chemokines (RANTES, MCP-1, MIP-1α, and MIP-1β), C—X—C chemokines (IL-8), C chemokines (Lymphotactin), and CXXXC chemokines (Fractalkine).

The therapeutic compositions of the invention can further include PD-1/PD-L1 inhibitors (cytotoxic T cell lymphocyte (CTLs) immunotherapy), CTLA-4 immunotherapy, CDK4/6 inhibitors (target therapy), PI3K inhibitors (target therapy), mTOR inhibitors (target therapy), AKT inhibitors (target therapy), Pan-Her inhibitors (target therapy). These inhibitors can be modified to generate the respective monoclonal antibody as well. Such antibodies can be included in therapeutic compositions of the invention.

The therapeutic compositions can include other anti-cancer/anti-proliferative or chemotherapeutic agents. In some embodiments, examples of such agents are found in Cancer Principles and Practice of Oncology by V. T. Devita and S. Hellman (editors), 6th edition (Feb. 15, 2001), Lippincott Williams & Wilkins Publishers. Such anti-cancer agents include, but are not limited to, the following: hormonal therapeutic agents (e.g., selective estrogen receptor modulators, androgen receptor modulators), monoclonal antibody therapy, chemotherapy, retinoid receptor modulators, cytotoxic/cytostatic agents, antineoplastic agents, antiproliferative agents, prenyl-protein transferase inhibitors, HMG-CoA reductase inhibitors, nitrogen mustards, nitroso ureas, angiogenesis inhibitors (e.g., bevacizumab), inhibitors of cell proliferation and survival signaling pathway, apoptosis inducing agents, agents that interfere with cell cycle checkpoints, agents that interfere with receptor tyrosine kinases (RTKs), mammalian target of rapamycin (mTOR) inhibitors, human epidermal growth factor receptor 2 (HER2) inhibitors, epidermal growth factor receptor (EGFR) inhibitors, integrin blockers, NSAIDs, PPAR agonists, inhibitors of inherent multidrug resistance (MDR), anti-emetic agents, agents useful in the treatment of anemia, agents useful in the treatment of neutropenia, immunologic-enhancing drugs, biphosphonates, aromatase inhibitors, agents inducing terminal differentiation of neoplastic cells, γ-secretase inhibitors, cancer vaccines, and any combination thereof.

### Formulations of the Invention

The therapeutic compositions (also referred to herein as pharmaceutical compositions) generally include a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. A pharmaceutical composition is formulated to be compatible with its intended route of administration. Examples of routes of administration include parenteral, e.g., intravenous, intradermal, subcutaneous, intramuscular, intra-arterial, oral (e.g., inhalation), transdermal (topical), transmucosal, and rectal administration. Solutions or suspensions used for parenteral, intradermal, or subcutaneous application can include the following components: a sterile diluent such as water for injection, saline solution, phosphate buffered saline, tris-buffered saline, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates and agents for the adjustment of tonicity such as sodium chloride or dextrose. The pH value can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for an injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.), or phosphate buffered saline (PBS). In all cases, the composition should be sterile and should be fluid to the extent that easy syringability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying, which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Furthermore, for oral administration, the formulations of the invention can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. The compositions of the invention can be also introduced in microspheres or microcapsules, e.g., fabricated from poly-glycolic acid/lactic acid (PGLA) (see, U.S. Pat. Nos. 5,814,344; 5,100,669 and 4,849,222; PCT Publication Nos. WO 95/11010 and WO 93/07861). Liquid preparations for oral administration can take the form of, for example, solutions, syrups, emulsions or suspensions, or they can be presented as a dry product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound.

For administration by inhalation, the compounds are delivered in the form of an aerosol spray from pressured container or dispenser which contains a suitable propellant, e.g., a gas such as carbon dioxide, or a nebulizer.

Systemic administration can also be transmucosal or transdermal. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

According to implementations, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions (including liposomes targeted to infected cells with monoclonal antibodies to cell-specific antigens) can also be used as pharmaceutically acceptable carriers. These can be prepared according to methods known to those skilled in the art, for example, as described in U.S. Pat. No. 4,522,811, which is incorporated by reference herein.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

The immunogenic formulations of the invention can be delivered parenterally, i.e., by intravenous (i.v.), subcutaneous (s.c.), intraperitoneal (i.p.), intramuscular (i.m.), subdermal (s.d.), or intradermal (i.d.) administration, by direct injection, via, for example, bolus injection, continuous infusion, or gene gun (e.g., to administer a vector vaccine to a subject, such as naked DNA or RNA). Formulations for injection can be presented in unit dosage form, e.g., in ampoules or in multi-dose containers, with an added preservative. The compositions can take such forms as excipients, suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water, before use.

The present invention also contemplates various mucosal vaccination strategies.

### Dosage Forms

Toxicity and therapeutic efficacy of such therapeutic compositions may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutic compositions which exhibit high therapeutic indices are preferred. While compounds that exhibit toxic side effects can be used, care should be taken to design a delivery system that targets such compounds to the site of affected location to minimize potential damage to uninfected cells and, thereby, reduce side effects.

Data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In the disclosed compositions, both the antigen and the adjuvant are present in immunogenically effective amounts. For each specific antigen, the optimal immunogenically effective amount should be determined experimentally (taking into consideration specific characteristics of a given patient and/or type of treatment). Generally, this amount is in the range of 0.01 μg-250 mg of an antigen per kg of the body weight. For certain exemplary adjuvant of the present invention, the immunogenically effective amount can be in the range of 10-250 μg of the adjuvant per kg of the body weight.

In some embodiments, a therapeutically effective amount of a therapeutic composition (i.e., an effective dosage) may range from about 0.001 μg/kg to about 250 g/kg, 0.01 μg/kg to 10 g/kg, or 0.1 μg/kg to 1 g/kg or about or at least: 0.001, 0.002, 0.003, 0.004, 0.005, 0.006, 0.007, 0.008, 0.009; 0.01, 0.02, 0.03, 0.04, 0.05, 0.06, 0.07, 0.08, 0.09; 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, or 250 grams or micrograms per kilogram of patient body weight, or any range between any of the numbers listed herein, or other ranges that would be apparent and understood by artisans without undue experimentation. The skilled artisan will appreciate that certain factors can influence the dosage and timing required to effectively treat a subject, including but not limited to the severity of the disease or disorder, previous treatments, the general health or age of the subject, and other diseases present.

In other embodiments, a therapeutically effective amount of Globo-H moiety in the therapeutic composition (i.e., an effective dosage) may range from about 0.001 μg/kg to about 250 g/kg, 0.01 μg/kg to 10 g/kg, or 0.1 μg/kg to 1 g/kg or about or at least: 0.001, 0.002, 0.003, 0.004, 0.005, 0.006, 0.007, 0.008, 0.009; 0.01, 0.02, 0.03, 0.04, 0.05, 0.06, 0.07, 0.08, 0.09; 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, or 250 grams or micrograms per kilogram of patient body weight, or any range between any of the numbers listed herein, or other ranges that would be apparent and understood by artisans without undue experimentation. The skilled artisan will appreciate that certain factors can influence the dosage and timing required to effectively treat a subject, including but not limited to the severity of the disease or disorder, previous treatments, the general health or age of the subject, and other diseases present.

In certain embodiments, the therapeutic compositions disclosed herein contain or are associated with, at least one therapeutic conjugate or therapeutic antibody whereby each at least one therapeutic conjugate or therapeutic antibody is present in single dose at a concentration of about, at least or more than: 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99 each times 10⁻⁹, 10⁻⁸, 10⁻⁷ 10⁻⁷ 10⁻⁶ 10⁻⁵ 10⁻⁴ 10⁻³ 10⁻² 10⁻¹ molar per dose. Preferably, the therapeutic conjugate is present in single dose at a concentration between about: 1-100, 2-60, 3-50, 4-40, 5-30, 6-20, 7-15, 8-10, 2-18, 3-16, 4-14, 5-12, 6-10 or 7-8 M, or any range between any of the numbers listed herein.

In some embodiments, the therapeutic compositions disclosed herein contain or are associated with, at least one therapeutic conjugate or therapeutic antibody whereby each at least one therapeutic conjugate or therapeutic antibody is present in single dose at a concentration of about, at least or more than: 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99 each times 10⁻³, 10⁻², 10⁻¹, or 10 micrograms. In certain embodiments about or at least 10, 20, 30, 40, 50, 60, 70, 80, 90, 100, 110, 120, 130, 140, 150, 200, 250 or more micrograms of one therapeutic conjugate or therapeutic antibody is included per dose.

In certain embodiments, the therapeutic compositions disclosed herein are administered in a dose about or at least or more than: 1, 2, 3, 4, 5, 6, 7, 8, 9, 10, 11, 12, 13, 14, 15, 16, 17, 18, 19, 20, 21, 22, 23 or 24 times per day, week or month over a period of about or at least or more than: 1, 2, 3, 4, 5, 6, 7, 8, 9, 10, 11, 12, 13, 14, 15, 16, 17, 18, 19, 20, 21, 22, 23 or 24 days, weeks, months, or years.

Effective Dose and Safety Evaluations

According to the methods of the present invention, the pharmaceutical and vaccine compositions described herein are administered to a patient at immunogenically effective doses, preferably, with minimal toxicity. As recited in the Section entitled “Definitions”, “immunogenically effective dose” or “therapeutically effective dose” of disclosed formulations refers to that amount of an antigen and/or adjuvant that is sufficient to produce an effective immune response in the treated subject and therefore sufficient to result in a healthful benefit to said subject.

### Kits

According to another aspect, one or more kits of parts can be envisioned by the person skilled in the art, the kits of parts to perform at least one of the methods herein disclosed, the kit of parts comprising one or more therapeutic conjugates, anti-cancer/anti-proliferative agents, adjuvants, cytokines and/or chemokines. The therapeutic compositions comprising alone or in combination an effective amount of the therapeutic compositions disclosed herein according to the at least one of the above mentioned methods. The aforementioned agents may come in a single container or in different containers in the kit.

The compositions may, if desired, be presented in a pack or dispenser device which may contain one or more unit dosage forms containing the active ingredient (i.e., an antigen and/or a glycosphingolipids (GSLs)—containing adjuvant). The pack may, for example, comprise metal or plastic foil, such as a blister pack. The pack or dispenser device may be accompanied by instructions for administration. Compositions of the invention formulated in a compatible pharmaceutical carrier may also be prepared, placed in an appropriate container, and labeled for treatment of an indicated condition.

The kits possibly include also identifiers of a biological event, or other compounds identifiable by a person skilled upon reading of the present disclosure. The kit can also comprise at least one composition comprising an effective amount of the therapeutic compositions disclosed herein. The therapeutic compositions of the kits to perform the at least one method herein disclosed according to procedure identifiable by a person skilled in the art.

The disclosure also includes methods of treating proliferative diseases utilizing the therapeutic compositions disclosed herein. In one embodiment, the methods involve the treatment of cancer, e.g., breast cancer. The methods generally involve providing the therapeutic compositions disclosed herein to a patient in need thereof in an amount effective to treat the proliferative disorder.

In some embodiments, the therapeutic compositions of the invention are administered to a subject in need thereof (e.g., one having a cancer such as breast cancer) in a method that on average extends progression free survival or overall survival over a control placebo, e.g., a phosphate buffered saline placebo, by about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 days, weeks, months, or years.

In some embodiments, the therapeutic compositions are given subcutaneously on week 0-2, 6, 14, and 26 in the absence of unacceptable toxicity or disease progression.

In some embodiments, the therapeutic compositions of the invention are administered to a subject in need thereof (e.g., one having a cancer such as breast cancer) in a method that on average shrinks the volume of a tumor in the patient relative to a control placebo, e.g., a phosphate buffered saline placebo, by about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 52 53 54 55 56 57 58 59 60 61 62 63 64 65 66 67 68 69 70 71 72 73 74 74 76 77 78 79 80 81 82 83 84 85 86 87 88 89 90 91 92 93 94 95 96 97 98 99 or more percent over the course of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 days, weeks, months, or years.

In some embodiments, tumors volumes may be accurately measured in at least one dimension (longest diameter in the plane of measurement is to be recorded) with a minimum size of 10 mm by CT scan (CT scan slice thickness recommended to be in between 2.5 mm and 5 mm).

### Methods of Synthesizing the Compositions of the Invention

The Globo H hexasaccharide portion of the therapeutic compositions of the invention was chemically synthesized as the allyl glycoside and then prepared for conjugation with KLH.

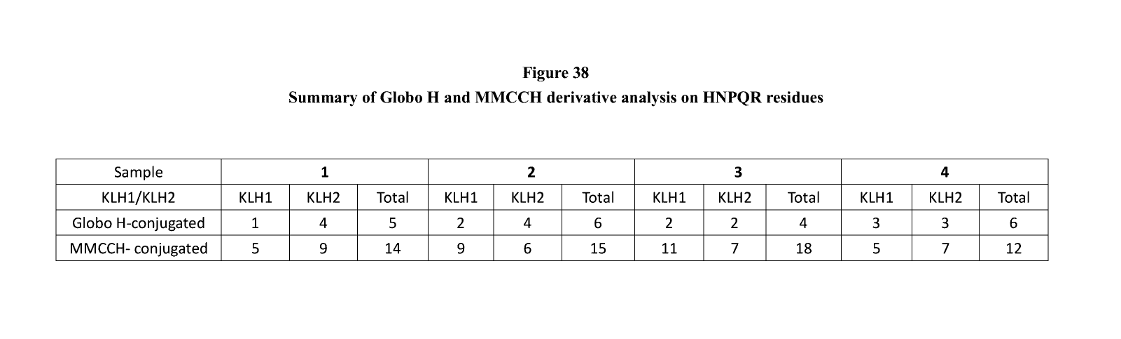

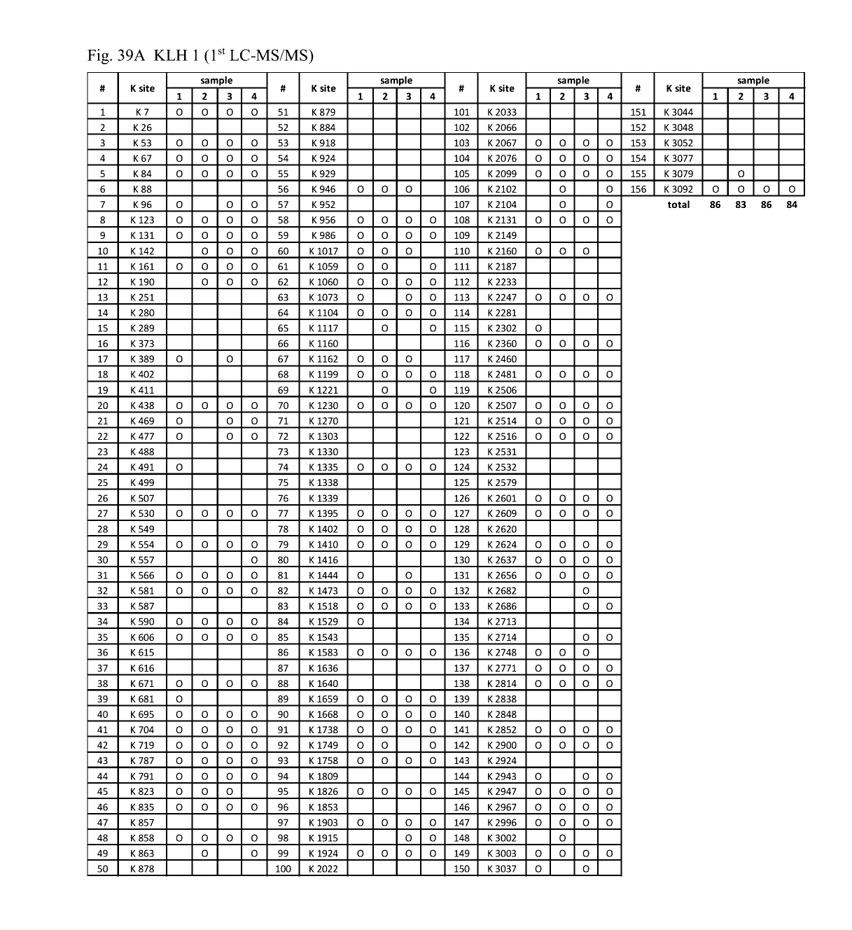

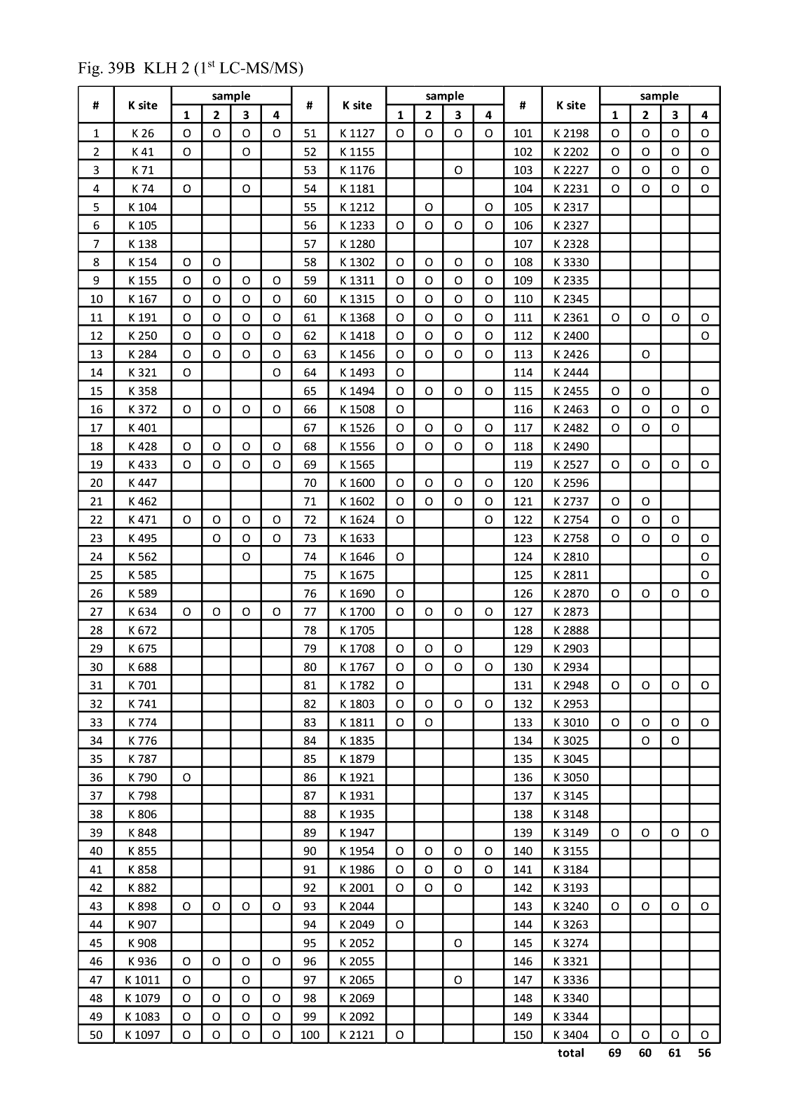

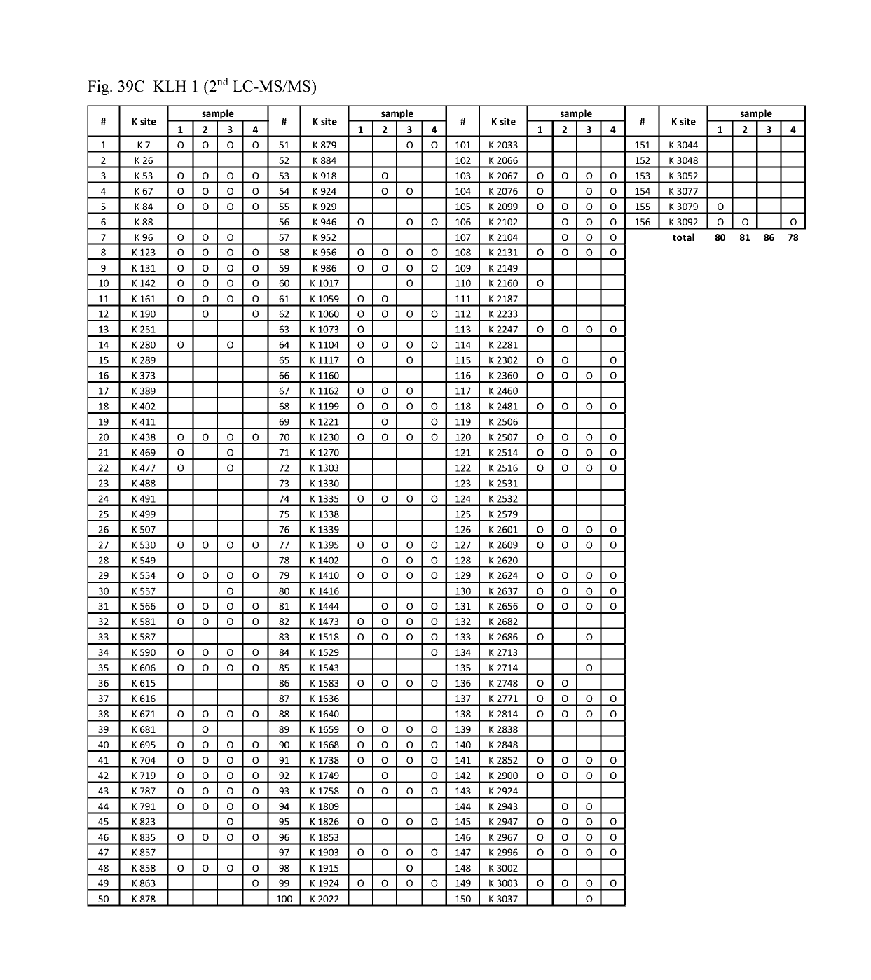

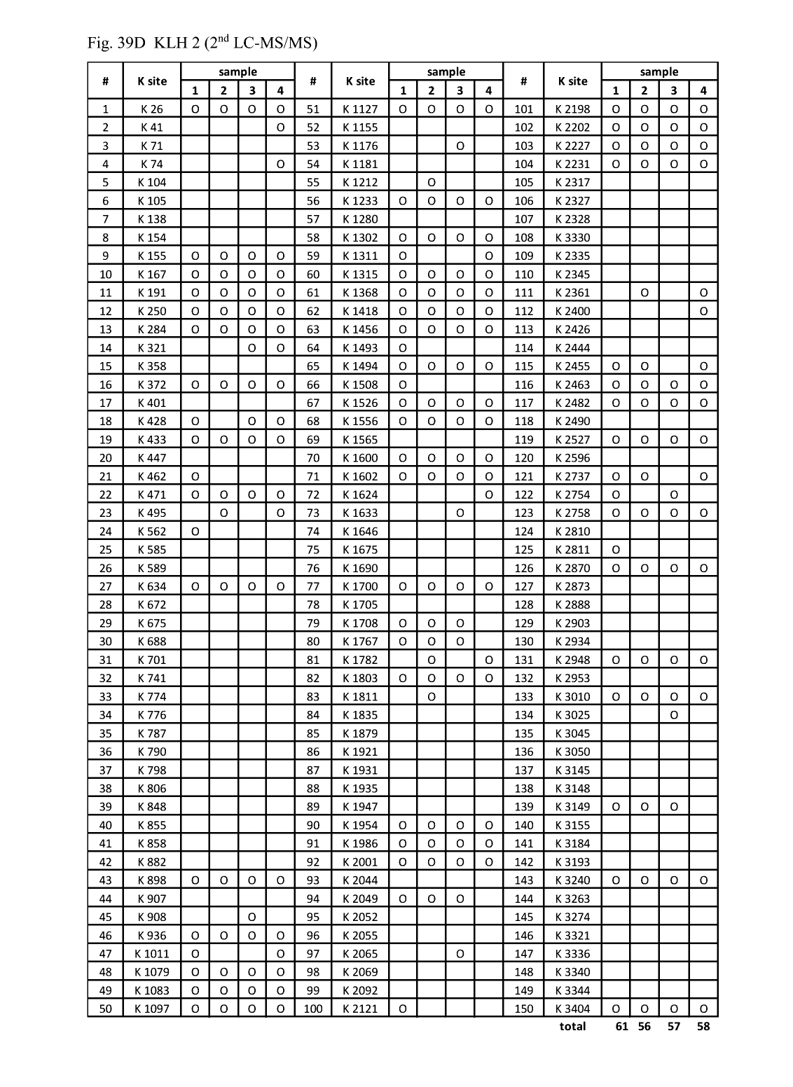

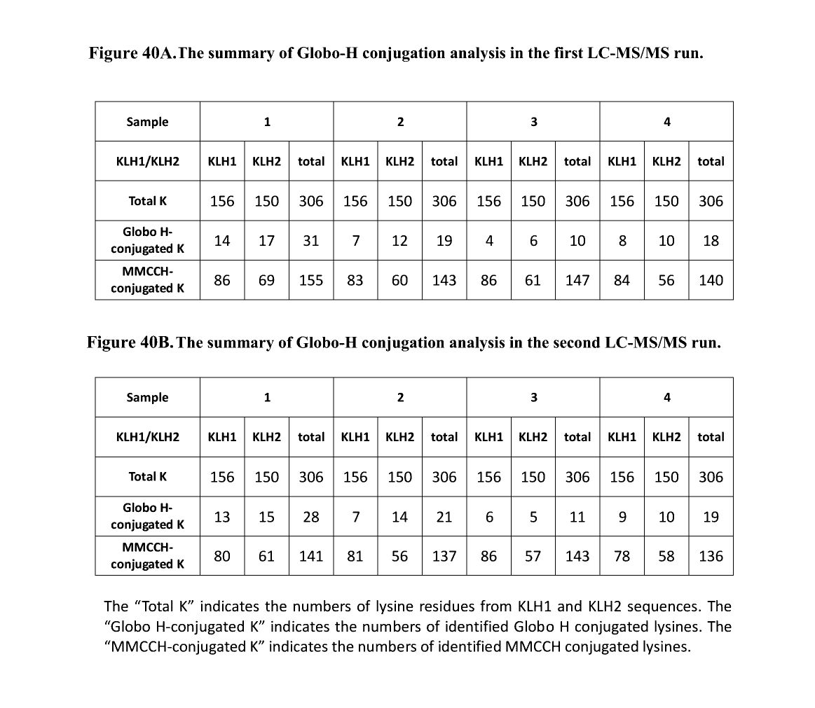

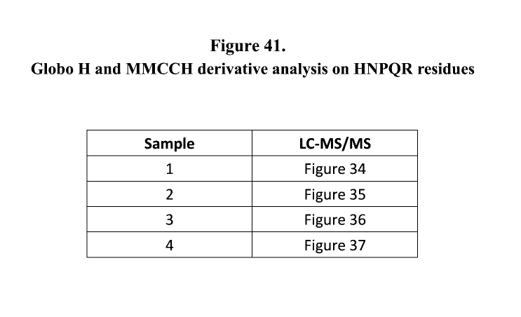

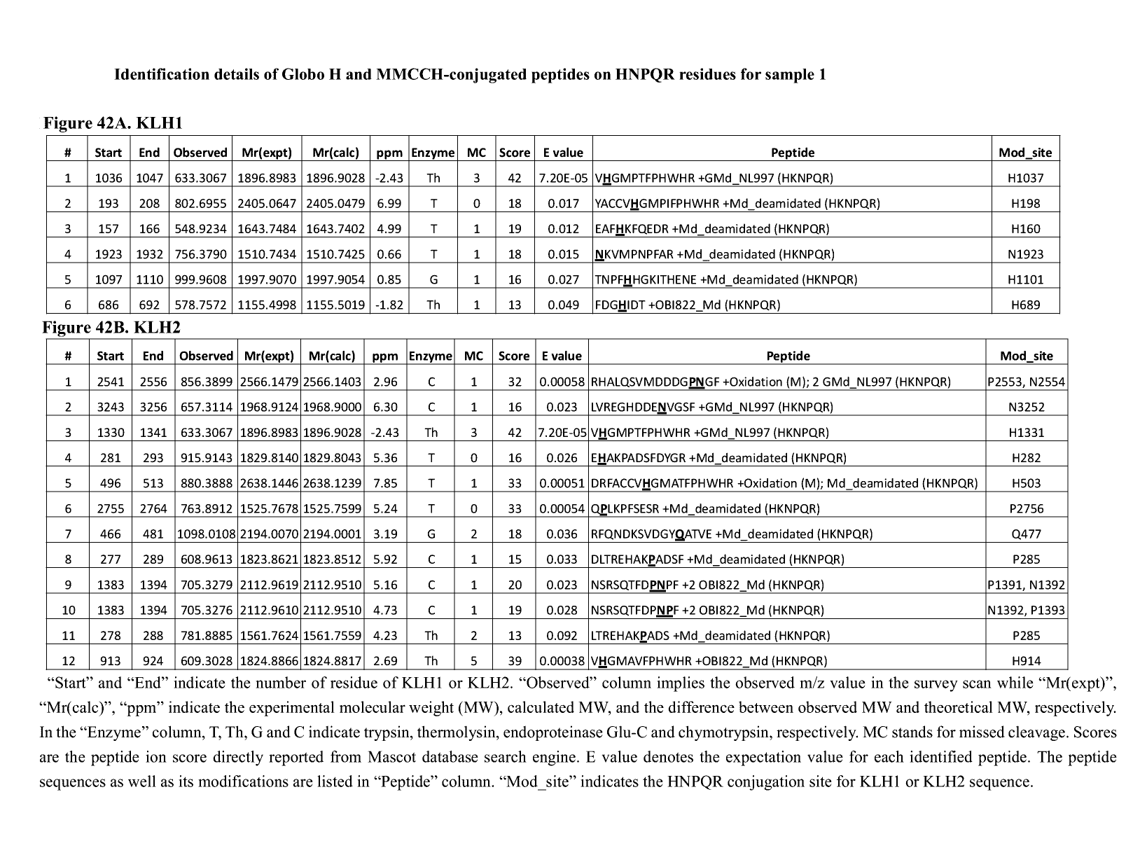

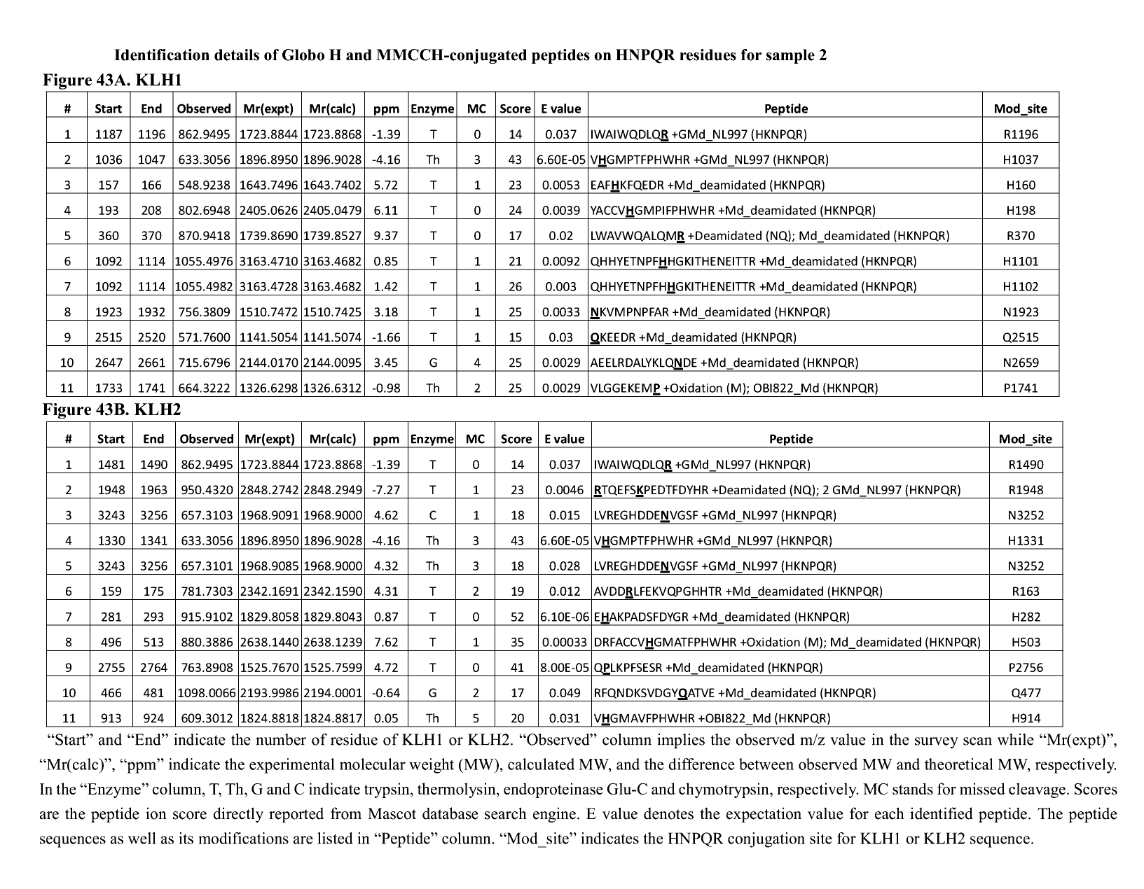

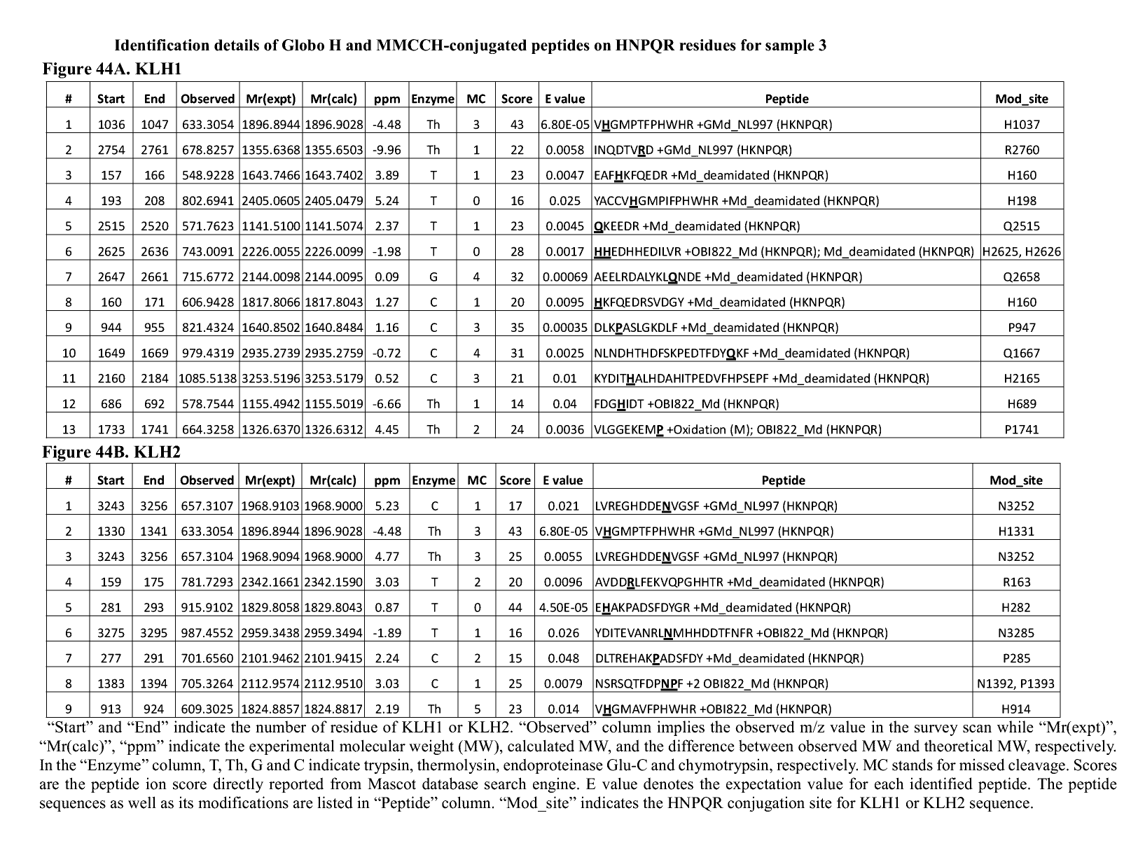

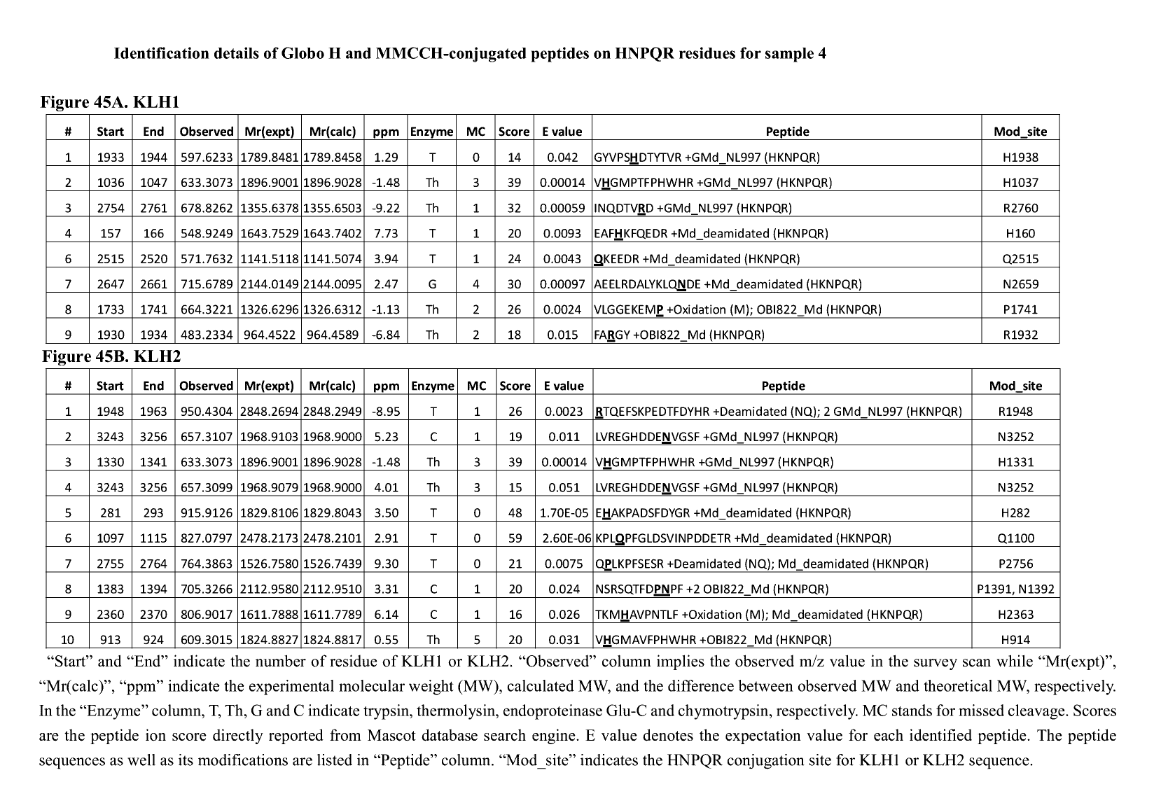

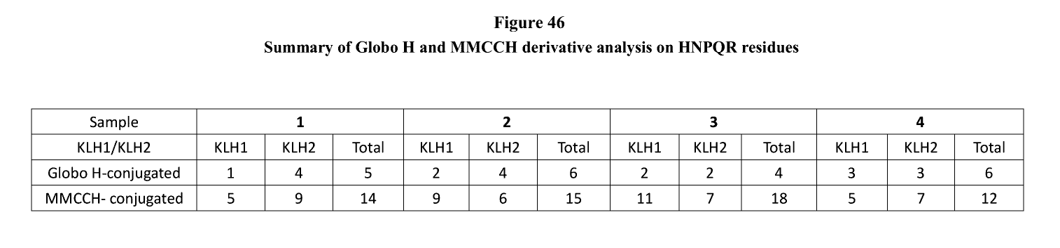

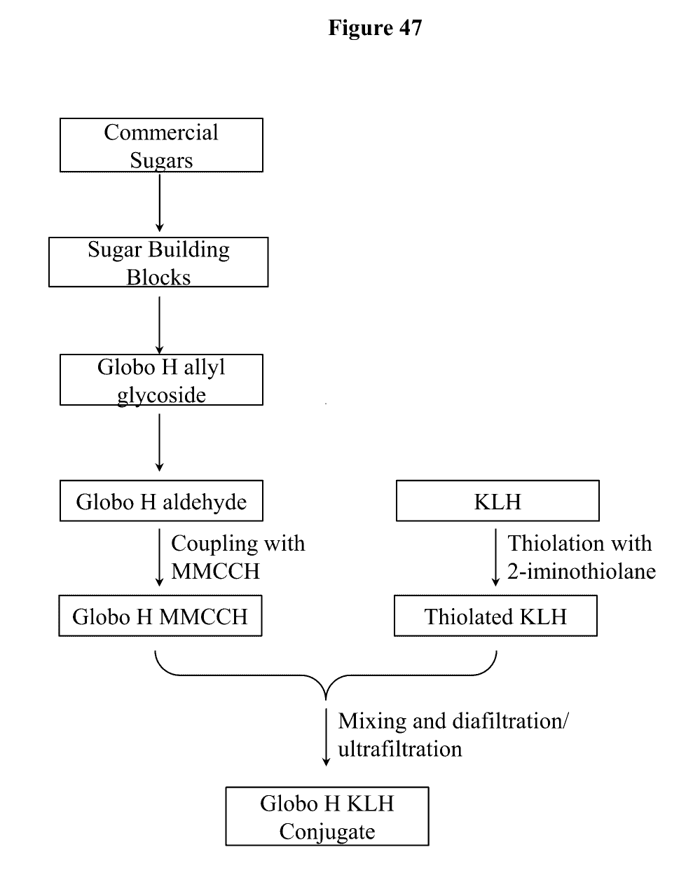

In one illustrative embodiment, the chemical synthesis of Globo H involves the general steps shown in FIG. 39.

KLH was treated with 2-iminothiolane in an aqueous buffer. The thiolated KLH was then isolated from the unreacted 2-iminothiolane, via a size exclusion column of Sephadex G-15 column. The thiolated KLH was stored under inert gas (nitrogen or argon) atmosphere and used immediately for the conjugation with Globo H-MMCCH.

### EXAMPLES

### Example 1

### Preparation of Glycoconjugate of the Invention (Globo H-KLH)

Globo H allyl glycoside (commercially available) was converted to an aldehyde by ozonolysis. Globo H aldehyde was reacted with MMCCH linker and NaCNBH₃ to give Globo H-MMCCH. The mixture was purified with a column to receive Globo H-MMCCH. The fraction with Globo H-MMCCH positive was confirmed by high performance liquid chromatography (HPLC) and then pooled together. KLH was dissolved in thiolation buffer and 2-iminothiolane was added into the reaction by portion. The reaction was incubated to completion and then KLH-SH was purified by a column. Globo H-MMCCH and KLH-SH were combined. The reaction was stirred to completion. Globo H-KLH glycoconjugate was then purified to provide the final product.

### Example 2

### Analysis of Weight Ratio of Globo H to KLH in the Glycoconjugate

The weight ratio of Globo H and KLH in the glycoconjugate as prepared was confirmed by high performance anion exchange chromatography with pulsed amperometric detection (HPAEC-PAD). The result was shown in Table 3.

TABLE 3
The weight ratio of Globo H and KLH in glycoconjugate

| Glycoconjugate epitope | Epitope ratio | Globo H Weight |
|---|---|---|
| ratio (KLH Didecamer | (KLH monomer | to KLH moiety |
| MW: 8,600,000 Da) | MW: 400 kDa) | Weight (mg/mg) |
| 3000 | 150 | 0.368 |
| 1950 | 97.5 | 0.239 |
| 1500 | 75 | 0.184 |
| 1050 | 52.5 | 0.129 |
| 300 | 15 | 0.037 |
| 100 | 5 | 0.012 |
| 20 | 1 | 0.002 |

### Example 3

### Analysis of Epitope Ratio of Globo H to KLH in the Glycoconjugate

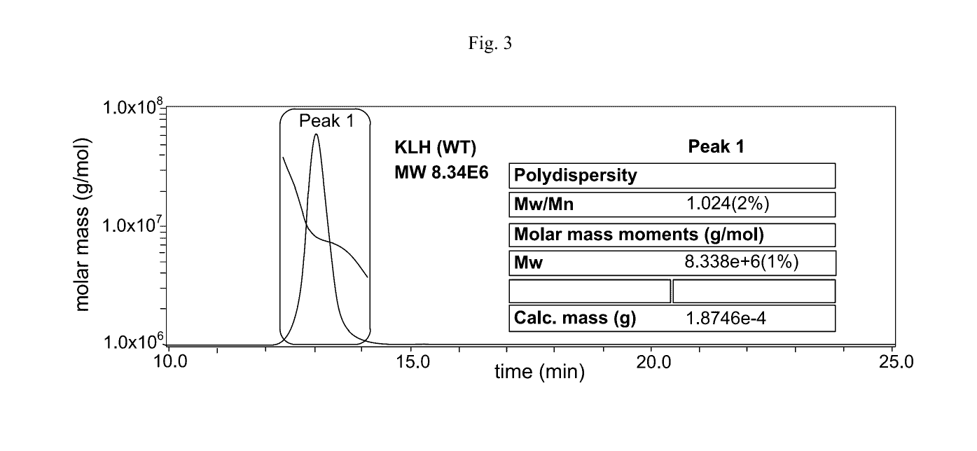

The molecular weight of a KLH didecamer (the naturally aggregated form) is around 7.5 MDa˜8.6 MDa, as described in literatures, such as Micron 30 (1999) 597-623. The native KLH was confirmed by the size exclusion chromatography and multi-angle laser scattering spectrometry (MALS), having the molecular weight of around 8.6 MDa (see FIG. 3).

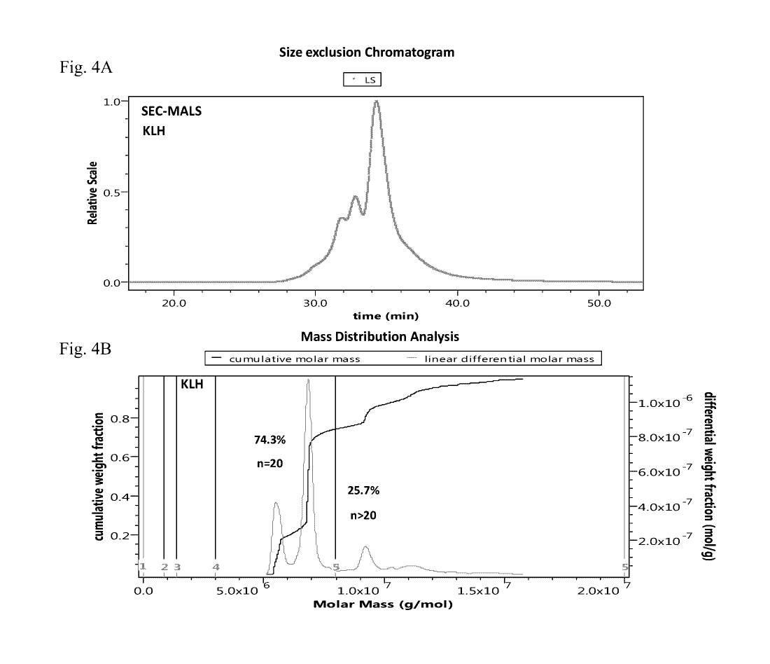

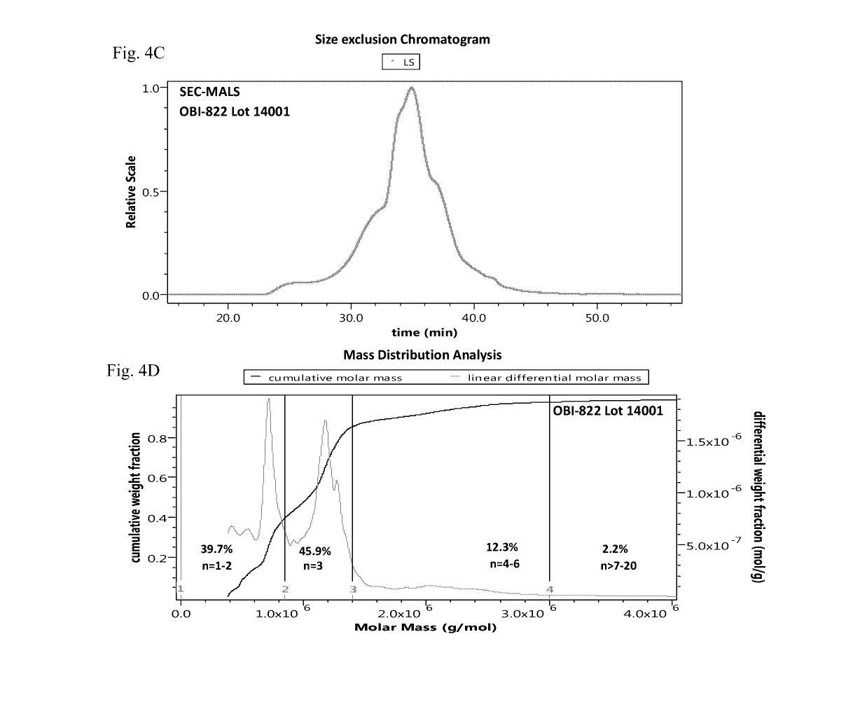

The mass distribution of KLH and Globo H-KLH glycoconjugate (OBI-822, Lot No. 14001) was estimated and derived by size exclusion chromatography using multi-angle laser scattering spectrometer (SEC-MALS). The molecular weight was calculated based on protein content (8=1.39) (see FIG. 4). In FIG. 4A, didecamer (n=20) and multi-decamer (n>20) of KLH were observed. FIG. 4B showed the peak area of didecamer was 74.3% and multi-decamer was 25.7%. In FIG. 4C, monomer to hexamer (n=1-6) and multimer (n>7-20) of Globo H-KLH glycoconjugate (OBI-822) were observed. FIG. 4D showed the peak area of monomer to hexamer (n=1-6) was the major component (>97%) and multimer (n>7-20) was only a small amount (2.2%). The summary of mass distribution analysis of KLH and OBI-822 oligomer was shown as in Table 4.

TABLE 4
The summary of mass distribution analysis of KLH and OBI-822
oligomer

| | Range 1 | Range 2 | Range 3 | Range 4 | Range 5 |
|---|---|---|---|---|---|
| Mass Distribution | (%) | (%) | (%) | (%) | (%) |
| MW Start (kDa)* | 0 | 850 | 1400 | 3000 | 8000 |
| MW Stop (kDa) | 850 | 1400 | 3000 | 8000 | 20000 |
| KLH Oligomer** | | | | Didecamer | Multi-decamer |
| | | | | n = 20 | n > 20 |
| KLH amount (%) | 0 | 0 | 0 | 74.3% | 25.7% |
| OBI-822 Oligomer*** | Monomer | Trimer | Tetramer to | Multimer | Oligomer |
| | to Dimer | n = 3 | Hexamer | n > 7-20 | n > 20 |
| | n = 1-2 | | n = 4-6 | | |
| OBI-822 Lot 14001 | 39.7% | 45.9% | 12.3% | 2.2% | 0 |
| amount (%) | | | | | |

*MW Start/Stop: Molecular Weight (MW) range in kDa
**For KLH, monomer is estimated about 350-400 kDa
***For OBI-822, monomer is estimated about 400-500 kDa

The results showed that the glycoconjugate of the invention exhibited a reduced mass in molecular weight as compared with native aggregated KLH didecamers. The molecular ratio was calculated as in Table 5.

TABLE 5
Calculation of molecular ratio of Globo H to KLH

| Experimental | Mass of 75 Globo H | | |
|---|---|---|---|
| Molecular Weight | per KLH subunit in | | Assumption of (Globo H) - |
| from Globo H-KLH | Globo H-KLH: | Mass of KLH | (KLH multimer) from the |
| (kDa) | 75 kDa | subunit: 400 kDa | experimental data |
| 8000 | 75 × 20 = 1500 | 400 × 20 = 8000 | 1500 + 8000 = 9500 |
| | | | KLH forms a Didecamer after |
| | | | Globo H-KLH conjugation. |
| 3000 | 75 × 6 = 450 | 400 × 6 = 2400 | 450 + 2400 = 2850 |
| | | | KLH forms a Hexamer after |
| | | | Globo H-KLH conjugation. |
| 1400 | 75 × 3 = 225 | 400 × 3 = 1200 | 225 + 1077 = 1425 |
| | | | KLH forms a Trimer after |
| | | | Globo H-KLH conjugation. |
| 850 | 75 × 2 = 150 | 400 × 2 = 800 | 150 + 800 = 950 |
| | | | KLH forms a Dimer after |
| | | | Globo H-KLH conjugation. |

* The above molecular ratio is calculated based on the formula as below:
$molecular ratio = \frac{Globo H Weight / Globo H M . W .}{\frac{KLH Weight}{KLH Molecular {Weight}^{ **}}}$ ** The molecular weight of KLH depends on the forms of dimer, trimer, tetramer, pentamer, hexamer to didecamer

Given the above, in certain glycoconjugate embodiments of the invention, the KLH monomeric units form monomers, dimers, trimers, tetramers, pentamers and/or hexamers after conjugation to Globo H.

### Example 4

### Preparation of Vaccine Compositions and Immunization in Rats

Different samples of the glycoconjugates (Globo H-KLH) as prepared in Example 1 were stored at 4° C., and mixed with a saponin adjuvant under a laminar flow hood. The resultant vaccine compositions were placed on ice and transported to animal facility for subsequent immunization.

Three groups of Lewis rats were immunized with the various vaccine compositions as shown in Table 6.

TABLE 6
Groups of immunized rats

| | | Animal | |
|---|---|---|---|
| Group | Vaccine compositions | number | Route |
| I | Globo H-KLH* (25 μg) + saponin | 8 | subcutaneous |
| | adjuvant (25 μg) | | |
| II | Globo H-KLH* (7.5 μg) + saponin | 4 | injection |
| | adjuvant (25 μg) | | |
| III | PBS (Phosphate Buffered Saline) | 4 | |

*Globo H:KLH = 0.17:1 (w/w)

The rats were immunized on day 0, 7, 14, and 21. Peripheral blood mononuclear cells (PBMC) and plasma were collected on day 0, 3, 10, 17, 24, and 31. Spleen, lymph node, and peritoneal wash were harvested on day 31.

### Example 5

### Assays for Induction of Humoral and Cellular Immune Responses in Rats

### Example 5.1

### Analysis of Immune Effector Cell Subpopulations by Flow Cytometry

The peripheral blood mononuclear cells (PBMCs) were isolated from the animals and then various immune effector cell subpopulations in the PBMC were identified by flow cytometry using specific antibodies against different cell markers The PBMCs, isolated from the immunized rats on day 0, 3, 10, 17, 24, and 31, were stained with different fluorescence (FITC)-conjugated antibodies and placed on ice for 30 minutes. After incubation, cells were washed with the washing buffer (1% bovine serum albumin (Sigma) and 0.1% NaN₃ (Sigma) in phosphate buffered saline (UniRegion Biotech) and centrifuged at 350 g for 5 minutes. Cells were resuspended in washing buffer for determination of fluorescence by FACS Canto (BD Bioscience). The data were analyzed with BD FACSDiva (BD Bioscience) software. The results show that in the immunized rats by the glycoconjugate of the present invention, B cells and T cells were significantly expanded when compared to the PBS control group. Specifically, the B cell population first appeared at day 3 and subsequent CD3T, CD4T and CD8T cells appeared at day 24. See FIG. 5A to FIG. 5D.

### Example 5.2

### Globo H-Specific Antibody Test by ELISA

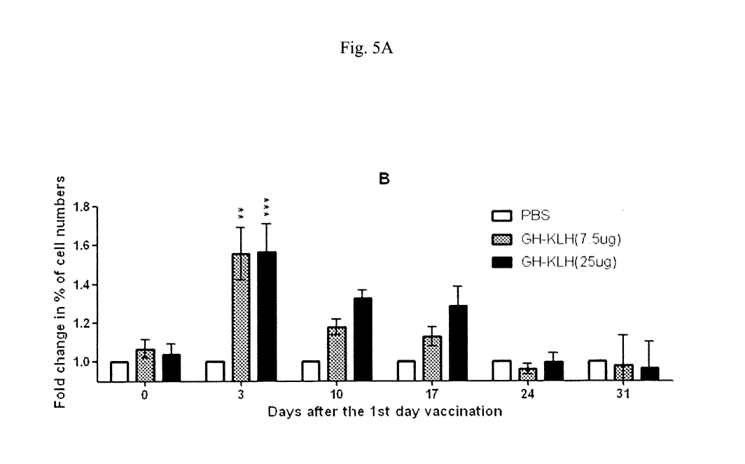

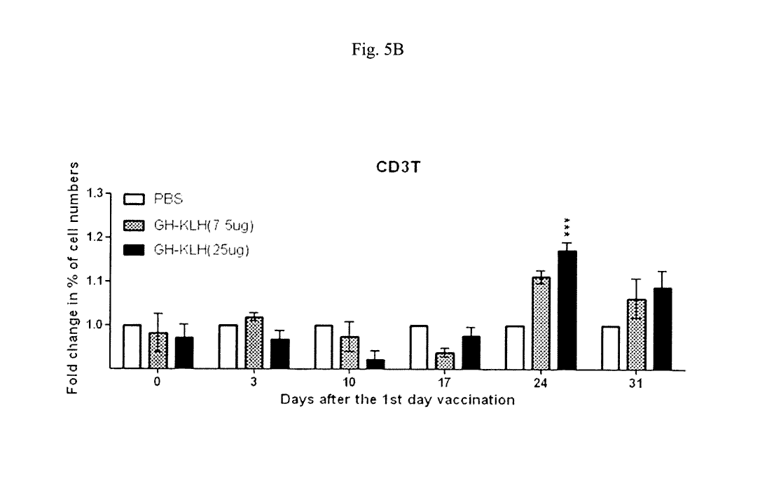

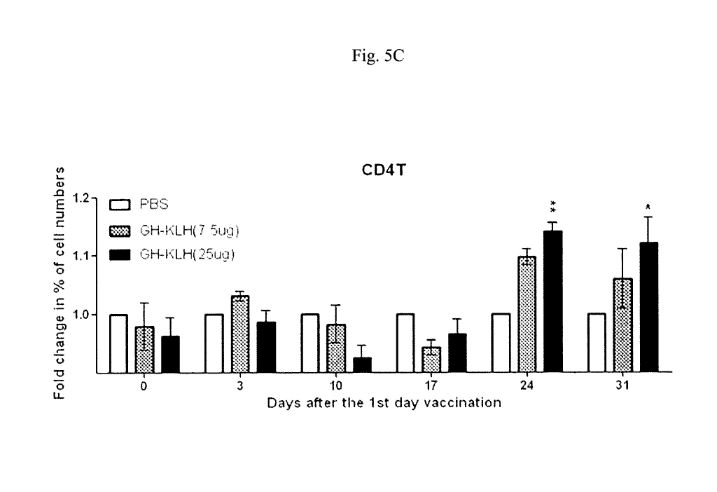

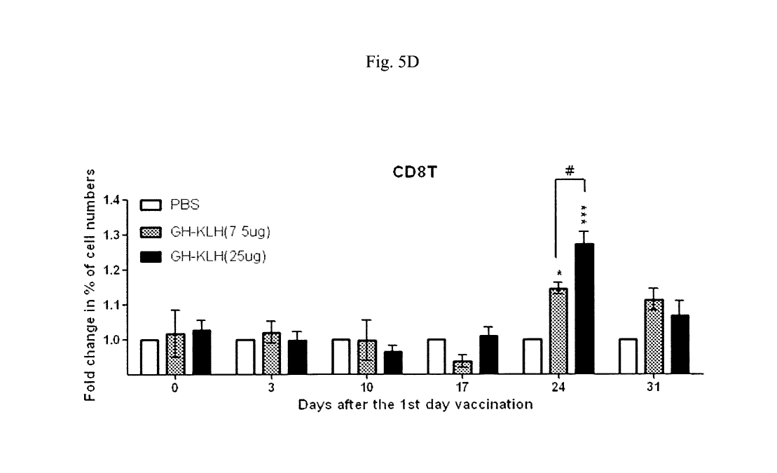

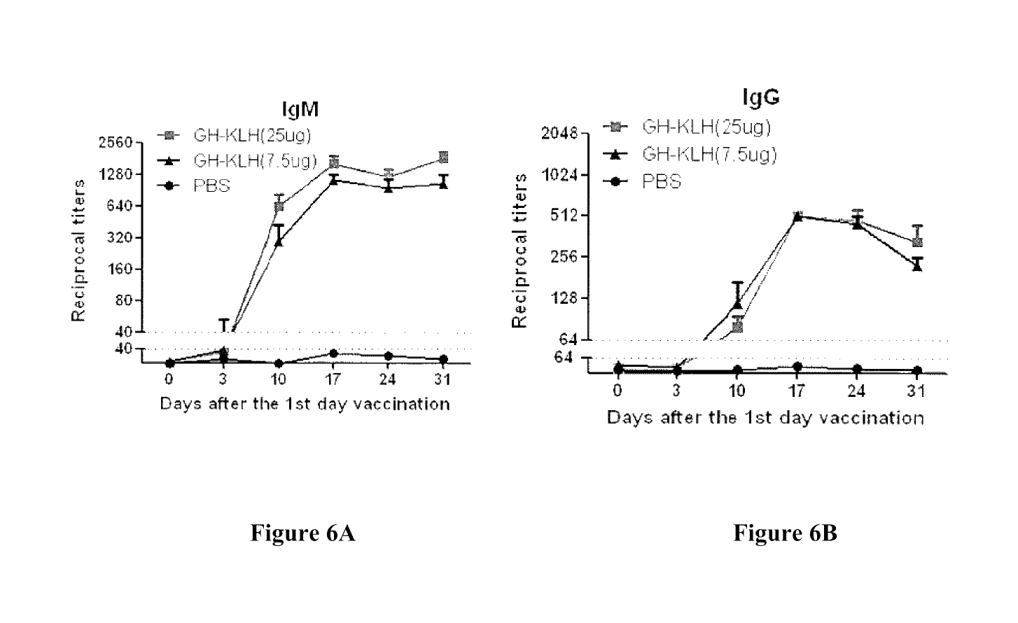

The production of Globo H-specific antibodies in the plasma from the immunized rats was determined by ELISA assay. The results showed the titers of Globo H-specific IgG began to rise at 10 days and peaked at 17 days after immunization. Similar patterns were observed in the production of Globo H-specific IgM antibody. See FIG. 6A to FIG. 6B. There was no response of Globo H-specific IgG and IgM antibody in rats treated with PBS only.

In summary, in the immunized rats, B cell production appears at day 3, followed by production of IgM and IgG antibody against Globo H which appears at day 10 and subsequent CD3T, CD4T, and CD8T cell which appears at day 24. The glycoconjugate (Globo H-KLH) of the invention was effective to induce both humoral and cellular responses.

### Example 6

### Immunization in Mice and Antibody Test by ELISA

Different samples of the glycoconjugates (Globo H-KLH) were stored at 4° C., and mixed with a saponin adjuvant under a laminar flow hood. The resultant vaccine compositions were placed on ice and transported to animal facility for subsequent immunization.

Balb/c mice of approximately eight weeks old were given Globo H-KLH glycoconjugate with different carbohydrate-to-protein (Globo H: KLH) ratios once every week for four weeks (Day 0, 7, 14, and 21) via subcutaneous injection. Blood samples were collected through retro-orbital or facial vein without anticoagulant pre-immune or Day 0, and three days after each vaccination (Day 10, 17 and 24). Samples were then centrifuged to separate sera and red cells. Sera were collected and stored at −20° C., which were later analyzed by ELISA. Mann-Whitney t-test was used for statistical analysis.

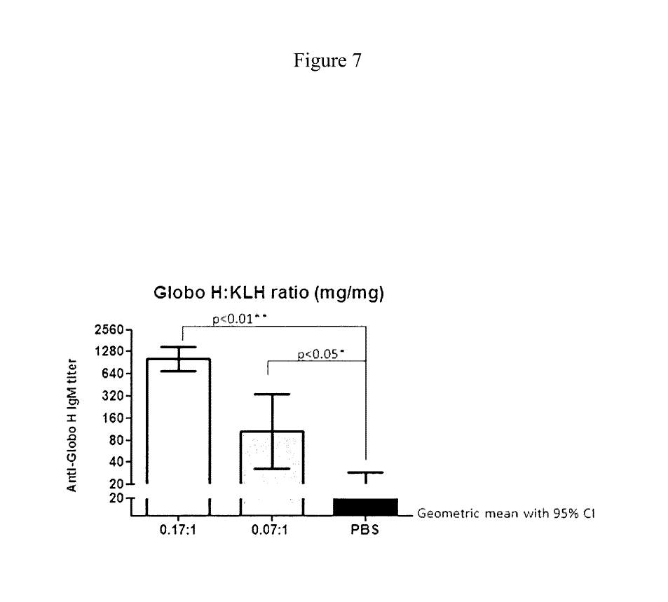

As shown in FIG. 7, the glycoconjugate (Globo H-KLH), in combination with a saponin adjuvant, according to the invention, has been demonstrated to significantly induce the Globo H-specific IgM antibody responses in the animal model, as compared with the PBS control group. Specifically, the glycoconjugate with a weight ratio of 0.17:1 (Globo H: KLH) induced a better antibody titer than the glycoconjugate with a weight ratio of 0.07:1 (Globo H: KLH).

In summary, the Globo H-KLH glycoconjugate (OBI-822), in combination with a proper adjuvant, according to the invention, has been demonstrated to induce unexpectedly superior humoral and cellular immune response in the animal model, particularly expansion of B cells and T cells including CTLs and IgM and IgG responses, which are important in cancer immunotherapy.

### Example 7

### Immunogenicity Study of Globo H-KLH with Different Adjuvants in Mice

The ability of Globo H composition of the invention, when paired with different adjuvants (OBI-821/OBI-834), to elicit an immune response in mice has been performed. Viable Lewis Lung carcinoma (LL/2, ATCC CRL-1642) cells (provided from Eurofins Panlabs Taiwan Ltd., 5.0×10⁶ in 0.1 mL), syngeneic for C57BL/6 mice, were injected subcutaneously into the abdominal region toward the lateral side of experimental mice on day 16. Six groups of 6-8 weeks old female C57BL/6 mice were immunized subcutaneously with Globo H-KLH (OBI-822) and different adjuvants. OBI-822 dose levels were the equivalent of amount in each group. Each injection contained a doses equivalent to 2.0 μg vaccine (OBI-822) and 20 μg adjuvants (OBI-821/OBI-834) administered subcutaneously in 0.2 mL/mouse (at both left and right abdominal sties, 0.1 mL/site). The immunizations occurred on Days 0, 5, 11, 19, 29, 34 and 39, and serum was collected on Day 0 (pre-injection) and Day 42 for comparative analysis by ELISA. Serological responses were measured by ELISA to determine the titer of IgM and IgG antibodies against Globo H.

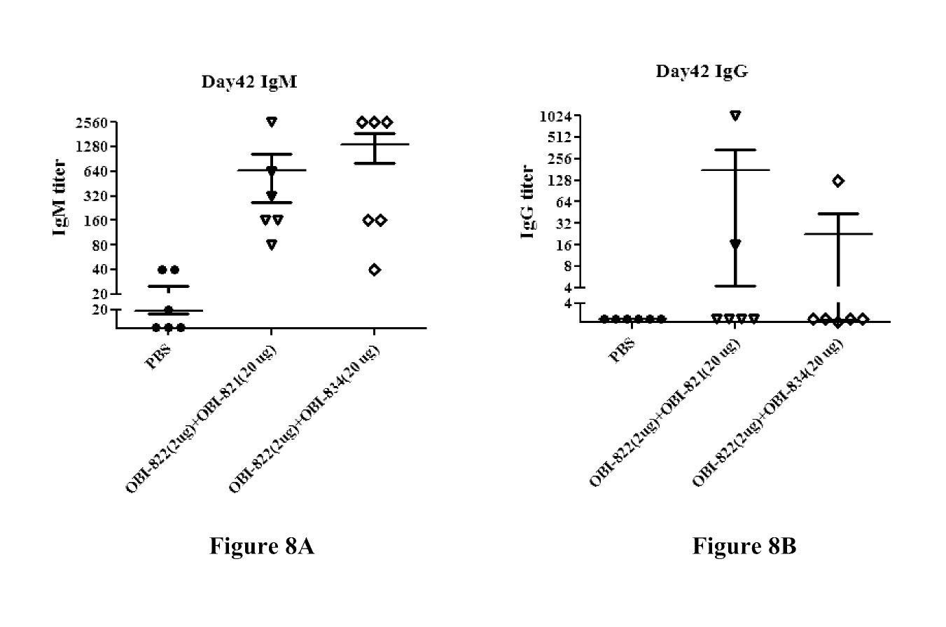

As shown in FIG. 8, there was no response in mice treated with Phosphate Buffered Saline (PBS). In contrast, the mice treated with OBI-822+OBI821 and OBI-822+OBI-834 responded with significant IgM anti-Globo H titers (FIG. 8A). However, the average IgG titers were lower than IgG (FIG. 8B). It indicated that both OBI-821 and OBI-834 adjuvants could induce the immunogenicity of Globo H-KLH (OBI-822). Vaccination with Globo H-KLH and an adjuvant has demonstrated to elicit both IgG and IgM anti-Globo H responses in female C57BL/6 mice.

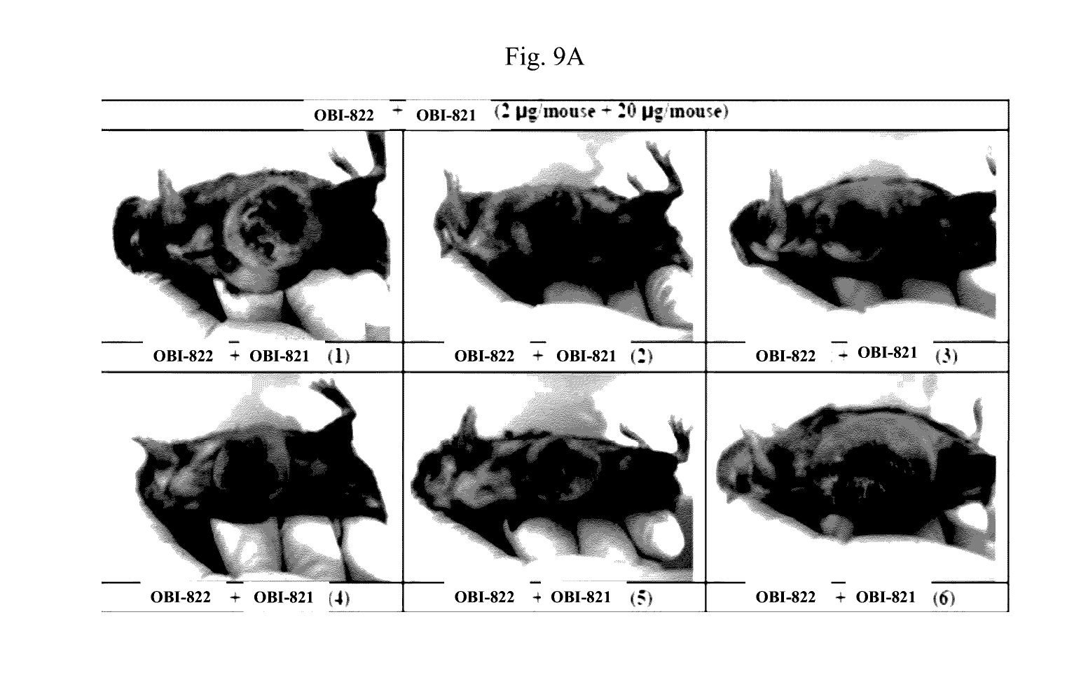

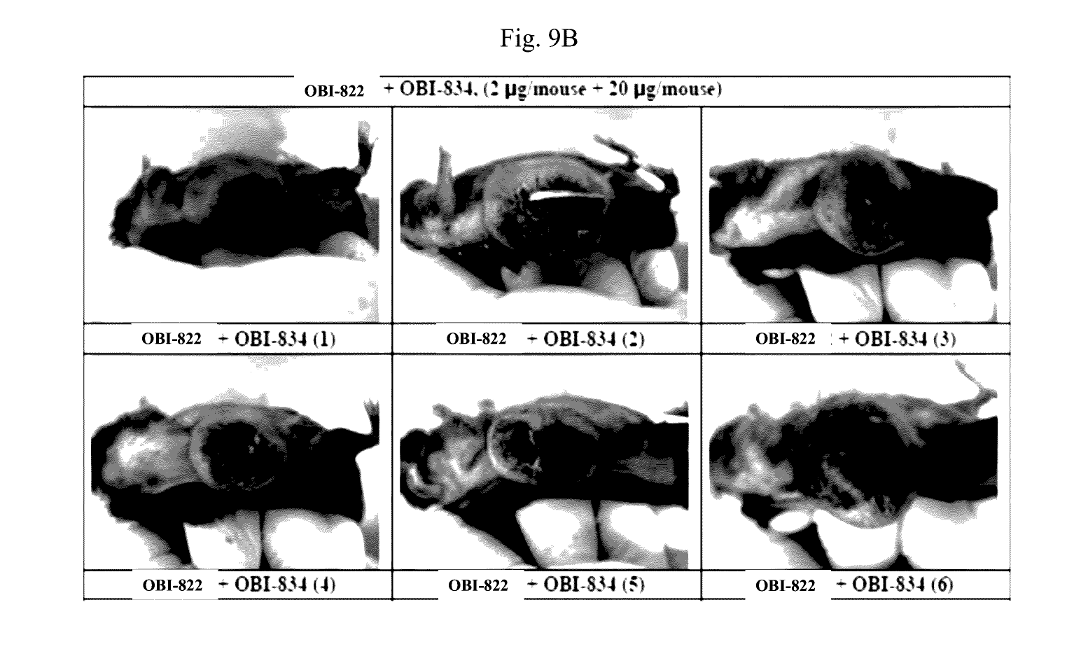

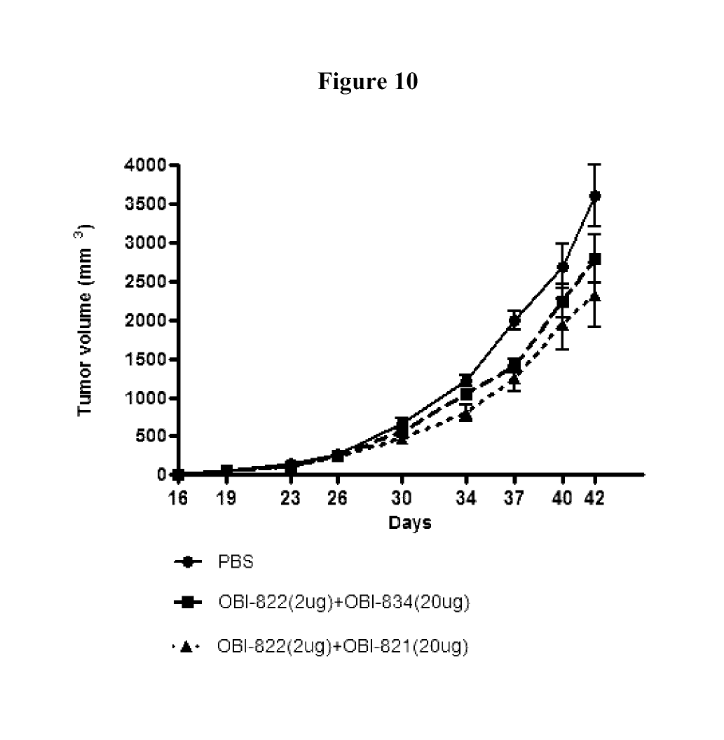

As shown in FIG. 9, the six groups of C57BL/6 mice were monitored for body weight and tumor size on days 16, 19, 23, 26, 30, 34, 37, 40 and 42. The pictures including tumor with whole body were taken after sacrifice on day 42. As shown in FIG. 10, tumor volume (mm³) is estimated according to the formula for a prolate ellipsoid: length (mm)×[width (mm)]²×0.5. Tumor growth in compound treated animals is calculated as T/C (Treatment/Control)×100%; one-way ANOVA followed by Dunnetts test or unpaired Student's t-test was used to determine the significant inhibition in tumor growth demonstrating antitumor activity. The tumor inhibition rate on day 42 of OBI-822+OBI-821 was about 35% and OBI-822+OBI-834 was about 22%. It seemed that both OBI-821 and OBI-834 adjuvants could inhibit tumor growth with Globo H-KLH (OBI-822).

### Example 8

### LC-MS/MS Analysis of Globo-H Conjugation Sites (lysine) on KLH

Globo H conjugation sites in four KLH samples using multiple enzyme digestion and LC-MS/MS were identified. The four Globo H-conjugated KLH samples were first digested with four different enzymes and then analyzed by LC-MS/MS and Mascot database search. Two types of derivatives were identified: Globo H derivative (Globo H+MMCCH) and the MMCCH derivative (MMCCH alone). The Globo H derivative and its neutral loss forms were taken into account for Globo H conjugation site identification. The MMCCH form and its deamidated form were taken into account for MMCCH conjugation site identification. Only those peptides with high quality MS/MS spectra and Mascot score were taken into account. For Globo H conjugation analysis, 31 and 28 conjugated lysines were observed from the two replicate LC-MS/MS analyses of OBI-822-13001-DP (sample 1); 19 and 21 conjugated lysines were observed from the two replicate LC-MS/MS analyses of OBI-822-13002-DP (sample 2); 10 and 11 conjugated lysines were observed from the two replicate LC-MS/MS analyses of OBI-822-13003-DP (sample 3); 18 and 19 conjugated lysines were observed from the two replicate LC-MS/MS analyses of OBI-822-13004-DS (sample 4). For MMCCH conjugation analysis, 155 and 141 conjugated lysines were found from the two replicate LC-MS/MS analyses of OBI-822-13001-DP (sample 1); 143 and 137 conjugated lysines were found from the two replicate LC-MS/MS analyses of OBI-822-13002-DP (sample 2); 147 and 143 conjugated lysines were found from the two replicate LC-MS/MS analyses of OBI-822-13003-DP (sample 3); 140 and 136 conjugated lysines were found from the two replicate LC-MS/MS analyses of OBI-822-13004-DS (sample 4).

### Example 8

### Materials and Methods

The abbreviations were listed as follows:

### K=Lysine; LC-MS/MS=Liquid Chromatography-Tandem Mass Spectrometry;

DTT=Dithiolthreitol; IAM=Iodoacetamide; ACN=Acetonitrile; FA=Formic Acid; Glu-C=Endoproteinase Glu-C; ABC=Ammonium bicarbonate; RT=Room temperature; MW=Molecular weight.

The four KLH samples, sample 1-4, were first processed for buffer exchange into 50 mM ammonium bicarbonate buffer solution by 100 kDa cut-off Amicon Ultra Centrifugal Filters and denatured with 6 M urea. The samples were then reduced with 10 mM DTT at 37° C. for 1 hr, alkylated using 50 mM IAM for 30 mins in dark at RT and quenched with 50 mM DTT at RT for 5 mins. The resulting proteins were diluted until the urea concentration is 1 M and subjected to in-solution digestion with different enzymes as described in the following section.

In-solution digestion with different enzymes was performed with the following digestion conditions: (1) trypsin digestion at 37° C. for 24 hrs (protein: enzyme=40:1) (2) Glu-C digestion at 37° C. for 24 hrs (protein: enzyme=25:1); (3) chymotrypsin digestion at RT for 24 hrs (protein: enzyme=25:1); (4) thermolysin digestion at 37° C. for 24 hrs (protein: enzyme=25:1).

Digestion reactions were terminated by adding formic acid and all four digested samples were subjected to LC-MS/MS analysis. Samples were analyzed with Q Exactive mass spectrometer (Thermo Scientific) coupled with Ultimate 3000 RSLC system (Dionex). The LC separation was performed using the C 18 column (Acclaim PepMap RSLC, 75 μm×150 mm, 2 μm, 100 Å) with the gradient shown in Table 7.

TABLE 7
The LC separation gradient

| | | | Flow |
|---|---|---|---|
| Time (min) | Mobile phase A* % | Mobile phase B** % | (μL/min) |
| 0 | 99 | 1 | 0.25 |
| 5 | 99 | 1 | 0.25 |
| 35 | 90 | 10 | 0.25 |
| 67 | 65 | 35 | 0.25 |
| 77 | 15 | 85 | 0.25 |
| 82 | 99 | 1 | 0.25 |
| 90 | 99 | 1 | 0.25 |

*Mobile phase A: 5% Acetonitrile/0.1% Formic acid
**Mobile phase B: 95% Acetonitrile/0.1% Formic acid

In-source CID was set at 45 eV. Full MS scan was performed with the range of m/z 350-2000, and the ten most intense ions from MS scan were subjected to fragmentation for MS/MS spectra. Raw data were processed into peak lists by Proteome Discoverer 1.4 for Mascot database search.

Database search was performed with Mascot version 2.4.1 and Thermo Proteome Discoverer version 1.4 against the KLH1 sequence (SEQ ID NO. 1) and KLH2 sequence (SEQ ID NO. 2). The parameters used were as follows:

Enzyme: Trypsin, Glu-C, Chymotrypsin and Thermolysin according to the digestion method; Fixed modification: Carbamidomethyl (C);

Variable modifications for MMCCH derivatives (MMCCH alone): Deamidated (NQ), Oxidation (M), dK_MMCCH-1 (K), dK_MMCCH-2 (K);

Variable modifications for Globo H derivatives (Globo H+MMCCH): Deamidated (NQ), Oxidation (M), Globo_H_MMCCH (K), dK_MMCCH_NL997 (K), dK_MMCCH_NL835 (K), dK_MMCCH_NL673 (K), dK_MMCCH_NL511 (K), dK_MMCCH_NL308 (K);

Peptide Mass Tolerance: ±10 ppm; Fragment Mass Tolerance: ±0.05 Da; Max Missed Cleavages: 5; Instrument type: ESI-TRAP; Ion cut-off score: 13.

“dK_MMCCH-1” in the search parameters indicates the MMCCH-conjugated lysine with the MW addition of 352.1569 Da.

“dK_MMCCH-2” in the search parameters indicates the deamidated form of MMCCH-conjugated lysine with the MW addition of 338.1300 Da.

“Globo_H_MMCCH (K)” in the search parameters indicates the Globo H-conjugated lysine with the MW addition of 1393.5317 Da.

“dK_MMCCH_NL997” in the search parameters indicates the neutral loss form of Globo H-conjugated lysine with the MW addition of 396.1831 Da.

“dK_MMCCH_NL835” in the search parameters indicates the neutral loss form of Globo H-conjugated lysine with the MW addition of 558.2360 Da.

“dK_MMCCH_NL673” in the search parameters indicates the neutral loss form of Globo H-conjugated lysine with the MW addition of 720.2888 Da.

“dK_MMCCH_NL511” in the search parameters indicates the neutral loss form of Globo H-conjugated lysine with the MW addition of 882.3416 Da.

“dK_MMCCH_NL308” in the search parameters indicates the neutral loss form of Globo H-conjugated lysine with the MW addition of 1085.4210 Da.

The “MW addition” implies the molecular weight addition compared to intact lysine residue.

### Example 8

### Results

LC-MS/MS based techniques have emerged as an important tool for identification of protein and characterization of amino acid modification. Detailed information regarding peptide sequences and modification sites can be obtained by the assignment of fragment ions provided by MS/MS spectra. Mascot is a search engine and its probability based scoring algorithm has been well accepted. Mascot score was adopted in this study as a reference of confidence for protein sequencing and Globo H or MMCCH conjugation site identification. To extensively analyze the distribution of Globo H or MMCCH conjugation sites in sample 1-4, these samples were digested with multiple enzymes followed by LC-MS/MS analyses.

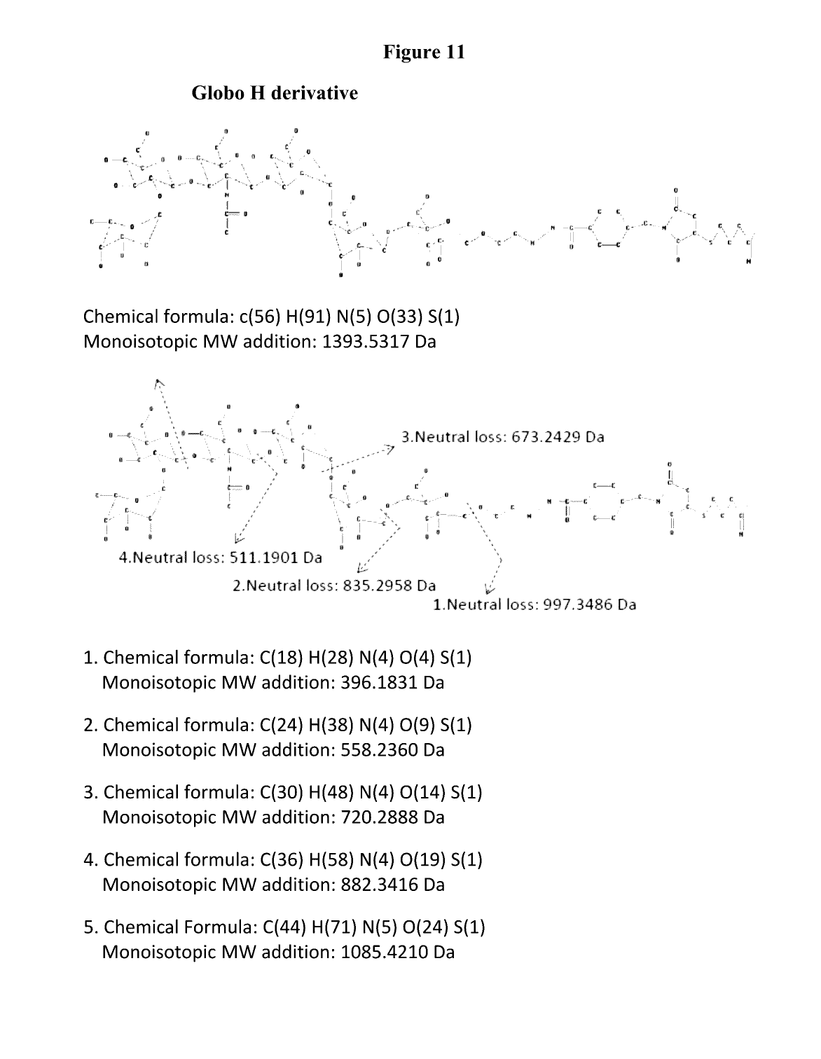

The expected chemical structure for Globo H derivative (Globo H+MMCCH) was shown in FIG. 11A and the corresponding MW addition of 1393.53 Da on lysine-containing peptides can be observed among the results. Besides, the labile polysaccharides tend to fall off via neutral loss during the electrospray ionization in LC-MS analysis. Therefore, the molecular weight addition of 396.18 Da, 558.24 Da, 720.29 Da, 882.34 Da and 1085.42 Da resulted from neutral loss can also be observed for glycoconjugated peptides, as shown in FIG. 11B. All the derivitization forms were considered for the identification of Globo H conjugation sites.

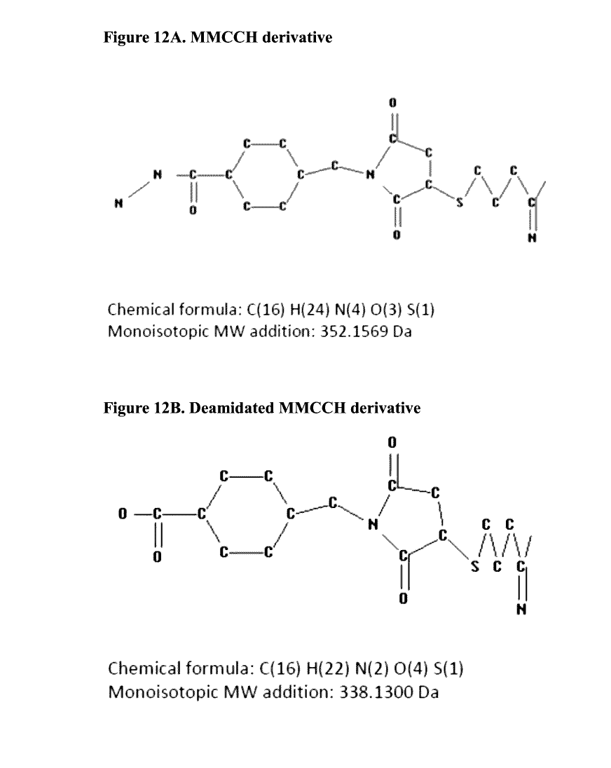

In addition, the MMCCH derivative is also observed (MMCCH alone) in these Globo H conjugated KLH samples. The expected chemical structure for MMCCH derivative and its deamidated form were shown in FIG. 12A and FIG. 12B and the corresponding MW addition of 352.16 Da and 338.13 Da respectively on lysine-containing peptides can be observed among the results. Both derivitization forms were considered for the identification of MMCCH conjugation sites. The conversion from Globo H derivative to MMCCH is not clear but it is supposed to happen during the sample treatment.

The mass accuracy of ±10 ppm for precursor ion and ±0.05 Da for fragment ion were used as the criteria for protein identification and spectra interpretation. The Globo H derivative as well as its neutral loss forms was chosen as variable modification for Globo H conjugation site identification, and MMCCH derivative as well as its deamidated form was chosen as variable modification for MMCCH conjugation site identification. Database search was performed against the sequence of KLH1 (SEQ ID NO. 1) and KLH2 (SEQ ID NO. 2). Only those peptides with high quality MS/MS spectra (ion score≧13, p<0.05) were listed in the report.

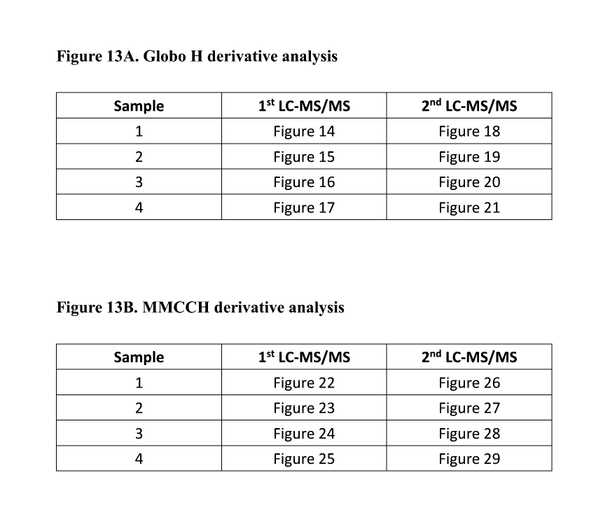

To demonstrate a repeatable result, the LC-MS/MS analysis was performed twice followed by individual Mascot database search for both Globo H conjugation site identification and MMCCH conjunction site identification, as summarized in FIG. 13A and FIG. 13B.

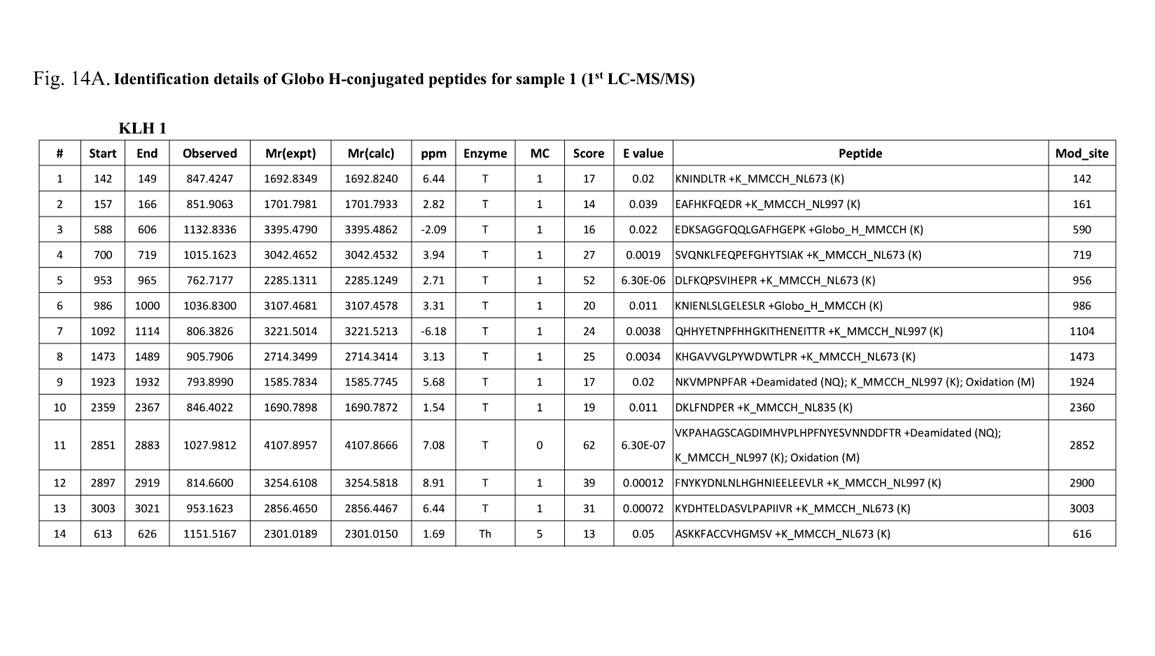

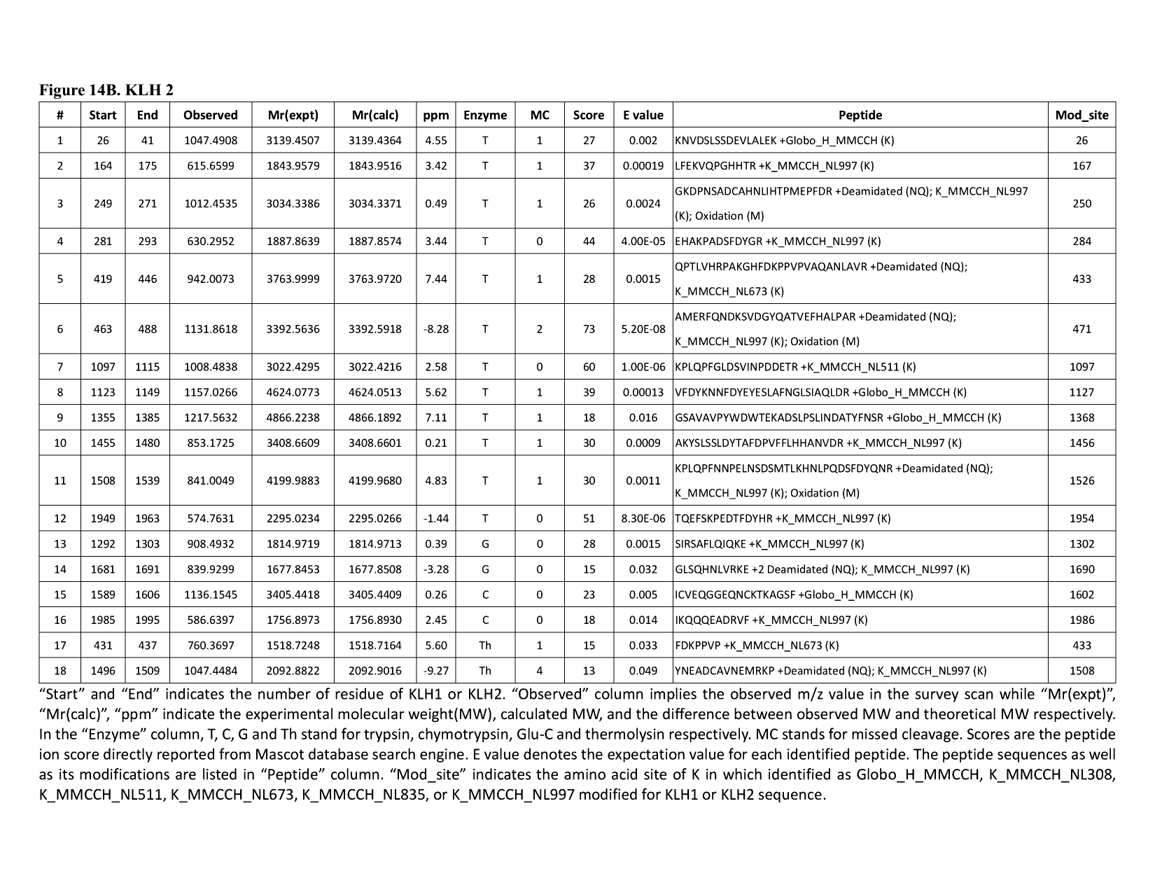

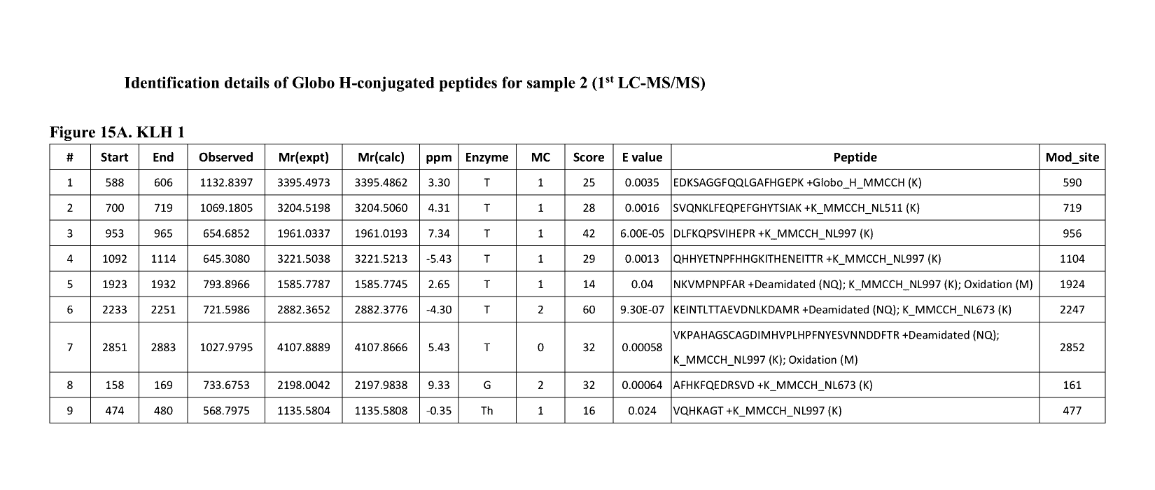

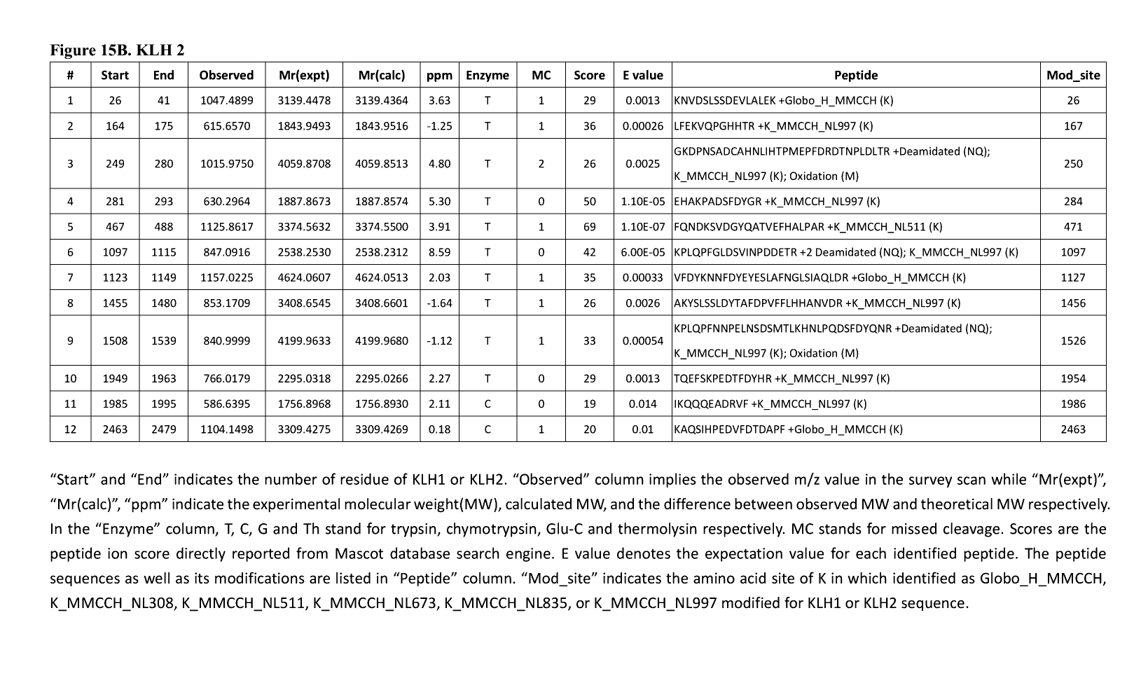

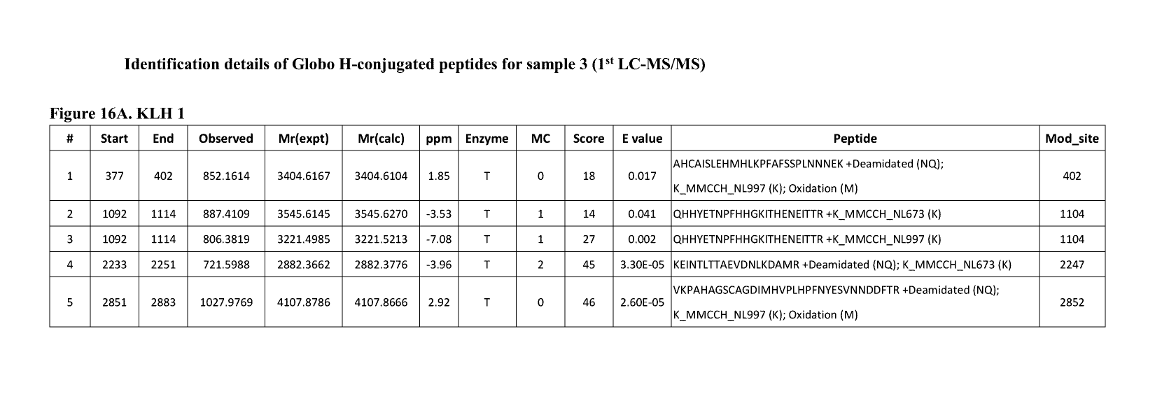

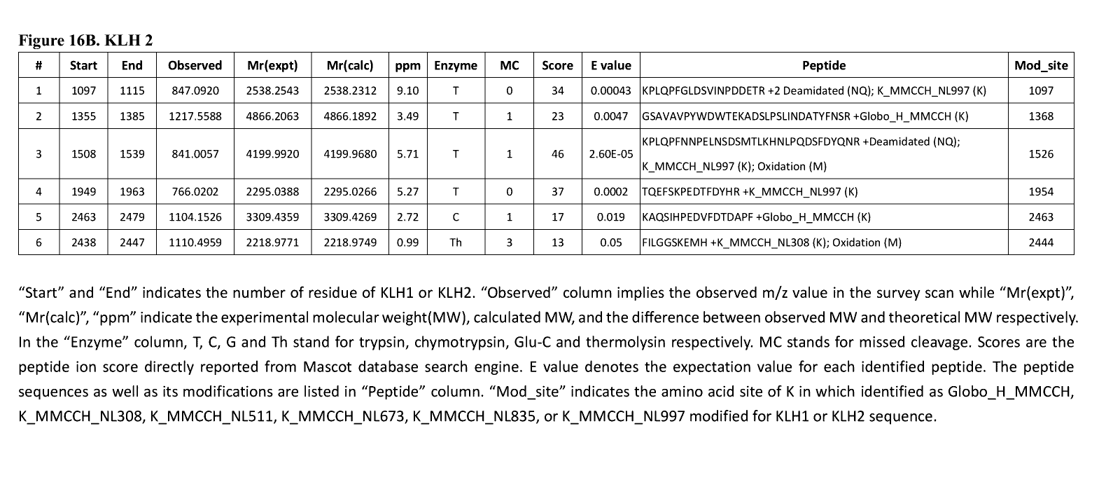

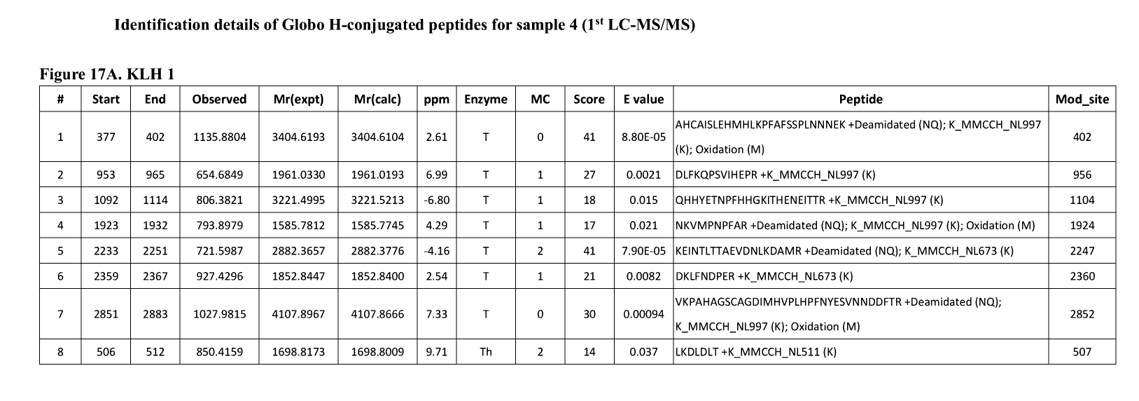

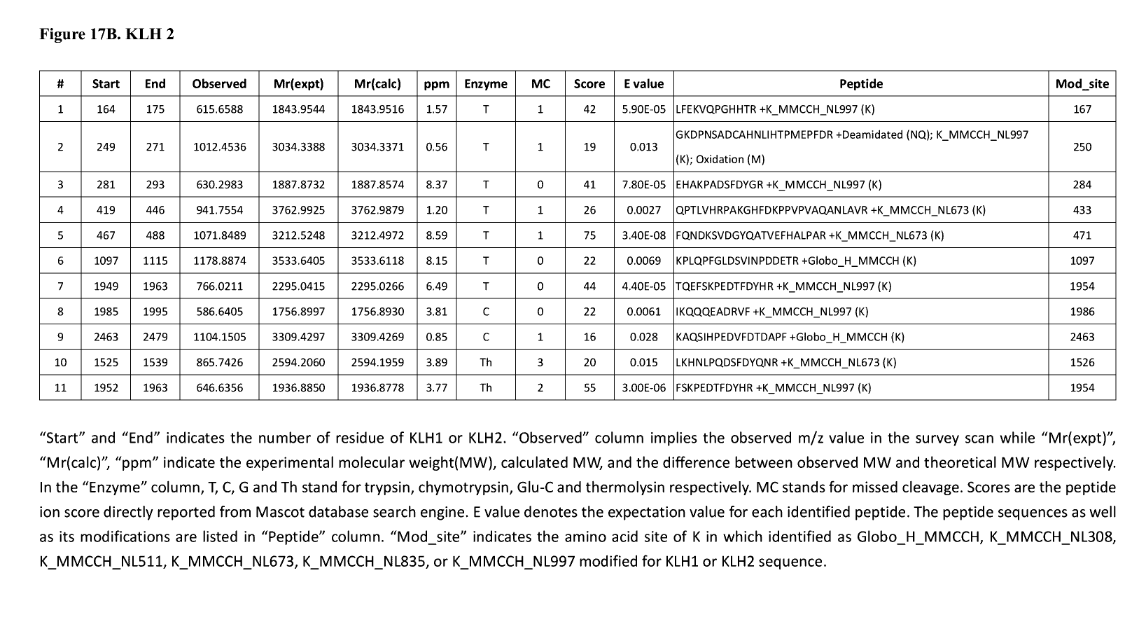

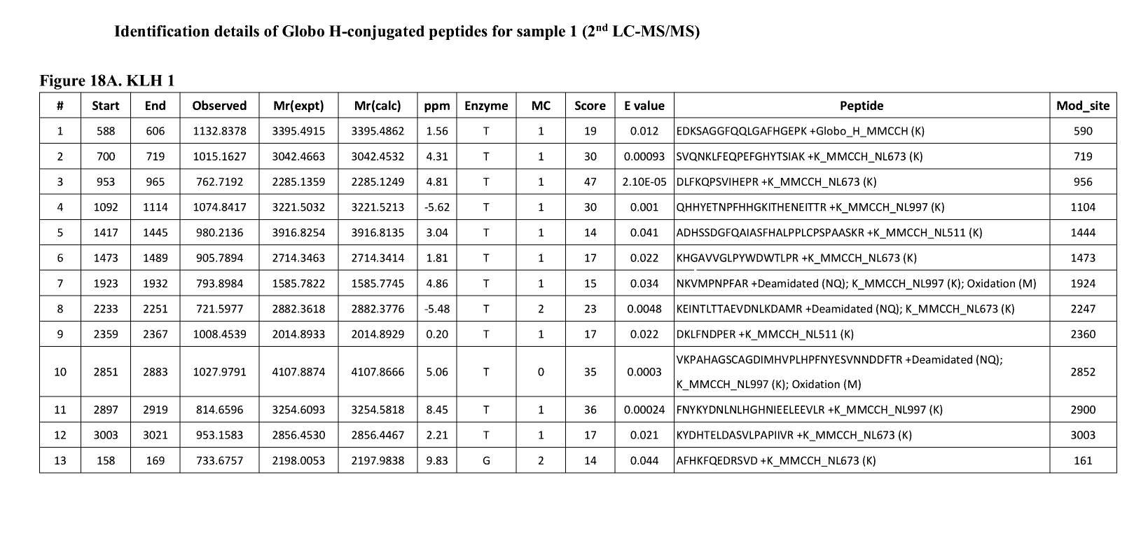

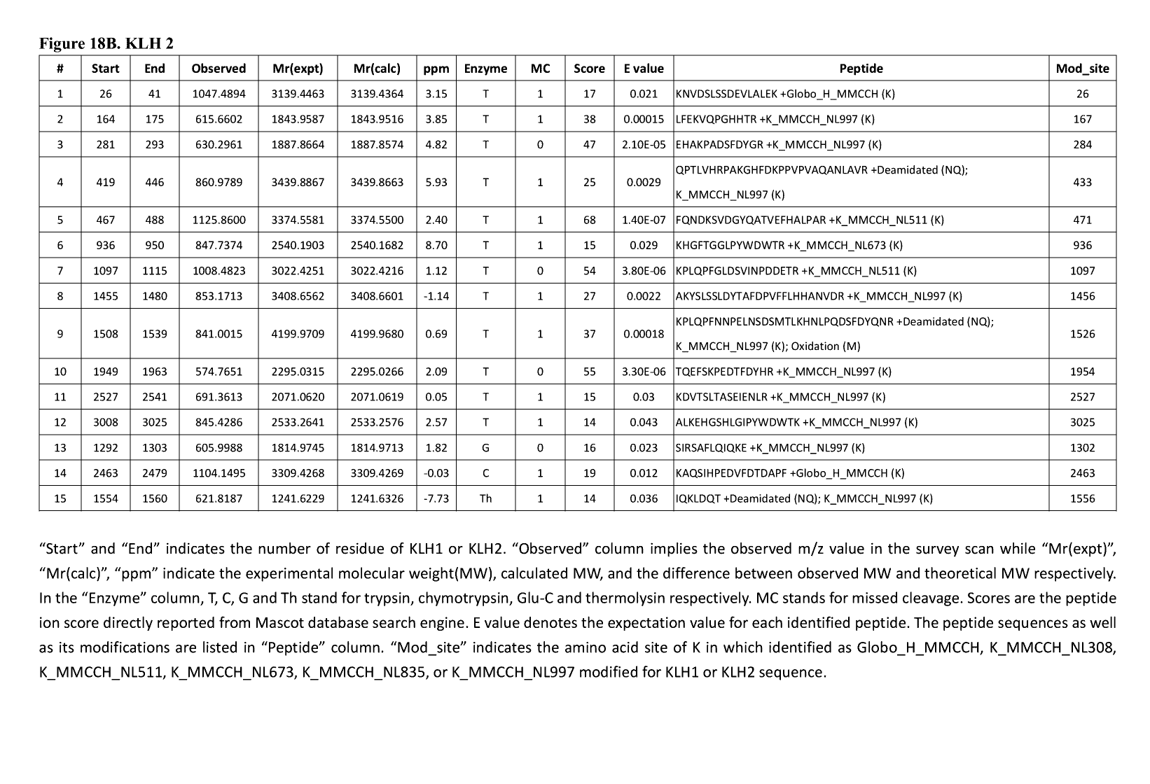

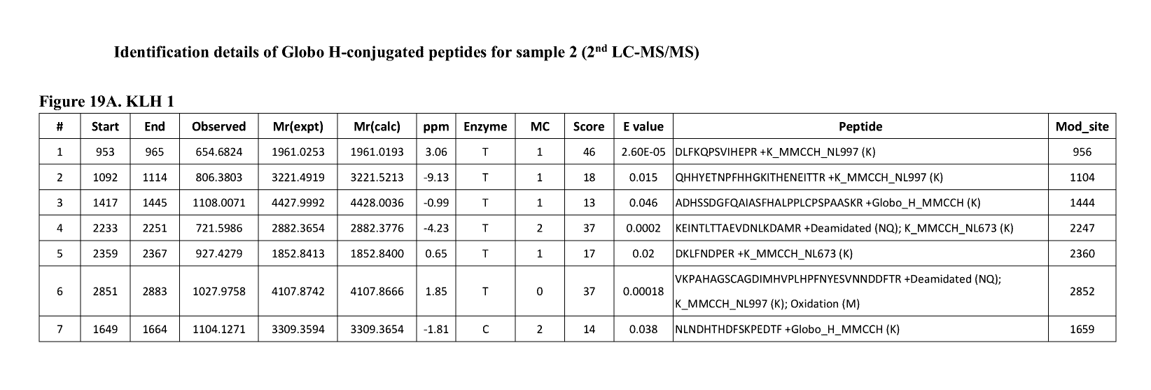

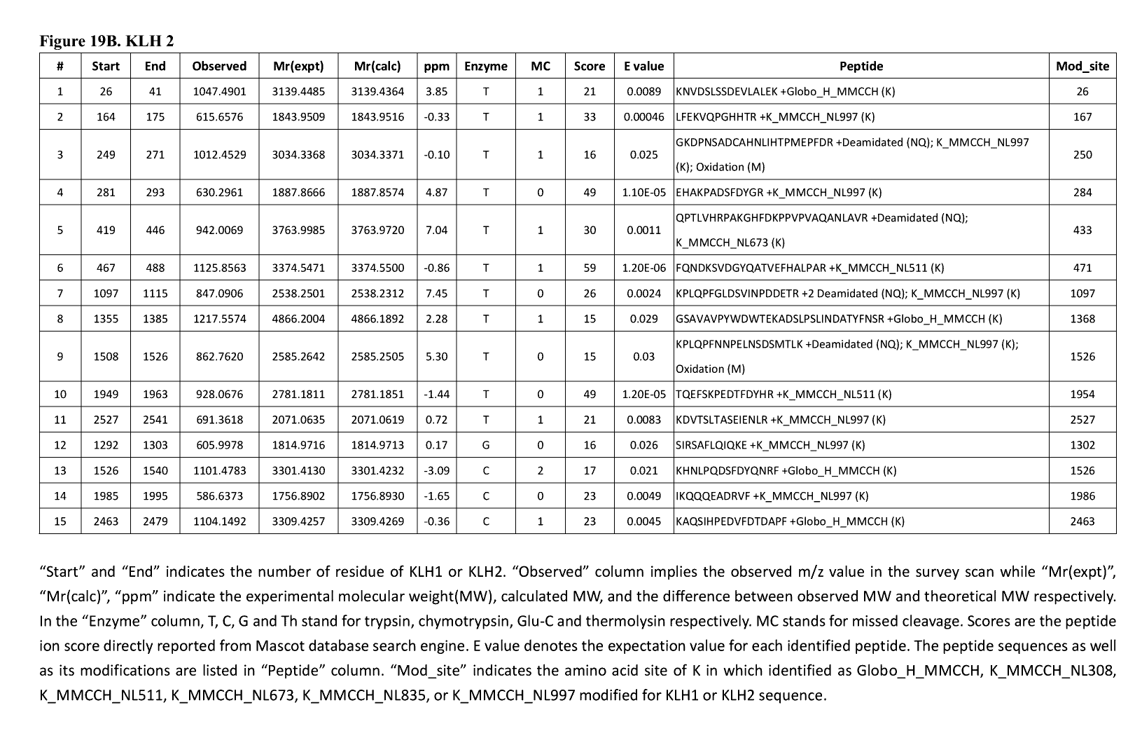

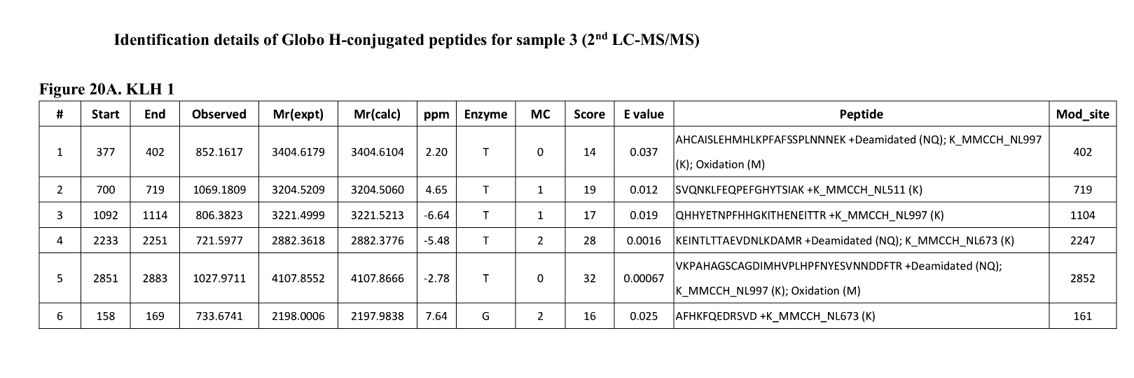

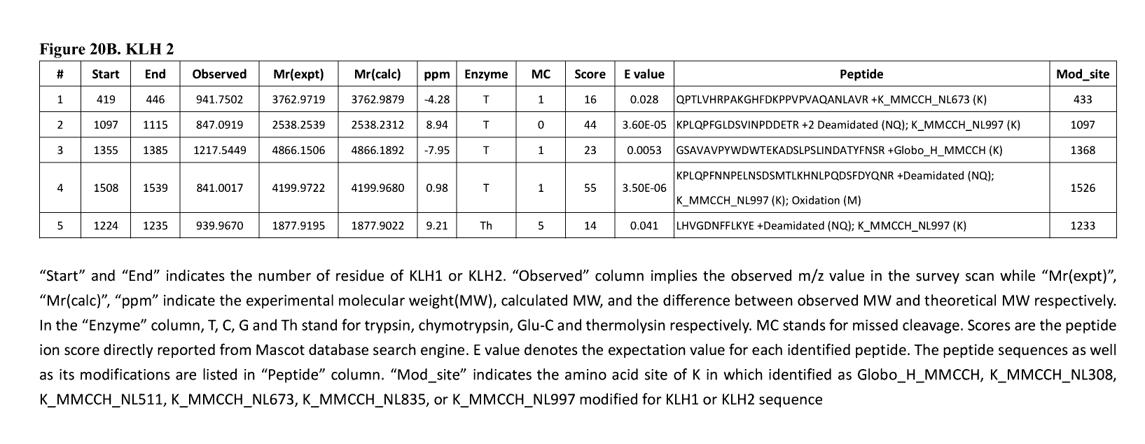

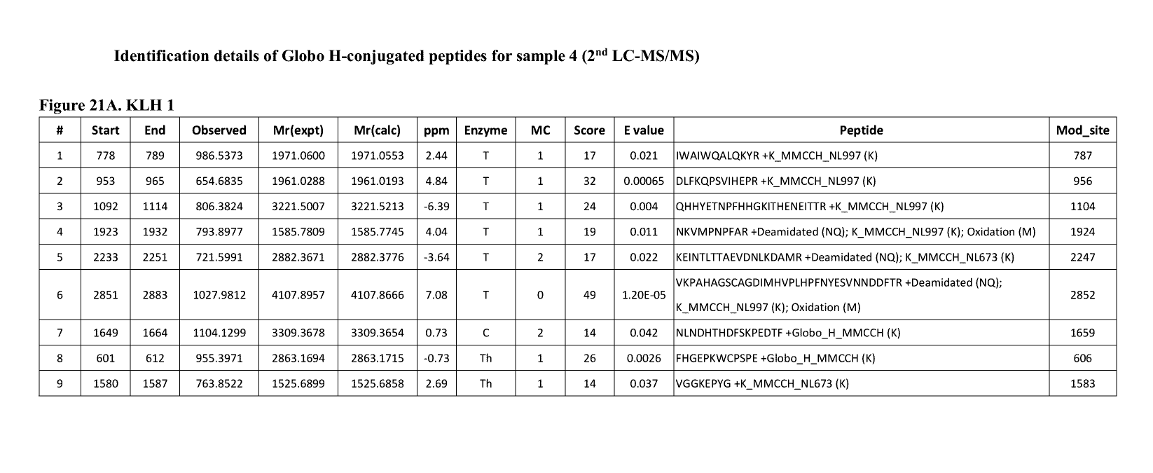

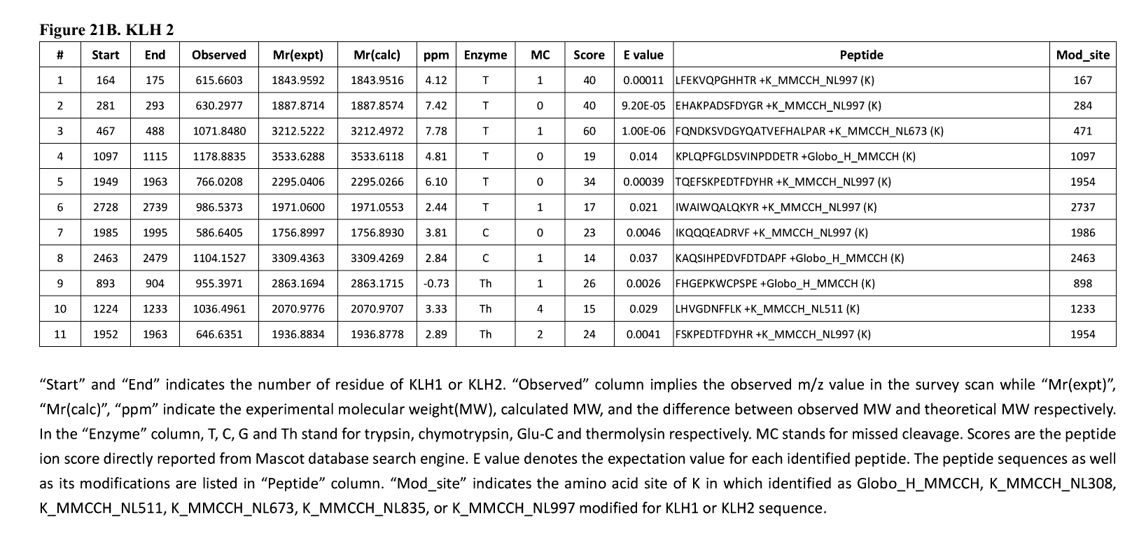

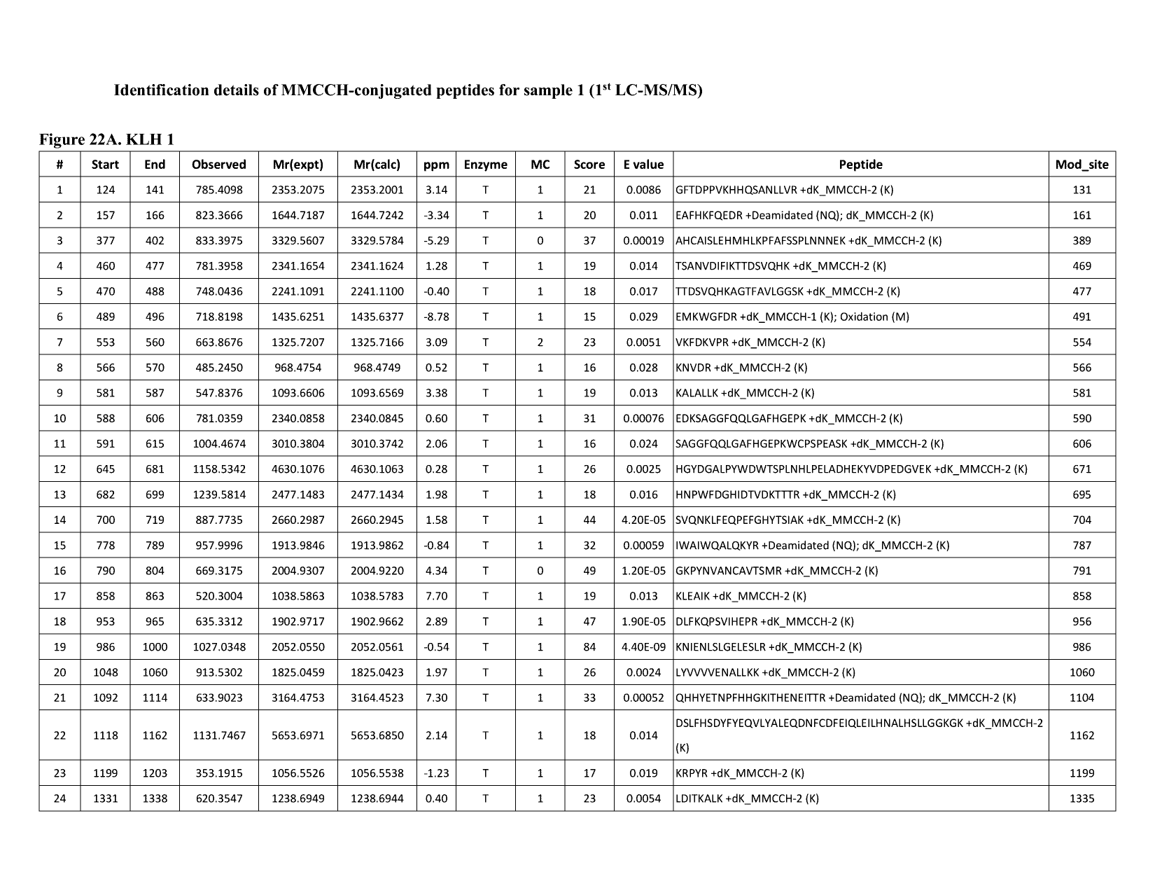

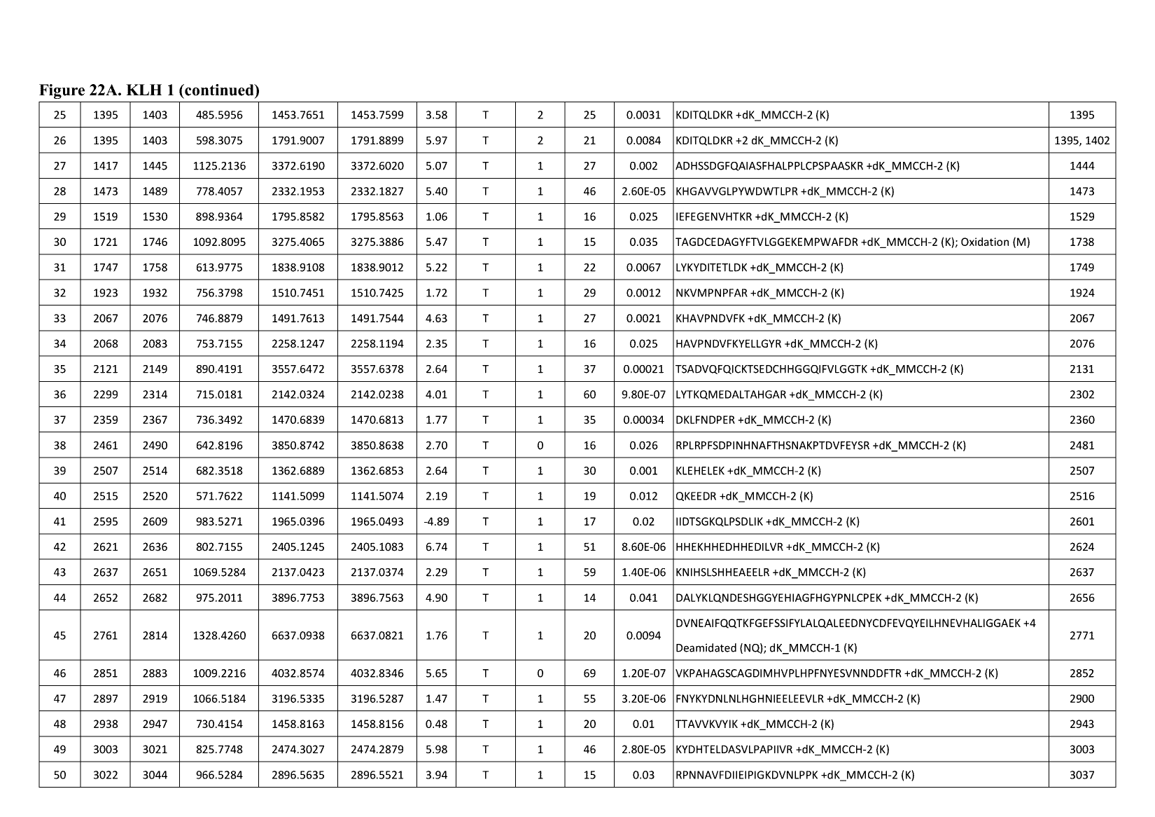

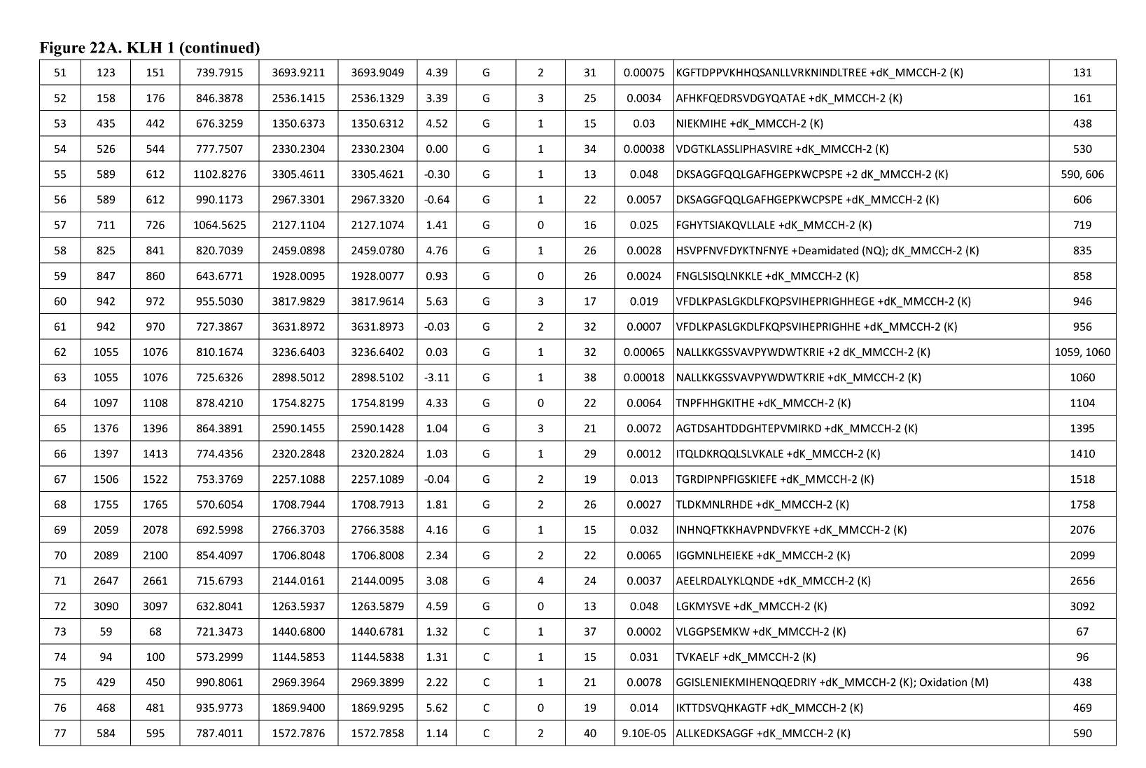

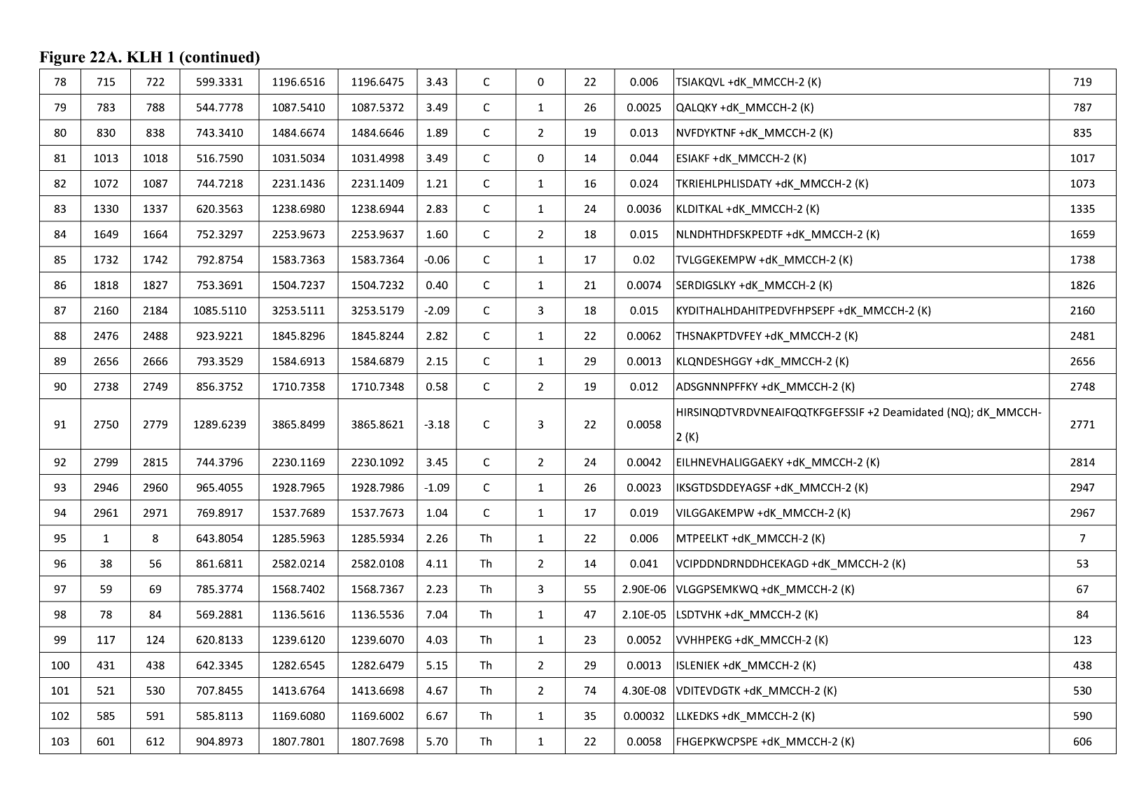

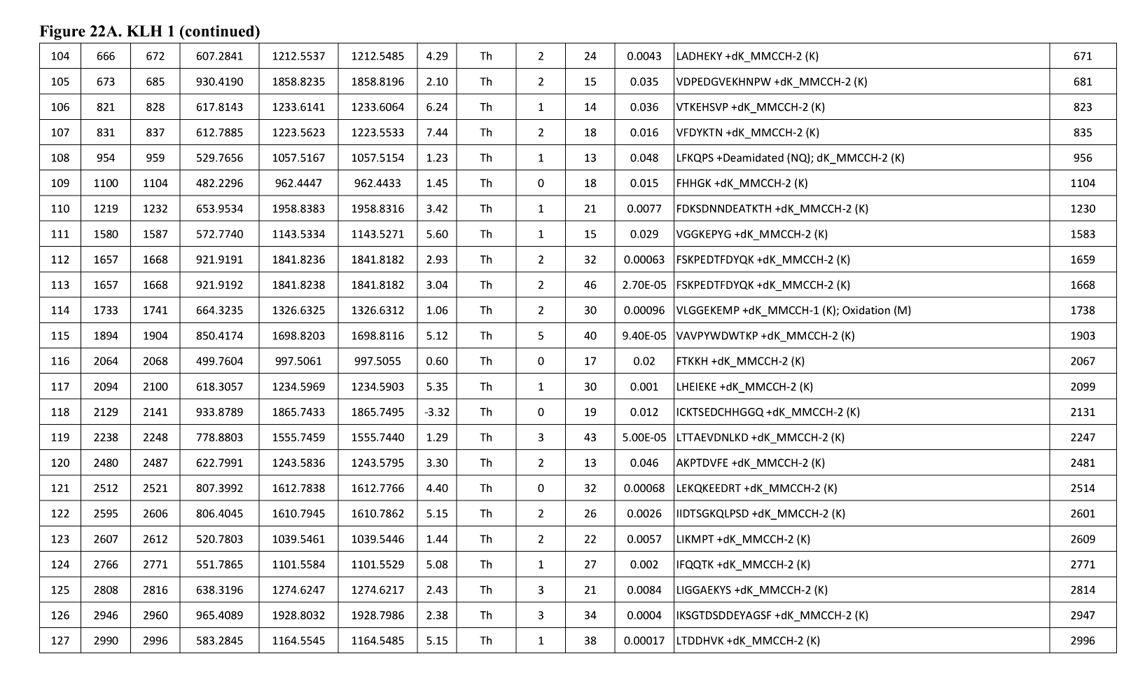

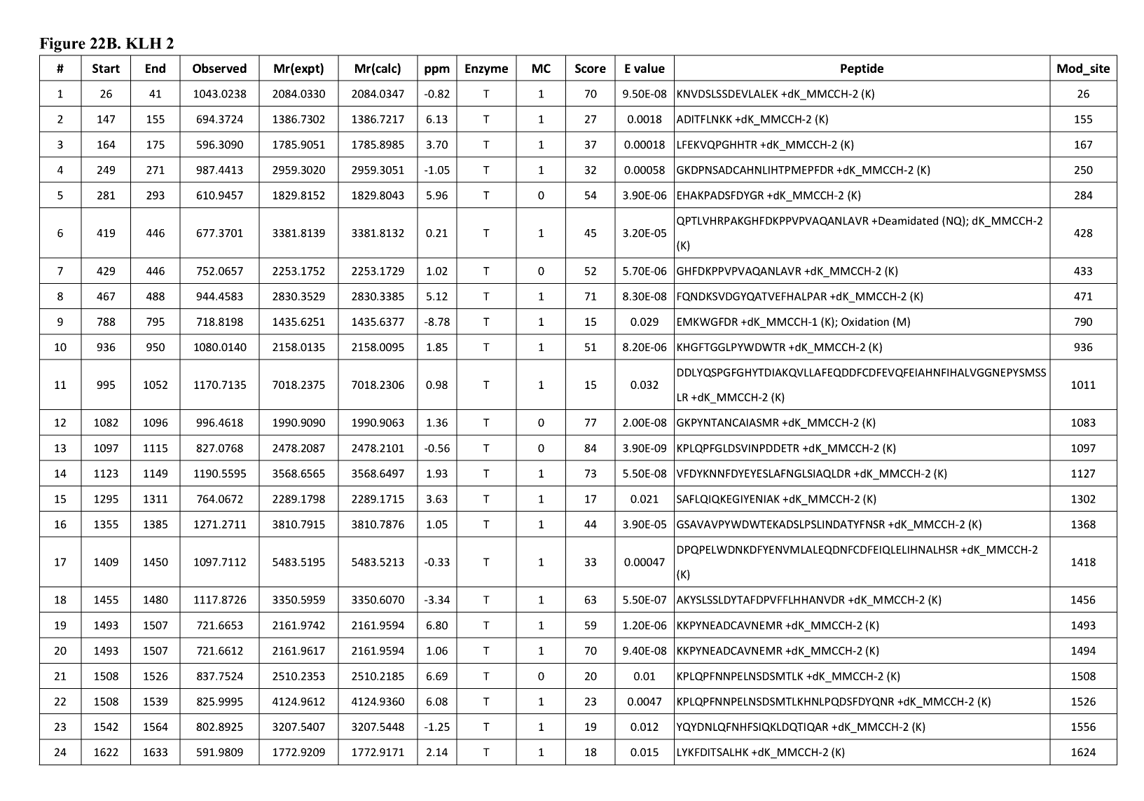

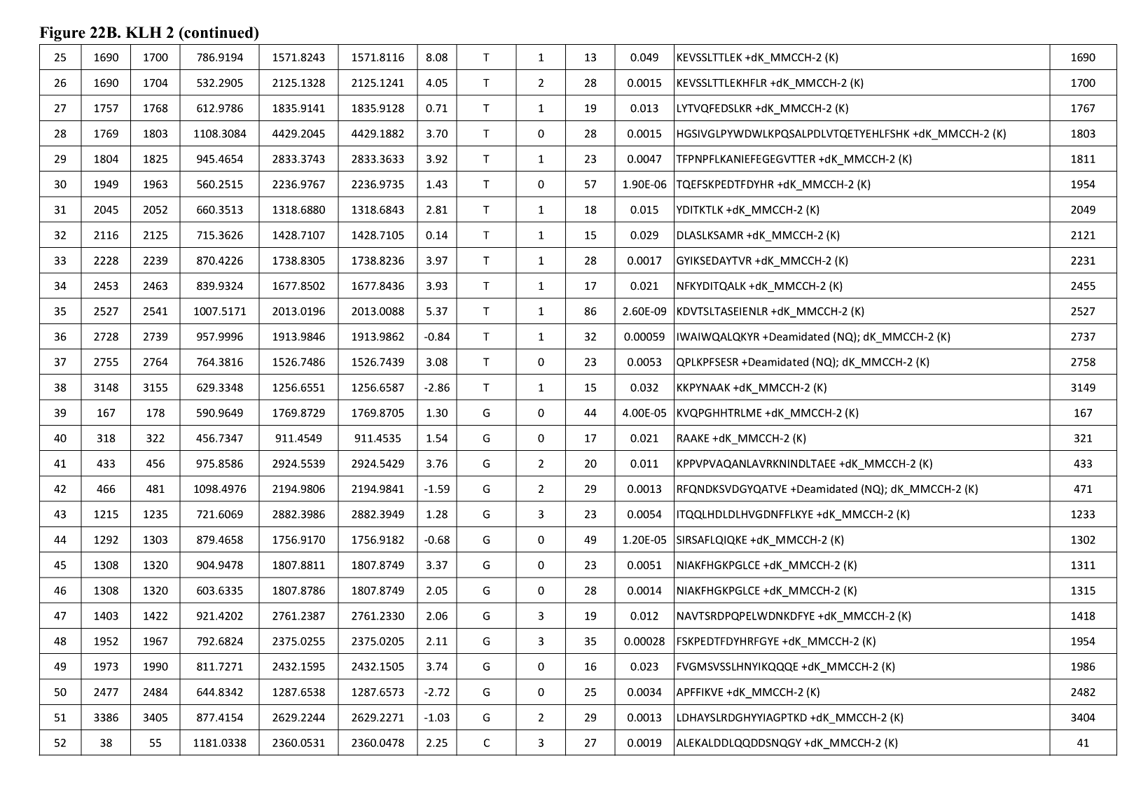

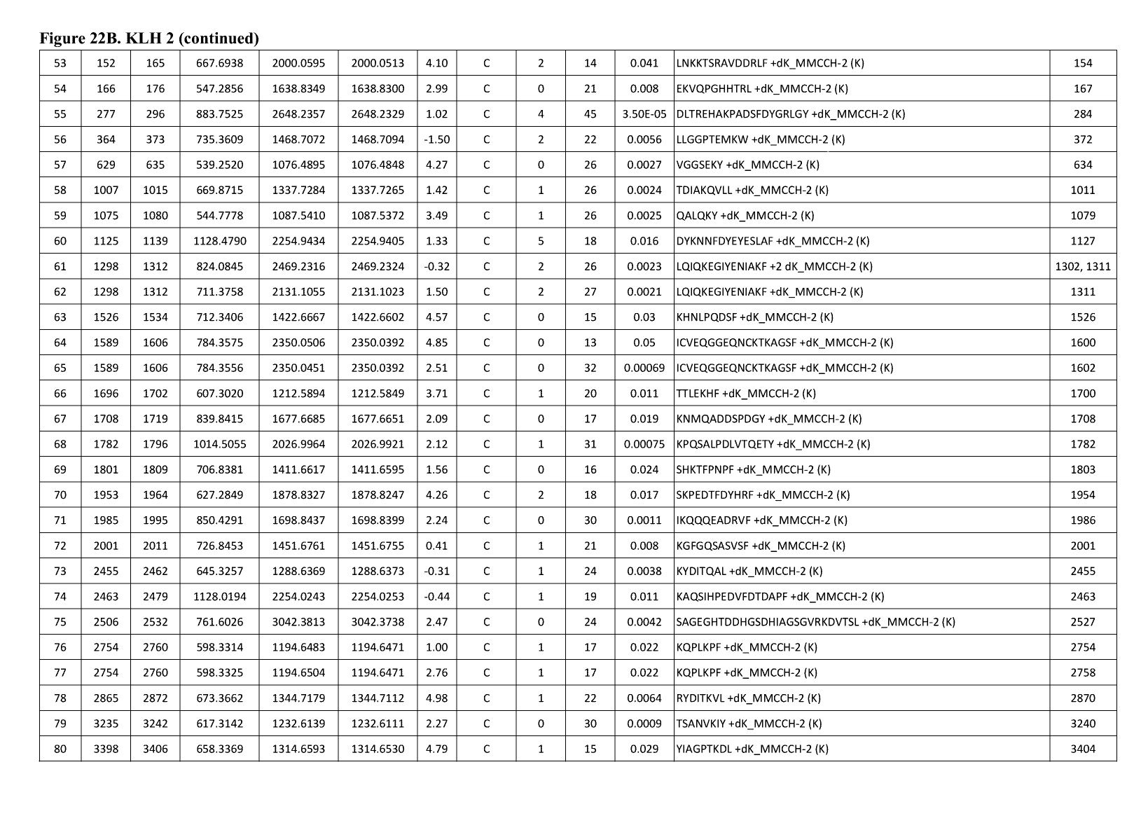

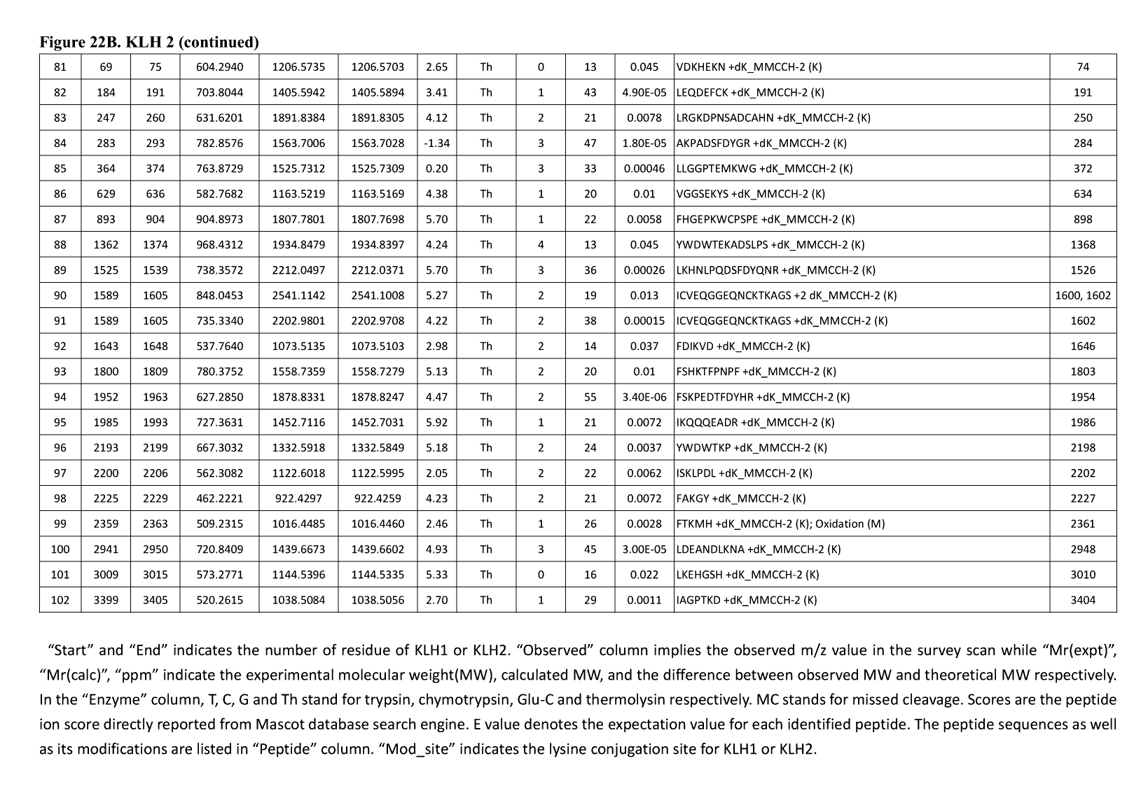

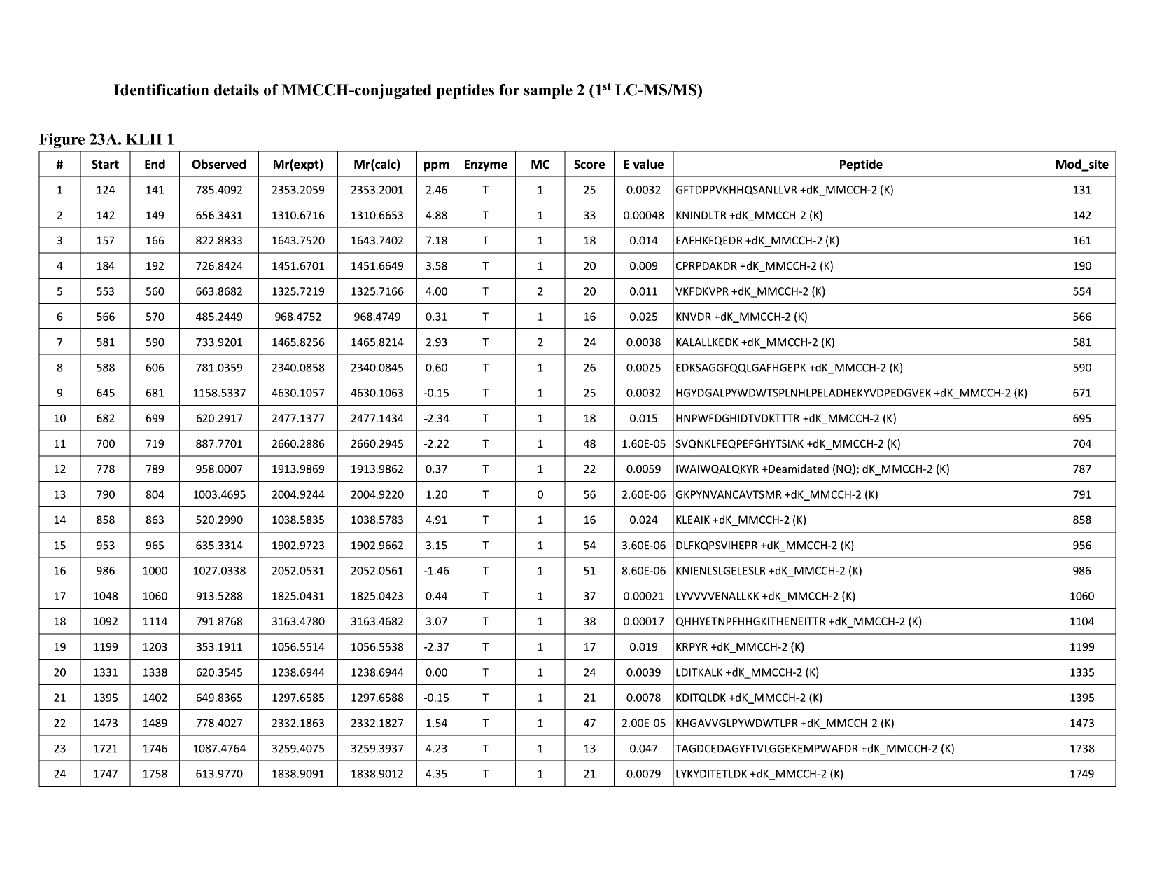

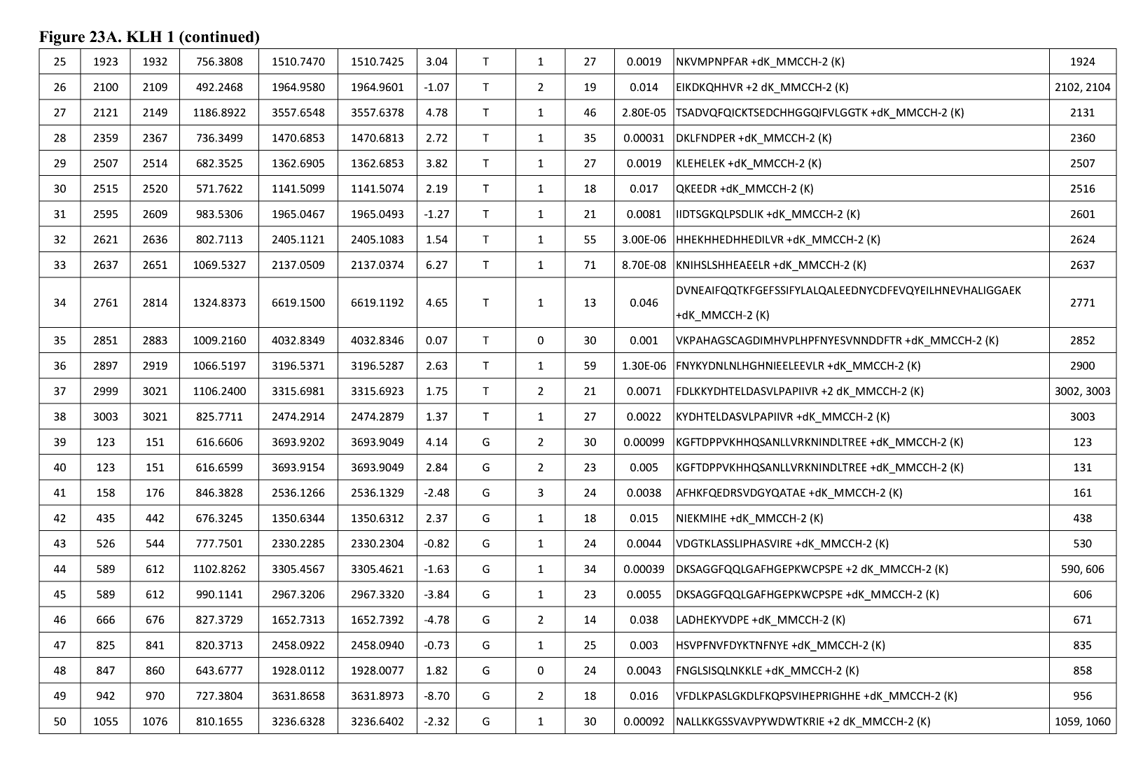

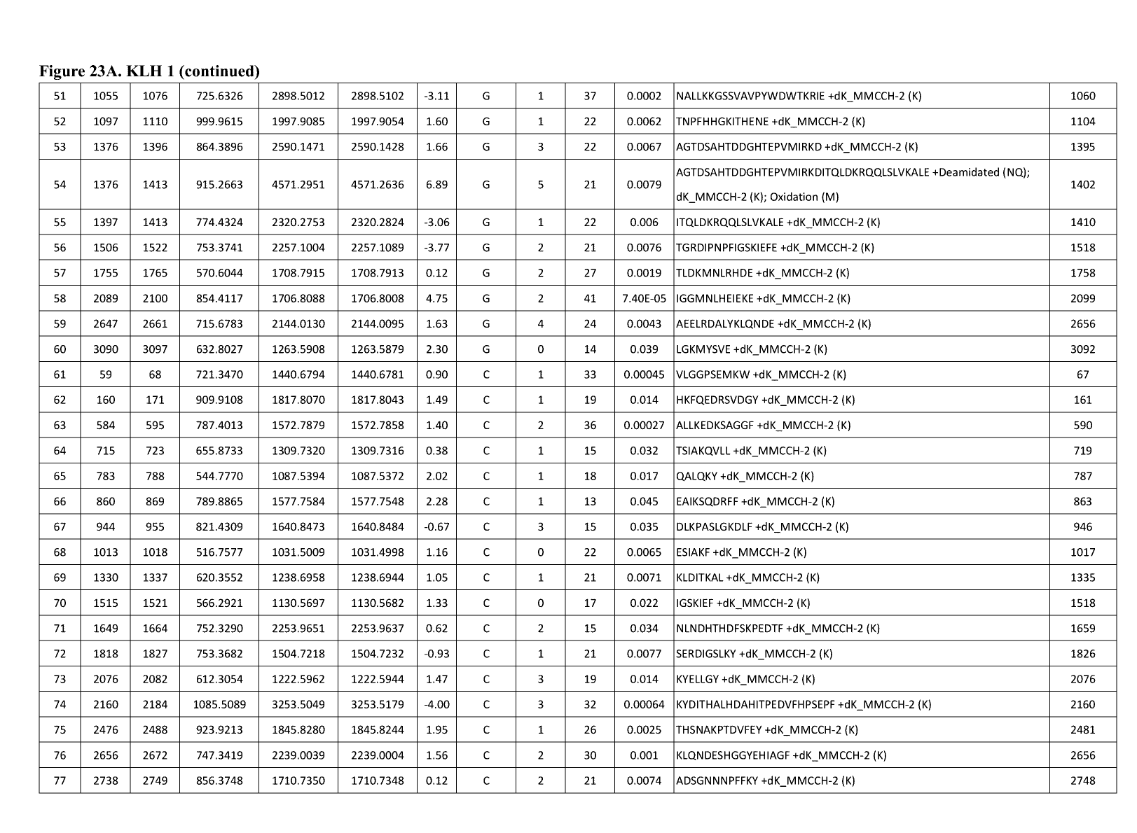

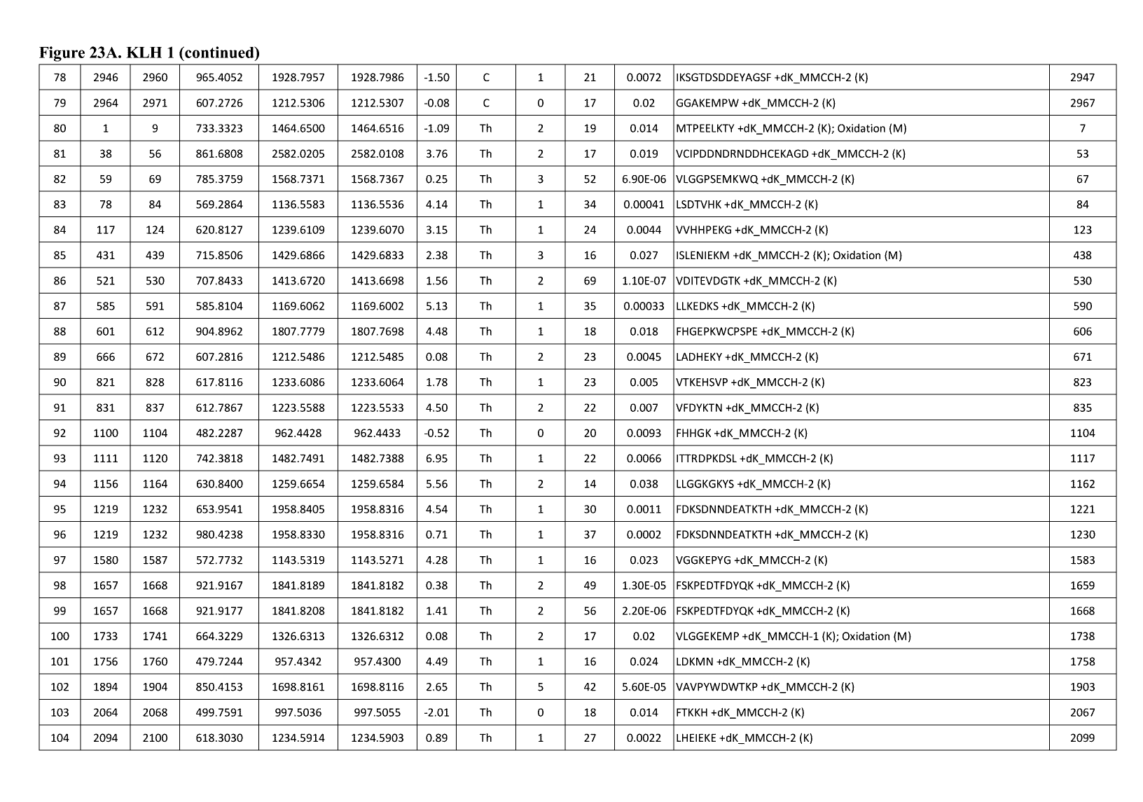

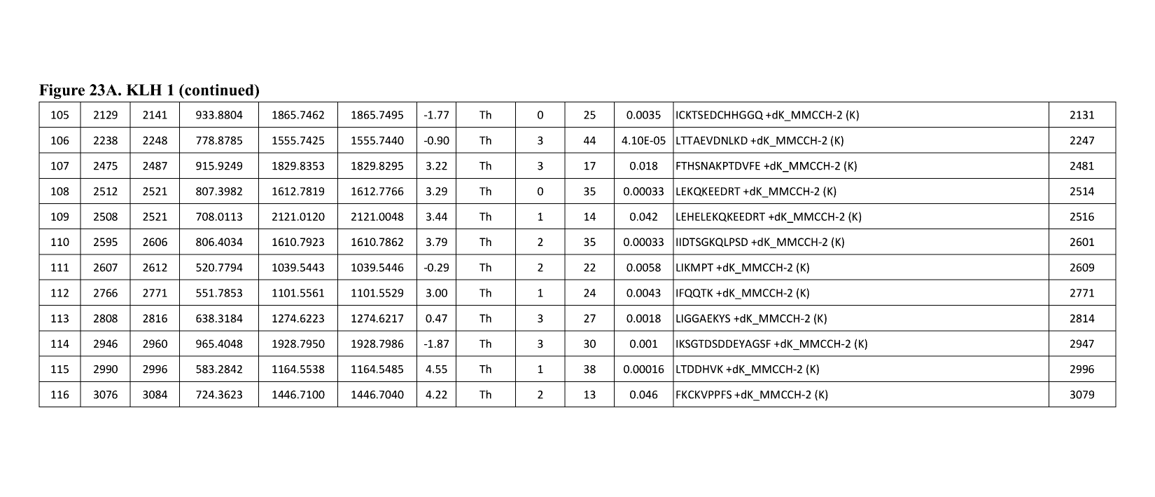

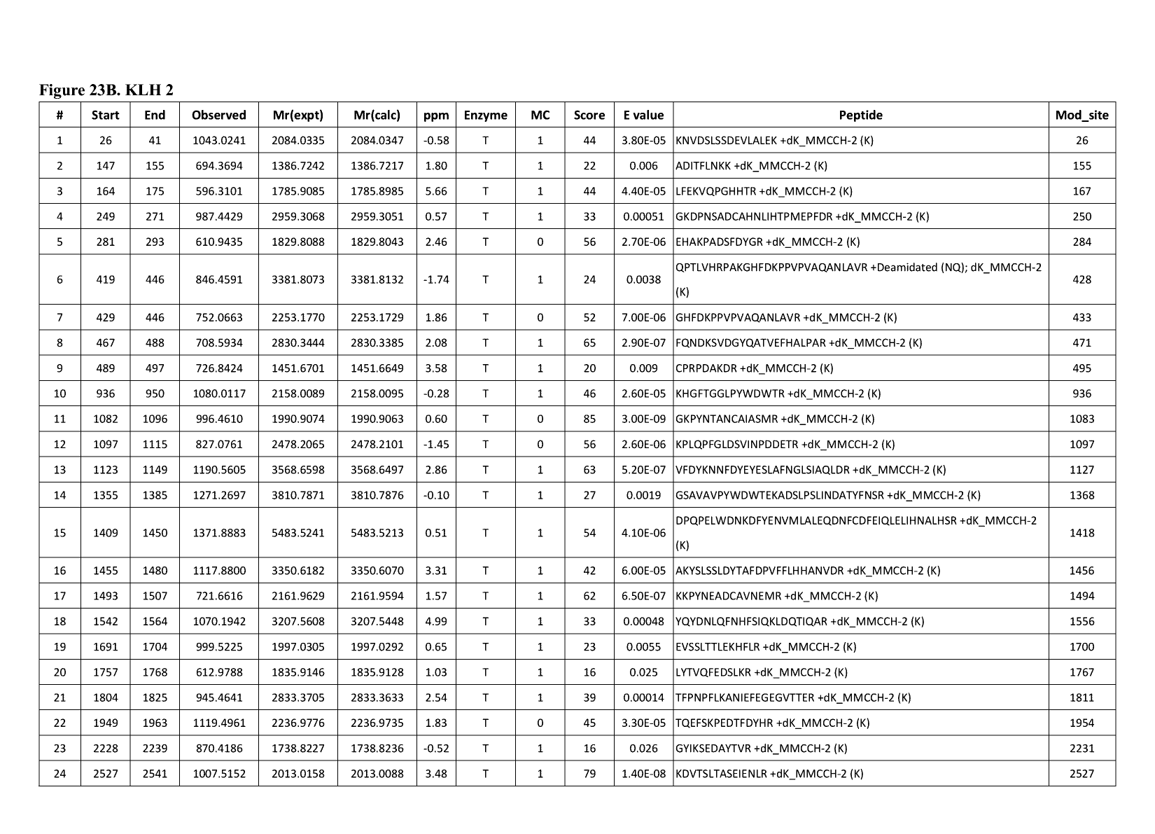

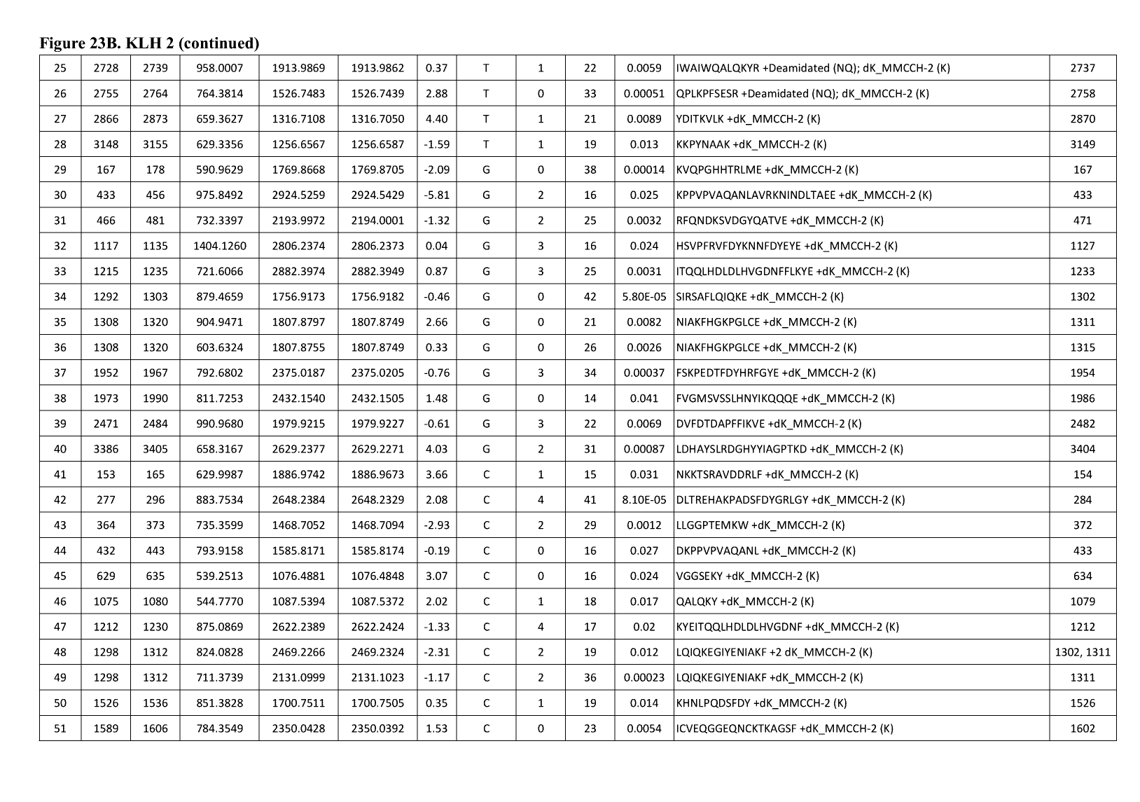

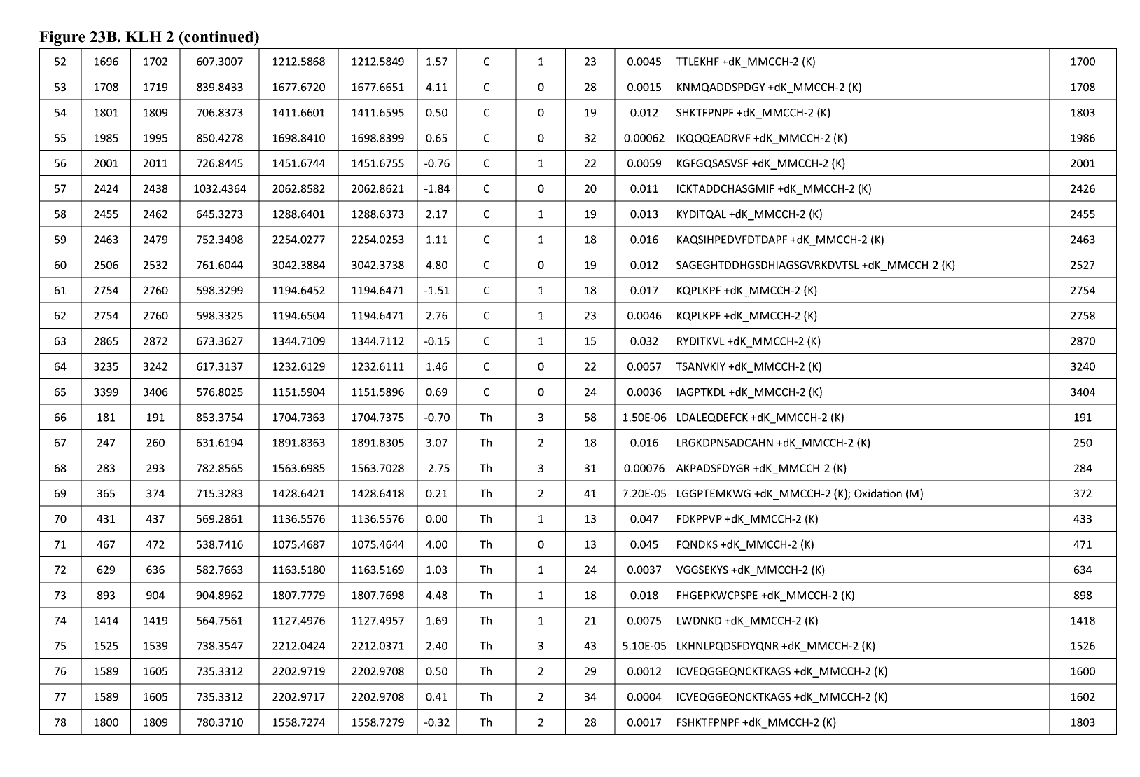

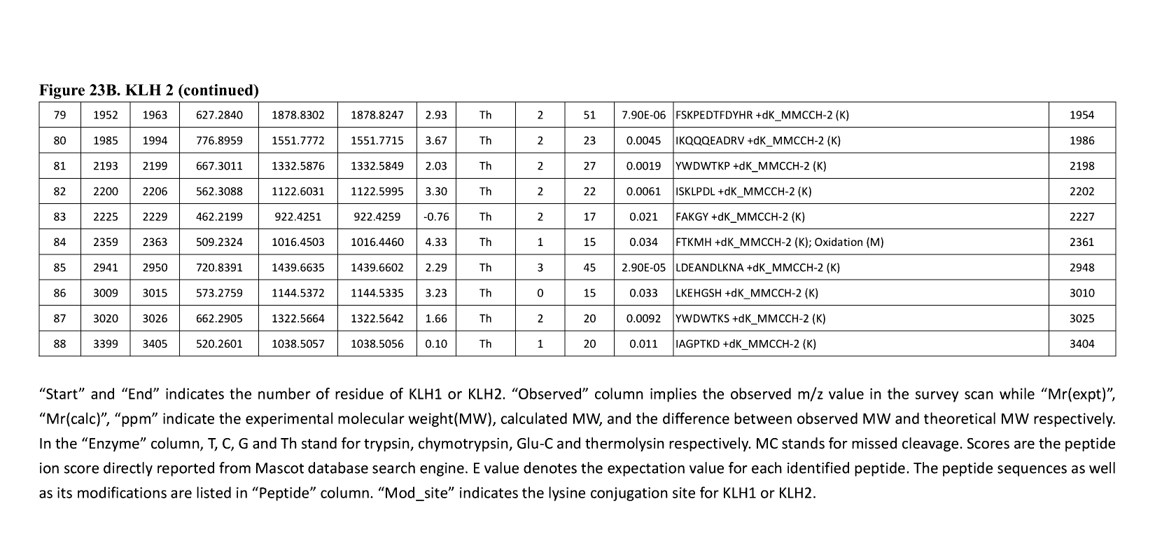

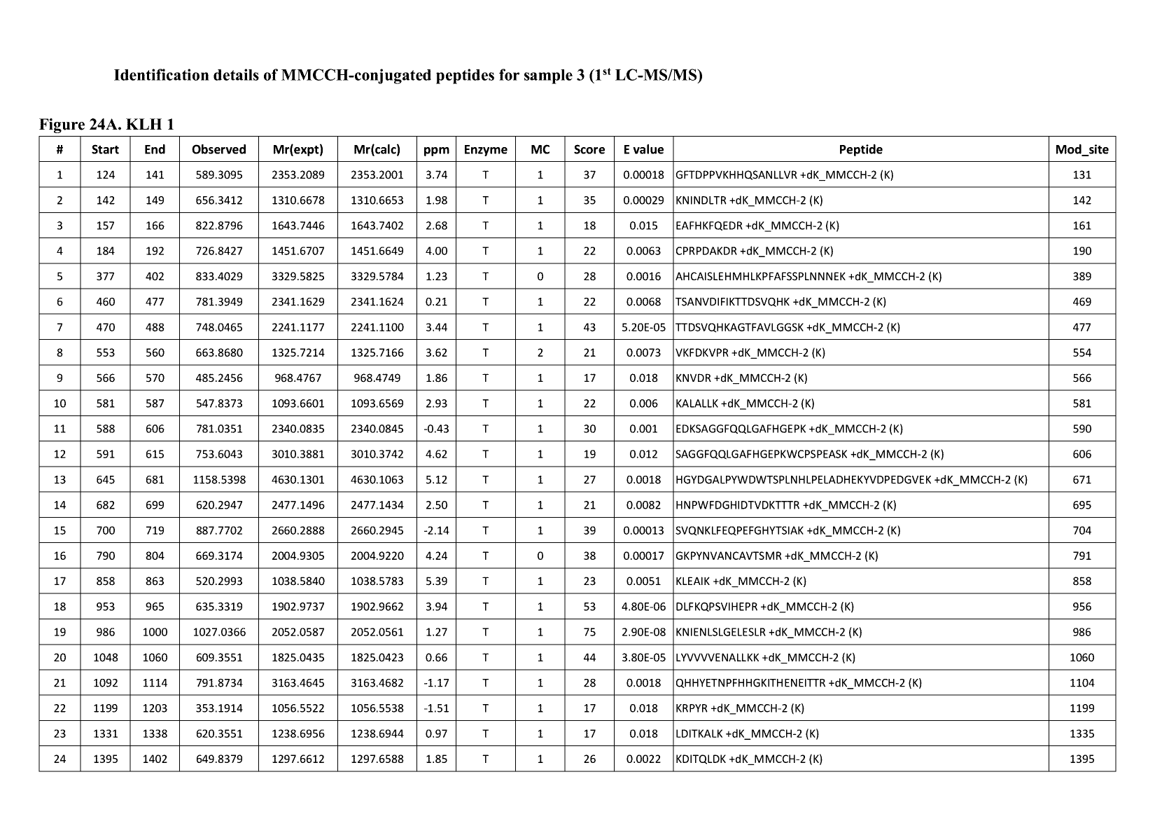

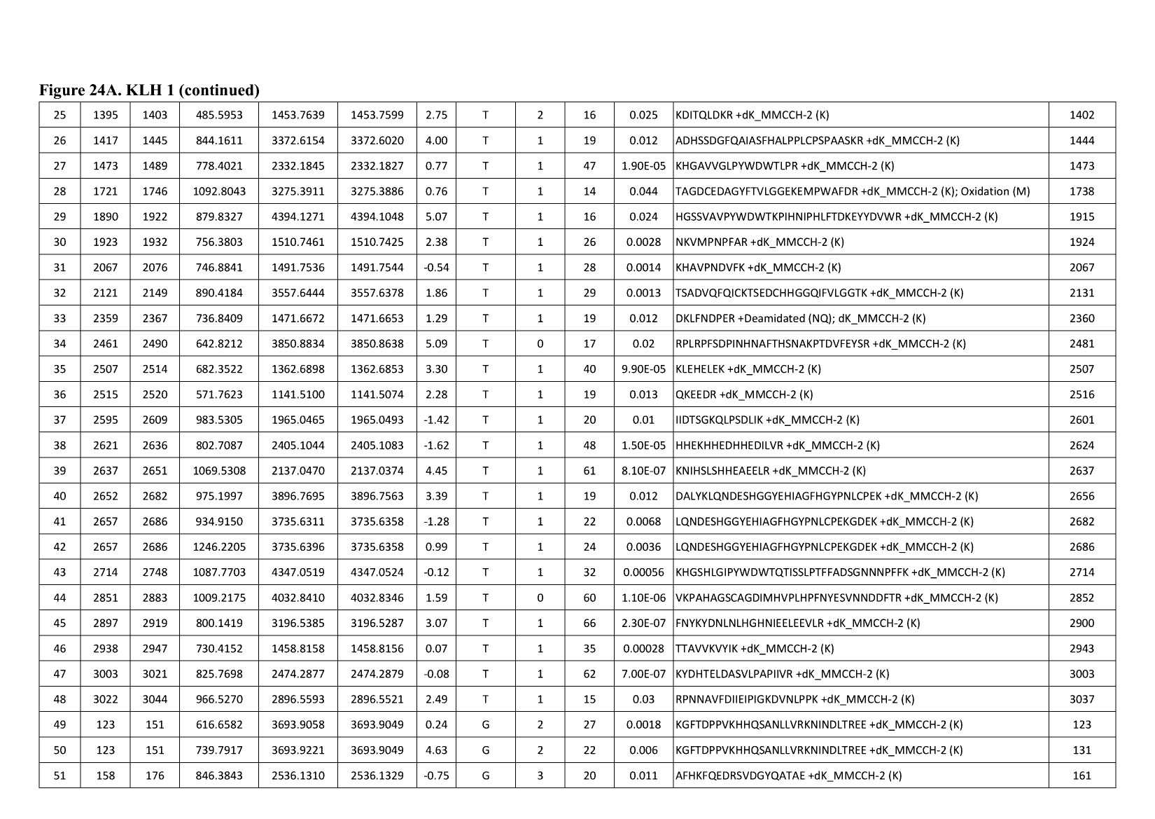

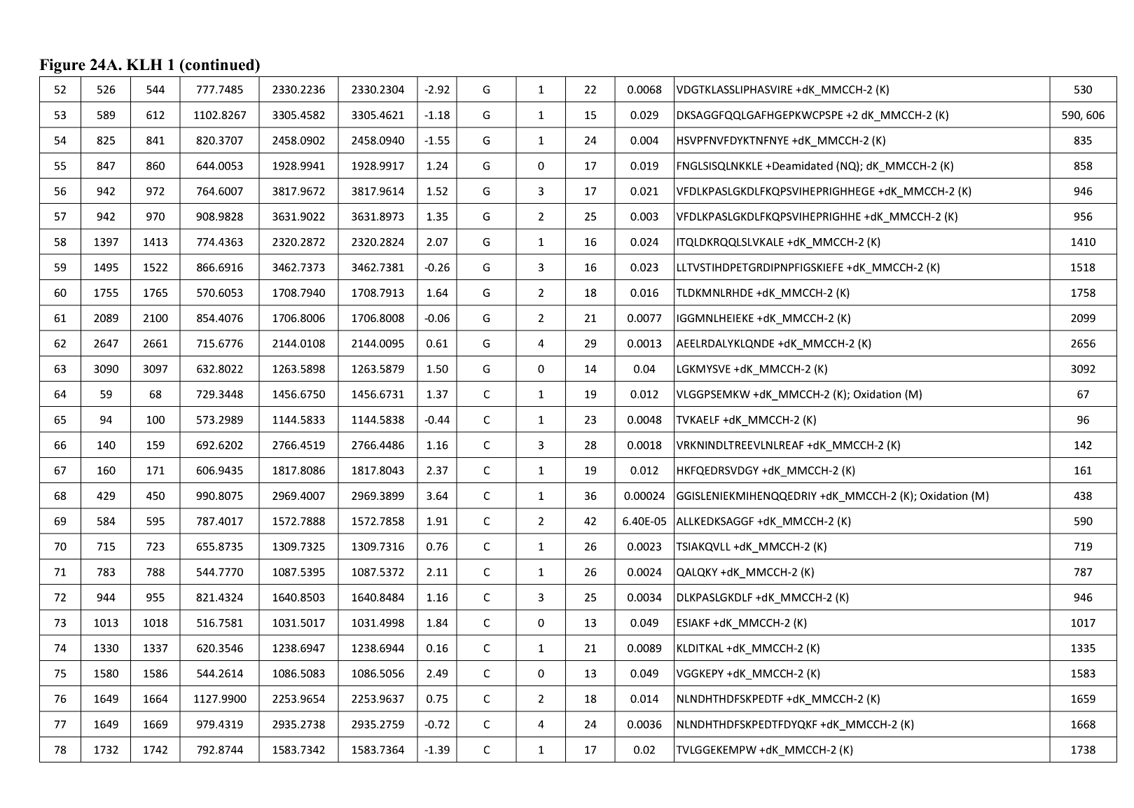

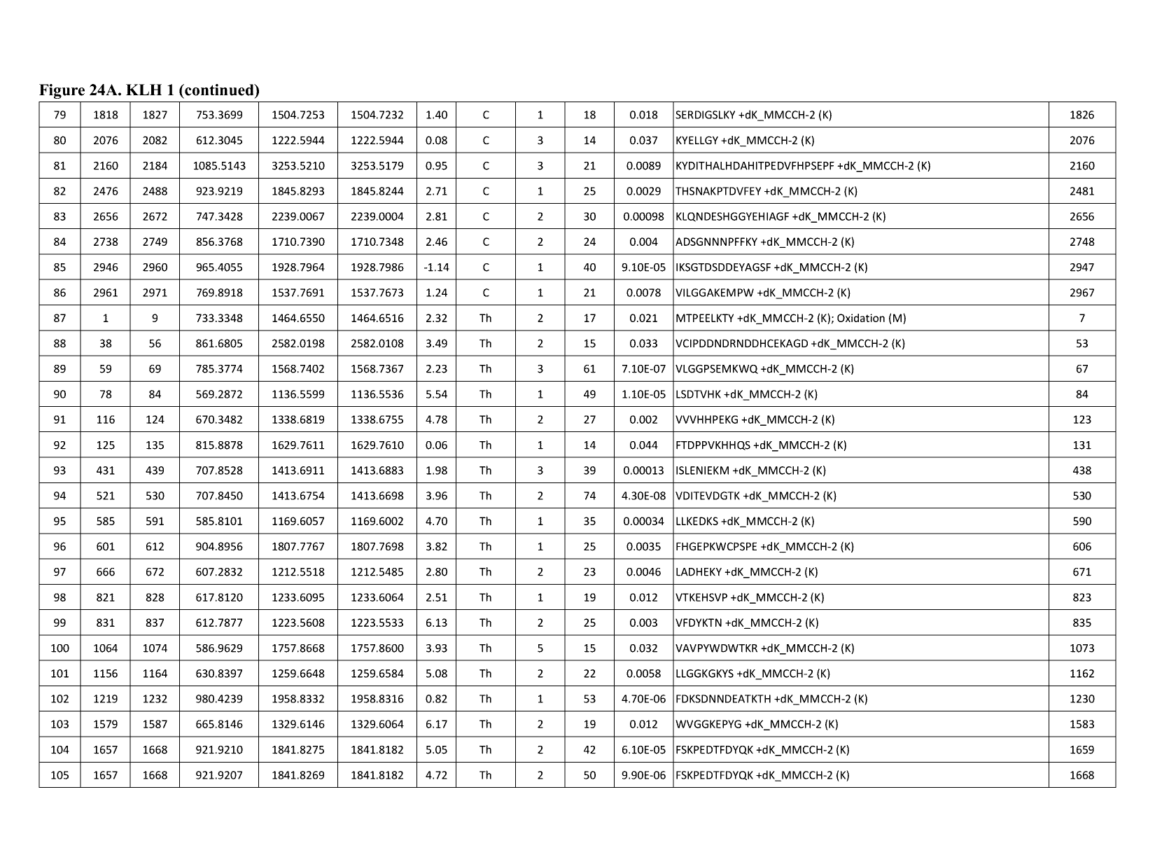

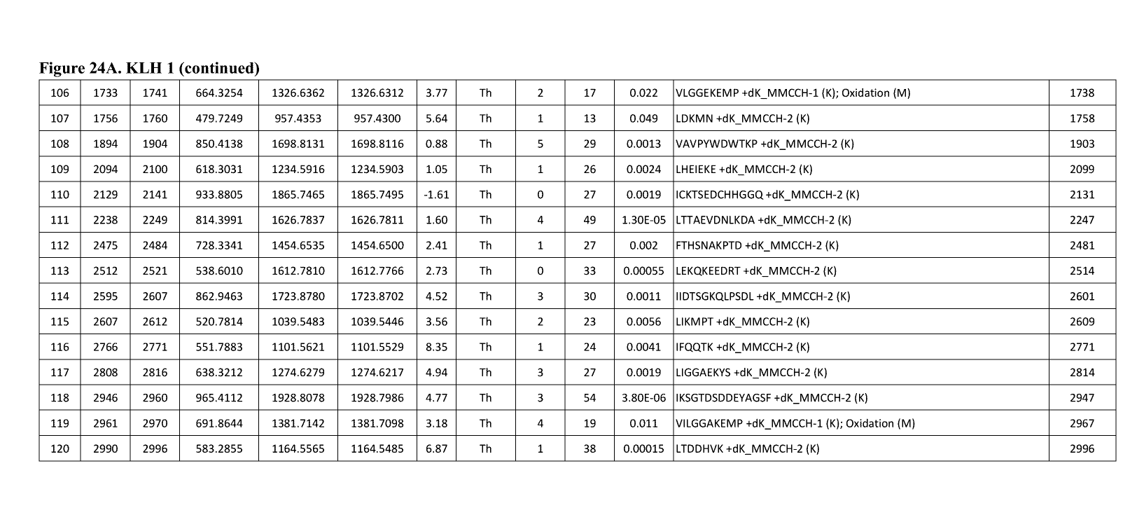

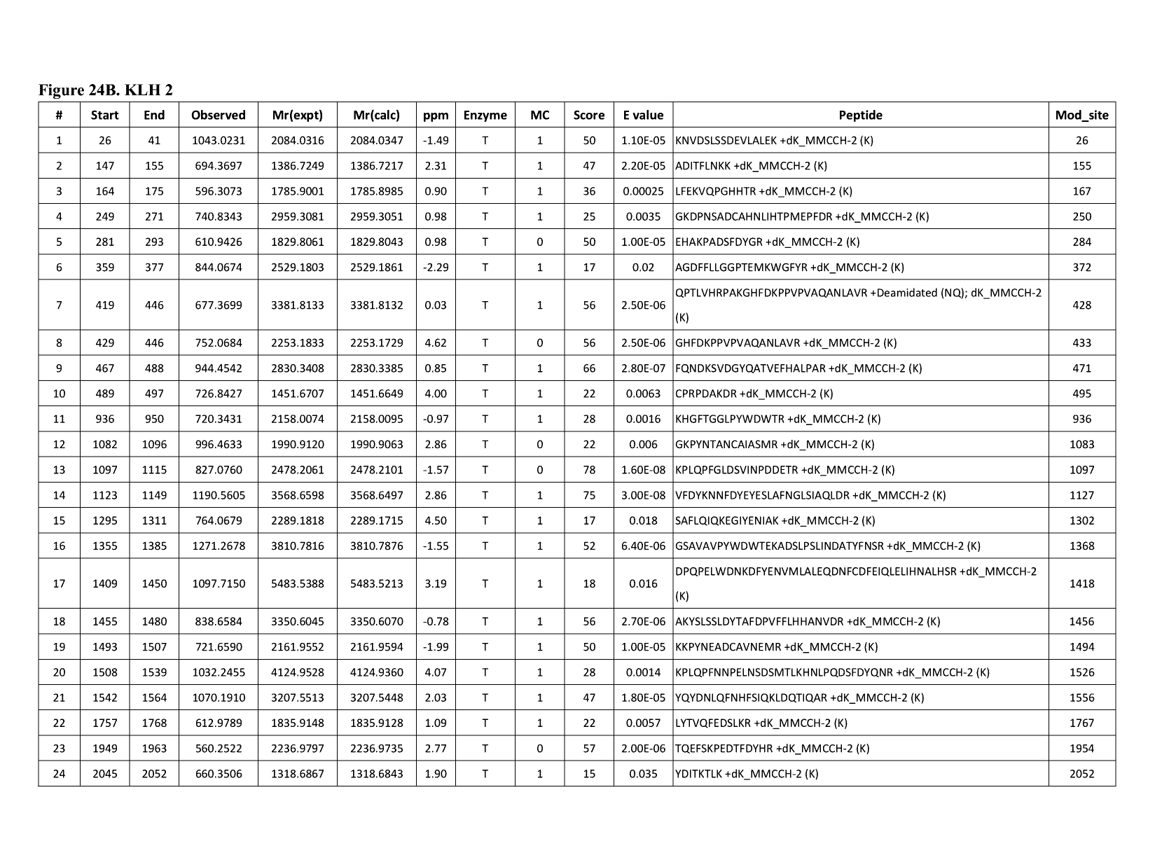

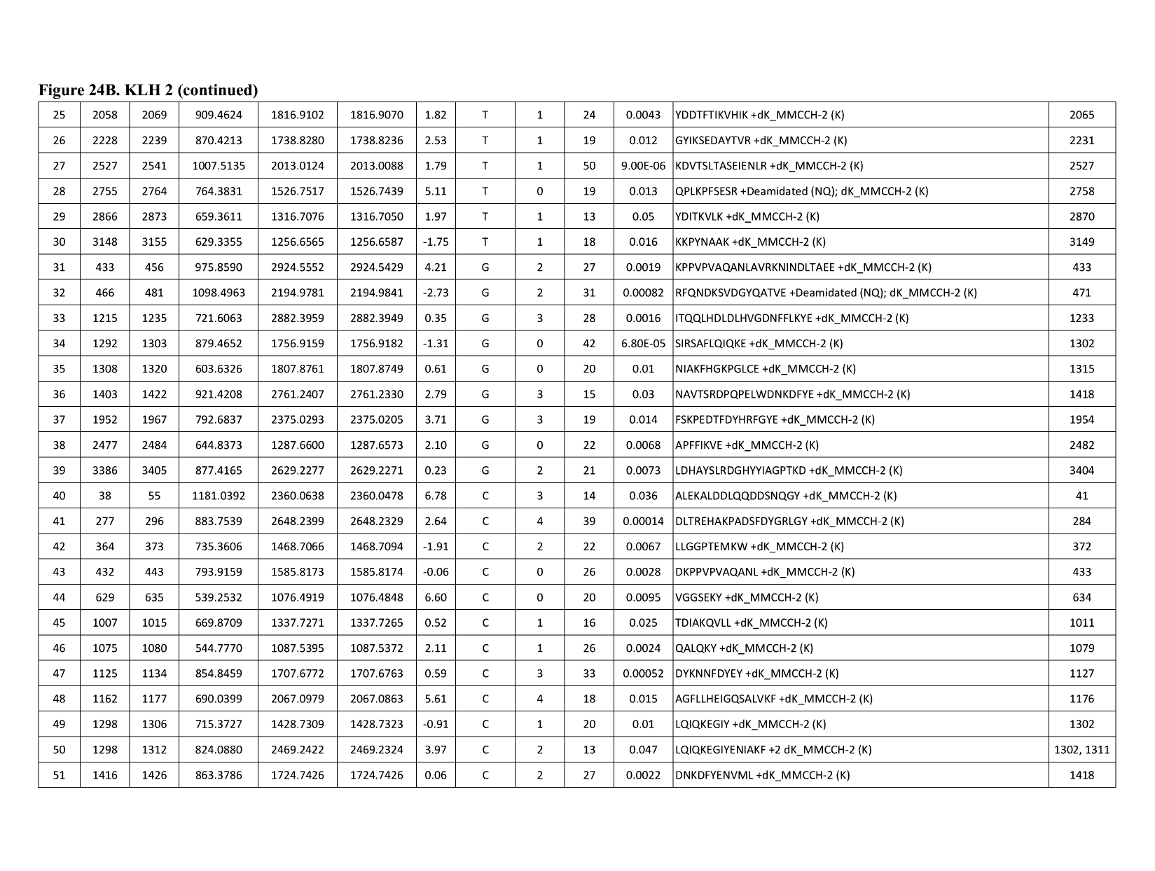

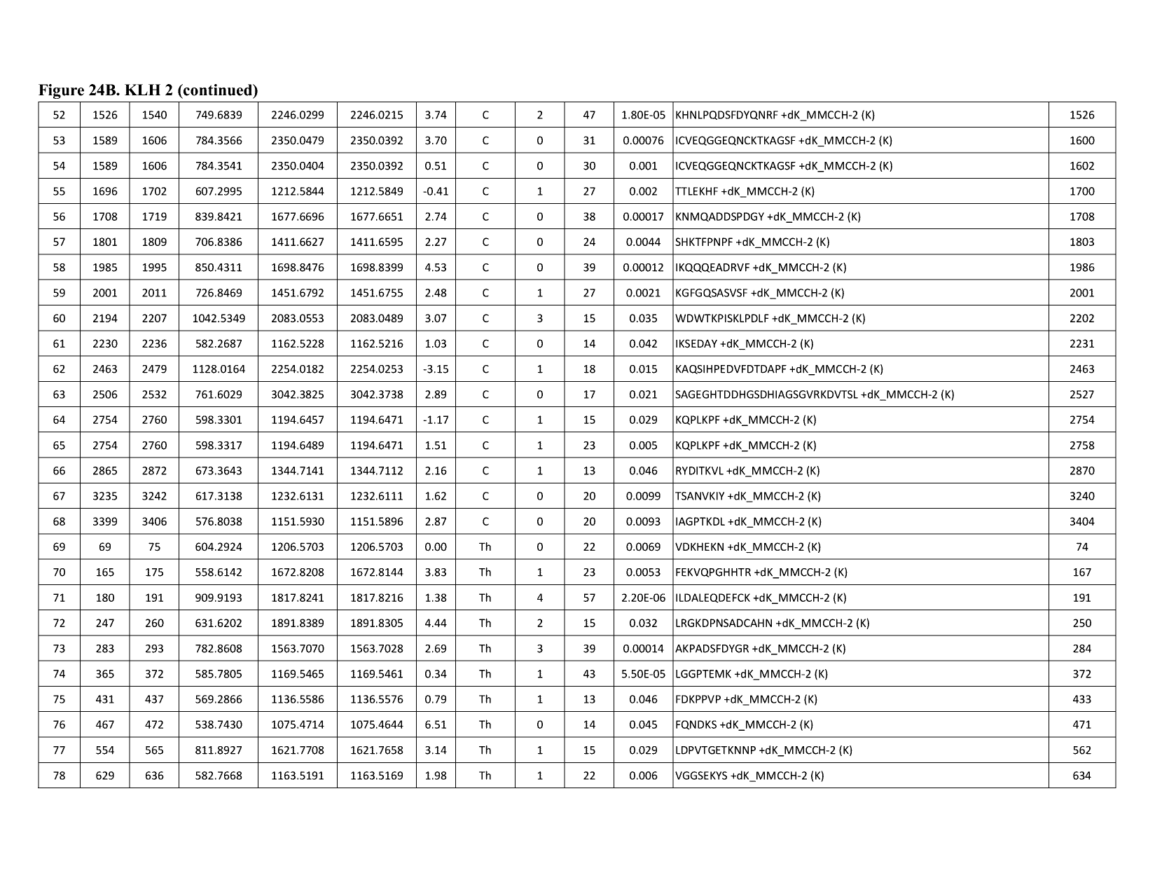

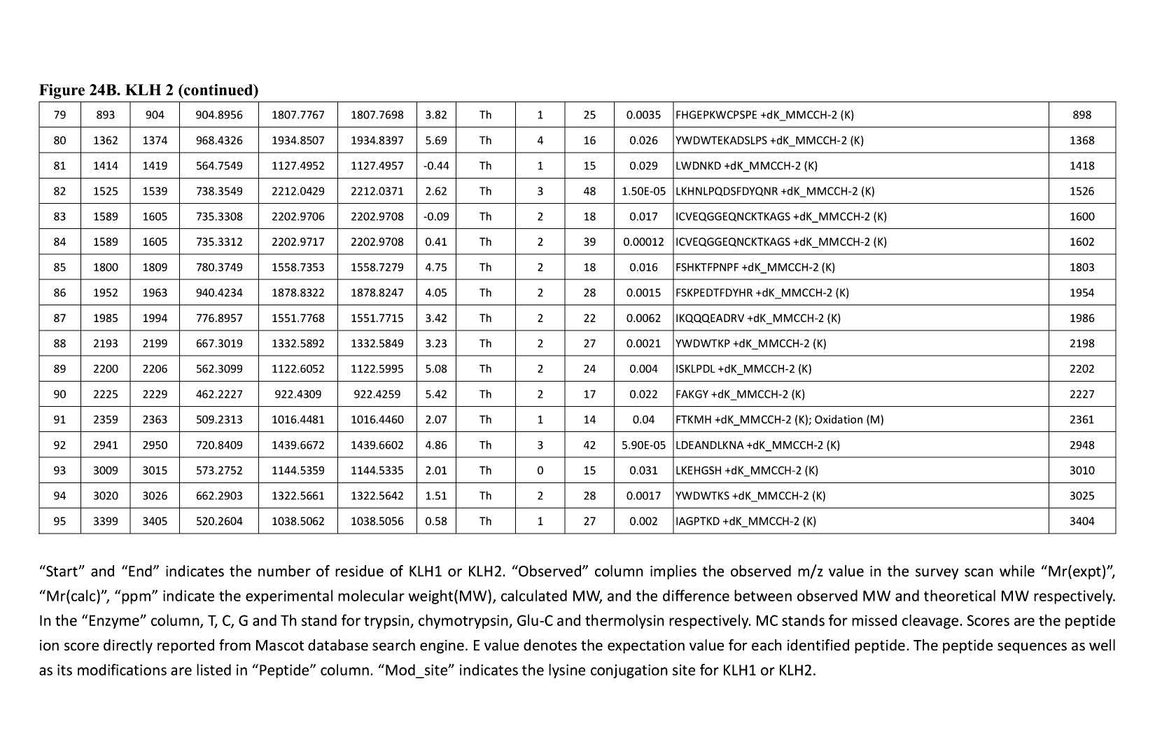

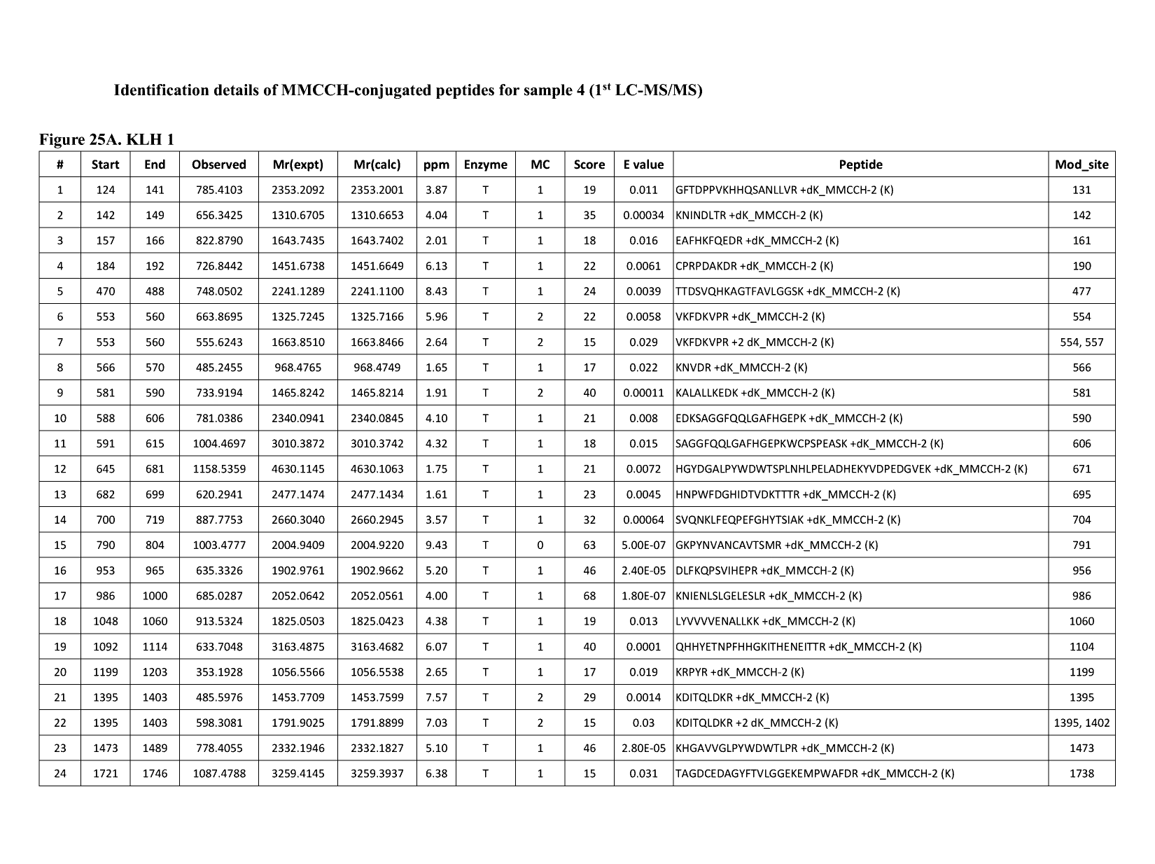

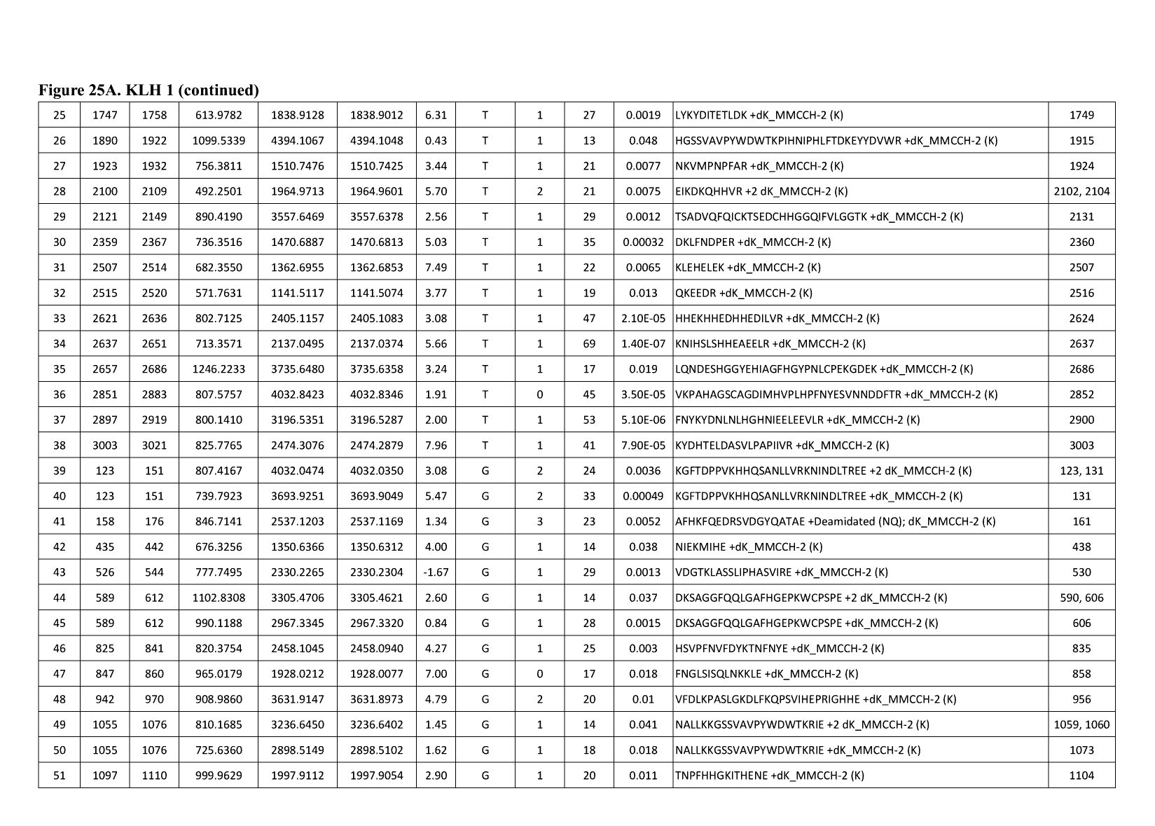

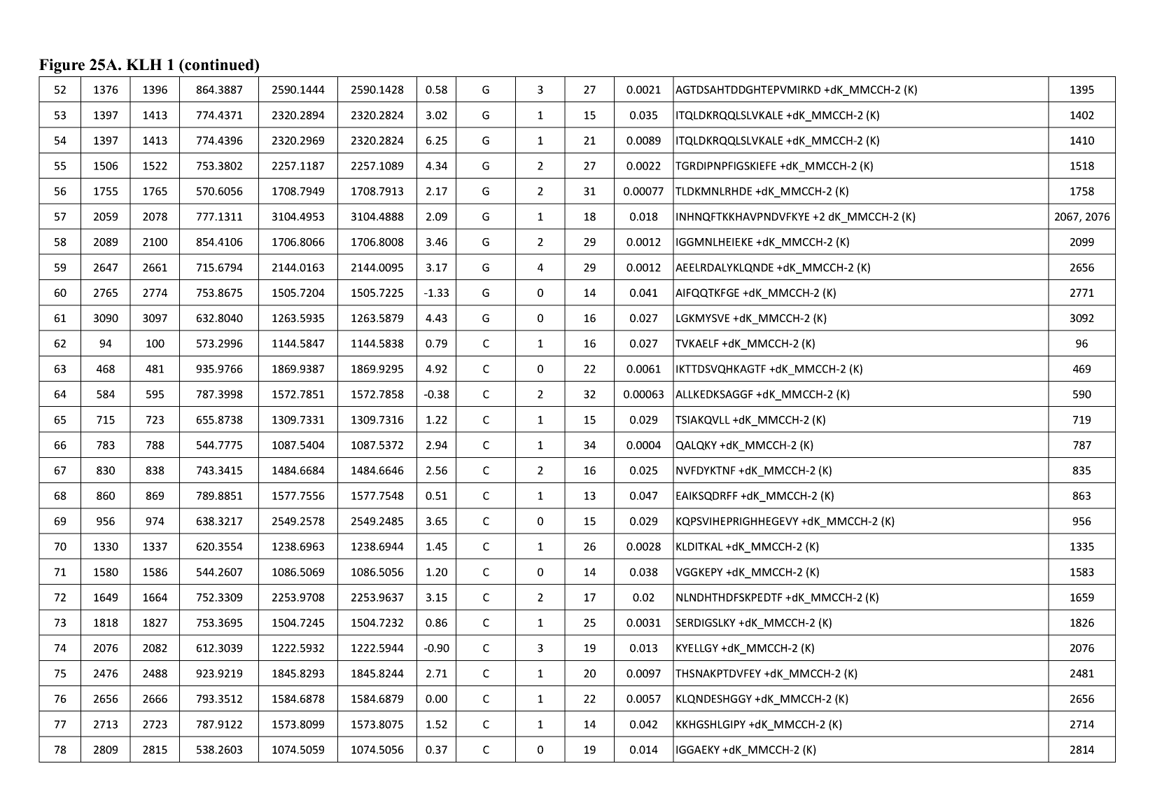

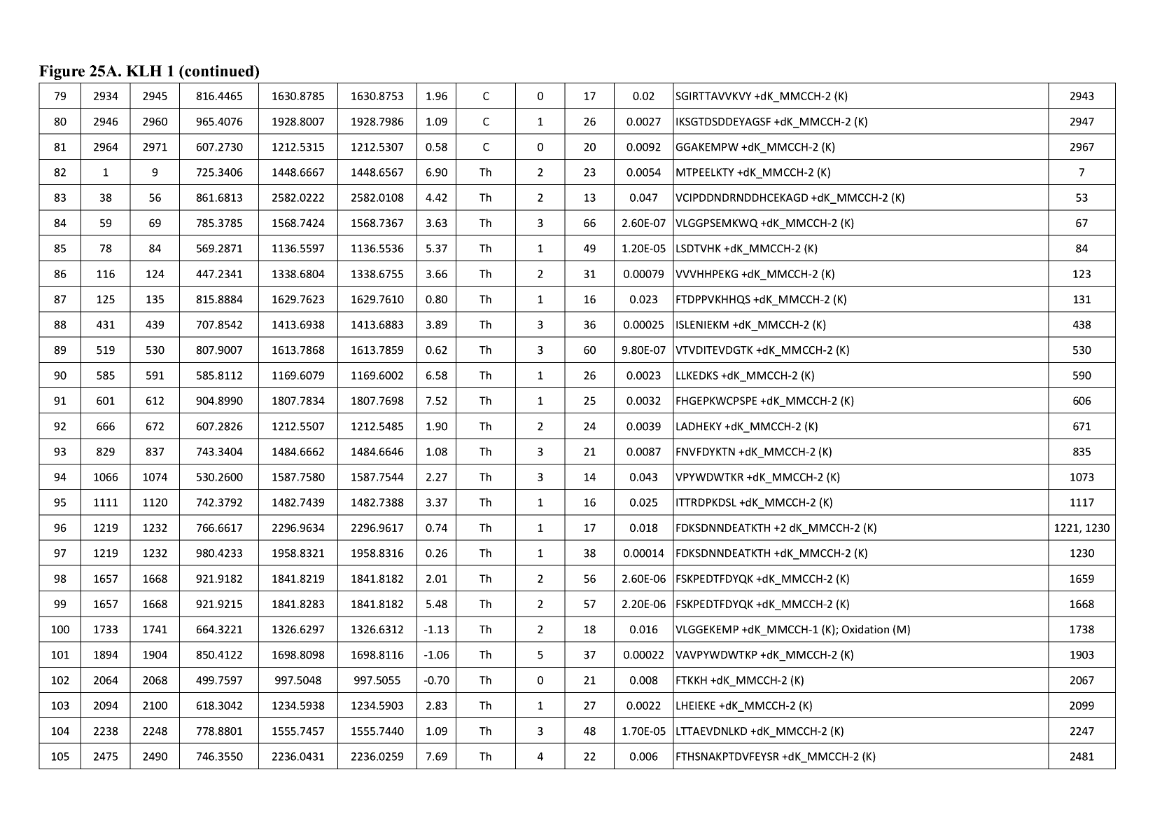

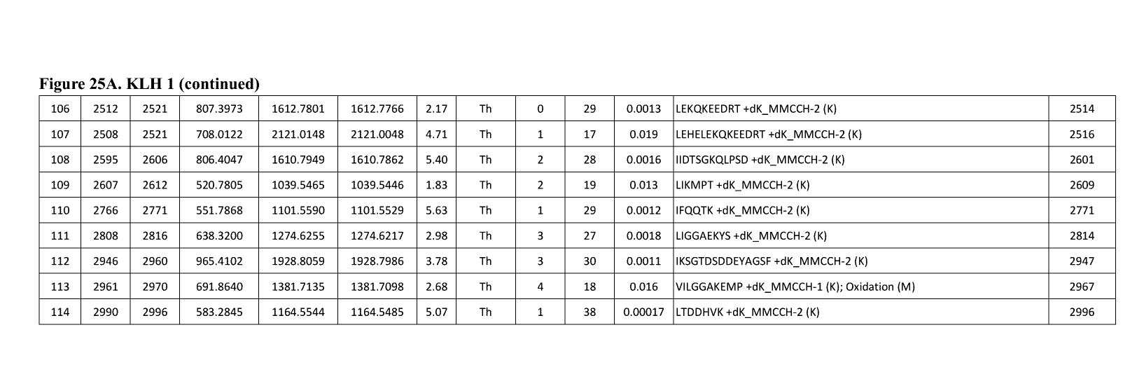

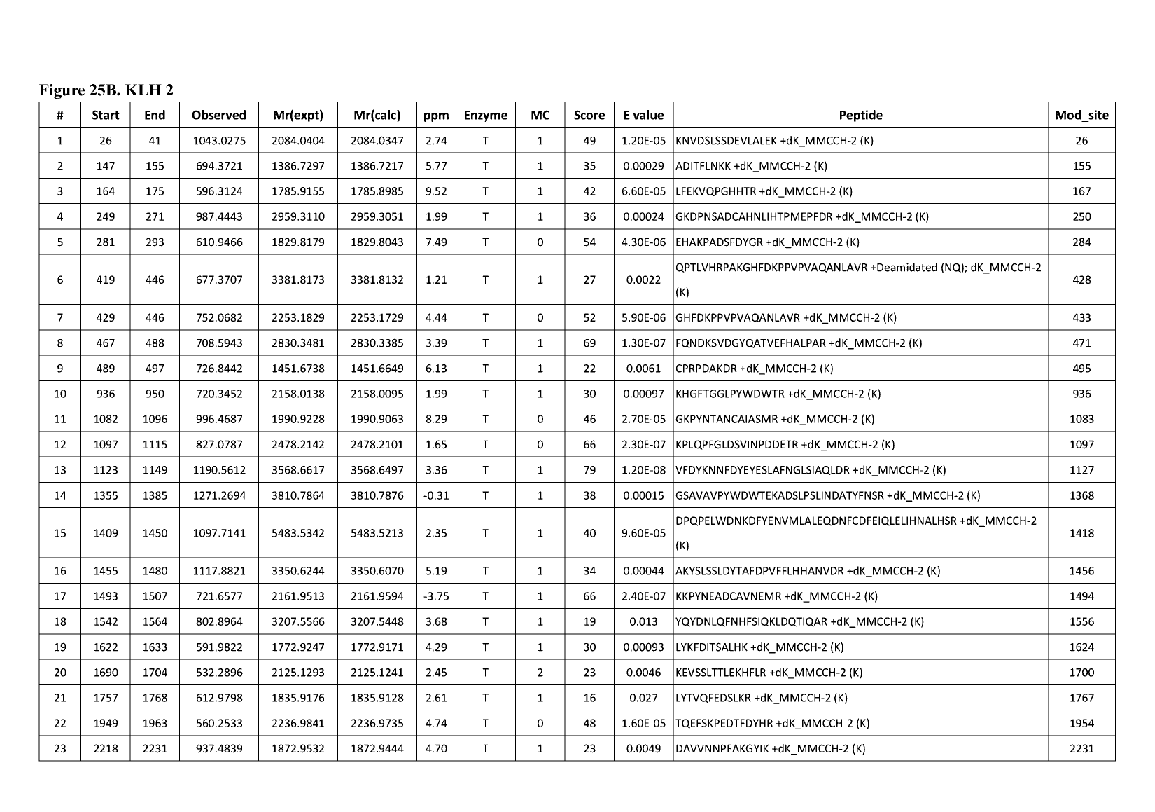

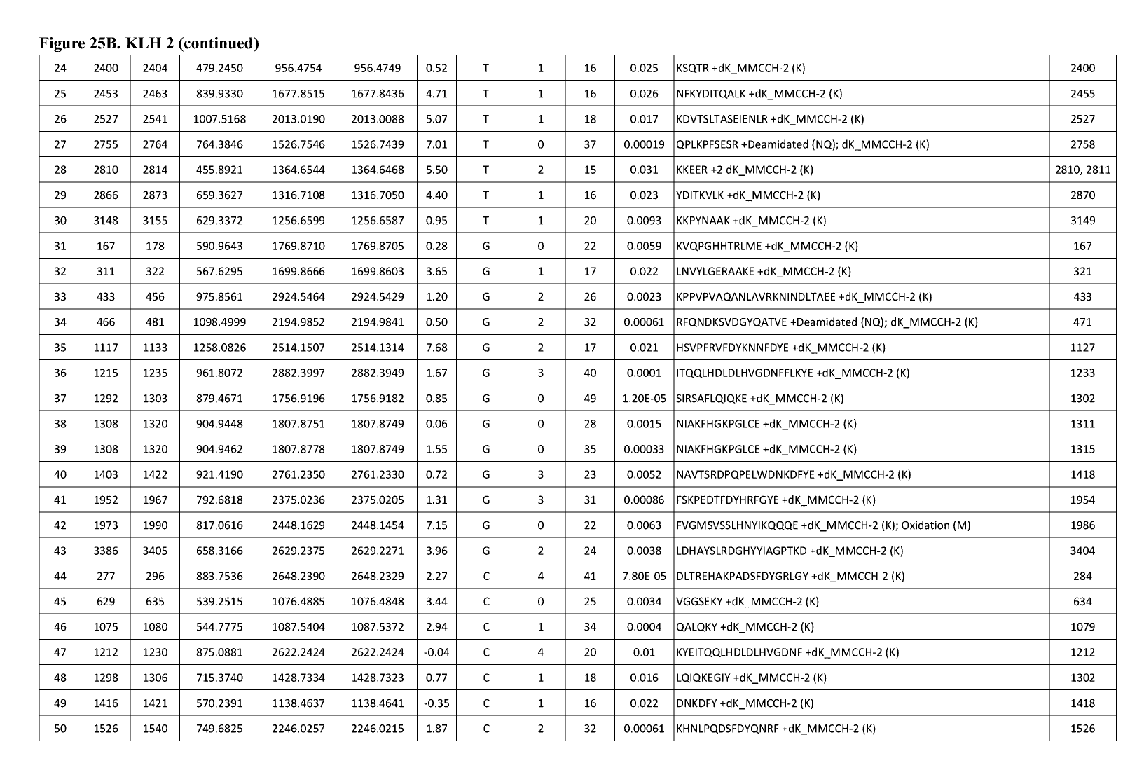

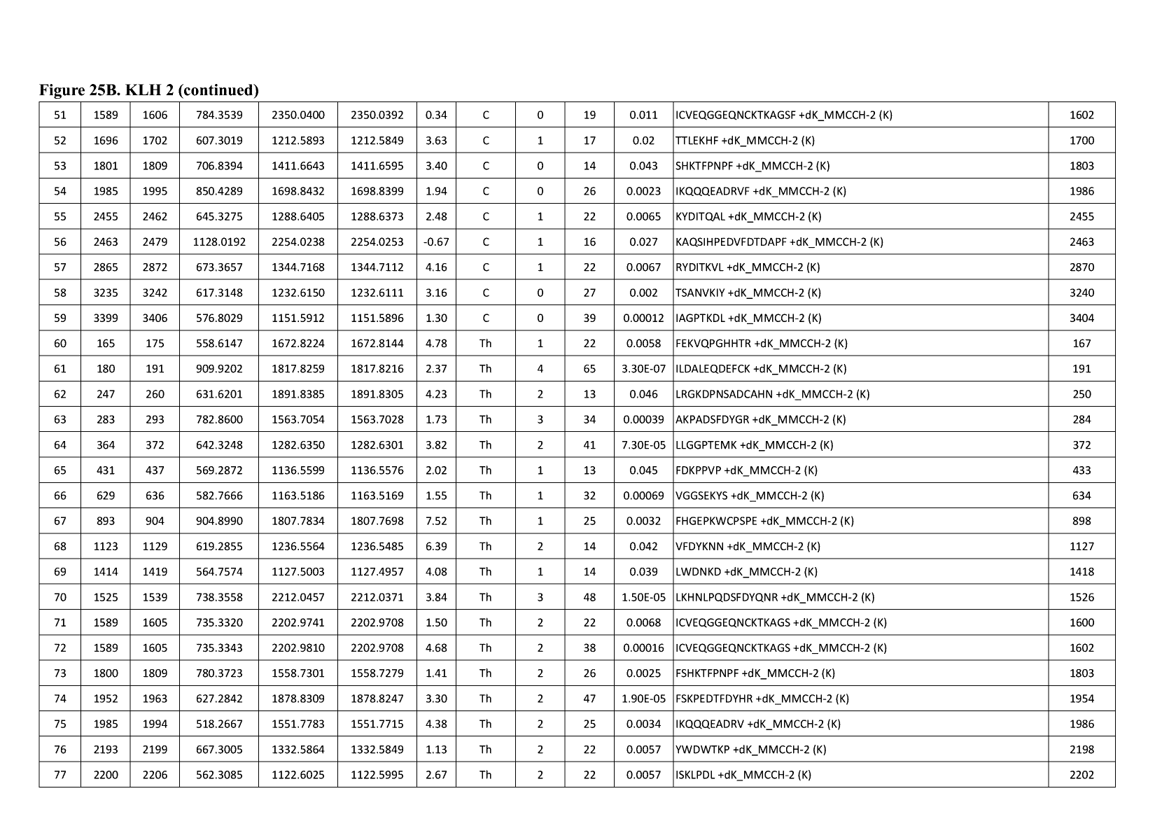

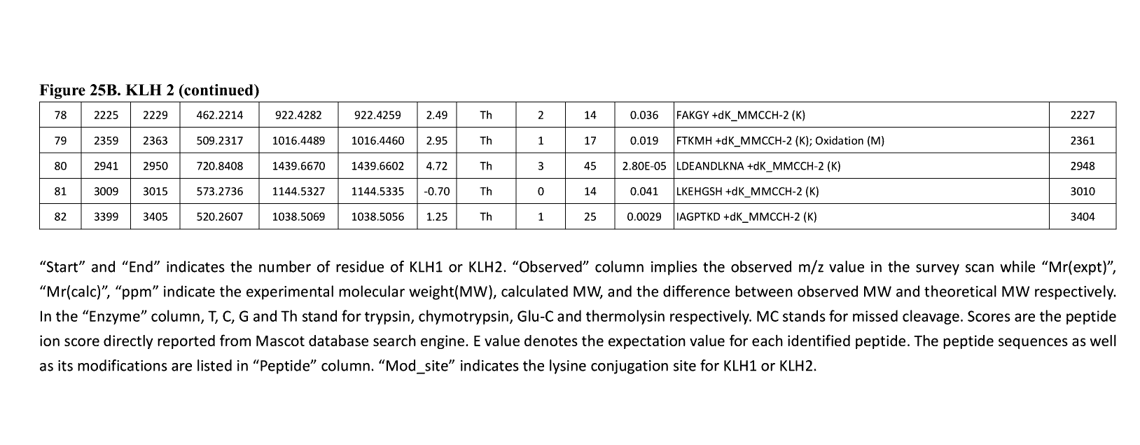

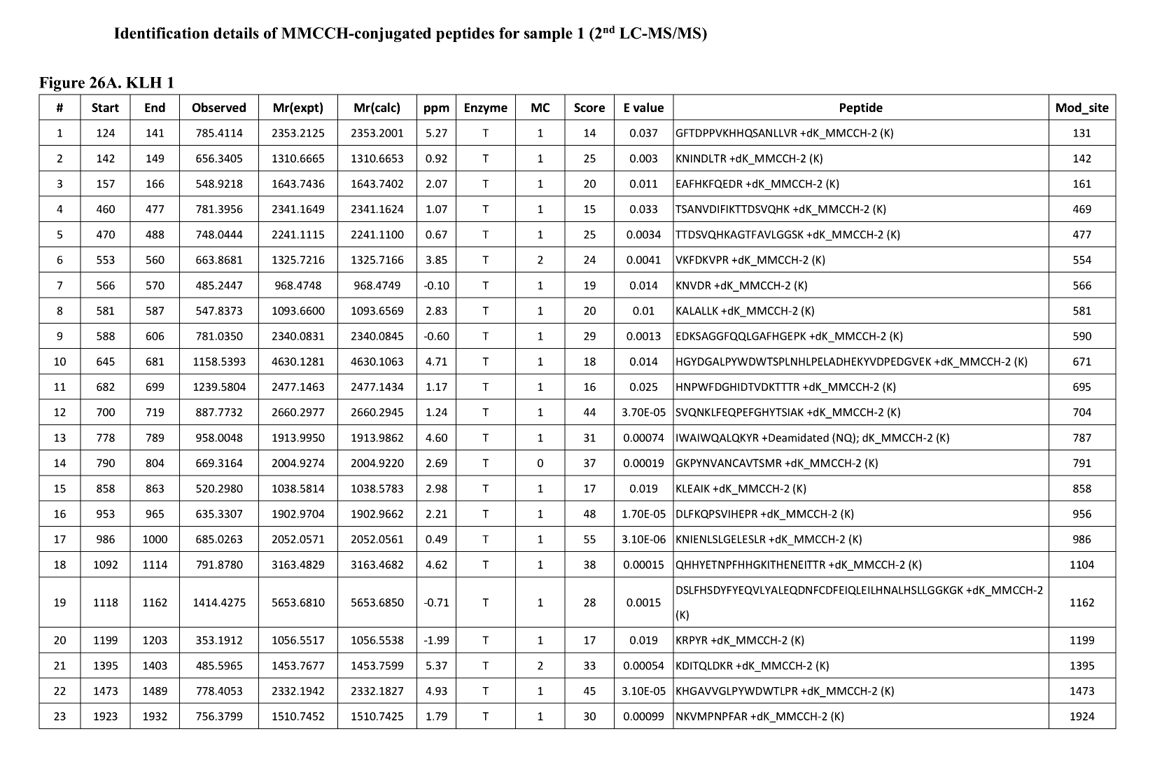

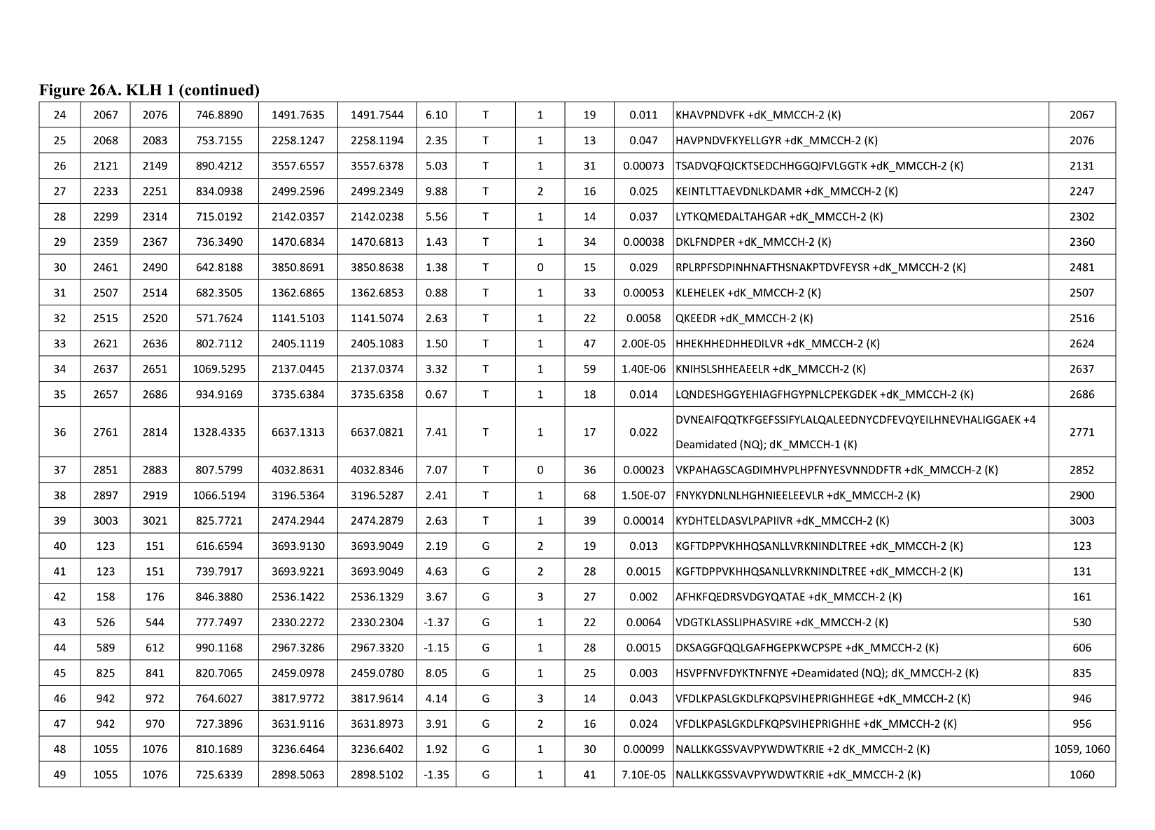

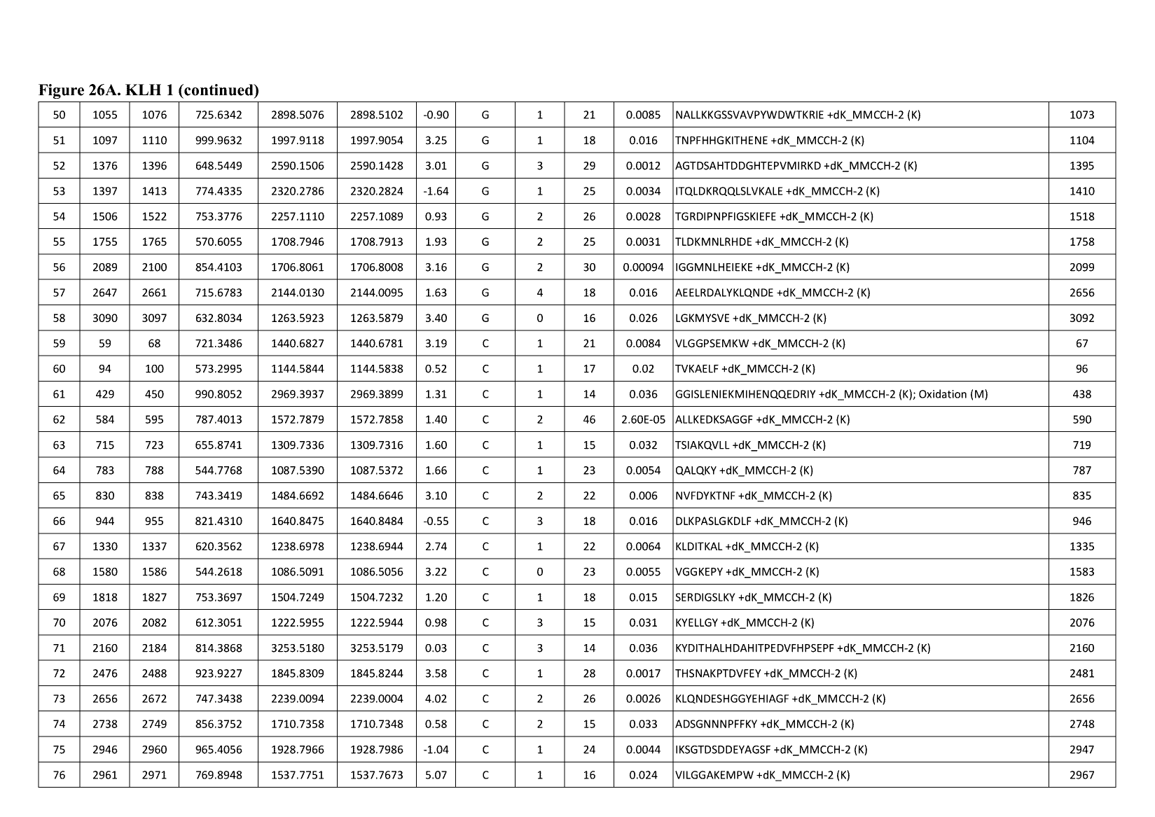

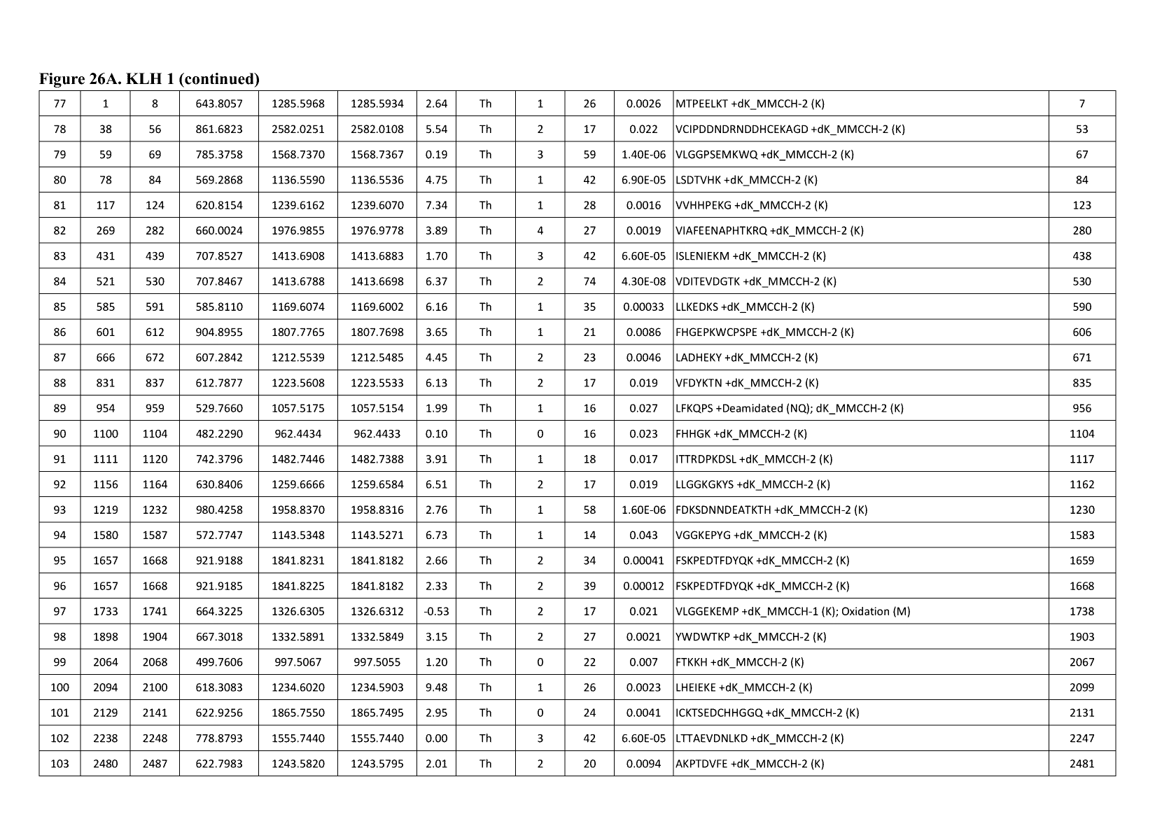

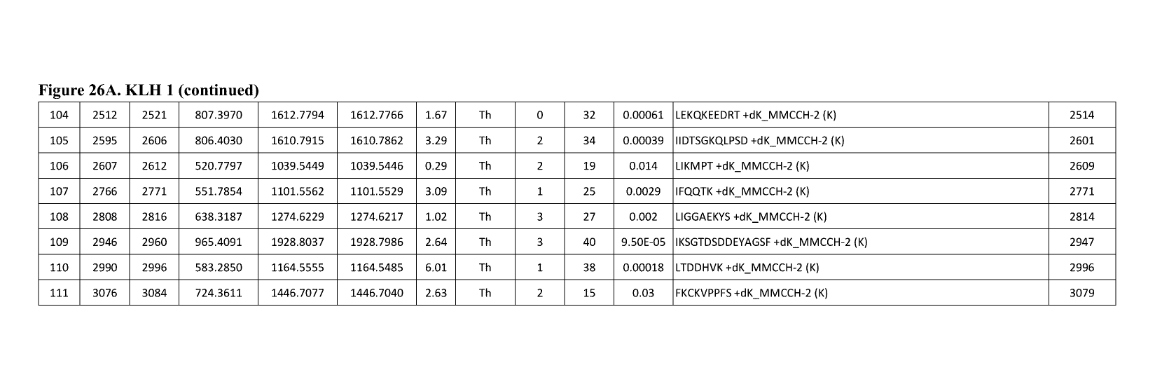

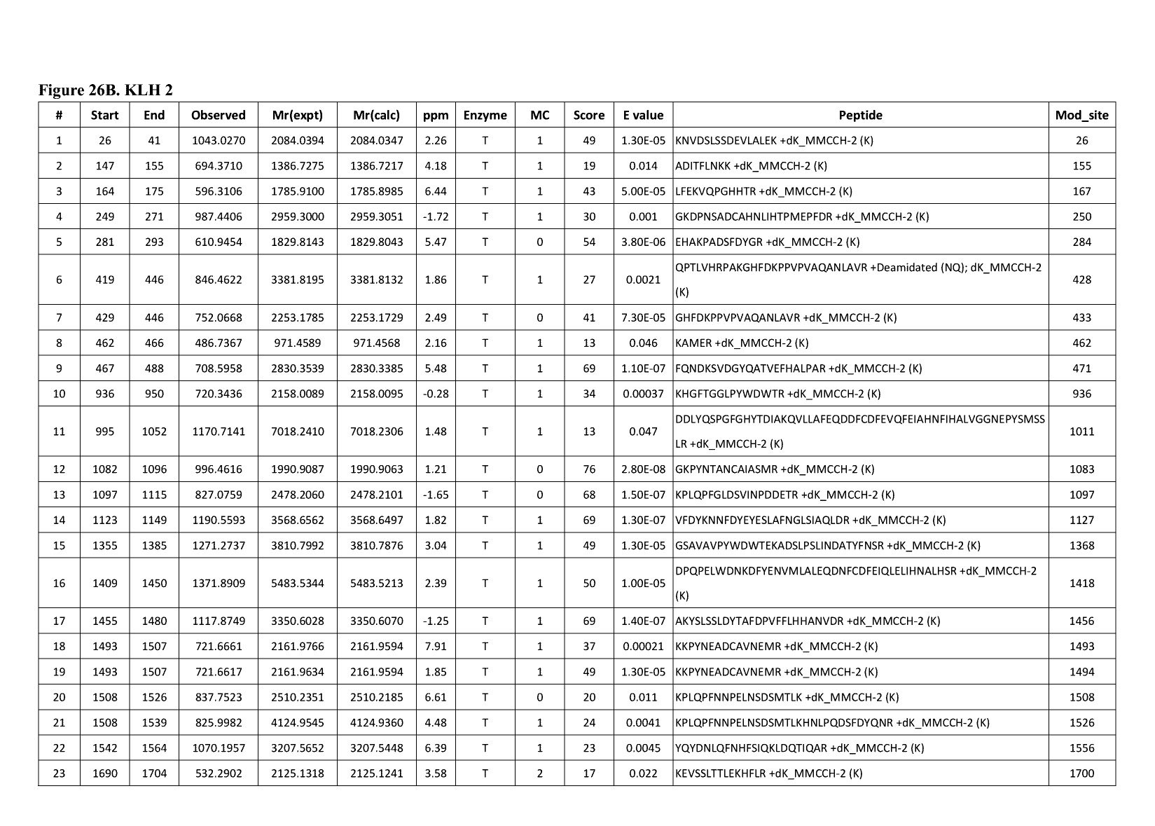

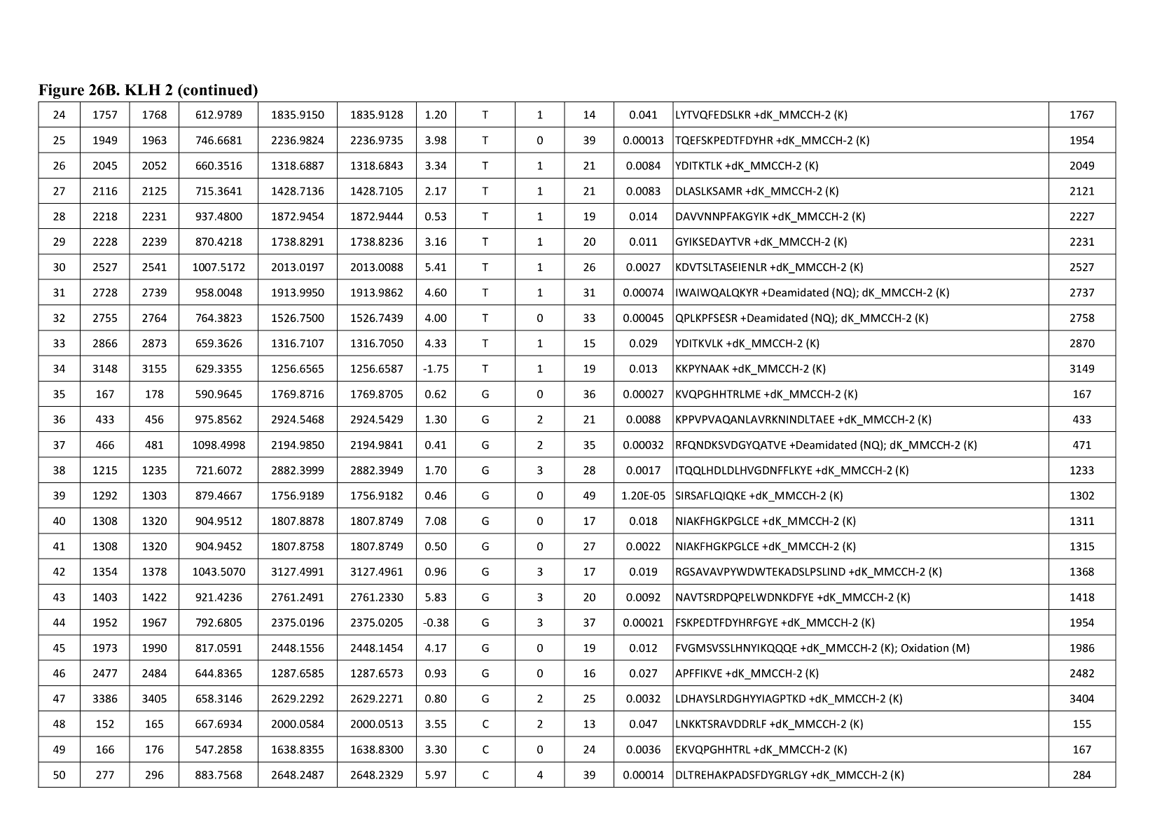

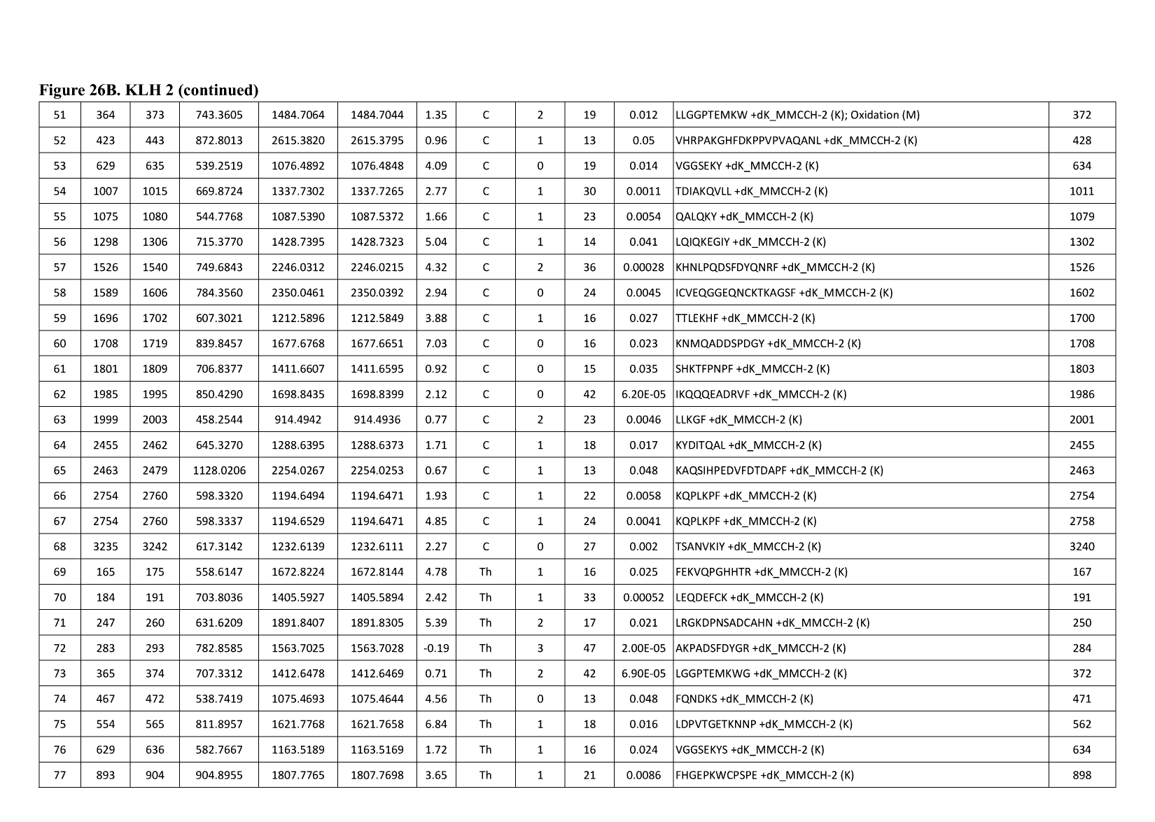

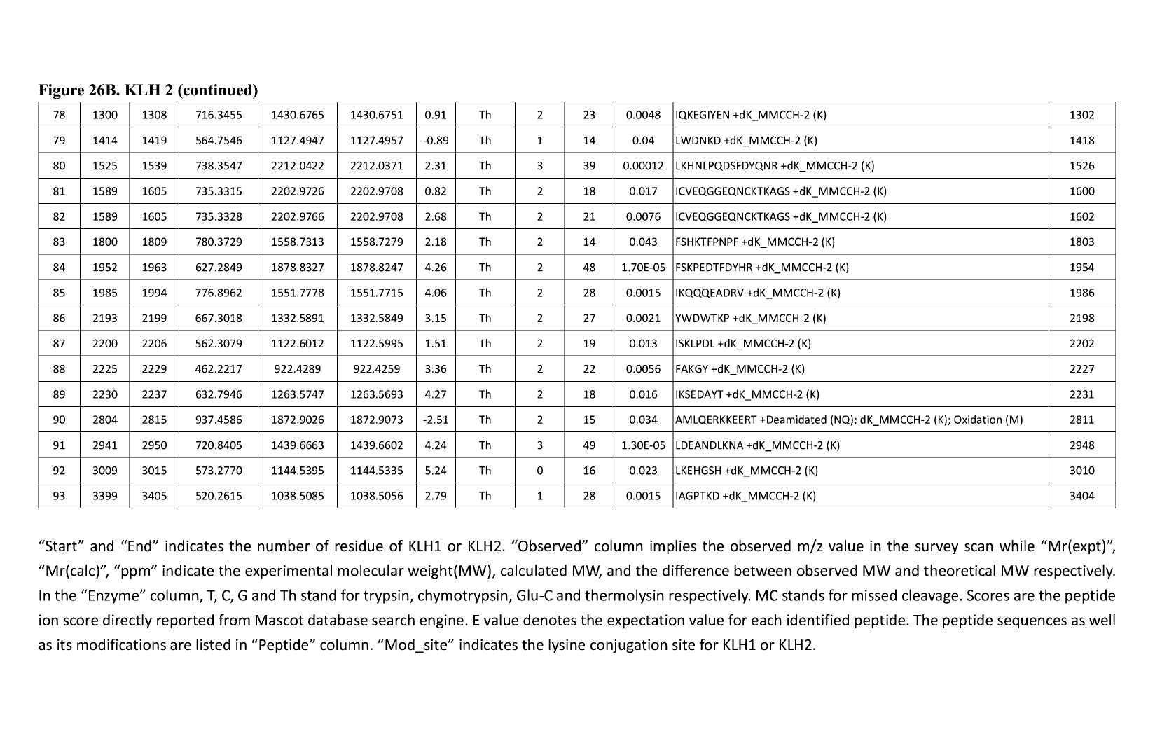

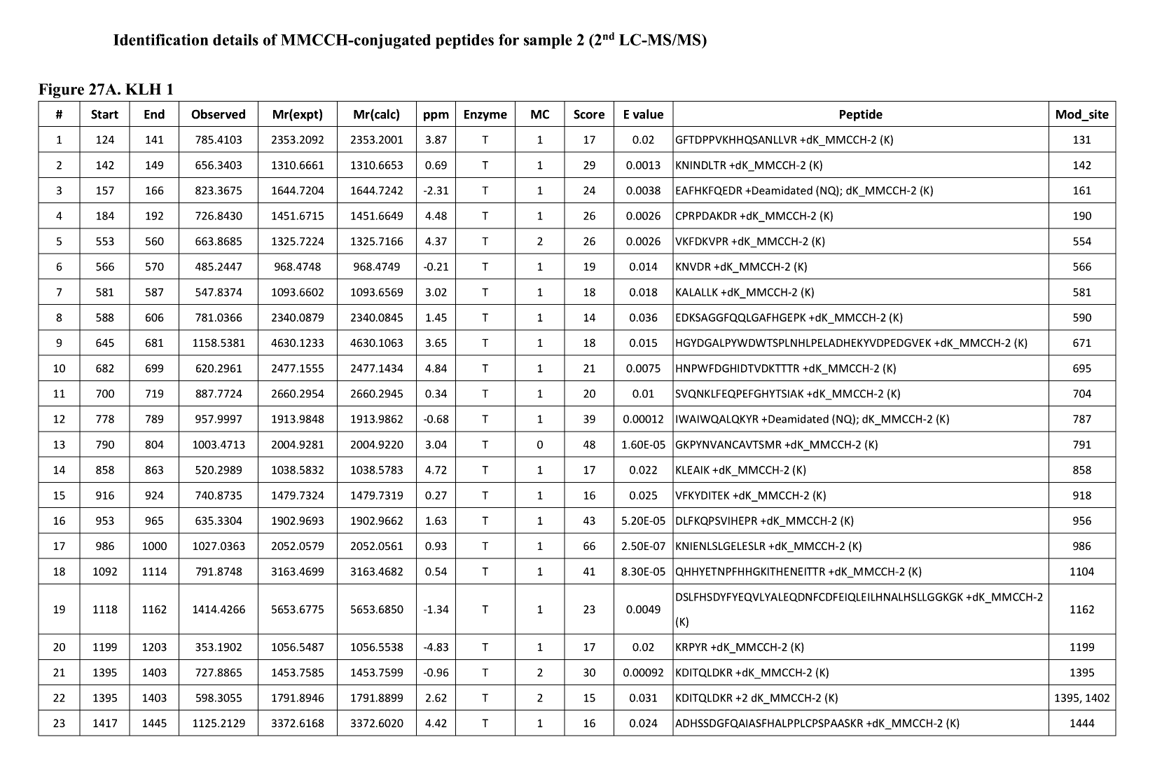

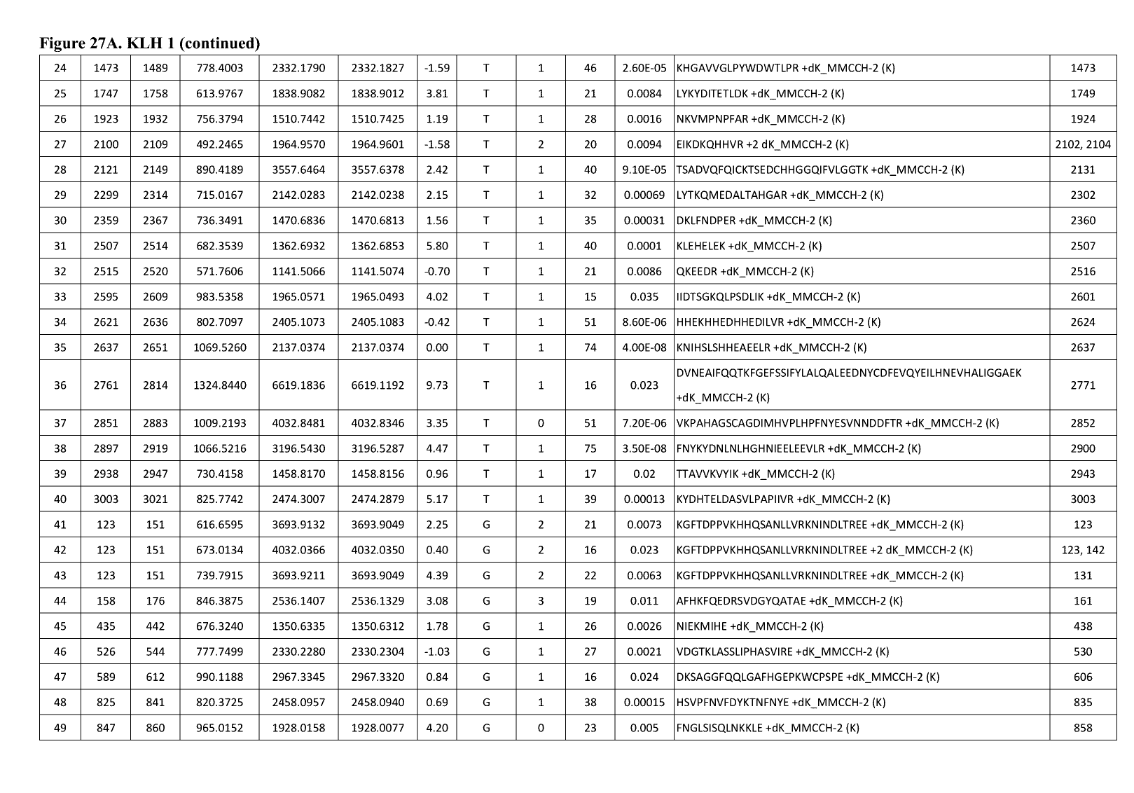

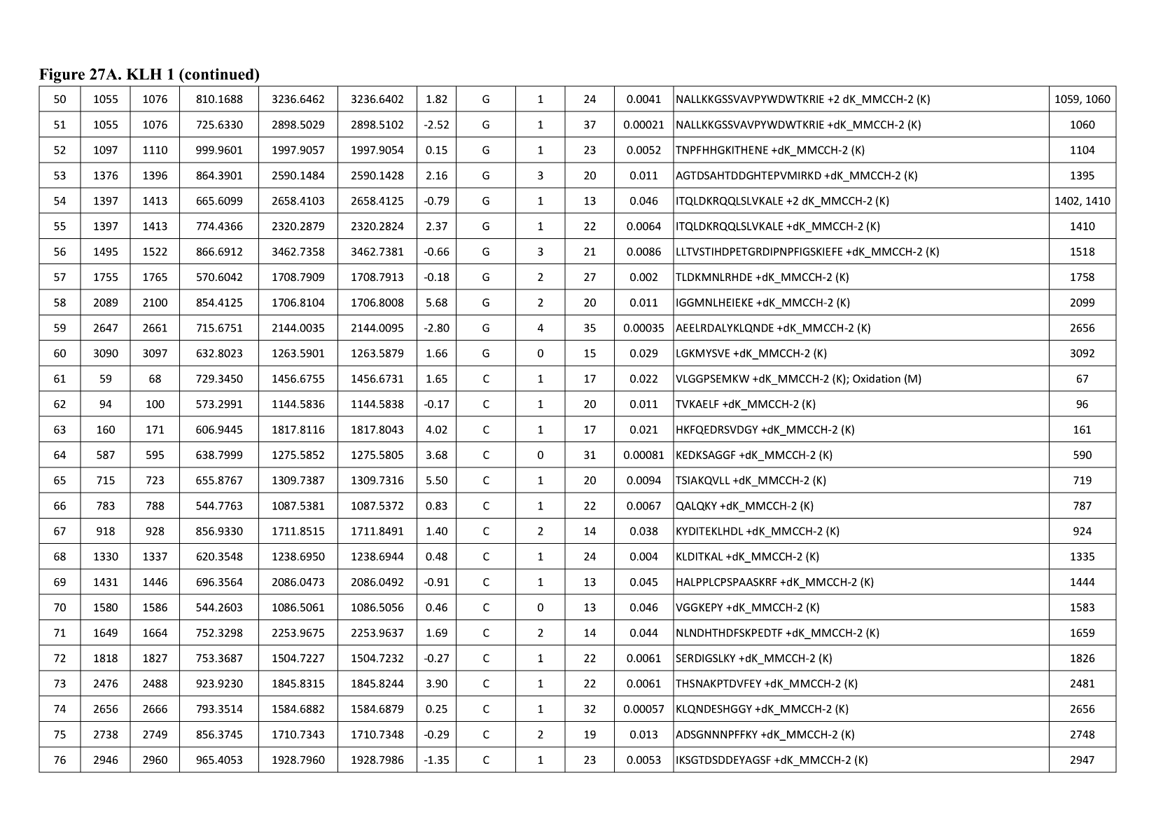

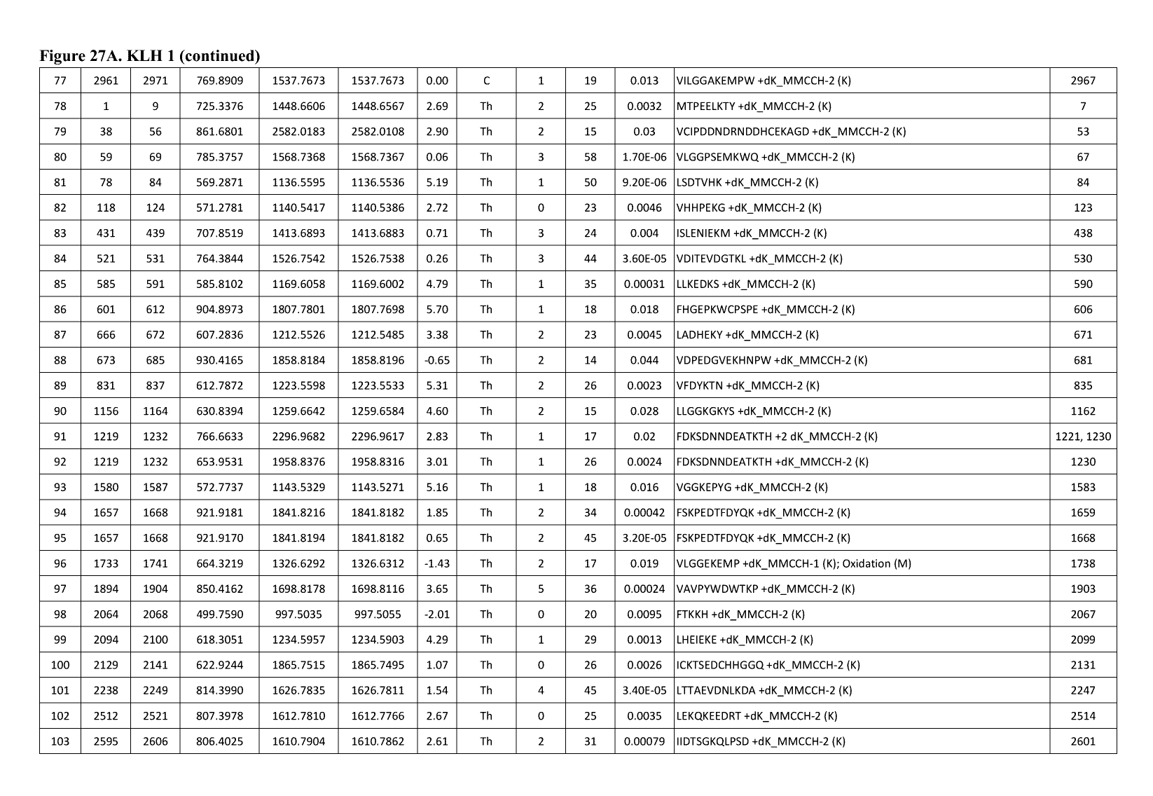

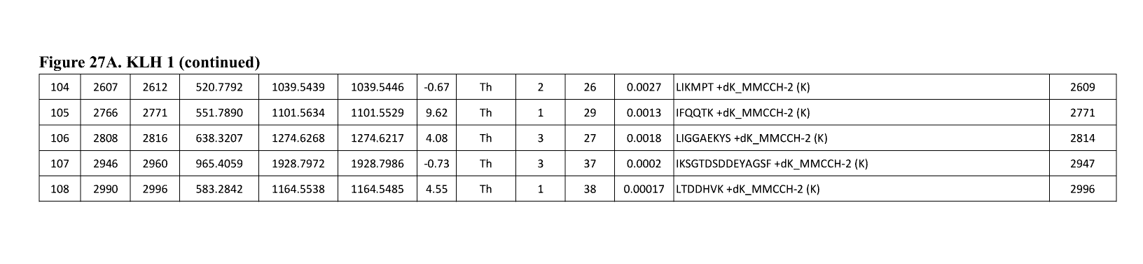

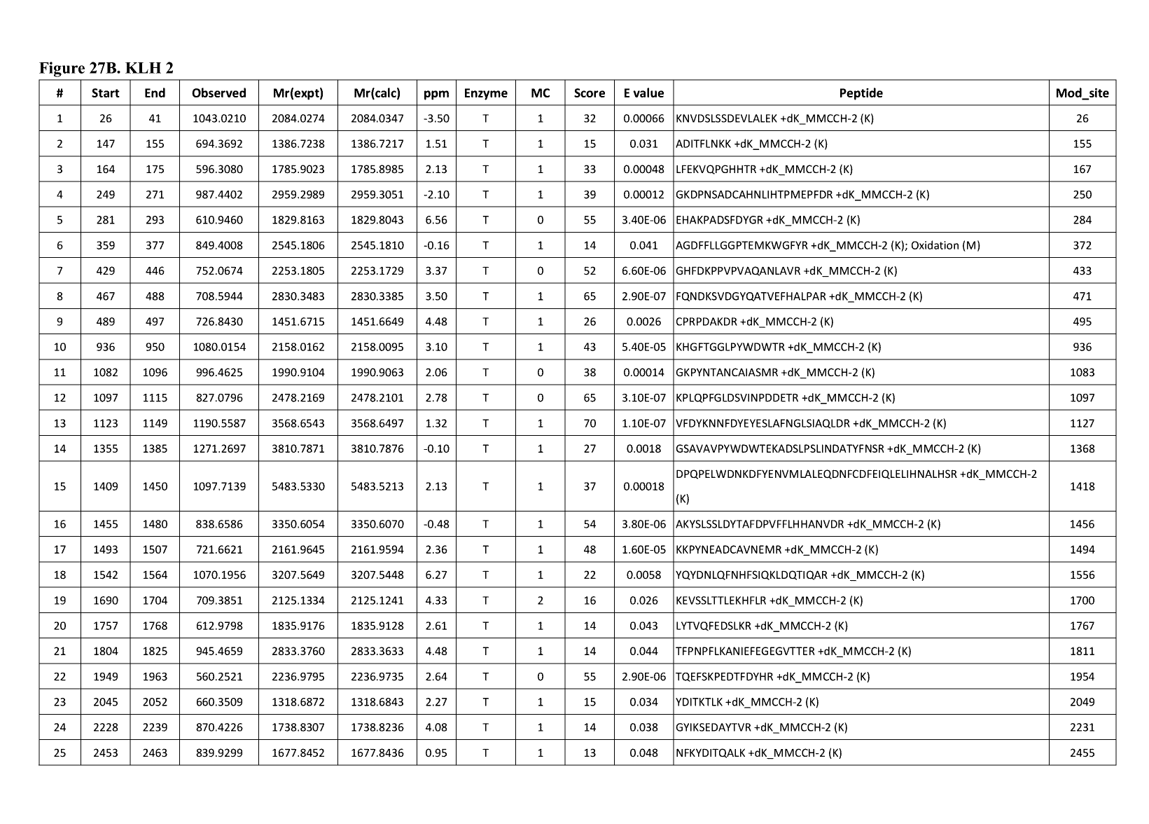

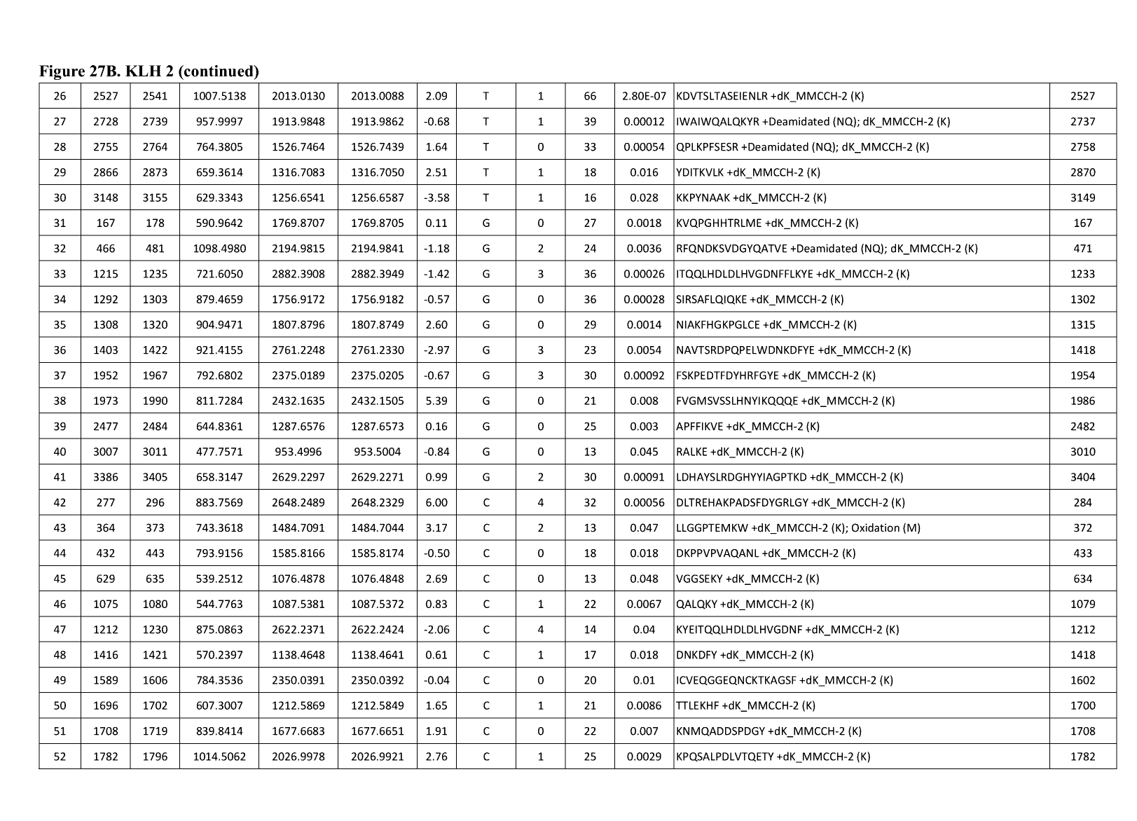

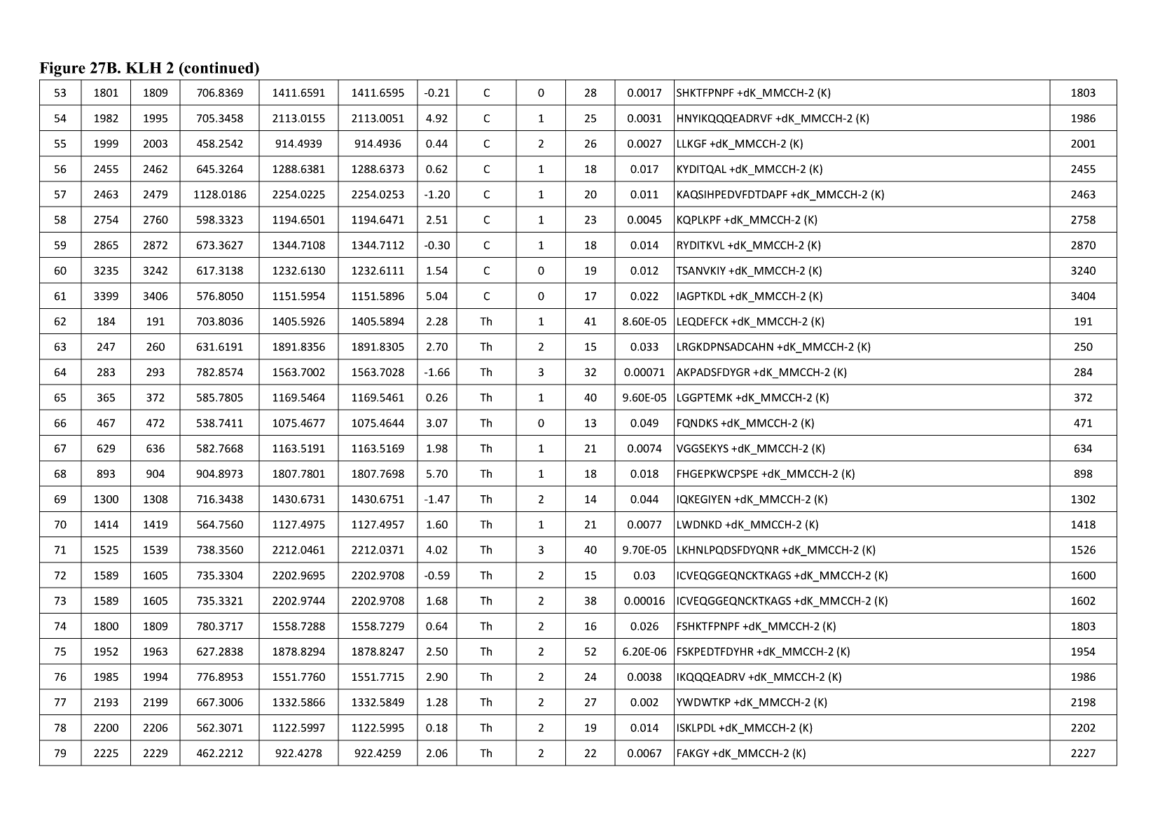

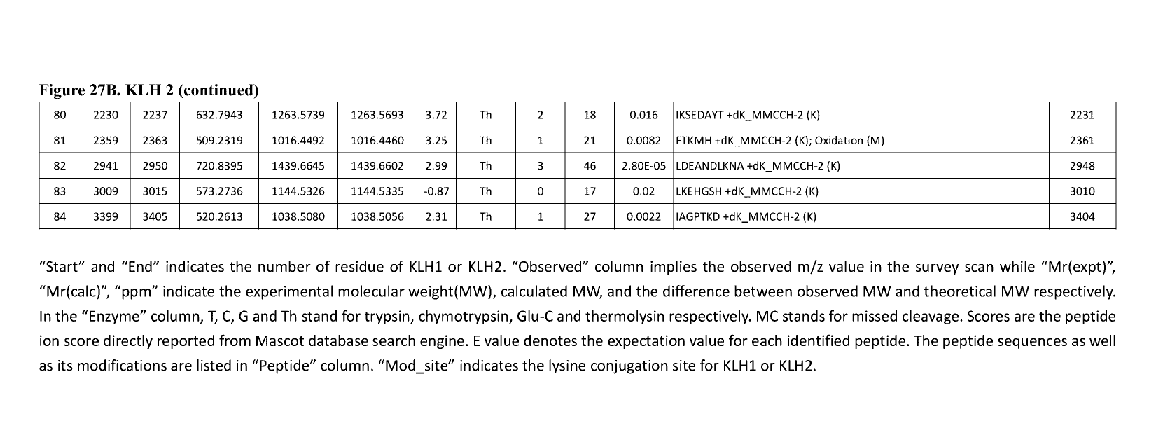

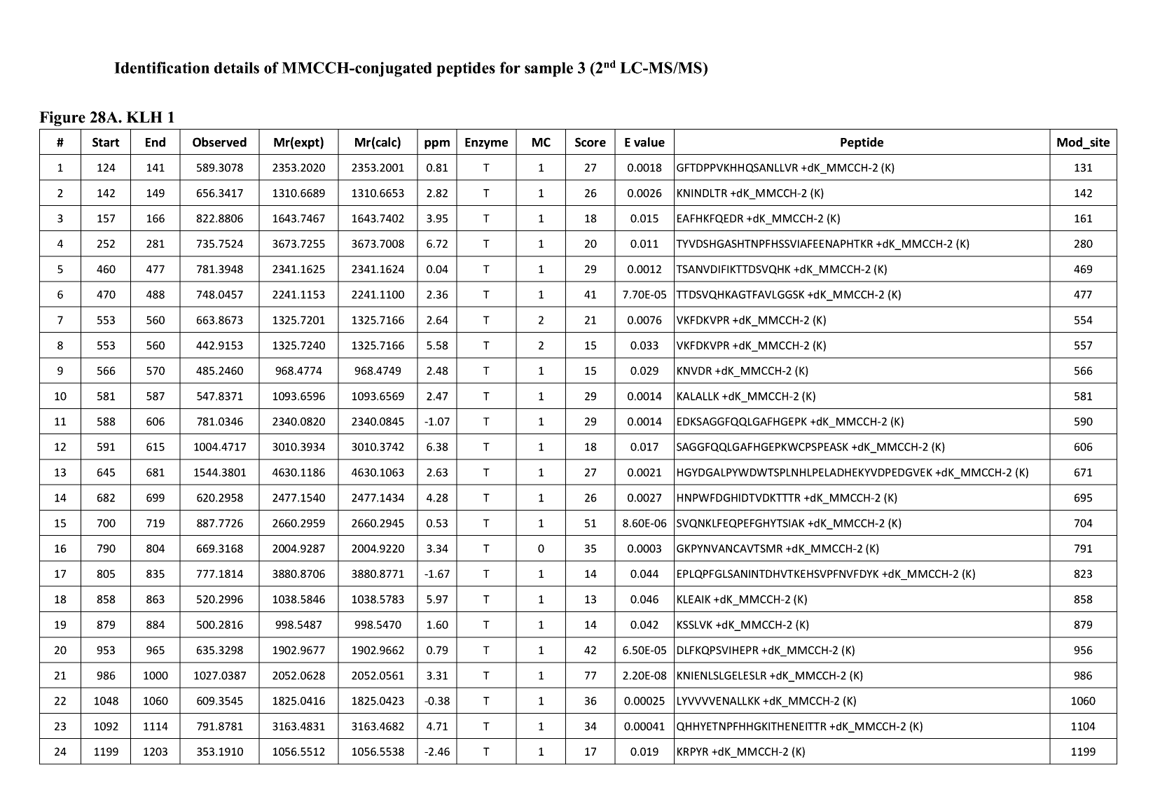

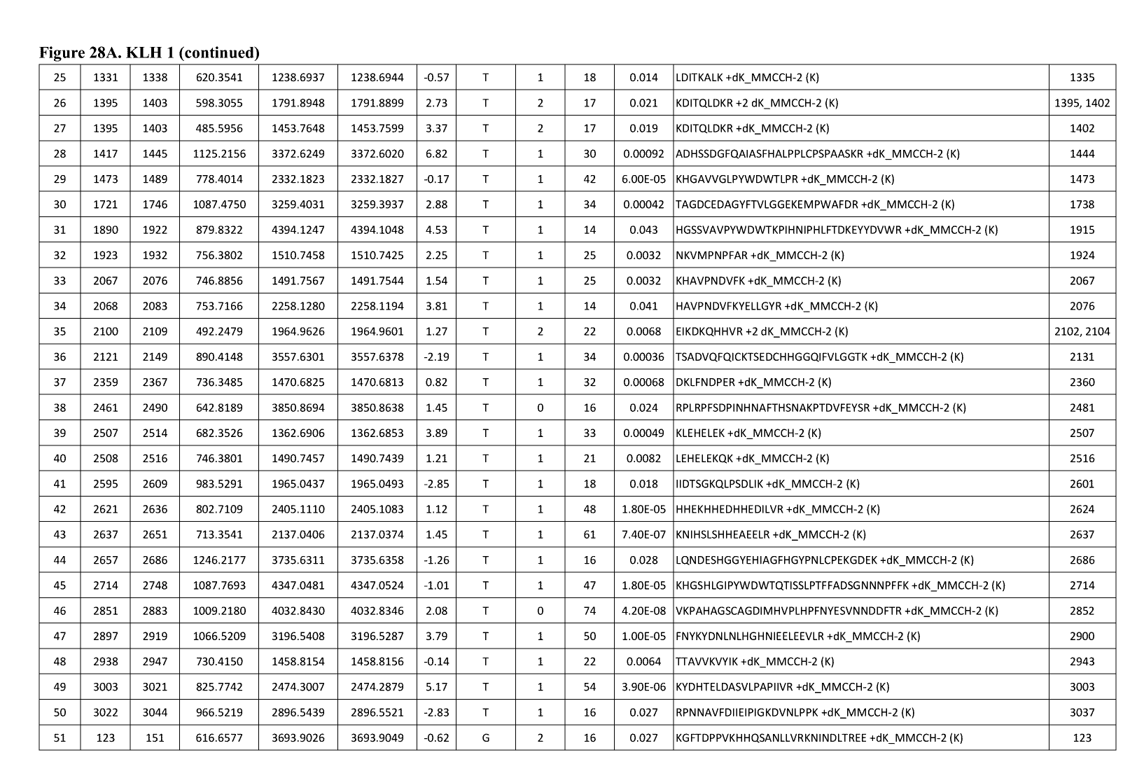

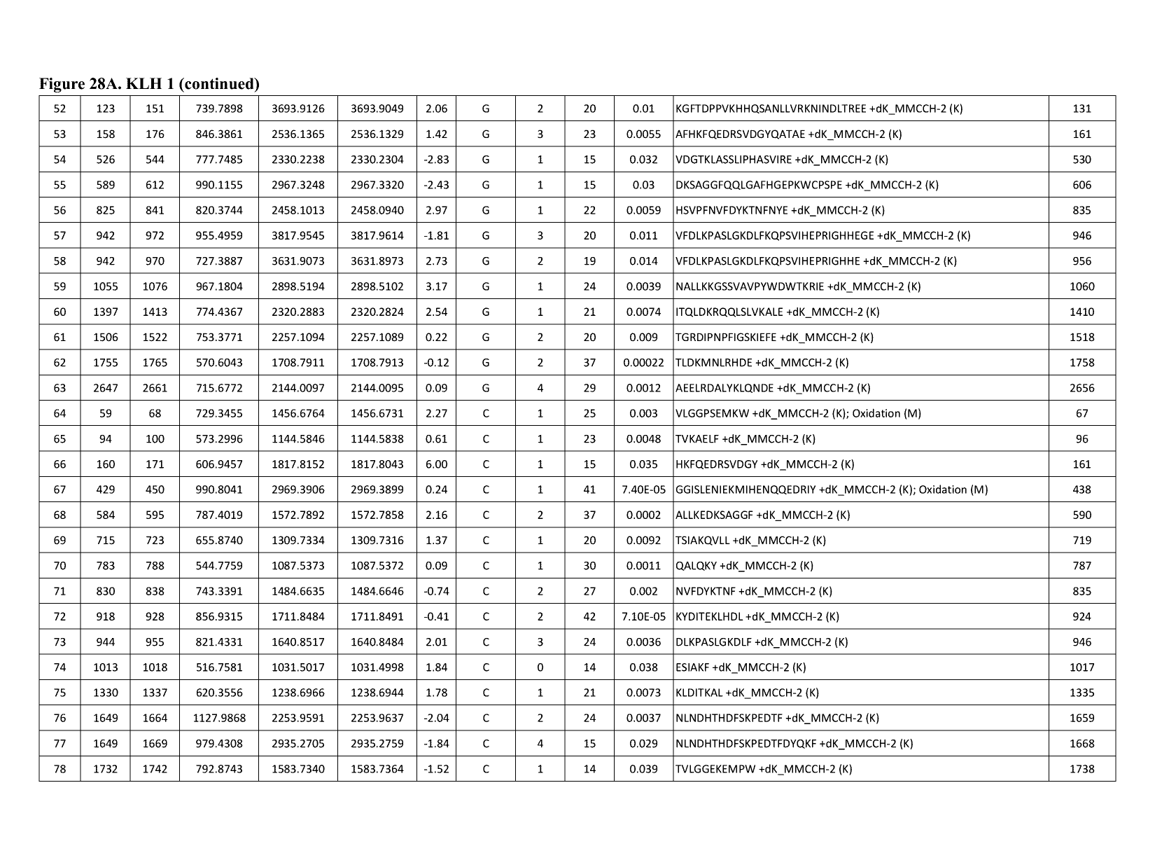

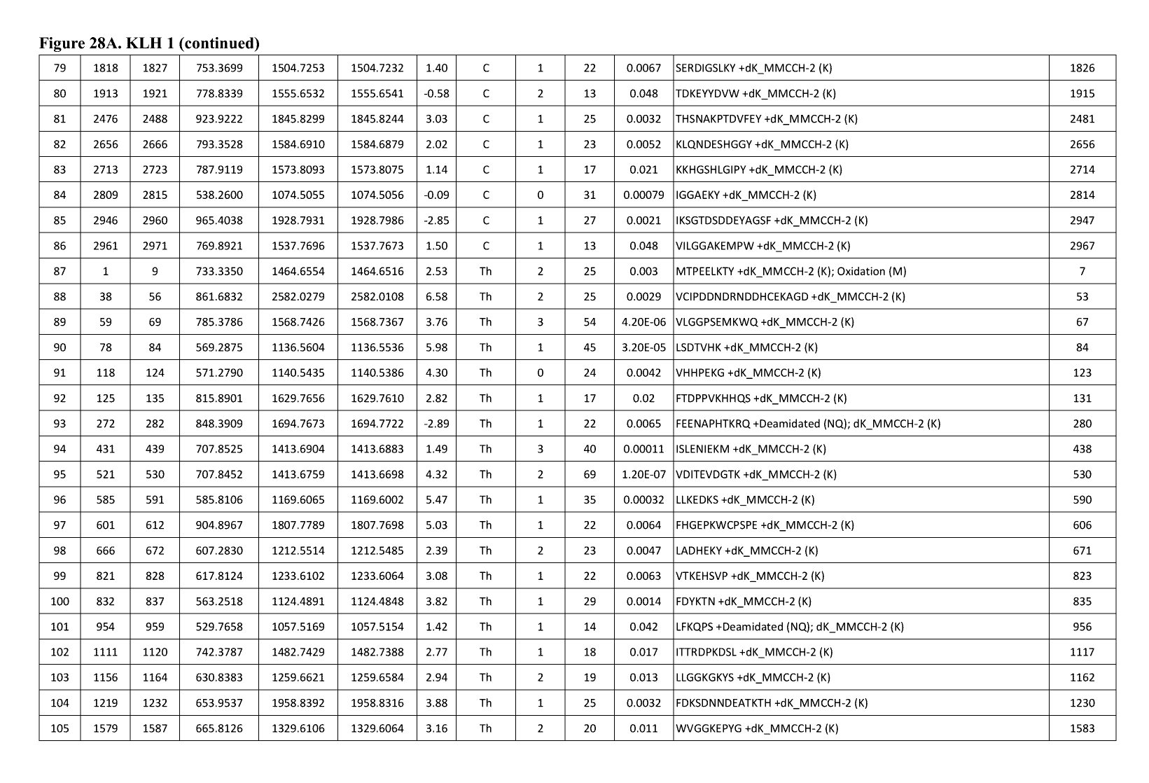

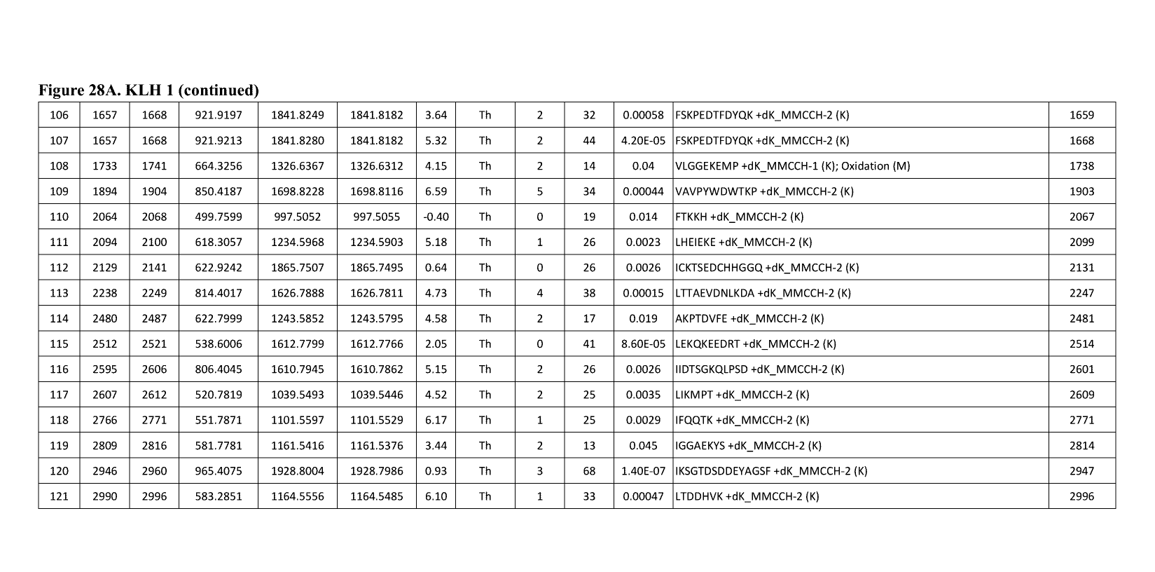

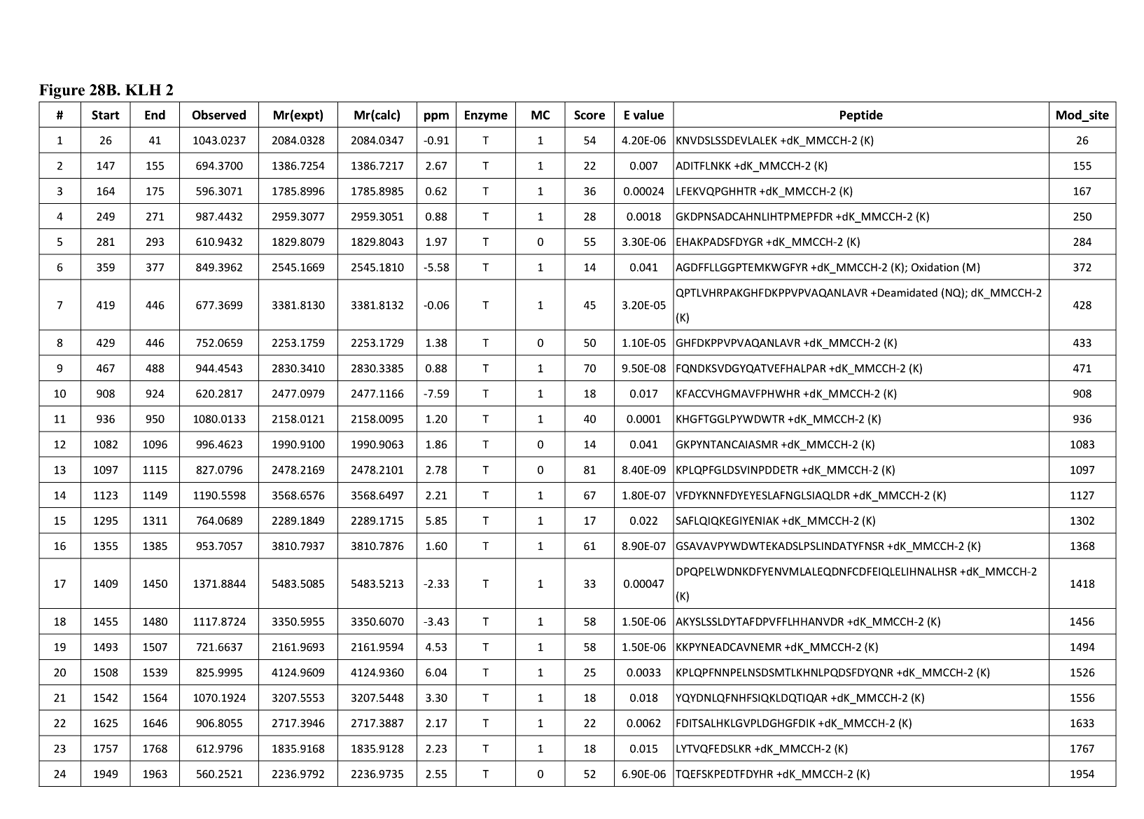

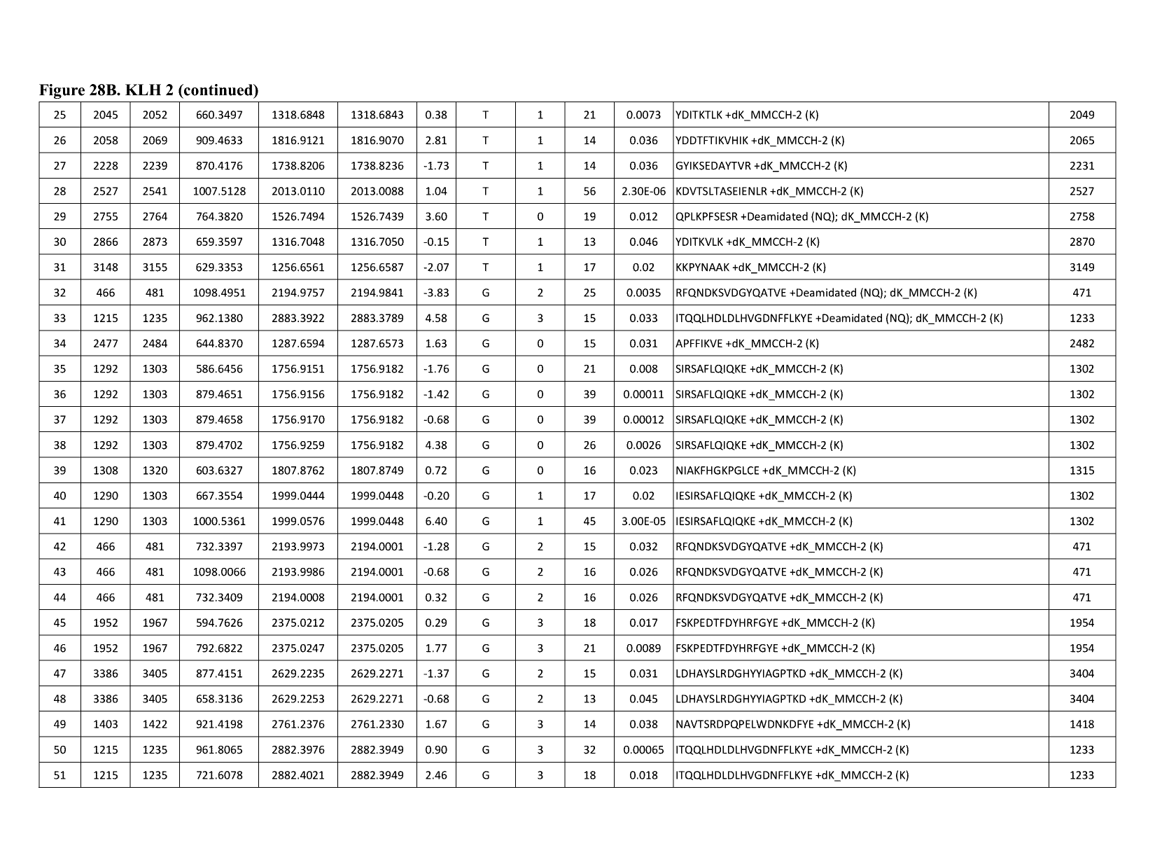

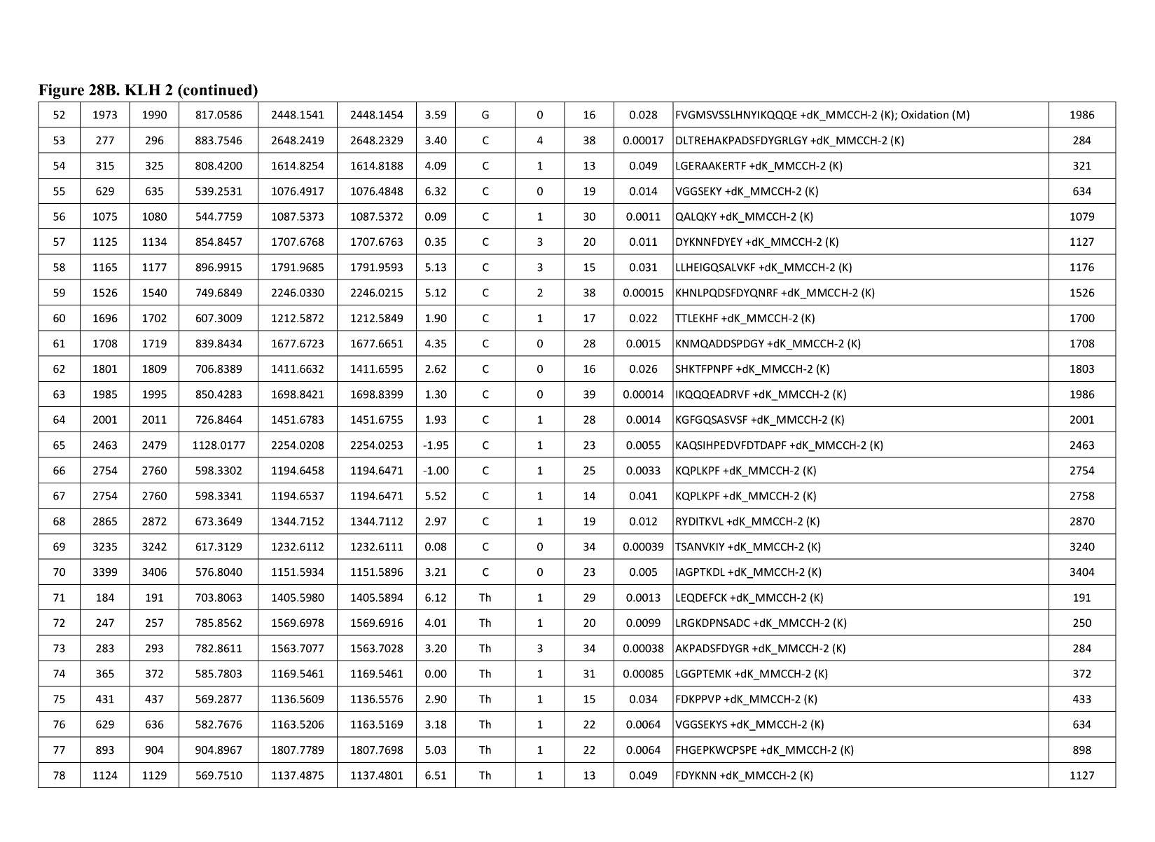

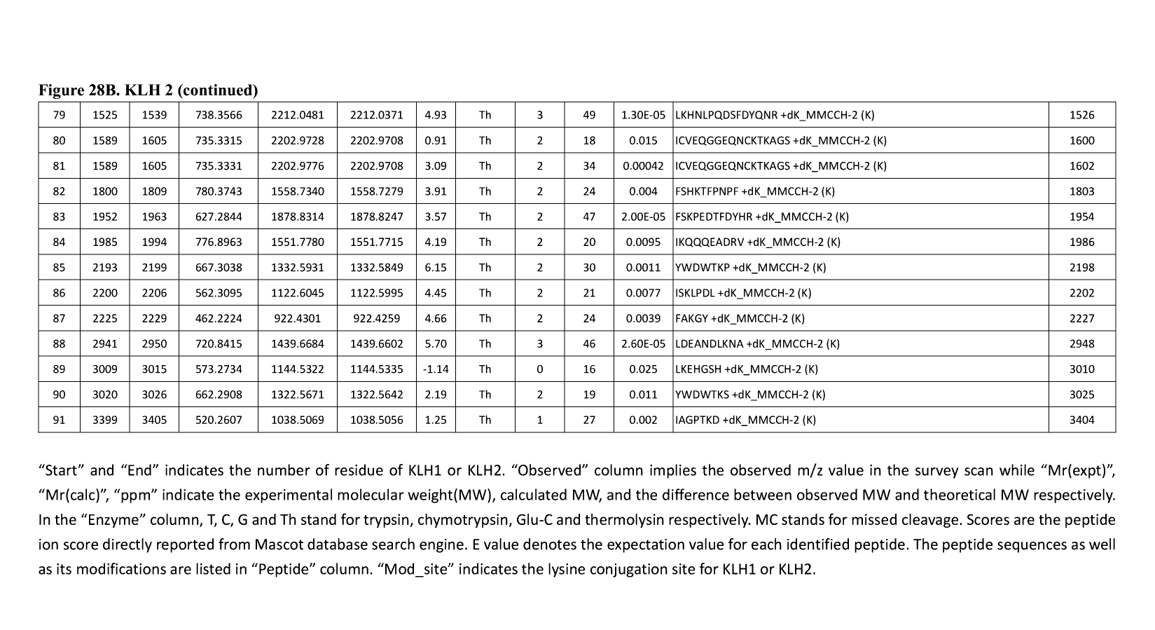

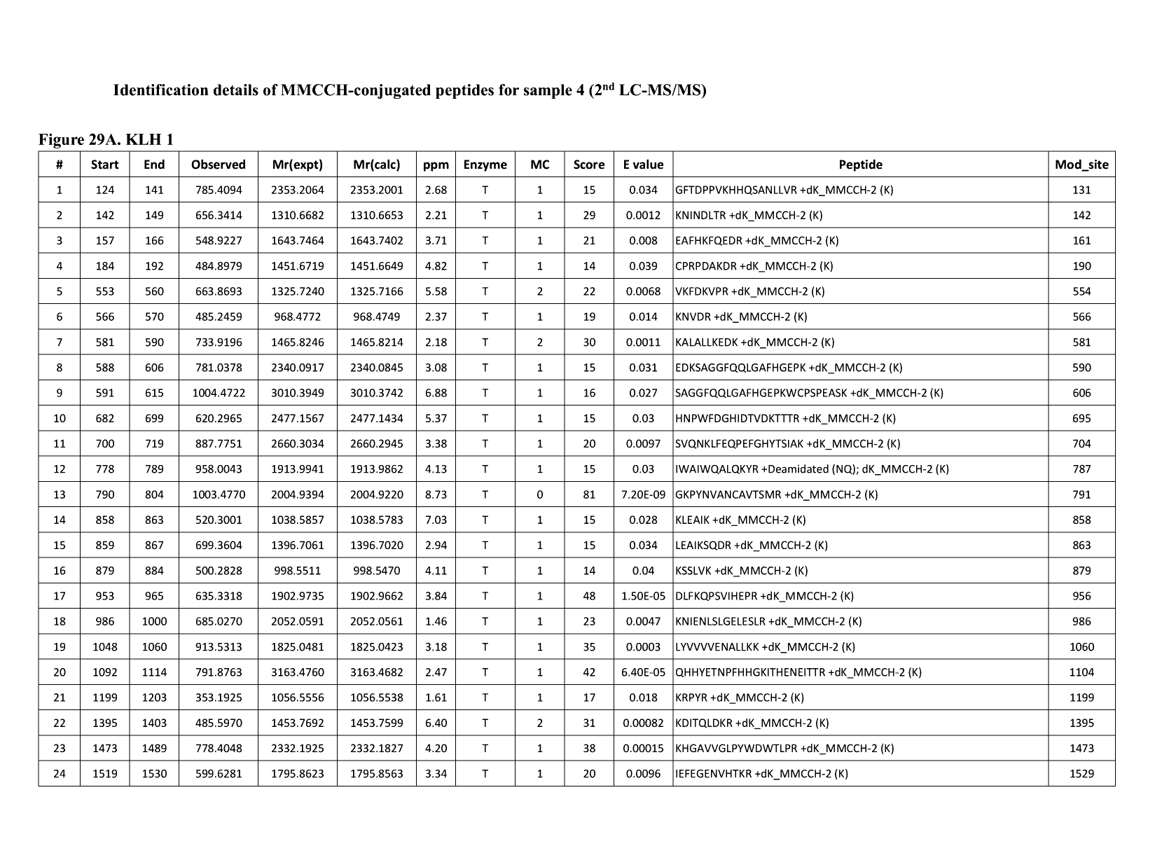

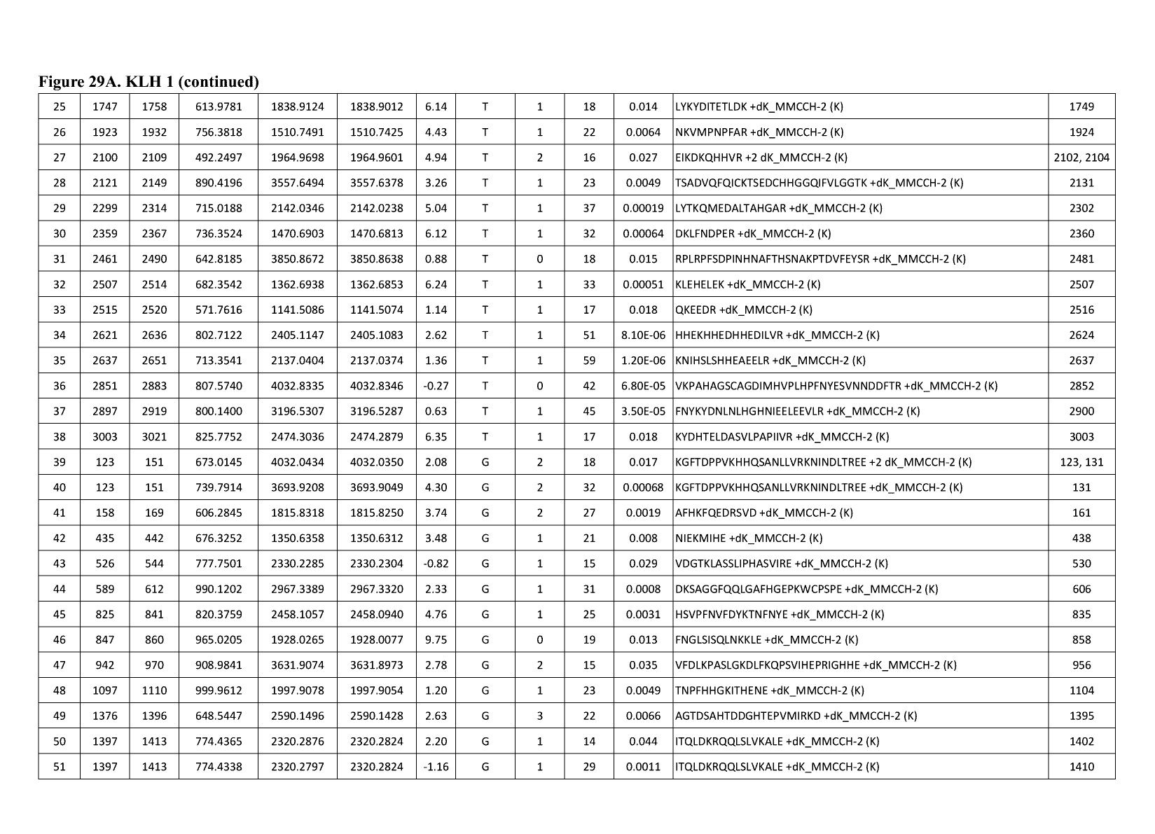

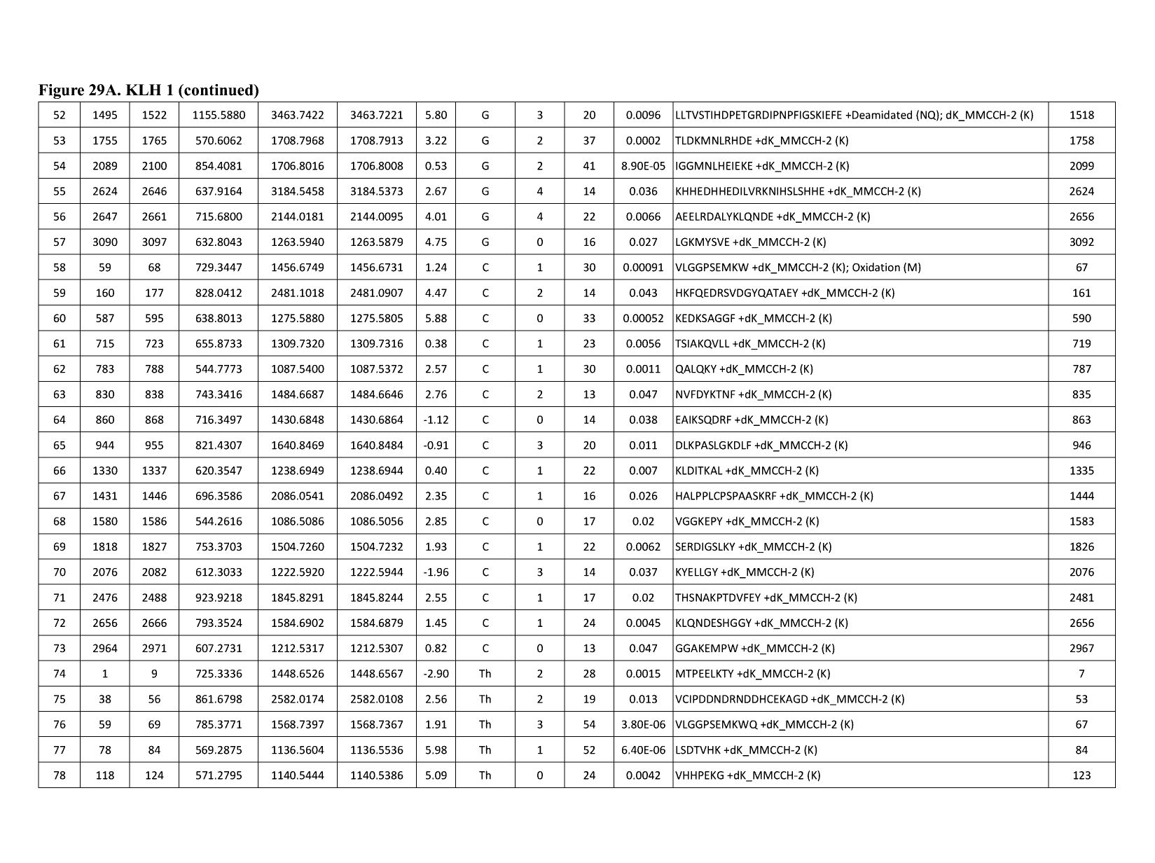

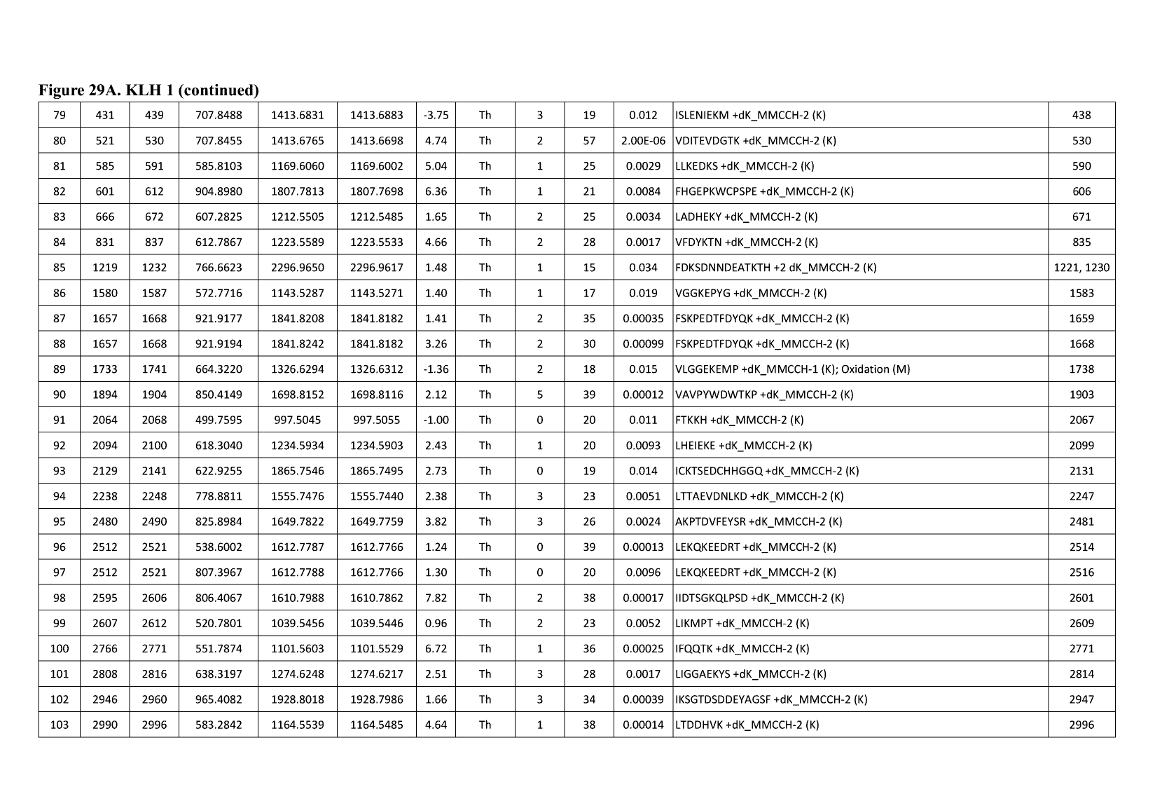

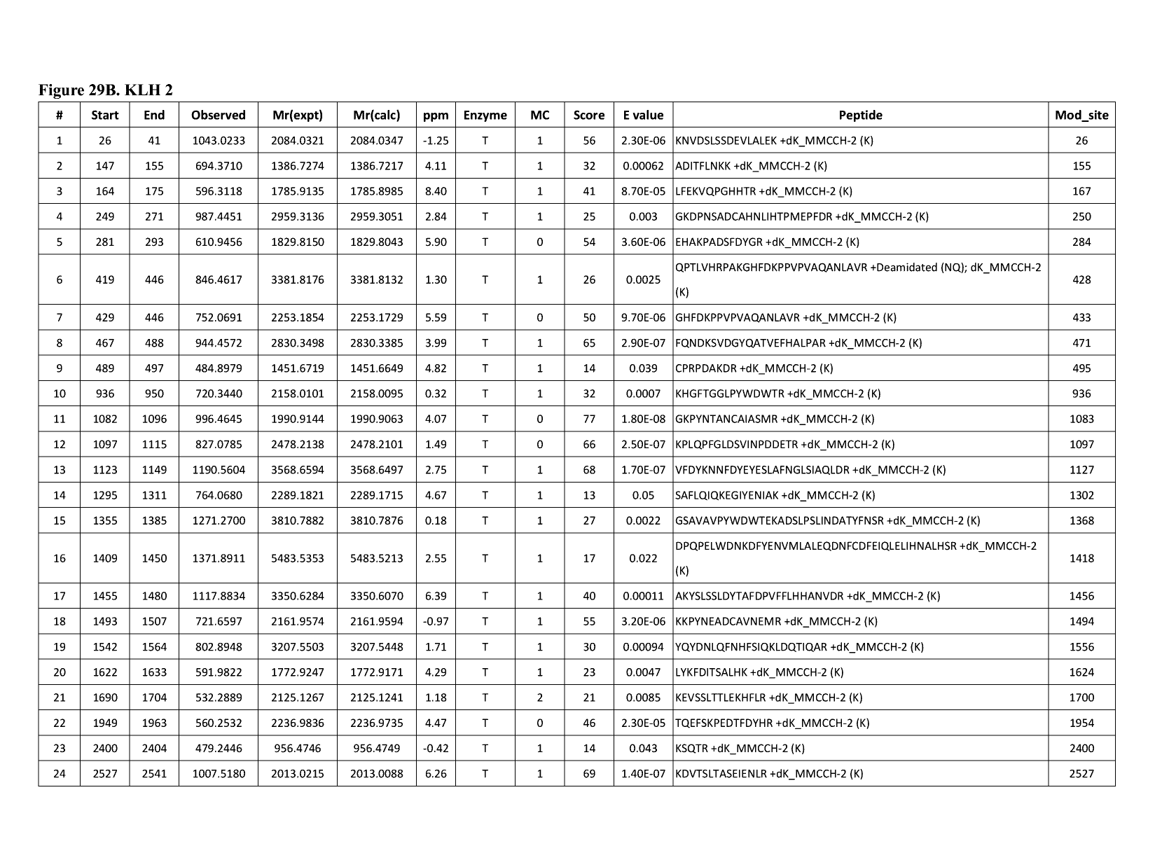

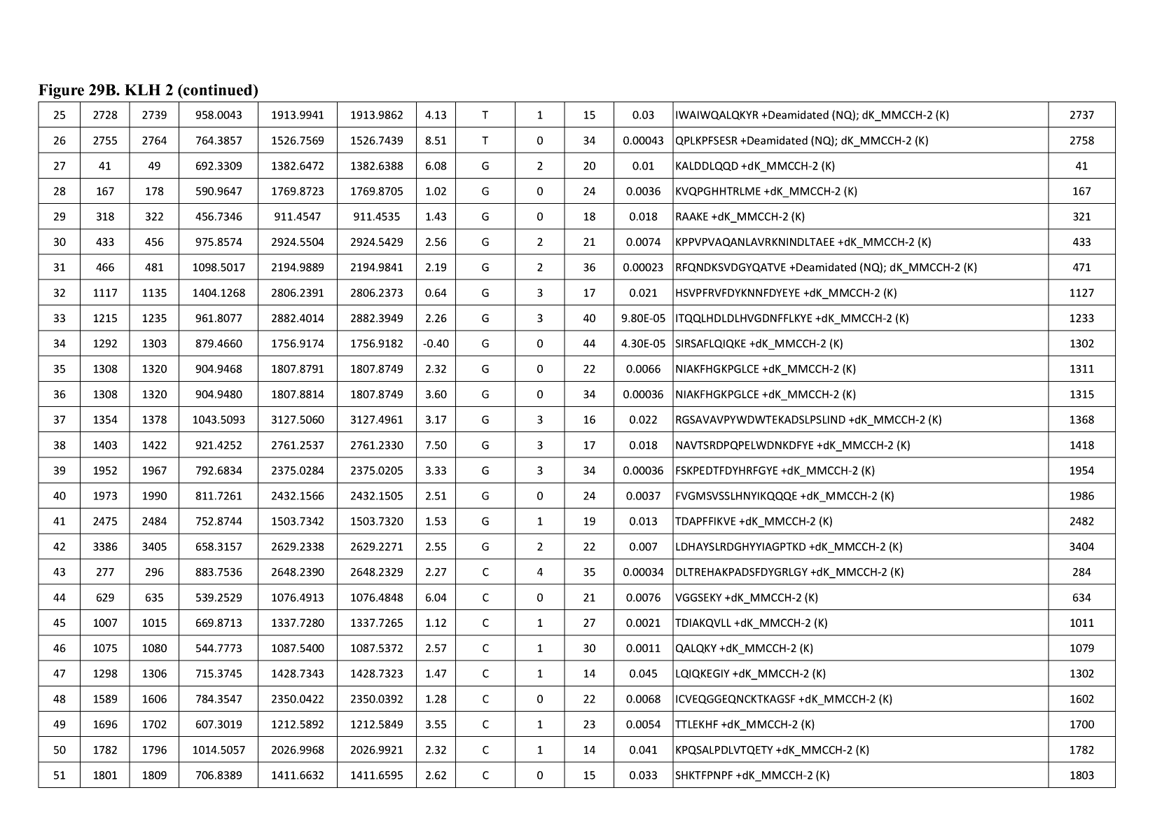

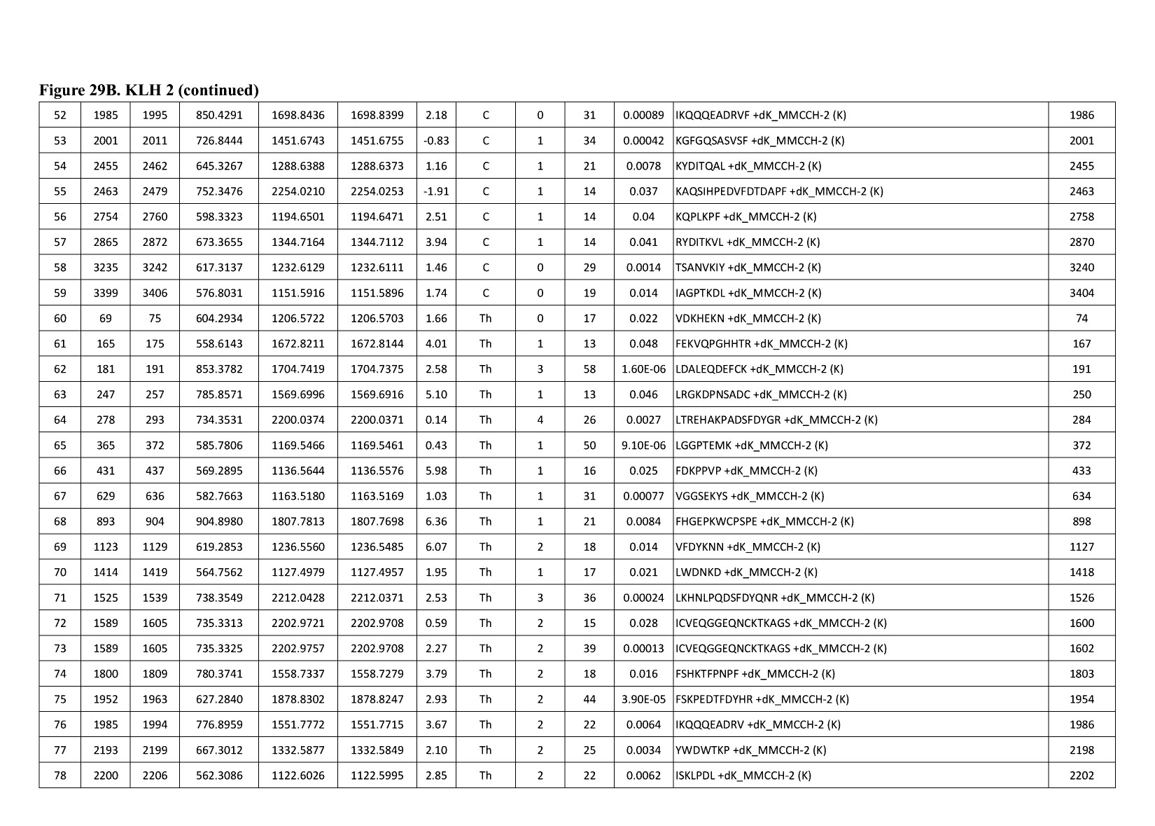

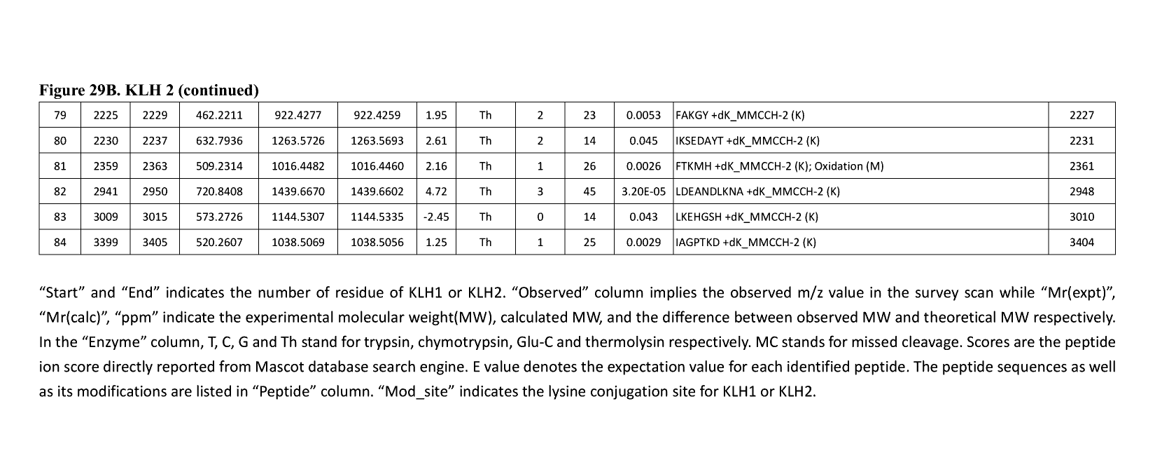

In Globo H conjugation site analysis, 31 and 28 lysines were found respectively in 1^{st} LC-MS/MS and 2^{nd} LC-MS/MS for sample 1; 19 and 21 lysines were found respectively in 1^{st} LC-MS/MS and 2^{nd} LC-MS/MS for sample 2; 10 and 11 lysines were found respectively in 1^{st} LC-MS/MS and 2^{nd} LC-MS/MS for sample 3; 18 and 19 lysines were found respectively in 1^{st} LC-MS/MS and 2^{nd} LC-MS/MS for sample 4. The identification details were listed in FIGS. 14 to 21. In MMCCH conjugation analysis, 155 and 141 lysines were found respectively in 1^{st} LC-MS/MS and 2^{nd} LC-MS/MS for sample 1; 143 and 137 lysines were found respectively in 1^{st} LC-MS/MS and 2^{nd} LC-MS/MS for sample 2; 147 and 143 lysines were found respectively in 1^{st} LC-MS/MS and 2^{nd} LC-MS/MS for sample 3; 140 and 136 lysines were found respectively in 1^{st} LC-MS/MS and 2^{nd} LC-MS/MS for sample 4. The identification details were listed in FIGS. 22 to 29.

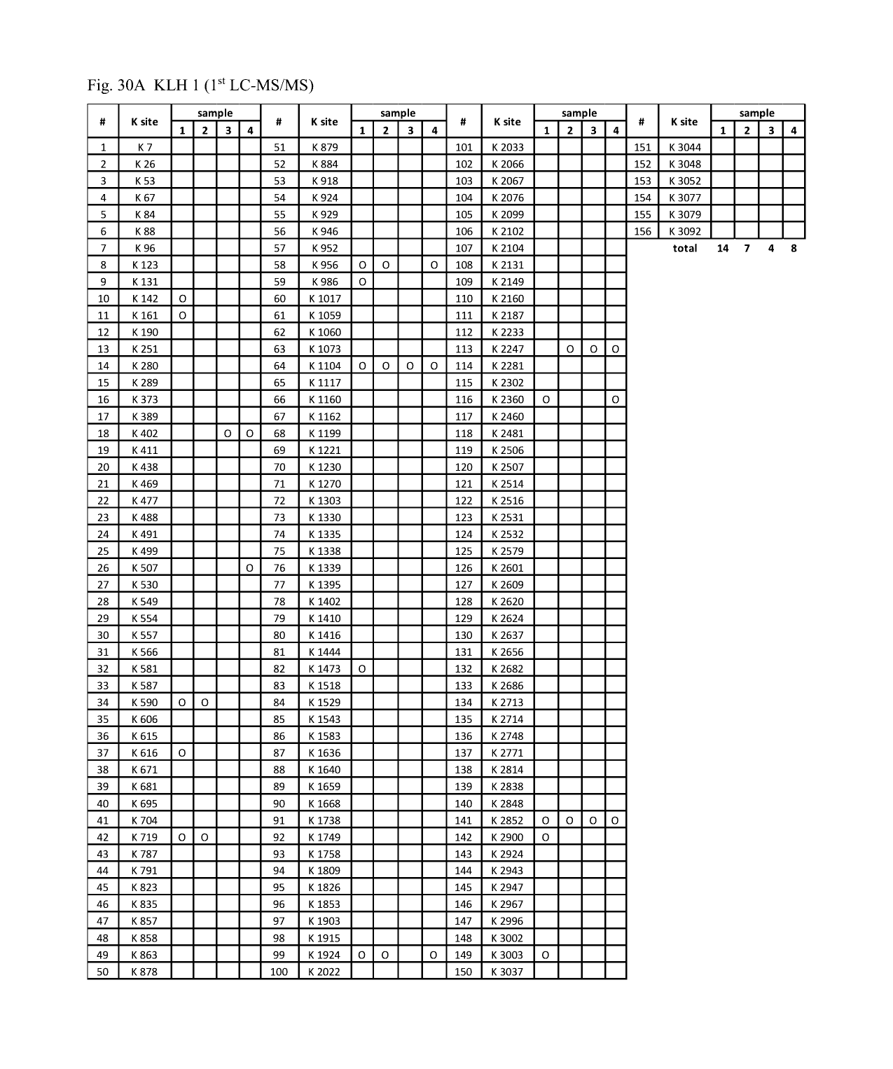

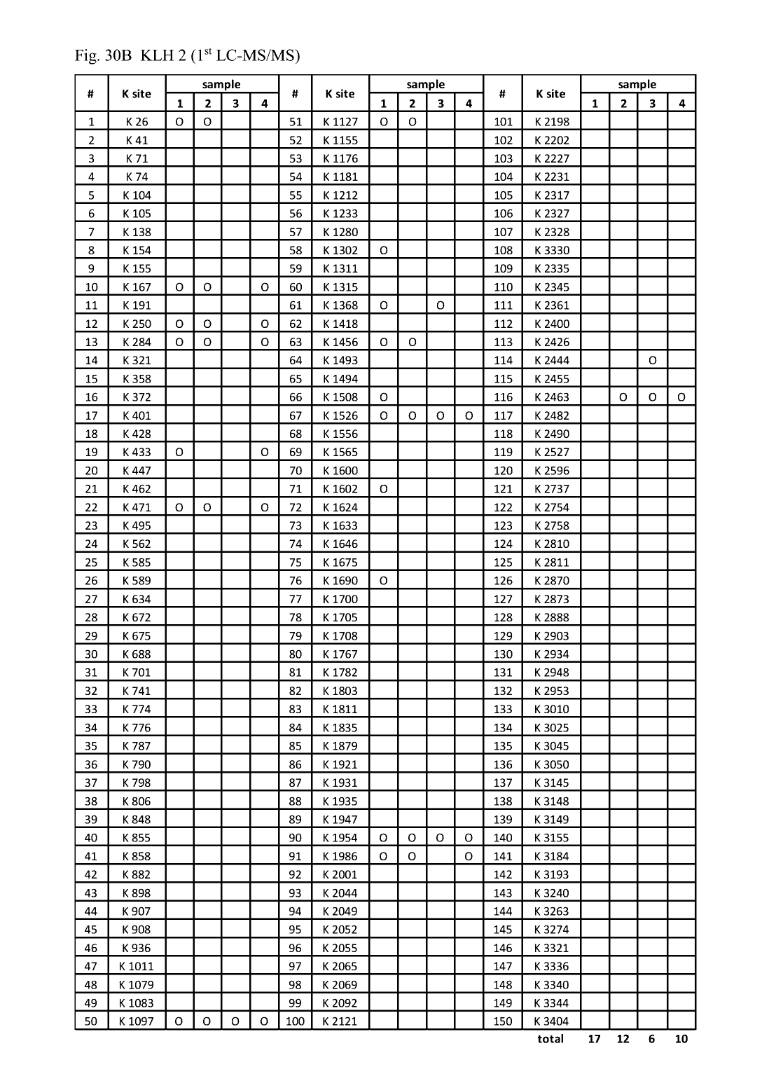

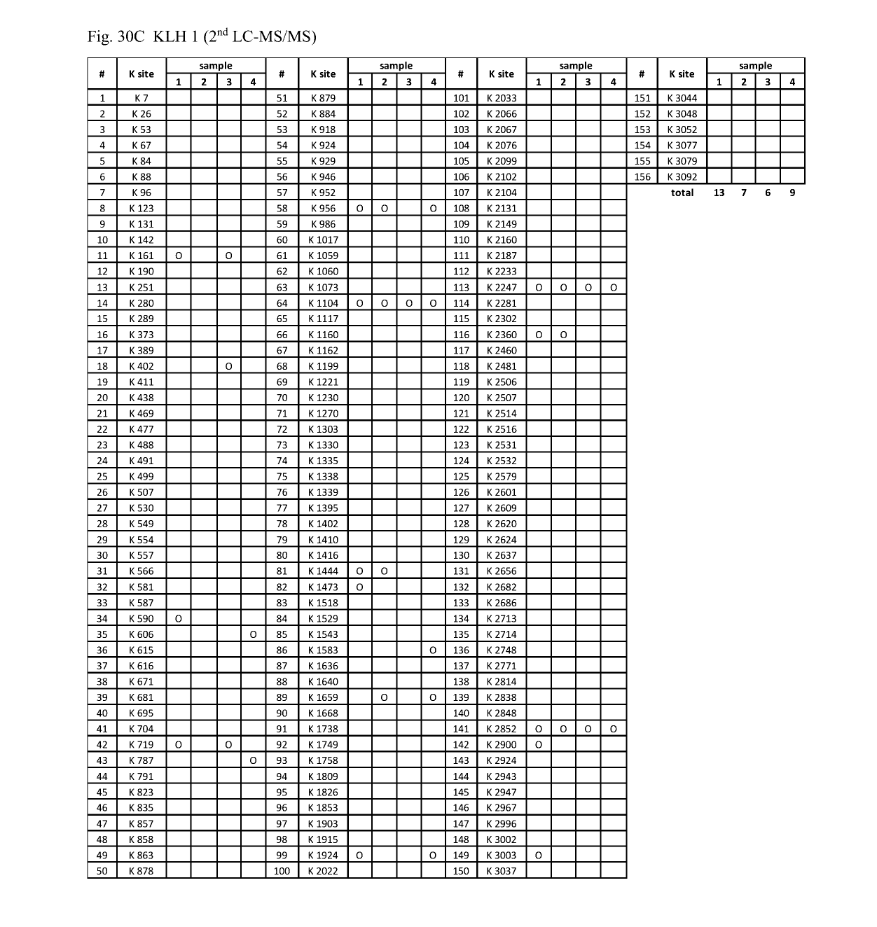

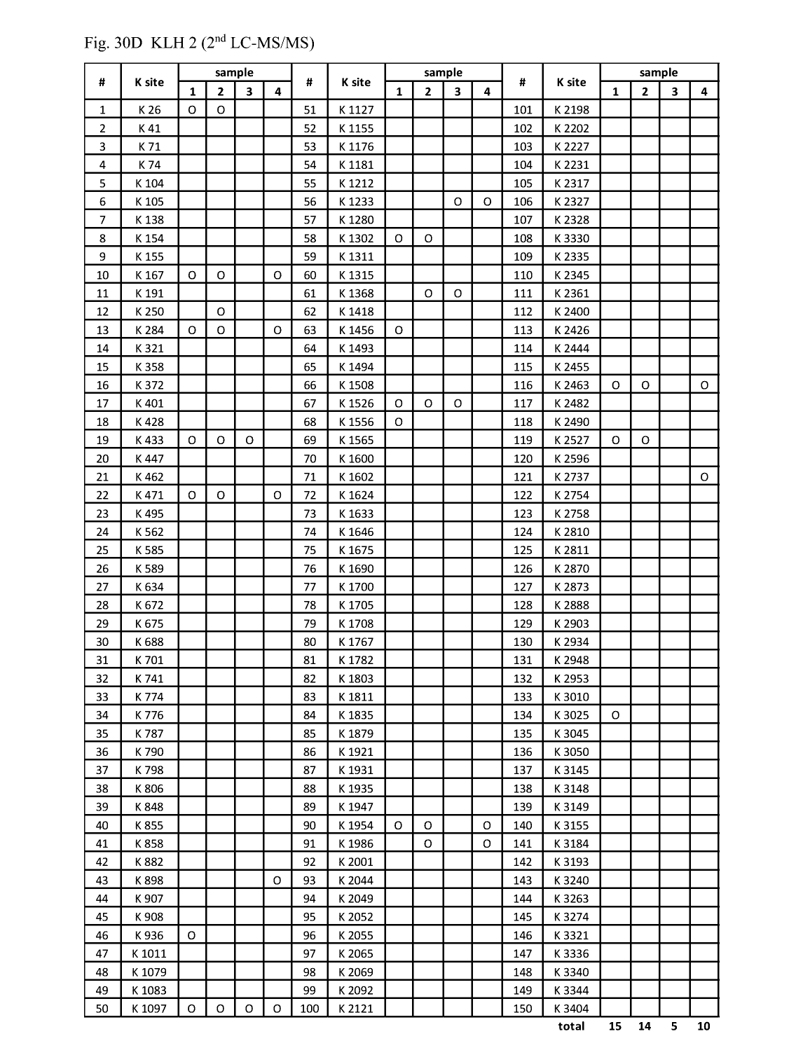

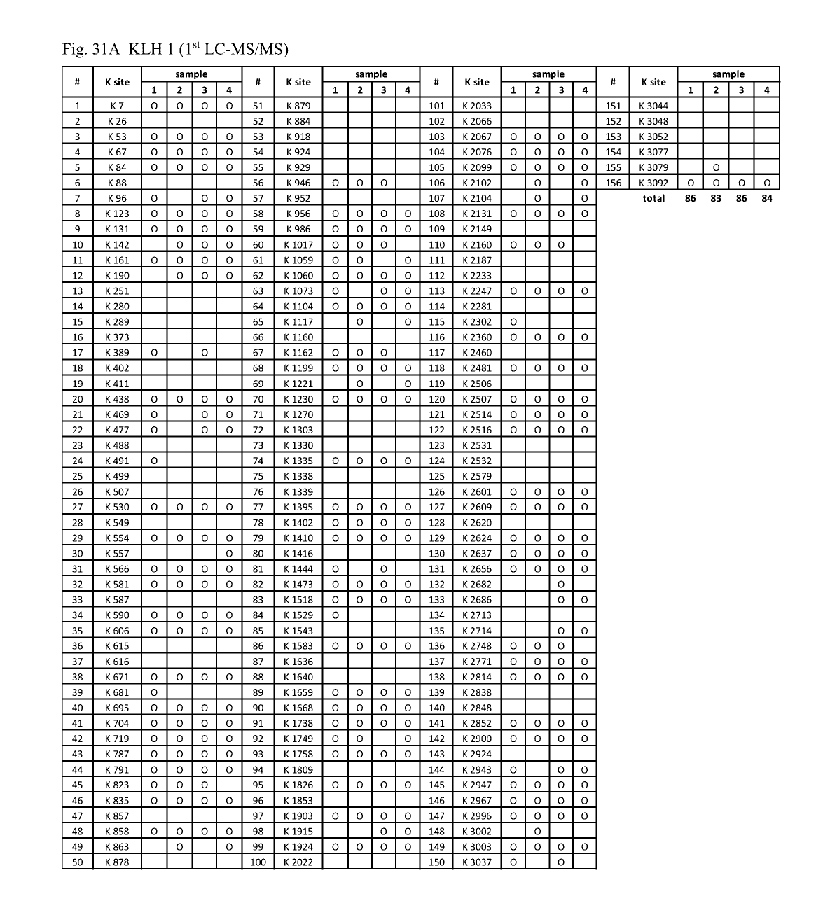

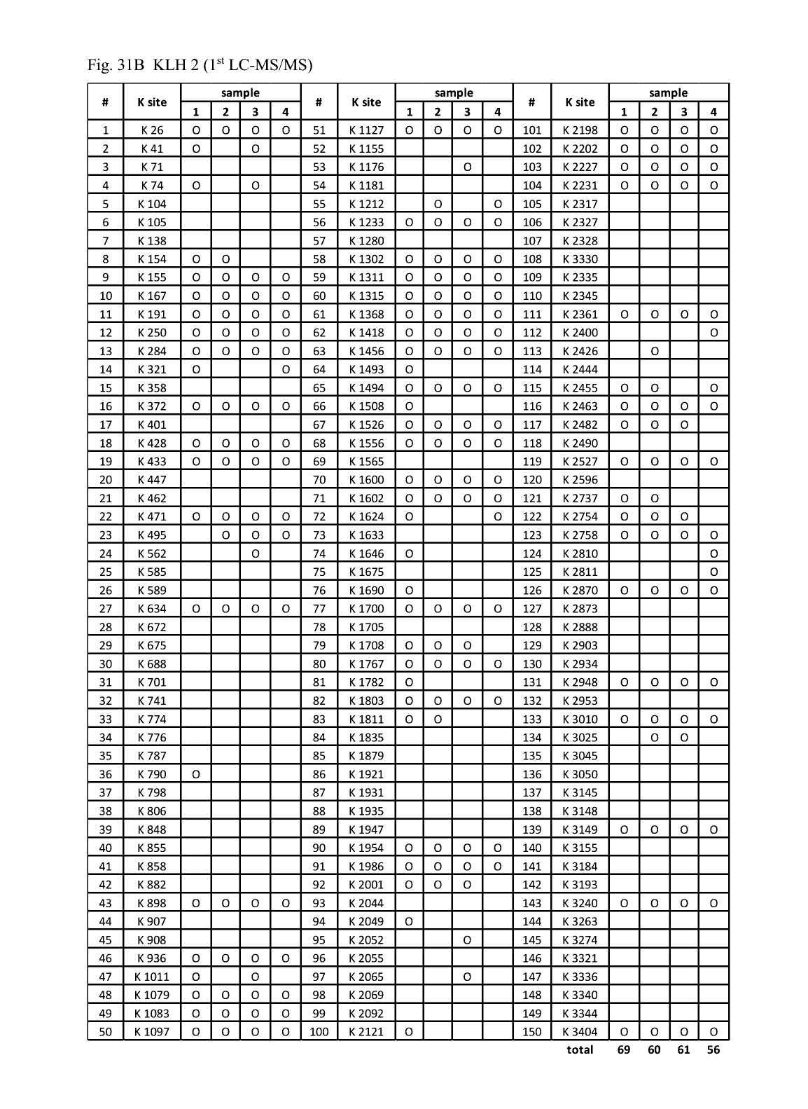

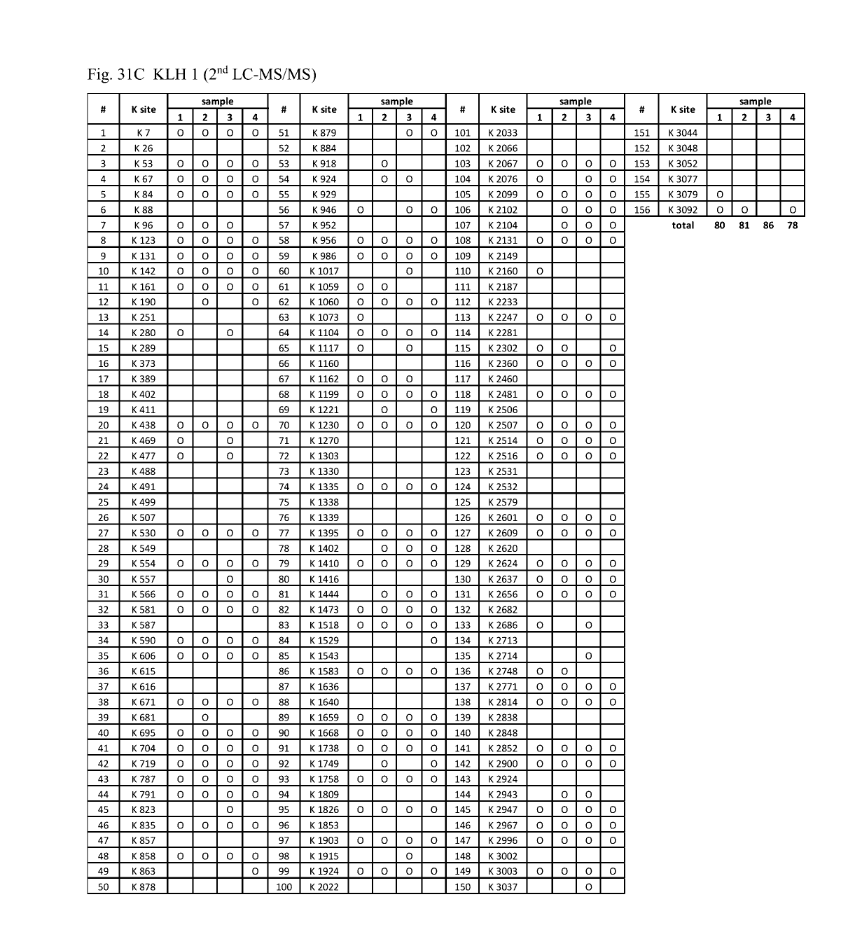

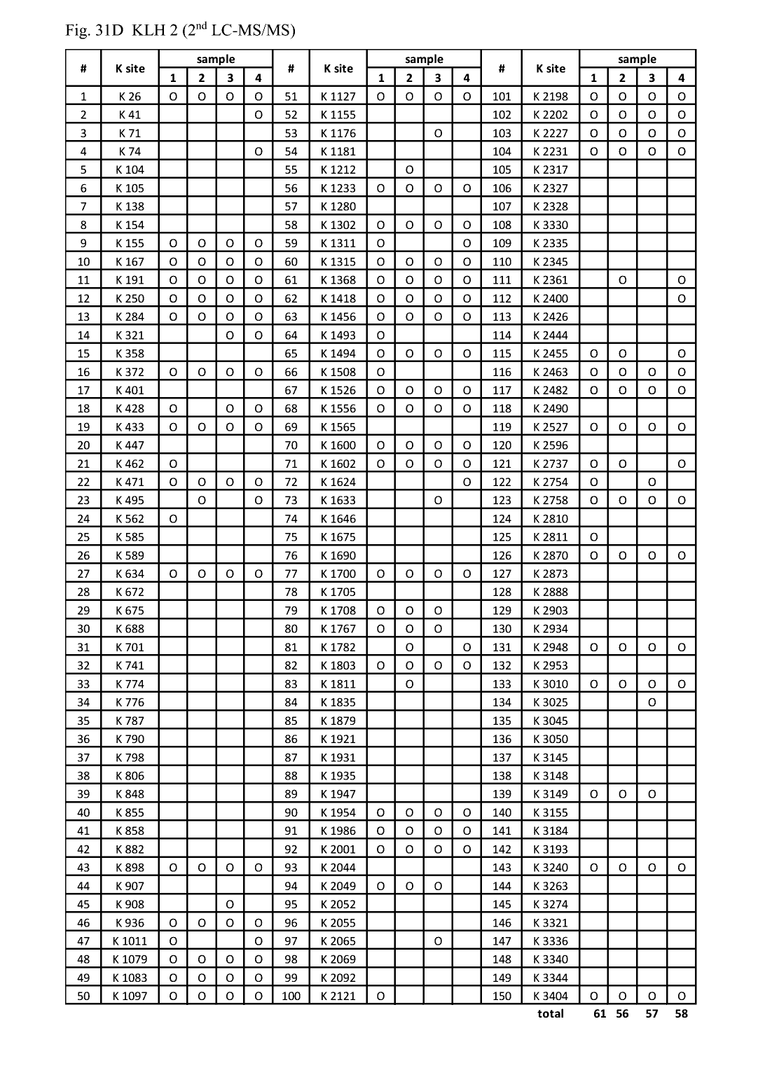

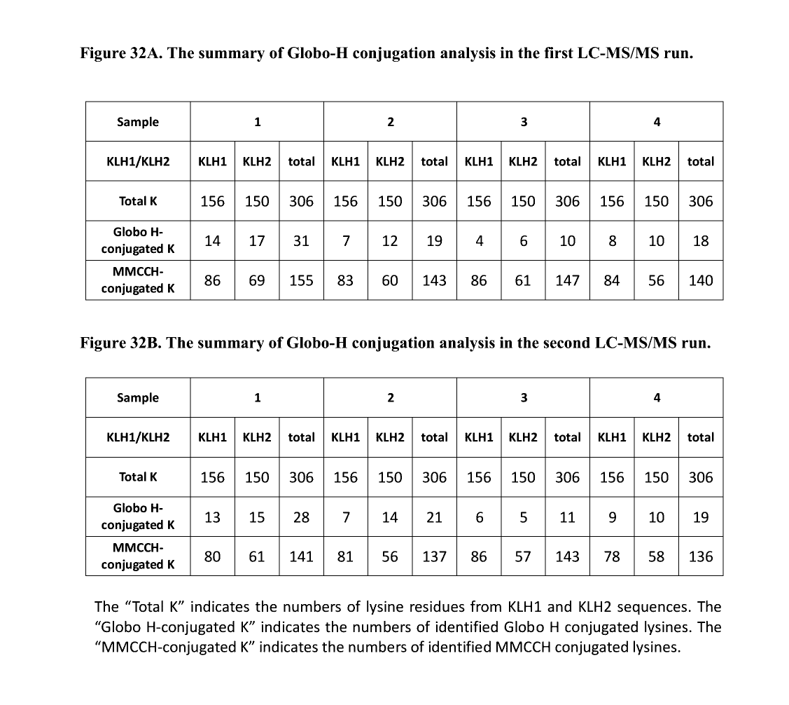

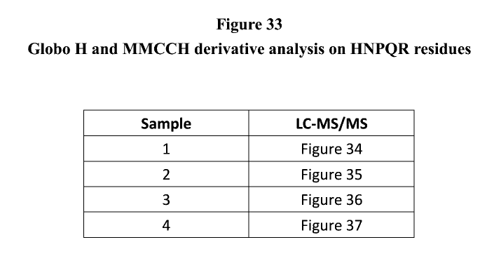

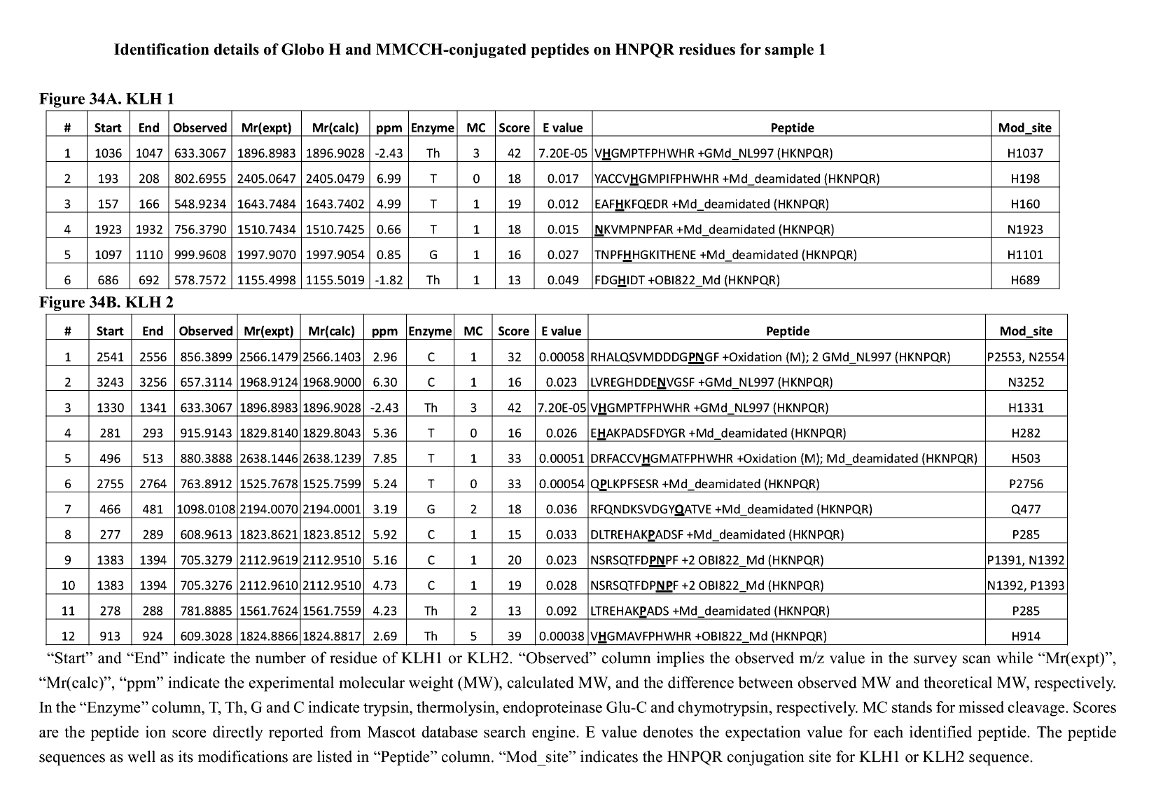

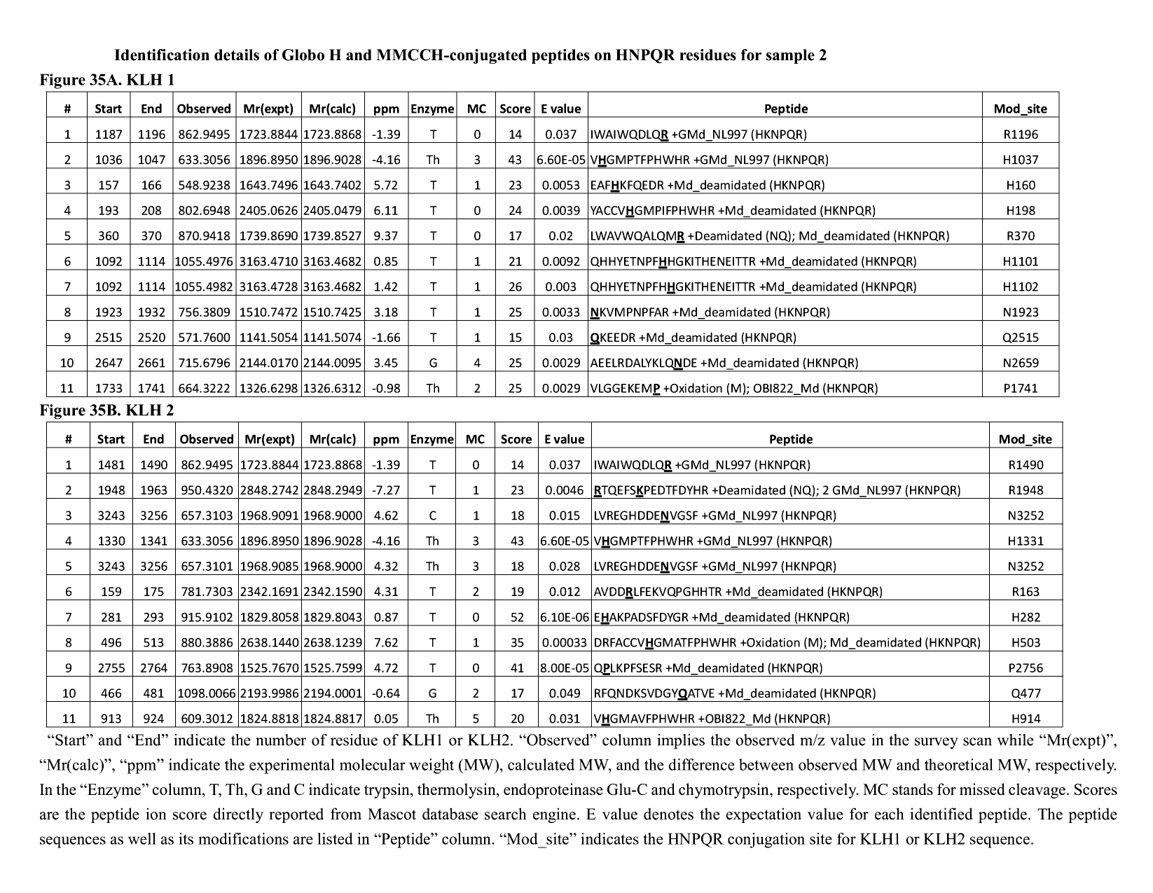

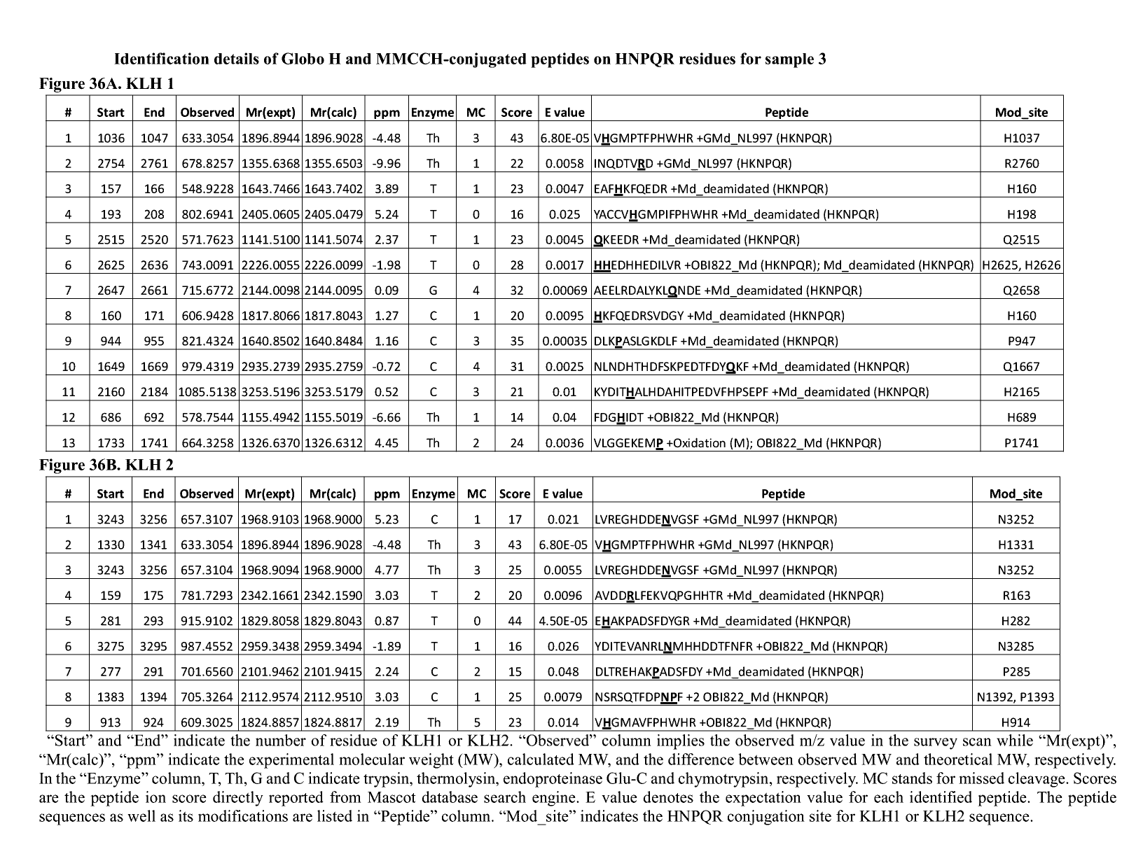

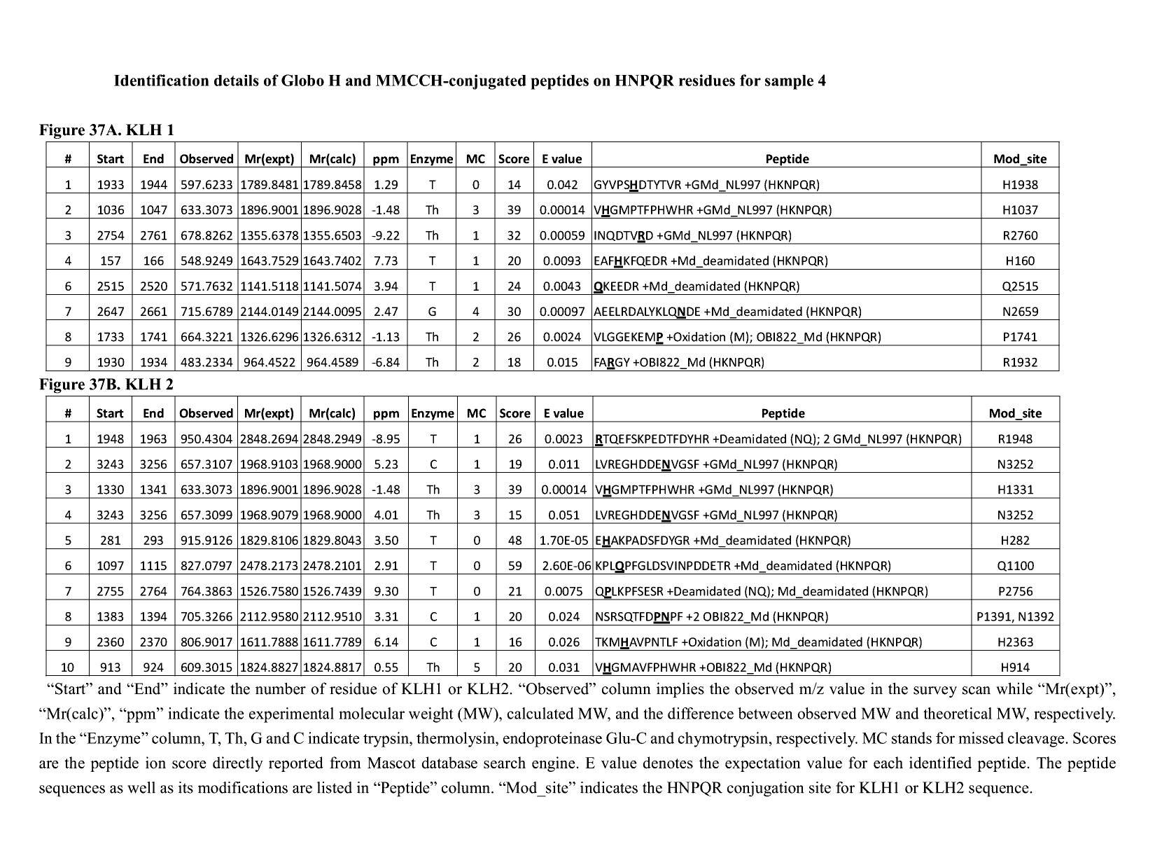

Globo H conjugation sites from multiple enzyme experiments were summarized in FIG. 30 and MMCCH conjugation sites were summarized in FIG. 31. The overall conjugation site analysis results for Globo H conjugated samples were summarized in FIG. 32.

### Example 8

### Conclusion

The mass spectrometric signals of Globo H derivative conjugated peptides are lower than those of MMCCH conjugated peptides due to the multiple neutral loss forms and lower ionization efficiency of polysaccharide, which makes the direct identification of Globo H conjugation more difficult. This is why the numbers of identified peptides are higher for MMCCH conjugation analysis. Therefore, the MMCCH results may be used to represent the Globo H conjugation.

The conjugation site analysis suggests that there are 155, 143, 147 and 140 lysine conjugation sites identified among the total 306 lysine residues in KLH1/KLH2 for sample 1-4, respectively. In the replicate analysis, 141, 137, 143 and 136 conjugation sites were identified for samples 1-4, respectively.

### Example 9

### LC-MS/MS Analysis of Globo-H Conjugation Sites (Histidine, Asparagine, Proline, Glutamine and Arginine) on KLH

Two types of derivatives on the five residues, H, N, P, Q, R were examined: GMd (Globo H+MMCCH linker+lysine derivative) and Md (MMCCH linker+lysine derivative).

Modification analysis on HNPQR (GMd and Md) yielded 6 and 13 conjugated HNPQR sites in KLH1 and KLH2, respectively, for OBI-822-13001-DP (sample 1); 11 and 10 conjugated HNPQR sites in KLH1 and KLH2, respectively, for OBI-822-13002-DP (sample 2); 13 and 9 conjugated HNPQR sites in KLH1 and KLH2, respectively, for OBI-822-13003-DP (sample 3); and 8 and 10 conjugated HNPQR sites in KLH1 and KLH2, respectively, for OBI-822-13004-DS (sample 4).

### Example 9

### Materials and Methods

The abbreviations were listed as follows:

K=Lysine; H=Histidine; N=Asparagine; P=Proline; Q=Glutamine; R=Arginine; LC-MS/MS=Liquid Chromatography-Tandem Mass Spectrometry; MW=Molecular weight.

The database search was performed with Mascot version 2.4.1 against the KLH1 sequence (SEQ ID NO. 1) and KLH2 sequence (SEQ ID NO. 2). The parameters used were as follow:

Enzyme: Trypsin, Glu-C, Chymotrypsin and Thermolysin according to the digestion method;

Fixed modification: Carbamidomethyl (C);
Variable modifications: Oxidation(M), Deamidation (NQ);
1. OBI822_GMd (HKNPQR), GMd_NL997 (HKNPQR);
2. OBI822_Md (HKNPQR), Md_deamidated (HKNPQR);
Peptide Mass Tolerance: ±10 ppm; Fragment Mass Tolerance: ±0.05 Da; Max Missed Cleavages: 5; Instrument type: ESI-TRAP; Ion cut-off score: 13.

“OBI822_GMd” in the search parameters indicates the GMd-conjugated residues (HKNPQR) with the MW addition of 1393.5317 Da.

“GMd_NL997” in the search parameters indicates the neutral loss form of GMd-conjugated residues (HKNPQR) with the MW addition of 396.1831 Da.

“OBI822_Md” in the search parameters indicates the Md-conjugated residues (HKNPQR) with the MW addition of 352.1569 Da.

“Md_deamidated” in the search parameters indicates the deamidated form of Md-conjugated residues (HKNPQR) with the MW addition of 338.1300 Da.

### Example 9

### Results

LC-MS/MS-based techniques have emerged as an important tool for the identification of proteins and characterization of amino acid modifications. Detailed information regarding peptide sequences and modification sites could be obtained by the assignment of fragment ions provided by the MS/MS spectra. Mascot was currently the most popular search engine, with a well-accepted probability-based scoring algorithm. To identify different modifications on Lysine (K) and other specific residues (HNPQR) in samples 1-4, all of the search results underwent subsequent manual checked to screen for only those peptides with high quality MS/MS spectra.

During the MASCOT search for the GMd (Globo H+MMCCH linker+lysine derivative) and Md (MMCCH linker+lysine derivative) modifications on HNPQR residues, there were false peptides identified. If the assigned modification site was close to a lysine residue, it was likely that the lysine is the actual modification site. These peptides were excluded from the identification lists. If there was no nearby lysine residue can be observed, the modification on HNPQR can be confirmed. As shown in FIG. 38, GMd modification site analysis yielded 1, 2, 2 and 3 conjugated HNPQR sites in KLH1 for samples 1-4, respectively and 4, 4, 2 and 3 conjugated HNPQR sites in KLH2 for samples 1-4, respectively. Md modification site analysis yields 5, 9, 11 and 5 conjugated HNPQR sites in KLH1 for samples 1-4, respectively and 9, 6, 7 and 7 conjugated HNPQR sites in KLH2 for samples 1-4, respectively.

### Example 9

### Conclusion

Among these four samples, the GMd and Md modifications analysis on specific residues (HNPQR) suggests that there were 6, 11, 13 and 8 conjugated HNPQR sites in KLH1 for samples 1-4, respectively, and 13, 10, 9 and 10 conjugated HNPQR sites in KLH2 for samples 1-4, respectively.

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

What is claimed is:

1. A composition comprising a plurality of Globo H moieties covalently linked to one to twenty (n=1 to 20) keyhole limpet hemocyanin (KLH) moiety(ies), wherein the Globo H moieties are covalently bound to the KLH moieties on one or more amino acid residues.

2. The composition of claim 1, wherein the amino acid residues are basic, neutral, hydrophobic amino acid residues and/or a combination thereof.

3. The composition of claim 1, wherein the amino acid residues are lysine, arginine, histidine, asparagine, proline, glutamine and/or a combination thereof.

4. The composition of claim 1, wherein the KLH moiety is a monomer (n=1).

5. The composition of claim 1, wherein the KLH moiety is a dimer (n=2).

6. The composition of claim 1, wherein the KLH moiety is a trimer (n=3).

7. The composition of claim 1, wherein the KLH moiety is a tetramer (n=4).

8. The composition of claim 1, wherein the KLH moiety is a pentamer (n=5).

9. The composition of claim 1, wherein the KLH moiety is a hexamer (n=6).

10. The composition of claim 1, wherein the KLH moiety is a heptamer (n=7).

11. The composition of claim 1, wherein the KLH moiety is an octamer (n=8).

12. The composition of claim 1, wherein the KLH moiety is a nonamer (n=9).

13. The composition of claim 1, wherein the KLH moiety is a decamer (n=10).

14. The composition of claim 1, wherein the KLH moiety is an undecamer (n=11).

15. The composition of claim 1, wherein the KLH moiety is a dodecamer (n=12).

16. The composition of claim 1, wherein the KLH moiety is a tridecamer (n=13).

17. The composition of claim 1, wherein the KLH moiety is a tetradecamer (n=14).

18. The composition of claim 1, wherein the KLH moiety is a pentadecamer (n=15).

19. The composition of claim 1, wherein the KLH moiety is a hexadecamer (n=16).

20. The composition of claim 1, wherein the KLH moiety is a heptadecamer (n=17).

21. The composition of claim 1, wherein the KLH moiety is an octadecamer (n=18).

22. The composition of claim 1, wherein the KLH moiety is a nonadecamer (n=19).

23. The composition of claim 1, wherein the KLH moiety is a didocamer (n=20).

24. The composition of claim 1, wherein the KLH moieties are the same.

25. The composition of claim 1, wherein the KLH moieties are different.

26. The composition of claim 1, wherein each KLH moiety has the same number of Globo H moieties.

27. The composition of claim 1, wherein each KLH moiety has a different number of Globo H moieties.

28. The composition of claim 1, wherein the Globo H moiety comprises (Fucα1→2 Galβ1→3 GalNAcβ1→3 Galα1→4 Galβ1→4 Glc).

29. The composition of claim 1, wherein the KLH moiety subunit is of SEQ ID NO. 1.

30. The composition of claim 1, wherein the KLH moiety subunit is at least 95% identical to a corresponding KLH moiety of SEQ ID NO. 1.

31. The composition of claim 1, wherein the KLH moiety subunit is at least 90% identical to a corresponding KLH moiety of SEQ ID NO. 1.

32. The composition of claim 1, wherein the KLH moiety subunit is at least 80% identical to a corresponding KLH moiety of SEQ ID NO. 1.

33. The composition of claim 1, wherein the KLH moiety subunit is of SEQ ID NO. 2.

34. The composition of claim 1, wherein the KLH moiety subunit is at least 95% identical to a corresponding KLH moiety of SEQ ID NO. 2.

35. The composition of claim 1, wherein the KLH moiety subunit is at least 90% identical to a corresponding KLH moiety of SEQ ID NO. 2.

36. The composition of claim 1, wherein the KLH moiety subunit is at least 80% identical to a corresponding KLH moiety of SEQ ID NO. 2.

37. The composition of claim 1, wherein the Globo H moiety covalently linked to a keyhole limpet hemocyanin (KLH) moiety subunit is linked by a 4-(4-N-maleimidomethyl) cyclohexane-1-carboxyl hydrazide (MMCCH) linkage.

38. The isolated therapeutic conjugate of claim 1 having an epitope ratio of at least 150:1.

39. The composition of claim 1 having an epitope ratio of at least 125:1.

40. The isolated therapeutic conjugate of claim 1 having an epitope ratio of at least or about 100:1.

41. The composition of claim 1 having an epitope ratio of at least 75:1.

42. The composition of claim 1 having an epitope ratio of at least 50:1.

43. The composition of claim 1 having an epitope ratio of at least 25:1

44. The composition of claim 1 having an epitope ratio of at least 10:1.

45. The composition of claim 1 having an epitope ratio of at least 1:1.

46. A method of treating cancer in a patient in need thereof comprising administering to the patient a therapeutically effective amount of the composition of claim 1.

47. The method of claim 46, wherein the cancer is breast cancer, lung cancer, gastric cancer, colon cancer, pancreatic cancer, prostate cancer, ovarian cancer, epithelial cancer or endometrial cancer.

48. The method of claim 46, wherein the cancer is a Globo H expressing cancer.

49. A method of inducing antibodies in a subject for the purpose of creating monoclonal antibodies for therapeutic or diagnostic uses comprising administering to the subject an effective amount of the composition of claim 1.

50. The method of claim 49, wherein the subject is an animal or human.

51. A pharmaceutical composition comprising
(a) a plurality of Globo H moieties covalently linked to one or more KLH moiety subunit; and
(b) an α-galactosyl-ceramide (α-GalCer) adjuvant.

52. The composition of claim 51, wherein the α-GalCer adjuvant has the following structure:

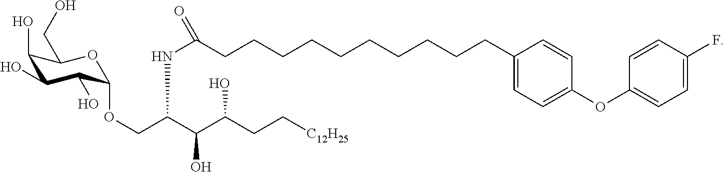

53. The composition of claim 51, wherein the KLH moieties can form a monomer, dimer, trimer, tetramer, pentamer, hexamer, hexamer, heptamer, octamer, nonamer, decamer, undecamer, dodecamer, tridecamer, tetradecamer, pentadecamer, hexadecamer, heptadecamer, octadecamer, nonadecamer or didocamer

54. A method of inducing antibodies in a subject for the purpose of creating monoclonal antibodies for therapeutic or diagnostic uses comprising administering to the subject an effective amount of the composition of claim 51.

55. The method of claim 54, wherein the subject is an animal or human.
